(19) United States
(12) Patent Application Publication
(10) Pub. No.: US 2022/0096649 A1
(43) Pub. Date: Mar. 31, 2022

(54) IMMUNOMODULATING POLYNUCLEOTIDE CONJUGATES AND METHODS OF USE

(71) Applicant: Tallac Therapeutics, Inc., Burlingame, CA (US)

(72) Inventors: Jaume PONS, San Francisco, CA (US); Hong I. WAN, Foster City, CA (US); Curt W. BRADSHAW, San Diego, CA (US); Bang Janet SIM, Brisbane, CA (US); Tracy Chia-Chien KUO, San Mateo, CA (US)

(21) Appl. No.: 17/283,919

(22) PCT Filed: Oct. 16, 2019
(86) PCT No.: PCT/US2019/056619

§ 371 Date: Apr. 8, 2021

Related U.S. Application Data
(60) Provisional application No. 62/747,070, filed on Oct. 17, 2018.
(60) Provisional application No. 62/747,611, filed on Oct. 18, 2018.

(51) Int. Cl. A61K 47/68; C07K 16/28; A61P 35/00

(52) U.S. Cl. CPC A61K 47/6849; A61K 47/6807; A61P 35/00; C07K 16/2803; C07K 16/2896

(57) ABSTRACT

Provided herein is a conjugate for modulating a natural killer cell or myeloid cell, comprising a targeting moiety and an immunomodulating polynucleotide. Also provided herein is a pharmaceutical composition for modulating a natural killer cell or myeloid cell, comprising a conjugate comprising a targeting moiety and an immunomodulating polynucleotide, and a pharmaceutically acceptable excipient. Additionally provided herein are methods of their use for modulating a natural killer cell or myeloid cell and treating a proliferative disease.

### CROSS REFERENCE TO RELATED APPLICATIONS

This application claims the priority benefit of U.S. Provisional Application Ser. No. 62/747,070, filed Oct. 17, 2018, and U.S. Provisional Application Ser. No. 62/747,611, filed Oct. 18, 2018, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 186492000340SEQLIST.TXT, date recorded: Oct. 16, 2019, size: 346 KB).

### FIELD

Provided herein is a conjugate for modulating a natural killer cell or myeloid cell, comprising a targeting moiety and an immunomodulating polynucleotide. Also provided herein is a pharmaceutical composition for modulating a natural killer cell or myeloid cell, comprising a conjugate comprising a targeting moiety and an immunomodulating polynucleotide, and a pharmaceutically acceptable excipient. Additionally provided herein are methods of their use for modulating a natural killer cell or myeloid cell and treating a proliferative disease.

### BACKGROUND

Natural killer cells (NK cells) are cytotoxic lymphocytes critical to the innate immune system, where NK cells rapidly respond to virally infected cells and tumor formation in the absence of antibodies and MHC. NK cells can also function as an interface to the adaptive immune response and play a major role in cancer immunotherapies that involve tumor-antigen targeting by antibodies. In the adaptive immune-response, NK cells function as effector cells of the immune system and actively lyse target cells that have their membrane-surface antigens marked by specific antibodies. This mechanism of cell mediated immune defense is known as the antibody-dependent-cell-mediated-cytotoxicity (ADCC). Hashimoto et al., J. Infect. Dis. 1983, 148, 785-794. The ADCC mediated by NK cells is a major mechanism of therapeutic efficacy of many anti-cancer antibodies used in treating various cancers overexpressing unique antigens, such as neuroblastoma, breast cancer, and B cell lymphoma. Wang et al., Front. Immunol. 2015, 6, 368; Zahavi et al., Antibody Therapeut. 2018, 1.7-12. Approaches to enhance NK cell activity would increase ADCC and may enhance the efficacy of such anti-cancer therapeutics. In addition, NK cells bear natural cytotoxicity receptors that detect the altered expression of ligands on the surface of tumor cells, which ultimately triggers NK cell activation and lysis of tumor cells. NK cells have been reported to develop prolonged, and highlight specific memory to various antigens Paust et al., Nat. Immunol. 2011, 12, 500-508. Studies have indicated that NK cells are frequently deficient and dysfunctional in patients with malignancy, indicating that this may be key factor in cancer immunoevasion and progression. Moreover, low cancer cell function was found to predict an increased risk of developing cancer. Berrien-Elliot et al., Curr. Opin. Organ Transplant. 2015, 20, 671-680; Imai et al., Lancet 2000, 356, 1795-1799. In essence, developing strategies to activate and expand NK cells will be advantageous in treating malignancies.

NK cells are derived from the common lymphoid progenitor that generates B and T lymphocytes. They differentiate and mature in the bone marrow, lymph nodes, spleen, tonsils, and thymus before entering the circulation. NK cells exist as classical and non-classical subsets that commonly express CD16 and CD56 surface markers. CD56 (also known as neural-cell adhesion molecule (NCAM)) is a homophilic binding glycoprotein, which has been implicated in cell-cell adhesion, neurite outgrowth, synaptic plasticity, and learning and memory. Normal cells that stain positively for CD56 include NK cells, activated T-cells, brain and cerebellum, and neuroendocrine tissues. Tumors that are CD56-positive include myeloma, myeloid leukemia, neuroendocrine tumors, Wilm's tumor, adult neuroblastoma, NK/T cell lymphomas, pancreatic acinar-cell carcinoma, pheochromocytoma, and small-cell lung carcinoma. Van Acker et al., Front. Immunol. 2017, 8, 892.

Myeloid cells are derived from sequential myeloid cell progenitors originated from hematopoietic stem cells (HSC₅) in the bone marrow. Myeloid cells are the most abundant nucleated hematopoietic cells in the body, consisting of several types of cells, including neutrophils, monocytes, macrophages, dendritic cells (DC), eosinophils, and mast cells. Upon pathogen invasion, myeloid cells are rapidly recruited into local tissues via various chemokine receptors, where they are activated for phagocytosis as well as secretion of inflammatory cytokines, thereby playing major roles in the innate immunity. Macrophages can directly kill tumor cells via antibody-dependent cellular phagocytosis (ADCP). Myeloid cells also play a key role in linking the innate and adaptive immunity, primarily through antigen presentation by DC and macrophage and recruitment of adaptive immune cells. Subsets of myeloid cells also include tumor-associated macrophages (TAM) and myeloid-derived suppressor cells (MDSC). TAMs are tissue macrophages with heterogeneous function and phenotype present in high numbers in the microenvironment of solid tumors. TAMs can promote initiation and metastasis of tumor cells, inhibit antitumor immune responses mediated by T cells, and stimulate tumor angiogenesis and subsequently tumor progression. Yang and Zhang, J. Hematol. Oncol. 2017, 10, 58. Moreover, TAMs contribute to the suppression of the adaptive immunity in progressing cancer. MDSC₅, comprising monocytic and granulocytic subpopulations, contribute to an immunosuppressive network that drives cancer escape by disabling the T cell adaptive immunity. MDSC₅ accumulate throughout cancer progression and are linked to poor clinical outcomes as well as resistance to chemotherapy, radiation, and immunotherapy in murine tumor systems. Waight et al., J. Clin. Investig. 2013, 123, 4464-4478; Alizadeh et al., Cancer Res. 2014, 74, 104-118. Modulating myeloid cell activities, such as increasing ADCP by macrophage, enhance APC function by dendritic cells, reducing immunosuppressive activities of TAMs and MDSC₅, may promote ant-tumor innate and adaptive immunity and enhance efficacy of other anti-cancer agents such as checkpoint inhibitors, vaccines and T-cell directed immunotherapeutics.

Signal regulatory proteins (SIRP) comprised of several membrane glycoproteins expressed mainly by immune cells, including SIRPa, SIRP3, and SIRPy. SIRPa is expressed mainly by myeloid cells. SIRPa acts as inhibitory receptor via its cytoplasmic immunoreceptor tyrosine-based inhibition motifs (ITIM) domain and interacts with a broadly expressed transmembrane protein CD47. This interaction negatively controls effector function of innate immune cells. SIRPa diffuses laterally on the macrophage membrane and accumulates at a phagocytic synapse to bind CD47 and signal ‘self,’ which inhibits the cytoskeleton-intensive process of phagocytosis by the macrophage. This is analogous to the self signals provided by MHC class I molecules to NK cells via Ig-like or Ly49 receptor. SIRPα is also expressed in other myeloid cells such as neutrophils, dendritic cells, and MDSC₅; and may serve as an inhibitory receptor to regulate activation and maturation of these cell populations. Compared to SIRPα, SIRPβ has overlapping expression in myeloid cells but has different cytoplasmic domain and may interact with different ligands other than CD47. SIRPγ is expressed in lymphoid cells such as T cell and MK cells. SIRPγ also interact with CD47 but has a short cytoplasmic domain that is unlikely to have similar signaling properties as SIRPα. Barclay and Brown, Nat. Rev. Immunol. 2006, 6, 457-64.

Toll-like receptors (TLRs) are critical pattern recognition receptors of the innate immunity, which recognize pathogens through sensing pathogen-associated molecular patterns (PAMPs) derived from bacteria, virus, fungi, and protozoa. Akira et al., Nat. Rev. Immunol. 2004, 4, 499-511; Zhang et al., Science 2004, 303, 1522-1526. Each TLR contains transmembrane domain, extracellular PAMPs binding domain with leucine-rich repeats motif, and intracellular Toll-IL-1 receptor domain that initiates signaling cascade. Gay and Gangloff, Annu. Rev. Biochem. 2007, 76, 141-165. Recognition of microbial invaders by TLRs leads to activation of downstream signaling cascade to secret cytokines and chemokines and finally results in activation of both the innate and adaptive immune response to clean pathogens. Takeda and Akira, Semin. Immunol. 2004, 16, 3-9; Shi et al., J. Biol. Chem. 2016, 291, 1243-1250. In humans, ten TLRs have been identified, including TLR-1, TLR-2, TLR-3, TLR-4, TLR-5, TLR-6, TLR-7/8, TLR-9, and TLR-10. D'Arpa and Leung, Adv. Wound Care 2017, 6, 330-343.

Toll-like receptor 9 (TLR9), also designated as CD289, is an important receptor expressed in immune system cells including dendritic cells (DC₅), B lymphocytes, macrophages, natural killer cells, and other antigen presenting cells. TLR9 activation triggers intracellular signaling cascades, leading to activation, maturation, proliferation and cytokine productions in these immune cells, thus bridges the innate and adaptive immunity. Martinez-Campos et al., Viral Immunol. 2016, 30, 98-105; Notley et al., Sci. Rep. 2017, 7, 42204. Natural TLR-9 agonists include unmethylated cytosine-guanine dinucleotide (CpG)-containing oligodeoxynucleotides (CpG ODNs).

CpG ODNs are generally divided into three classes: class A, class B, and class C. A class A CpG ODN typically contains poly-G tails with phosphorothioate backbones at 3′- and 5′-termini and a central palindromic sequence including a phosphate backbone. A class A CpG ODNs typically contains CpG within its central palindrome sequence. A class B CpG ODN typically includes a fully phosphorothioate backbone, and its sequence at the 5′ end is often critical for TLR9 activation. A class C CpG ODN includes a fully phosphorothioate backbone with a 3′-end sequence enabling formation of a duplex. However, CpG ODNs are often susceptible to degradation in serum and thus pharmacokinetics of CpG ODNs may be one of the limiting factors in their development as therapeutics. Also CpG ODNs often exhibit uneven tissue distribution in vivo, with primary sites of accumulation being in liver, kidney, and spleen. Such distribution can elicit off-target activity and local toxicity associated with PAMPs. Accordingly, there is a need for an effective method to stabilize and deliver a CpG ODN for therapeutic applications.

### SUMMARY

Provided herein is a conjugate for modulating a natural killer cell or myeloid cell, comprising a targeting moiety and an immunomodulating polynucleotide.

Also provided herein is a pharmaceutical composition for modulating a natural killer cell or myeloid cell, comprising a conjugate that comprises a targeting moiety and an immunomodulating polynucleotide; and a pharmaceutically acceptable carrier.

Additionally provided herein is a method of modulating a natural killer cell or myeloid cell, comprising contacting the cell with a conjugate comprising a targeting moiety and an immunomodulating polynucleotide.

Further provided herein is a method of treating a proliferative disease in a subject, comprising administering to the subject a conjugate comprising a targeting moiety and an immunomodulating polynucleotide.

Provided herein is a conjugate of Formula (C):

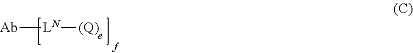

or a stereoisomer, a mixture of two or more diastereomers, a tautomer, or a mixture of two or more tautomers thereof; or a pharmaceutically acceptable salt, solvate, or hydrate thereof; wherein:

Ab is an anti-CD56 or anti-SIRP antibody;

each L^{N} is independently a linker;

each Q is independently an immunomodulating polynucleotide;

each e is independently an integer of about 1, about 2, about 3, or about 4; and

f is an integer of about 1, about 2, about 3, or about 4.

### BRIEF DESCRIPTION OF THE DRAWINGS

FIG. 1 shows the activation of NK cells as measured by an increase in CD69 expression upon 24-hour treatment of peripheral blood mononuclear cells (PBMC) with an anti-CD56-CpG nucleotide (SEQ. ID NO: 425) conjugate (anti-CD56-CPG) in comparison with controls: the CpG nucleotide (p425) alone, the anti-CD56 antibody (anti-CD56) alone, and media (the horizontal dashed line).

FIG. 2 shows the activation of NK cells as measured by an increase in CD69 expression upon 48-hour treatment of PBMC with an anti-CD56-CpG nucleotide (SEQ. ID NO: 425) conjugate (anti-CD56-CPG) in comparison with controls: the CpG nucleotide (p425) alone, the anti-CD56 antibody (anti-CD56) alone, and media (the horizontal dashed line).

FIG. 3 shows an increase in CD14⁺ cells upon treatment of PBMC with anti-SIRPα-CpG nucleotide (SEQ. ID NO: 425) conjugates (anti-Sirpα 1-CpG and anti-Sirpα 2-CpG) with a blocking anti-SIRPα antibody (anti-Sirpα 1) or a non-blocking anti-SIRPα antibody (anti-Sirpα 2) in comparison with controls: the CpG nucleotide (p425) alone, the anti-SIRPα antibodies (anti-Sirpα 1 and anti-Sirpα 2) alone, and media (the horizontal dashed line).

FIG. 4 shows an increase in CD14⁺ cells upon treatment of purified CD14⁺ cells with anti-SIRPα-CpG nucleotide (SEQ. ID NO: 425) conjugates (anti-Sirpα 1-CpG and anti-Sirpα 2-CpG) with a blocking anti-SIRPα antibody (anti-Sirpα 1) or a non-blocking anti-SIRPα antibody (anti-Sirpα 2) in comparison with controls: the CpG nucleotide (p425) alone, the anti-SIRPα antibodies (anti-Sirpα 1 and anti-Sirpα 2) alone, and media (the horizontal dashed line).

FIG. 5 shows a series of structures showing abbreviations with corresponding structures. The abbreviations are those used in Table 2.

FIG. 6 shows a series of structures showing abbreviations with corresponding structures. The abbreviations are those used in Table 2.

FIGS. 7A-7D show in vivo inhibition of tumor growth by anti-SIRPα-CpG nucleotide conjugates. FIG. 7A: measurement of average CT26 tumor size over time after treatment with 10 mg/kg anti-SIRPα 1 conjugate (blocking antibody) dosed twice, three days apart or unconjugated anti-SIRPα antibody dosed twice, three days apart, as compared to PBS control. FIG. 7B: measurement of average CT26 tumor size over time after treatment with 3 mg/kg anti-SIRPα 1 conjugate (blocking antibody) or anti-SIRPα 2 conjugate (non-blocking antibody), both dosed 2q3, as compared to PBS control. FIG. 7C: measurement of average CT26 tumor size over time after treatment with 1 mg/kg, 0.3 mg/kg, or 0.1 mg/kg anti-SIRPα 1 conjugate (blocking antibody), all dosed 2q3, as compared to PBS control. FIG. 7D: measurement of average MC38 tumor size over time after treatment with 10 mg/kg anti-SIRPα 1 conjugate (blocking antibody) dosed 2q3, as compared to PBS control. mpk=mg/kg. 2q3=2 doses, 3 days apart. Arrows indicate administration of conjugate or control.

FIGS. 8A & 8B show in vivo inhibition of tumor growth by anti-SIRPα-CpG nucleotide conjugates. FIG. 8A: measurement of average CT26 tumor size over time after treatment with 1 mg/kg anti-SIRPα 1 conjugate (blocking antibody) dosed twice, three days apart or dosed twice, seven days apart, as compared to PBS control. FIG. 8B: survival curve of mice in CT26 tumor model dosed as described in FIG. 8A. mpk=mg/kg. 2q3=2 doses, 3 days apart. 2q7=2 doses, 7 days apart. Arrows indicate administration of conjugate or control.

### DETAILED DESCRIPTION

### Definitions

To facilitate understanding of the disclosure set forth herein, a number of terms are defined below.

Generally, the nomenclature used herein and the laboratory procedures in biology, biochemistry, medicinal chemistry, organic chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, and mouse. The terms “subject” and “patient” are used interchangeably herein in reference, for example, to a mammalian subject, such as a human subject, in one embodiment, a human.

The term “abasic spacer,” as used herein, represents a divalent group of the following structure:

R¹-L¹-[-L²-(L¹)ₙ₁-]ₙ₂-R², (I)

wherein:
n1 is an integer of about 0 or about 1,
n2 is an integer from about 1 to about 6,
R¹ is a bond to a nucleoside in the immunomodulating polynucleotide,
R² is a bond to a nucleoside in the immunomodulating polynucleotide or to a capping group,
each L¹ is independently a phosphodiester or a phosphotriester, and
each L² is a sugar analogue,
   provided that,
   if the abasic spacer is an internucleoside, abasic spacer, each n1 is 1, and R² is a bond to a nucleoside, and
   if the abasic spacer is a terminal, abasic spacer, each n1 is independently an integer of about 0 or about 1, and R² is a bond to a capping group.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “alkane-tetrayl,” as used herein, represents a tetravalent, acyclic, straight or branched chain, saturated hydrocarbon group having from 1 to 16 carbons, unless otherwise specified. Alkane-tetrayl may be optionally substituted as described for alkyl.

The term “alkane-triyl,” as used herein, represents a trivalent, acyclic, straight or branched chain, saturated hydrocarbon group having from 1 to 16 carbons, unless otherwise specified. Alkane-triyl may be optionally substituted as described for alkyl.

The term “alkanoyl,” as used herein, represents hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group attached to aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. The alkenylene group may be unsubstituted or substituted (e.g., optionally substituted alkenylene) as described for alkyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; aryl; aryloxy; azido; cycloalkyl; cycloalkoxy; cycloalkenyl; cycloalkynyl; halo; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or, valency permitting, substituted with unsubstituted substituent(s) defined herein for each respective group.

The term “alkylamino,” as used herein, refers to a group having the formula —N(R^{N1})₂ or

—NHR^{N1}, in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkyl cycloalkylene,” as used herein, refers to a saturated divalent hydrocarbon group that is an alkyl cycloalkane, in which two valencies replace two hydrogen atoms. Preferably, at least one of the two valencies is present on the cycloalkane portion. The alkane and cycloalkane portions may be optionally substituted as the individual groups as described herein.

The term “alkylene,” as used herein, refers to a saturated divalent hydrocarbon group that is a straight or branched chain saturated hydrocarbon, in which two valencies replace two hydrogen atoms. The valency of alkylene defined herein does not include the optional substituents. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. The term “C_{x-y} alkylene” represents alkylene groups having between x and y carbons. Exemplary values for x are 1, 2, 3, 4, 5, and 6, and exemplary values for y are 2, 3, 4, 5, 6, 7, 8, 9, 10, 11, and 12. Alkylene can be optionally substituted as described herein for alkyl.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl). Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “5-alkynyluridine,” as used herein, represents a nucleoside, in which the nucleobase is 5-alkynyluracil of the following structure:

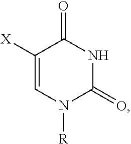

where R is a bond to the anomeric carbon of the pentafuranose of the nucleoside, and X is alkynyl. In some embodiments, X is ethynyl or propynyl (e.g., X is ethynyl).

The term “alkynylene,” as used herein, refers to a straight-chain or branched-chain divalent substituent including one or two carbon-carbon triple bonds and containing only C and H when unsubstituted. Non-limiting examples of the alkynylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. The alkynylene group may be unsubstituted or substituted (e.g., optionally substituted alkynylene) as described for alkynyl groups.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2},

—SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, —SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In certain embodiments, an amino group is —NHR^{N1}, in which R^{N1} is optionally substituted alkyl. Non-limiting examples of —NHR^{N1}, in which R^{N1} is optionally substituted alkyl, include: optionally substituted alkylamino, a proteinogenic amino acid, a non-proteinogenic amino acid, a C₁₋₆ alkyl ester of a proteinogenic amino acid, and a C₁₋₆ alkyl ester of a non-proteinogenic amino acid.

The term “aminoalkyl,” as used herein, represents an alkyl substituted with one, two, or three amino groups, as defined herein. Aminoalkyl may be further optionally substituted as described for alkyl groups.

The term “arene-tetrayl,” as used herein, represents a tetravalent group that is an aryl group, in which three hydrogen atoms are replaced with valencies. Arene-tetrayl can be optionally substituted as described herein for aryl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “aryl alkylene,” as used herein, represents an aryl alkyl group, in which one hydrogen atom is replaced with a valency. Aryl alkylene may be optionally substituted as described herein for aryl alkyl.

The term “arylene,” as used herein, represents an aryl group, in which one hydrogen atom is replaced with a valency. Arylene may be optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “auxiliary moiety,” as used herein, represents a monovalent group containing a hydrophilic polymer, a positively charged polymer, or a sugar alcohol.

The term “optionally substituted N,” as used herein, represents a divalent —N(R^{N1})— group or a trivalent —N=group. The aza group may be unsubstituted, where R^{N1} is H or absent, or substituted, where R^{N1} is as defined for “amino,” except R^{N1} is not H. Two aza groups may be connected to form “diaza.”

The term “optionally substituted N-protected amino,” as used herein, represents substituted amino, as defined herein, in which at least one substituent is an N-protecting group and the other substituent is H, if N-protected amino is unsubstituted, or a substituent other than H, if N-protected amino is substituted.

The term “azido,” as used herein, represents an —N₃ group.

The term “bulky group,” as used herein, represents any substituent or group of substituents as defined herein, in which the radical bonding to disulfide is a carbon atom that bears one hydrogen atom or fewer if the radical is sp³-hybridized carbon or bears no hydrogen atoms if the radical is sp²-hybridized carbon. The radical is not sp-hybridized carbon. The bulky group bonds to disulfide only through a carbon atom.

The term “5′-5′ cap,” as used herein, represents a group of formula R′-Nuc¹-O-(L^{P})ₙ-, where R′ is phosphate, phosphorothioate, phosphorodithioate, phosphotriester, phosphodiester, hydroxyl, or hydrogen; Nuc¹ is a nucleoside; each L_{P} is independently —P(═X^{E1})—X^{E2}—R^{E2A})—O—; and n is 1, 2, or 3;
where each X^{E1} and each X^{E2} is independently O or S, and each R^{E2A} is independently hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties; and
where R′ is bonded to the 3′-carbon of the nucleoside, and —O— is bonded to the 5′-carbon of the nucleoside.

The term “capping group,” as used herein represents a monovalent or a divalent group situated at the 5′- or 3′-terminus of a polynucleotide. The capping group is a terminal phosphoester; diphosphate; triphosphate; an auxiliary moiety; a bioreversible group; a non-bioreversible group; 5′ cap (e.g., 5′-5′ cap); solid support; a linker bonded to a targeting moiety and optionally to one or more (e.g., 1 to 6) auxiliary moieties; or a group —OR′, where R′ is selected from the group consisting of hydrogen, a bioreversible group, non-bioreversible group, solid support, and O-protecting group. Group —OR′, diphosphate, triphosphate, bioreversible group, non-bioreversible group, solid support, and auxiliary moiety are examples of monovalent capping groups. A terminal phosphoester is an example of a capping group that can be either monovalent, if the terminal phosphoester does not include a linker to a targeting moiety, or divalent, if the terminal phosphoester includes a linker to a targeting moiety. A linker bonded to a targeting moiety (with our without auxiliary moieties) is an example of a divalent capping group.

The term “carbocyclic,” as used herein, represents an optionally substituted C₃₋₁₆ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “cyano,” as used herein, represents —CN group.

The term “cycloaddition reaction” as used herein, represents reaction of two components in which a total of [4n+2]π electrons are involved in bond formation when there is either no activation, activation by a chemical catalyst, or activation using thermal energy, and n is 1, 2, or 3. A cycloaddition reaction is also a reaction of two components in which [4n]π electrons are involved, there is photochemical activation, and n is 1, 2, or 3. Desirably, [4n+2]π electrons are involved in bond formation, and n=1. Representative cycloaddition reactions include the reaction of an alkene with a 1,3-diene (Diels-Alder reaction), the reaction of an alkene with an α,β-unsaturated carbonyl (hetero Diels-Alder reaction), and the reaction of an alkyne with an azido compound (e.g., Huisgen cycloaddition).

The term “cycloalkenyl,” as used herein, refers to a non-aromatic carbocyclic group having at least one double bond in the ring and from three to ten carbons (e.g., a C₃-C₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent group that is a cycloalkenyl group, in which one hydrogen atom is replaced with a valency. Cycloalkenylene may be optionally substituted as described herein for cycloalkyl. A non-limiting example of cycloalkenylene is cycloalken-1,3-diyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent group that is a cycloalkyl group, in which one hydrogen atom is replaced with a valency. A non-limiting example of cycloalkylene is cycloalkane-1,3-diyl. Cycloalkylene may be optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

The term “dihydropyridazine group,” as used herein represents a divalent group obtainable through cycloaddition between 1,2,4,5-tetrazine group and a strained cycloalkenyl.

The term “halo,” as used herein, represents a halogen selected from bromine, chlorine, iodine, and fluorine.

The term “5-halouridine,” as used herein, represents a nucleoside, in which the nucleobase is 5-halouracil of the following structure:

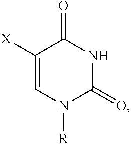

where R is a bond to the anomeric carbon of the pentafuranose of the nucleoside, and X is fluoro, chloro, bromo, or iodo. In some embodiments, X is bromo or iodo.

The term “heteroalkane-tetrayl,” as used herein refers to an alkane-tetrayl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{X-Y} heteroalkane-tetrayl contains from X to Y carbon atoms as well as the heteroatoms as defined herein. The heteroalkane-tetrayl group may be unsubstituted or substituted (e.g., optionally substituted heteroalkane-tetrayl), as described for heteroalkyl.

The term “heteroalkane-triyl,” as used herein refers to an alkane-triyl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{X-Y} heteroalkane-triyl contains from X to Y carbon atoms as well as the heteroatoms as defined herein. The heteroalkane-triyl group may be unsubstituted or substituted (e.g., optionally substituted heteroalkane-triyl), as described for heteroalkyl.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N-protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “(heterocyclyl)aza,” as used herein, represents a chemical substituent of formula —N(R^{N1})(R^{N2}), where R^{N1} is a heterocyclyl group, and R^{N2} is H, —OH, —NO₂, —N(R^{N2})₂, —SO₂OR^{N2},

—SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl. Preferably, R^{N2} is H.

The term “heterocyclylene,” as used herein, represents a heterocyclyl group, in which one hydrogen atom is replaced with a valency. The heterocyclylene may be optionally substituted in a manner described for heterocyclyl. A non-limiting example of heterocyclylene is heterocycle-1,3-diyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “immunomodulating polynucleotide” as used herein, represents a polynucleotide construct containing a total of from 6 to 50 contiguous nucleosides covalently bound together by internucleoside bridging groups independently selected from the group consisting of internucleoside phosphoesters and optionally internucleoside abasic spacers. The immunomodulating polynucleotides are capped at 5′- and 3′-termini with 5′- and 3′-capping groups, respectively. The immunomodulating polynucleotides are capable of modulating an innate immune response, as determined by, e.g., a change in the activation of NFκB or a change in the secretion of at least one inflammatory cytokine or at least one type I interferon in an antigen-presenting cell to which an immunomodulating polynucleotide was delivered (e.g., in comparison to another antigen-presenting cell to which an immunomodulating polynucleotide was not delivered). The immunomodulating polynucleotide may contain a conjugating group or, if the immunomodulating polynucleotide is part of a conjugate, a linker bonded to a targeting moiety and optionally to one or more (e.g., 1 to 6) auxiliary moieties (e.g., polyethylene glycols). The conjugating group or the linker may be part of the phosphotriester or the terminal capping group.

The term “immunostimulating polynucleotide” as used herein, represents an immunomodulating polynucleotide capable of activating an innate immune response, as determined by, e.g., an increase in the activation of NFκB or an increase in the secretion of at least one inflammatory cytokine or at least one type I interferon in an antigen-presenting cell to which an immunostimulating polynucleotide was delivered (e.g., in comparison to another antigen-presenting cell to which an immunostimulating polynucleotide was not delivered). In some embodiments, the immunostimulating polynucleotide contains at least one cytidine-p-guanosine (CpG) sequence, in which p is an internucleoside phosphodiester (e.g., phosphate or phosphorothioate) or an internucleoside phosphotriester or phosphothiotriester. As used herein, the CpG-containing immunostimulating polynucleotide can be naturally existing, such as CpG ODNs of bacterial or viral origins, or synthetic. For example, in some embodiments, the CpG sequence in the immunostimulating polynucleotide contains 2′-deoxyribose. In some embodiments, the CpG sequence in the immunostimulating polynucleotide is unmethylated. In some embodiments, the immunostimulating polynucleotide is a polynucleotide of Formula (A) as provided herein. In some embodiments, the immunostimulating polynucleotide is compound of Formula (B) as provided herein.

The term “immunosuppressive polynucleotide” as used herein, represents an immunomodulating polynucleotide capable of antagonizing an innate immune response, as determined by e.g., a reduction in the activation of NFκB or a reduction in the secretion of at least one inflammatory cytokine or at least one type I interferon in an antigen-presenting cell to which an immunosuppressive polynucleotide was delivered (e.g., in comparison to another antigen-presenting cell to which an immunosuppressive polynucleotide was not delivered).

The term “internucleoside bridging group,” as used herein, represents an internucleoside phosphoester or an internucleoside abasic spacer.

The term “5-modified cytidine,” as used herein represents a nucleoside, in which the nucleobase is of the following structure:

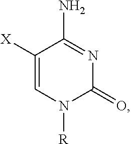

where R is a bond to the anomeric carbon of the pentafuranose of the nucleoside, and X is halogen, alkynyl, alkenyl, alkyl, cycloalkyl, heterocyclyl, or aryl. In some embodiments, 5-modified cytidine is 5-halo cytidine (e.g., 5-iodo cytidine or 5-bromo cytidine). In other embodiments, 5-modified cytidine is 5-alkynyl cytidine.

The term “5-modified uridine,” as used herein represents a nucleoside, in which the nucleobase is of the following structure:

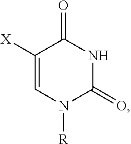

where R is a bond to the anomeric carbon of the pentafuranose of the nucleoside, and X is halogen, alkyl, alkenyl, alkynyl, cycloalkyl, heterocyclyl, or aryl, provided that the 5-modified uridine is not thymidine. In some embodiments, 5-modified uridine is 5-halouridine (e.g., 5-iodouridine or 5-bromouridine). In other embodiments, 5-modified uridine is 5-alkynyl uridine. In some embodiments, 5-modified uridine is a nucleoside containing 2-deoxyribose.

The term “non-bioreversible,” as used herein, refers to a chemical group that is resistant to degradation under conditions existing inside an endosome. Non-bioreversible groups do not contain thioesters and/or disulfides.

The term “nucleobase,” as used herein, represents a nitrogen-containing heterocyclic ring bound to the 1′ position of the sugar moiety of a nucleotide or nucleoside. Nucleobases can be unmodified or modified. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-iodo, 5-bromo, 5-trifluoromethyl and other 5-substituted uracils and cytosines, 5-alkynyl (e.g., 5-ethynyl) uracil, 5-acetamido-uracil, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; those disclosed in The Concise Encyclopedia Of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289 302, (Crooke et al., ed., CRC Press, 1993). Certain nucleobases are particularly useful for increasing the binding affinity of the hybridized polynucleotides of the invention, including 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications 1993, CRC Press, Boca Raton, pages 276-278). These may be combined, in particular embodiments, with 2′-O-methoxyethyl sugar modifications. United States patents that teach the preparation of certain of these modified nucleobases as well as other modified nucleobases include, but are not limited to, the above noted U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; and 5,681,941. For the purposes of this disclosure, “modified nucleobases,” as used herein, further represents nucleobases, natural or non-natural, which include one or more protecting groups as described herein.

The term “nucleoside,” as used herein, represents a pentafuranose-nucleobase combination. The pentafuranose is 2-deoxyribose or a modified version thereof, in which position 2 is substituted with OR, R, halo (e.g., F), SH, SR, NH₂, NHR, NR₂, or CN, where R is an optionally substituted C₁₋₆ alkyl (e.g., C₁₋₆ alkyl or (C₁₋₆ alkoxy)-C₁₋₆-alkyl) or optionally substituted (C₆₋₁₄ aryl)-C₁₋₄-alkyl. In certain embodiments, position 2 is substituted with OR or F, where R is C₁₋₆ alkyl or (C₁₋₆-alkoxy)-C₁₋₆-alkyl. The pentafuranose is bonded to a nucleobase at the anomeric carbon. In some embodiments, the term “nucleoside” refers to a divalent group having the following structure:

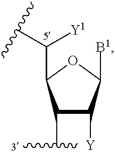

in which B¹ is a nucleobase; Y is H, halogen (e.g., F), hydroxyl, optionally substituted C₁₋₆ alkoxy (e.g., methoxy or methoxyethoxy), or a protected hydroxyl group; Y¹ is H or C₁₋₆ alkyl (e.g., methyl); and each of 3′ and 5′ indicate the position of a bond to another group.

The term “nucleotide,” as used herein, refers to a nucleoside that is bonded to a phosphate, phosphorothioate, or phosphorodithioate.

The term “phosphoester,” as used herein, represents a group containing a phosphate, phosphorothioate, or phosphorodithioate, in which, at least one valency is covalently bonded to a non-hydrogen substituent, provided that at least one non-hydrogen substituent is a group containing at least one nucleoside. A phosphoester, in which one and only one valency is covalently bonded to a group containing a nucleoside, is a terminal phosphoester. A phosphoester, in which two valencies are covalently bonded to nucleoside-containing groups, is an internucleoside phosphoester. A phosphoester may be a group of the following structure:

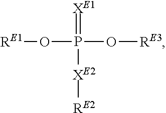

where:

each of X^{E1} and X^{E2} is independently O or S;

each or R^{E1} and R^{E3} is independently hydrogen or a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(═X^{E1})(—X^{E2}—R^{E2A})—O—,
where R^{E2A} is hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties; and

R^{E2} is hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties;

provided that at least one of R^{E1} and R^{E3} is a bond to a group containing at least one nucleoside.

If each of R^{E1} and R^{E3} is independently a bond to a group containing at least one nucleoside, the phosphoester is an internucleoside phosphoester. If one of R^{E1} and R^{E3} is a bond to a group that does not contain a nucleoside, the phosphoester is a terminal phosphoester.

The term “phosphodiester,” as used herein, refers to a phosphoester, in which, two of the three valencies are substituted with non-hydrogen substituents, while the remaining valency is substituted with hydrogen. The phosphodiester consists of phosphate, phosphorothioate, or phosphorodithioate; one or two bonds to nucleoside(s), abasic spacer(s), and/or phosphoryl group(s); and, if the phosphodiester contains only one bond to a nucleoside, an abasic spacer, or a phosphoryl group, one group independently selected from the group consisting of a bioreversible group; a non-bioreversible group; an auxiliary moiety; a conjugating group; a linker bonded to a targeting moiety; and a linker bonded to a targeting moiety and one or more (e.g., 1 to 6) auxiliary moieties. A terminal phosphodiester includes one bond to a group containing a nucleoside, and one group selected from the group consisting of a bioreversible group; a non-bioreversible group; an auxiliary moiety; a conjugating group; a phosphoryl group; and a linker bonded to a targeting moiety and optionally to one or more (e.g., 1 to 6) auxiliary moieties. An internucleoside phosphodiester includes two bonds to nucleoside-containing groups. A phosphodiester may be a group of the following structure:

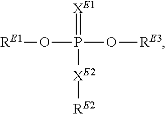

where:

each of X^{E1} and X^{E2} is independently O or S;

each or R^{E1} and R^{E3} is independently hydrogen or a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(═X^{E1})(—X^{E2}—R^{E2A})—O—,
where R^{E2A} is hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties; and

R^{E2} is hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties;

provided that one and only one of R^{E1}, R^{E2}, and R^{E3} is hydrogen; and

provided that at least one of R^{E1} and R^{E3} is a bond to a group containing at least one nucleoside.

If both R^{E1} and R^{E3} are bonds to groups containing at least one nucleoside, the phosphodiester is an internucleoside phosphodiester. If one and only one of R^{E1} and R^{E3} is a bond to a group containing a nucleoside, the phosphodiester is a terminal phosphodiester.

The term “phosphoryl,” as used herein, refers to a substituent of formula

—P(═X^{E1}—)(X^{E2}—R^{E2A})—O—R^{E3A},

where:

each of X^{E1} and X^{E2} is independently O or S;

R^{E2A} is hydrogen, a bioreversible group, a non-bioreversible group, an auxiliary moiety, a conjugating group, a linker bonded to a targeting moiety, or a linker bonded to a targeting moiety and one or more (e.g., 1 to 6) auxiliary moieties; and

R^{E3A} is hydrogen or an open valency.

When a group is identified as being bonded to a phosphoryl, the group is bonded to the phosphorus atom of the phosphoryl.

The term “phosphotriester,” as used herein, refers to a phosphoester, in which all three valences are substituted with non-hydrogen substituents. The phosphotriester consists of phosphate, phosphorothioate, or phosphorodithioate; one or two bonds to nucleoside(s), or abasic spacer(s), and/or phosphoryl group(s); and one or two groups independently selected from the group consisting of a bioreversible group; a non-bioreversible group; an auxiliary moiety; a conjugating group; and a linker bonded to a targeting moiety and optionally to one or more (e.g., 1 to 6) auxiliary moieties. A terminal phosphotriester includes one bond to a group containing a nucleoside and two groups independently selected from the group consisting of a bioreversible group; a non-bioreversible group; an auxiliary moiety; a conjugating group; a phosphoryl group; and a linker bonded to a targeting moiety and optionally to one or more (e.g., 1 to 6) auxiliary moieties. In some embodiments, a terminal phosphotriester contains 1 or 0 linkers bonded to a targeting moiety and optionally to one or more (e.g., 1 to 6) auxiliary moieties. An internucleoside phosphotriester includes two bonds to nucleoside-containing groups. A phosphotriester may be a group of the following structure:

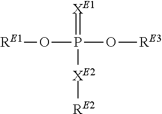

where:

each of X^{E1} and X^{E2} is independently O or S;

each or R^{E1} and R^{E3} is independently a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(═X^{E1})—X^{E2}—R^{E2A})—O—,
where R^{E2A} is hydrogen; a bioreversible group; a non-bioreversible group; an auxiliary moiety; a conjugating group; a linker bonded to a targeting moiety; or a linker bonded to a targeting moiety and one or more (e.g., 1 to 6) auxiliary moieties; and

R^{E2} is a bioreversible group; a non-bioreversible group; an auxiliary moiety; a conjugating group; a linker bonded to a targeting moiety; or a linker bonded to a targeting moiety and one or more (e.g., 1 to 6) auxiliary moieties;

provided that at least one of R^{E1} and R^{E3} is a bond to a group containing at least one nucleoside.

If both R^{E1} and R^{E3} are bonds to groups containing at least one nucleoside, the phosphotriester is an internucleoside phosphotriester. If one and only one of R^{E1} and R^{E3} is a bond to a group containing a nucleoside, the phosphotriester is a terminal phosphotriester.

The term “pyrid-2-yl hydrazone,” as used herein, represents a group of the structure:

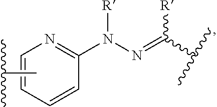

where each R′ is independently H or optionally substituted C₁₋₆ alkyl. Pyrid-2-yl hydrazone may be unsubstituted (i.e., each R′ is H).

The term “stereochemically enriched,” as used herein, refers to a local stereochemical preference for one stereoisomeric configuration of the recited group over the opposite stereoisomeric configuration of the same group. Thus, a polynucleotide containing a stereochemically enriched phosphorothioate is a strand, in which a phosphorothioate of predetermined stereochemistry is present in preference to a phosphorothioate of the opposite stereochemistry. This preference can be expressed numerically using a diastereomeric ratio for the phosphorothioate of the predetermined stereochemistry. The diastereomeric ratio for the phosphorothioate of the predetermined stereochemistry is the molar ratio of the diastereomers having the identified phosphorothioate with the predetermined stereochemistry relative to the diastereomers having the identified phosphorothioate with the opposite stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “Q-tag,” as used herein, refers to a portion of a polypeptide containing glutamine residue that, upon transglutaminase-mediated reaction with a compound containing —NH2 amine, provides a conjugate containing the portion of polypeptide, in which the glutamine residue includes a side chain modified to include the amide bonded to the compound. Q-tags are known in the art. Non-limiting examples of Q-tags are LLQGG (SEQ ID NO:582) and GGGLLQGG (SEQ ID NO:583).

The term “strained cycloalkenyl,” as used herein, refers to a cycloalkenyl group that, if the open valency were substituted with H, has a ring strain energy of at least 16 kcal/mol.

The term “sugar analogue,” as used herein, represents a divalent or trivalent group that is a C₃₋₆ monosaccharide or C₃₋₆ alditol (e.g., glycerol), which is modified to replace two hydroxyl groups with bonds to the oxygen atoms in phosphate, phosphorothioate, or phosphorodithioate, or a capping group. A sugar analogue does not contain a nucleobase capable of engaging in hydrogen bonding with a nucleobase in a complementary strand. A sugar analogue is cyclic or acyclic. Further optional modifications included in a sugar analogue are: a replacement of one, two, or three of the remaining hydroxyl groups or carbon-bonded hydrogen atoms with H; optionally substituted C₁₋₆ alkyl; -LinkA(-T)ₚ, as defined herein; a conjugating group; —(CH₂)₁₁—OR^{Z}, where t1 is an integer from 1 to 6, and R^{Z} is optionally substituted C₁₋₆ alkyl, optionally substituted C₂₋₆ alkenyl, optionally substituted C₂₋₆ alkynyl, optionally substituted C₆₋₁₄ aryl, optionally substituted C₃₋₈ cycloalkyl, optionally substituted (C₁₋₉ heterocyclyl)-C₁₋₆-alkyl, optionally substituted (C₆₋₁₀ aryl)-C₁₋₆-alkyl, or optionally substituted (C₃₋₈ cycloalkyl)-C₁₋₆-alkyl; introduction of one or two unsaturation(s) (e.g., one or two double bonds); and replacement of one, two, or three hydrogens or hydroxyl groups with substituents as defined for alkyl, alkenyl, cycloalkyl, cycloalkenyl, or heterocyclyl. Non-limiting examples of sugar analogues are optionally substituted C₂₋₆ alkylene, optionally substituted C₂₋₆ alkenylene, optionally substituted C₅ cycloalkane-1,3-diyl, optionally substituted C₅ cycloalkene-1,3-diyl, optionally substituted heterocycle-1,3-diyl (e.g., optionally substituted pyrrolidine-2,5-diyl, optionally substituted tetrahydrofuran-2,5-diyl, or optionally substituted tetrahydrothiophene-2,5-diyl), or optionally substituted (C₁₋₄ alkyl)-(C₃₋₈ cycloalkylene) (e.g., optionally substituted (C₁ alkyl)-(C₃ cycloalkylene)).

The term “sulfide,” as used herein, represents a divalent —S— or ═S group. Disulfide is —S—S—.

The term “targeting moiety,” as used herein, represents a moiety (e.g., a small molecule, e.g., a carbohydrate) that specifically binds or reactively associates or complexes with a receptor or other receptive moiety associated with a given target cell population (e.g., an antigen-presenting cell (APC; e.g., a professional APC (e.g., B-cell, pDC, or macrophage))). A conjugate provided herein comprises a targeting moiety. The targeting moiety can be an antibody or an antigen-binding fragment or an engineered derivative thereof (e.g., Fcab or a fusion protein (e.g., scFv)). The targeting moiety can be a polypeptide. Alternatively, the targeting moiety can be a small molecule (e.g., mannose) or a cluster of small molecules (e.g., a cluster of mannoses). A conjugate of the invention that includes the targeting moiety may exhibit K_{d} of less than 100 nM for the target, to which the targeting moiety bind. K_{d} is measured using methods known in the art, e.g., using surface plasmon resonance (SPR), e.g., using BIACORE^{T}M system (GE Healthcare, Little Chalfont, the United Kingdom).

The term “1,2,4,5-tetrazine group,” as used herein, represents a group of the following formula:

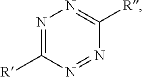

where R′ is optionally substituted alkyl, optionally substituted aryl, optionally substituted cycloalkyl, optionally substituted heterocyclyl; and R″ is optionally substituted alkylene, optionally substituted heteroalkylene, optionally substituted arylene, optionally substituted cycloalkylene, optionally substituted heterocyclylene, or a group —R^{a}—R^{b}—, in which each of R^{a} and R^{b}is independently optionally substituted alkylene, optionally substituted heteroalkylene, optionally substituted arylene, optionally substituted cycloalkylene, or optionally substituted heterocyclylene.

The term “therapeutic effect” refers to a local or systemic effect in a subject, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The term “therapeutically effective amount” or “therapeutically effective dose,” as used herein, represents the quantity of an immunomodulating polynucleotide or a conjugate necessary to ameliorate, treat, or at least partially arrest the symptoms of a disease to be treated. Amounts effective for this use depend on the severity of the disease and the weight and general state of the subject. Typically, dosages used in vitro may provide useful guidance in the amounts useful for in vivo administration of the pharmaceutical composition, and animal models may be used to determine effective dosages for treatment of a particular disease.

The term “thiocarbonyl,” as used herein, represents a C(═S) group.

The term “thioheterocyclylene,” as used herein, represents a group —S—R—, where R is heterocyclylene. Thioheterocyclylene may be optionally substituted in a manner described for heterocyclyl.

The term “thiol,” as used herein, represents an —SH group.

The term “treating” as used in reference to a disease or a condition in a patient, is intended to refer to obtaining beneficial or desired results, e.g., clinical results, in a patient by administering the polynucleotide or conjugate of the invention to the patient. Beneficial or desired results may include alleviation or amelioration of one or more symptoms of a disease or condition; diminishment of extent of a disease or condition; stabilization (i.e., not worsening) of a disease or condition; prevention of the spread of a disease or condition; delay or slowing the progress of a disease or condition; palliation of a disease or condition; and remission (whether partial or total). “Palliating” a disease or condition means that the extent and/or undesirable clinical manifestations of the disease or condition are lessened and/or time course of the progression is slowed, as compared to the extent or time course in the absence of the treatment with the polynucleotide or conjugate of the invention.

The term “triazolocycloalkenylene,” as used herein, refers to the heterocyclylenes containing a 1,2,3-triazole ring fused to an 8-membered ring, all of the endocyclic atoms of which are carbon atoms, and bridgehead atoms are sp²-hybridized carbon atoms. Triazocycloalkenylenes can be optionally substituted in a manner described for heterocyclyl.

The term “triazoloheterocyclylene,” as used herein, refers to the heterocyclylenes containing a 1,2,3-triazole ring fused to an 8-membered ring containing at least one heteroatom. The bridgehead atoms in triazoloheterocyclylene are carbon atoms. Triazoloheterocyclylenes can be optionally substituted in a manner described for heterocyclyl.

It is to be understood that the terms “immunomodulating polynucleotide,” “immunostimulating polynucleotide,” “immunosuppressive polynucleotide,” and “conjugate” encompass salts of the immunomodulating polynucleotide, immunostimulating polynucleotide, immunosuppressive polynucleotide and conjugate, respectively. For example, the terms “immunomodulating polynucleotide,” “immunostimulating polynucleotide,” “immunosuppressive polynucleotide,” and “conjugate” encompasses both the protonated, neutral form (P—XH moiety, where X is O or S) of a phosphate, phosphorothioate, or phosphorodithioate and the deprotonated, ionic form (P—X⁻ moiety, where X is O or S) of a phosphate, phosphorothioate, or phosphorodithioate. Accordingly, it is to be understood that the phosphoesters and phosphodiesters described as having one or more of R^{E1}, R^{E2}, and R^{E3} as hydrogen encompass salts, in which the phosphate, phosphorothioate, or phosphorodithioate is present in a deprotonated, ionic form.

The terms “innate immune response” and “innate immunity” are recognized in the art, and refer to non-specific defense mechanism a body's immune system initiates upon recognition of pathogen-associated molecular patterns, which involves different forms of cellular activities, including cytokine production and cell death through various pathways. As used herein, innate immune responses include cellular responses to a CpG-containing immunostimulating polynucleotide mediated by toll-like receptor 9 (TLR9), which include, without limitation, increased production of inflammation cytokines (e.g., type I interferon or IL-10 production), activation of the NFκB pathway, increased proliferation, maturation, differentiation and/or survival of immune cells, and in some cases, induction of cell apoptosis. Activation of the innate immunity can be detected using methods known in the art, such as measuring the (NF)-κB activation.

The terms “adaptive immune response” and “adaptive immunity” are recognized in the art, and refer to antigen-specific defense mechanism a body's immune system initiates upon recognition of a specific antigen, which include both humoral response and cell-mediated responses. As used herein, adaptive immune responses include cellular responses that is triggered and/or augmented by a CpG-containing immunostimulating polynucleotide. In some embodiments, the immunostimulating polynucleotide or a portion thereof is the antigen target of the antigen-specific adaptive immune response. In other embodiments, the immunostimulating polynucleotide is not the antigen target of the antigen-specific adaptive immune response, but nevertheless augments the adaptive immune response. Activation of an adaptive immune response can be detected using methods known in the art, such as measuring the antigen-specific antibody production, or the level of antigen-specific cell-mediated cytotoxicity.

The term “Toll-like receptor” (or “TLR”) is recognized in the art, and refers to a family of pattern recognition receptors that were initially identified as sensors of the innate immune system that recognize microbial pathogens. TLRs recognize distinct structures in microbes, often referred to as “PAMPs” (pathogen associated molecular patterns). Ligand binding to TLRs invokes a cascade of intra-cellular signaling pathways that induce an innate immune response and/or adaptive immune response. As used herein, the term “toll-like receptor” or “TLR” also refers to a functional fragment of a toll-like receptor protein expressed by a cell. In humans, ten TLRs have been identified, including TLR-1, -2, -3, -4, -5, -6, -7/8, -9, and -10. D'Arpa and Leung, Adv. Wound Care, 2017, 6, 330-343. Human genes encoding TLRs are known.

Toll-like receptor 9 (TLR9), also designated as CD289 (cluster of differentiation 289), is a member of the toll-like receptor (TLR) family. Du et al., Eur. Cytokine Netw., 11:362-371 (2000). TLR9 is an important receptor expressed in immune system cells including dendritic cells (DC₅), B lymphocytes, macrophages, natural killer cells, and other antigen presenting cells. TLR9 activation triggers signaling cascades that bridges the innate and adaptive immunity. Martinez-Campos et al., Viral Immunol., 30:98-105 (2016); Notley et al., Sci. Rep., 7:42204 (2017). Natural TLR-9 agonists include unmethylated cytosine-guanine dinucleotide (CpG)-containing oligodeoxynucleotides (CpG ODNs). TLR-9 ligand finding use in the present disclosure include, but are not limited to, naturally existing or synthetic CpG ODNs, and other CpG-containing immunostimulating polynucleotide and/or immunoconjugates as provided herein. Activation of the TLR9 signaling pathway can be detected using methods known in the art, such as measuring recruitment of myeloid differentiation antigen 88 (MyD88), activation of nuclear factor (NF)-κB, c-Jun N-terminal kinase (JNK), and p38 mitogen-activated protein kinase (MAPK) signaling pathways, activation of interferon regulatory factor-7, expression level of one or more of cytokines such as type I interferons (IFNs), interleukin (IL)-6, IL-10, and IL-12, activation of one or more immune cell populations such as NK cells, natural killer T cells, monocytes, and level of cytotoxic lymphocyte (CTL) and T helper-1 (Th1) responses, and the level of immunoglobulin secretion.

The term “TLR-expressing cell” as used herein refers to a cell that expresses a toll-like receptor and is capable of activating the toll-like receptor signaling pathway upon binding of the toll-like receptor to an agonist. The toll-like receptor may be expressed on the cell surface, and/or on the membrane of one or more intracellular compartments of the cell, such as the endosome or phagosome. A TLR-expressing cell may further express one or more cell surface antigens other than the toll-like receptor. Certain immune cells express TLRs, and activation of the TLR signaling pathway in the immune cells elicits an innate immune response, and/or an adaptive immune response. Immune cells activated by the TLR signaling pathway can help eliminate other diseased cells from the body. Certain diseased cells (e.g., cancer cells or viral-infected cells) express TLRs, and activation of the TLR signaling pathway in the diseased cells can results in death of the diseased cell, such as via induced apoptosis. Examples of TLR9-expressing cells include but are not limited to dendritic cells (DC₅), B cells, T cells, Langerhans cells, keratinocytes, mast cells, endothelial cells, myofibroblast cells, and primary fibroblast. Determining whether a cell expresses any toll-like receptor (e.g., TLR9) can be performed using methods known in the art, such as detecting mRNA of the toll-like receptor in a cell.

The term “immune cell” is recognized in the art, as used herein refers to any cell involved in a host defense mechanism, such as cells that produces pro-inflammatory cytokines, and cells that participate in tissue damage and/or disease pathogenesis. Examples of immune cells include, but are not limited to, T cells, B cells, natural killer cells, neutrophils, mast cells, macrophages, antigen-presenting cells (APC), basophils, and eosinophils.

The term “antigen presenting cell” or “APC” is recognized in the art, and refers to a heterogeneous group of immune cells that mediate the cellular immune response by processing and presenting antigens for recognition by certain lymphocytes such as T cells. Exemplary types of antigen presenting cells include, but are not limited to, professional antigen presenting cells including, for example, B cells, monocytes, dendritic cells, and Langerhans cells, as well as other antigen presenting cells including, for example, keratinocytes, endothelial cells, astrocytes, fibroblasts, and oligodendrocytes. As used herein, the term “antigen presenting cell” includes antigen presenting cells found in vivo and those found in in vitro cell cultures derived from the in vivo cells. As used herein, antigen presenting cells also include an APC that is artificially modified, such as genetically modified to express a toll-like receptor (e.g., TLR9) or to modulate expression level of a toll-like receptor (e.g., TLR9).

The term “dendritic cells” or “DC” is recognized in the art, and refers to a heterogeneous group of specialized antigen-sensing and antigen-presenting cells (APC₅). Human DC are divided into three major subsets: plasmacytoid DC (pDC), myeloid DC (mDC) and monocyte-derived DC (MDDC). Schraml et al., Curr. Opin. Immunol., 32:13-20 (2015). Subsets of DC₅ can be identified on the basis of distinct TLR expression patterns. By way of an example, the myeloid or “conventional” subset of DC (mDC) expresses TLRs 1-8 when stimulated, and a cascade of activation markers (e.g., CD80, CD86, MHC class I and II, CCR7), pro-inflammatory cytokines, and chemokines are produced. A result of this stimulation and resulting expression is antigen-specific CD4+ and CD8+ T cell priming. These DC₅ acquire an enhanced capacity to take up antigens and present them in an appropriate form to T cells. The plasmacytoid subset of DC (pDC) expresses TLR7 and TLR9 upon activation, with a resulting activation of NK cells as well as T-cells.

The term “antigen” as used herein, refers to a molecule or an antigenic fragment thereof capable of eliciting an immune response, including both an innate immune response and an adaptive immune response. As used herein, antigens can be proteins, peptides, polysaccharides, lipids, nucleic acids, especially RNA and DNA, nucleotides, and other biological or biochemical substances. The term “elicit an immune response” refers to the stimulation of immune cells in vivo in response to a stimulus, such as an antigen. The immune response consists of both cellular immune response, e.g., T cell and macrophage stimulation, and humoral immune response, e.g., B cell and complement stimulation and antibody production. Immune response may be measured using techniques well-known in the art, including, but not limited to, antibody immunoassays, proliferation assays, and others.

The terms “antigenic fragment” and “antibody binding fragment” are used interchangeably herein. An antigenic fragment as used herein is able to complex with an antigen binding molecule, e.g., an antibody, in a specific reaction. The specific reaction referred to herein indicates that the antigen or antigenic fragment will react, in a highly selective manner, with its corresponding antibody and not with the multitude of other antibodies which may be evoked by other antigens. The specificity of such reaction is determined by the presence of one or more epitopes (immunogenic determinants) in the antigen. As used herein, an antigen or antigenic fragment thereof may have one epitope, or have more than one epitopes.

The term “T cell epitope” as used herein, refers to any epitopes of antigens produced by a T cell.

The term “tumor associated antigen” or “TAA”, as used herein, refers to an antigen expressed by a cancer cell or in the stroma of a solid tumor in a cancer patient receiving the treatment or preventive care as provided herein (e.g., receiving a therapeutic dose of an immunostimulating polynucleotide or a CpG-Ab immunoconjugate). The TAA may or may not be targeted in the treatment or the preventive care provided herein. The TAA does not have to be overexpressed, mutated or misregulated on cancer cell but can have same features as the TAA would have in a normal cell. In some embodiments, the TAA can be overexpressed, mutated or misregulated in cancer cell. The TAA can be a protein, nucleic acid, lipid or other antigen. The TAA can be a cell-surface expressed TAA, an intracellular TAA or an intranuclear TAA. In the context of a solid tumor, the TAA can be expressed in the stroma of a solid tumor mass. The term “stroma” as used herein refers to components in a solid tumor mass other than a cancer cell. For example, the stroma can include fibroblasts, epithelial cells, other blood vessel components or extracellular matrix components. As used herein, the term “stroma” does not include components of the immune system, such as immune cells (e.g., B-cells, T-cells, dendritic cells, macrophages, natural killer cells, and the like)). Various TAAs are known in the art. Identifying TAA can be performed using methods known in the art, such as disclosed in Zhang et al., Methods Mol. Biol., 520:1-10 (2009).

The terms “antibody,” “immunoglobulin,” and “Ig” are used interchangeably herein, and are used in the broadest sense and specifically cover, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies. An antibody can be human, humanized, chimeric and/or affinity matured as well as an antibody from other species, for example, mouse and rabbit.

The term “antibody” is intended to include a polypeptide product of B cells within the immunoglobulin class of polypeptides that is able to bind to a specific antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxyl-terminal portion of each chain includes a constant region. See Borrebaeck (ed.) (1995) Antibody Engineering, Second Ed., Oxford University Press.; Kuby (1997) Immunology, Third Ed., W.H. Freeman and Company, New York. Antibodies also include, but are not limited to, synthetic antibodies, monoclonal antibodies, recombinant antibodies, multispecific antibodies (including bi-specific antibodies), human antibodies, humanized antibodies, camelized antibodies, chimeric antibodies, intrabodies, anti-idiotypic (anti-Id) antibodies, and functional fragments thereof, which refers a portion of an antibody heavy or light chain polypeptide that retains some or all of the binding activity of the antibody from which the fragment is derived. Non-limiting examples of functional fragments of an antibody include single-chain Fvs (scFv) (e.g., including monospecific or bispecific), Fab fragments, F(ab′) fragments, F(ab)₂ fragments, F(ab′)₂ fragments, disulfide-linked Fvs (sdFv), Fd fragments, Fv fragments, diabody, triabody, tetrabody, and minibody. In particular, antibodies provided herein include immunoglobulin molecules and immunologically active portions of immunoglobulin molecules, for example, antigen binding domains or molecules that contain an antigen-binding site that binds to the antigen (e.g., one or more complementarity determining regions (CDRs) of an anti-CD56 antibody or an anti-SIRPα antibody). Such antibody fragments are described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics 1993, 22, 189-224; Plückthun and Skerra, Meth. Enzymol. 1989, 178, 497-515; and Day, Advanced Immunochemistry, Second Ed., Wiley-Liss, Inc., New York, N.Y. (1990). The antibodies provided herein can be of any type (e.g., IgG, IgE, IgM, IgD, IgA, and IgY), any class (e.g., IgG1, IgG2, IgG3, IgG4, IgA1, and IgA2), or any subclass (e.g., IgG2a and IgG2b) of an immunoglobulin molecule.

The term “antigen” refers to a predetermined antigen to which an antibody can selectively bind. A target antigen can be a polypeptide, carbohydrate, nucleic acid, lipid, hapten, or other naturally occurring or synthetic compound. In one embodiment, the target antigen is a polypeptide.

The terms “antigen binding fragment,” “antigen binding domain,” and “antigen binding region” refer to a portion of an antibody that comprises the amino acid residues that interact with an antigen and confer on the binding agent its specificity and affinity for the antigen (e.g., complementarity determining regions (CDRs)).

The term “specific binding,” “specifically binds to,” or “specific for” a particular polypeptide or an epitope on a particular polypeptide target can be exhibited, for example, by a molecule (e.g., an antibody) having a dissociation constant (K_{d}) for the target of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, or at least about 10⁻¹² M. In one embodiment, the term “specific binding” refers to binding where a molecule binds to a particular polypeptide or epitope on a particular polypeptide without substantially binding to any other polypeptide or polypeptide epitope.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Stites et al. (eds.), Appleton & Lange, Norwalk, Conn., 1994, page 71 and Chapter 6.

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md., 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “variable region residue numbering as in Kabat” or “amino acid position numbering as in Kabat”, and variations thereof, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc, according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, including, for example, by AbM, Chothia, Contact, IMGT and AHon.

An “intact” antibody is one comprising an antigen-binding site as well as a CL and at least heavy chain constant regions, CH1, CH2 and CH3. The constant regions may include human constant regions or amino acid sequence variants thereof. Preferably, an intact antibody has one or more effector functions.

The term “antibody fragment” refers to a portion of an intact antibody, preferably the antigen binding or variable region of the intact antibody. Examples of antibody fragments include, without limitation, Fab, Fab′, F(ab′)2, and Fv fragments; diabodies and di-diabodies (see, e.g., Holliger et al., Proc. Natl. Acad. Sci. U.S.A. 1993, 90, 6444-8; Lu et al., J. Biol. Chem. 2005, 280, 19665-72; Hudson et al., Nat. Med. 2003, 9,129-134; WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 1999, 50, 98-101 Streltsov et al., Proc. Natl. Acad. Sci. U.S.A. 2004, 101, 12444-12449); and multispecific antibodies formed from antibody fragments.

The term “functional fragment,” “binding fragment,” or “antigen binding fragment” of an antibody refers to a molecule that exhibits at least one of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids and a carboxyl-terminal portion that includes a constant region. The constant region can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxyl-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a beta klotho epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., Nature 1975, 256, 495; or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature 1991, 352, 624-628 and Marks et al., J. Mol. Biol. 1991, 222, 581-597, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Exemplary methods of producing monoclonal antibodies are provided in the Examples herein.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 1986, 321, 522-525; Riechmann et al., Nature 1988, 332, 323-329; Presta, Curr. Opin. Biotechnol. 1992, 3, 394-398; Carter et al., Proc. Natl. Acad. Sci. U.S.A. 1992, 89, 4285-4289; and U.S. Pat. Nos. 6,800,738, 6,719,971, 6,639,055, 6,407,213, and 6,054,297.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 1991, 227, 381; Marks et al., J. Mol. Biol. 1991, 222, 581) and yeast display libraries (Chao et al., Nature Protocols 2006, 1, 755-768). Also available for the preparation of human monoclonal antibodies are methods described in Cole et al., Monoclonal Antibodies and Cancer Therapy, Alan R. Liss, p. 77 (1985); Boerner et al., J. Immunol. 1991, 147, 86-95. See also van Dijk and van de Winkel, Curr. Opin. Pharmacol. 2001, 5, 368-374. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 1995, 6, 561-566; Bruggemann and Taussing, Curr. Opin. Biotechnol. 1997, 8, 455-458; and U.S. Pat. Nos. 6,075,181 and 6,150,584 regarding XENOMOUSE™ technology). See also, for example, Li et al., Proc. Natl. Acad. Sci. U.S.A. 2006, 103, 3557-3562 regarding human antibodies generated via a human B-cell hybridoma technology.

A “CDR” refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL 3-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32,1-75. CDR region sequences also have been defined structurally by Chothia as those residues that are not part of the conserved 3-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods. 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “hypervariable region”, “HVR”, or “HV”, when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., Sequences of Proteins of Immunological Interest, 5th Ed. Public Health Service, National Institutes of Health, Bethesda, Md. (1991)). Chothia refers instead to the location of the structural loops. See, e.g., Chothia and Lesk, J. Mol. Biol. 1987, 196, 901-917. The end of the Chothia CDR-H1 loop when numbered using the Kabat numbering convention varies between H32 and H34 depending on the length of the loop (this is because the Kabat numbering scheme places the insertions at H35 A and H35B; if neither 35 A nor 35B is present, the loop ends at 32; if only 35 A is present, the loop ends at 33; if both 35 A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

Recently, a universal numbering system has been developed and widely adopted, ImMunoGeneTics (IMGT) Information System. Lafranc et al., Dev. Comp. Immunol. 2003, 27, 55-77. IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 2001, 309, 657-670. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

Hypervariable regions may comprise “extended hypervariable regions” as follows: 24-36 or 24-34 (L1), 46-56 or 50-56 (L2) and 89-97 or 89-96 (L3) in the VL and 26-35 or 26-35 A (H1), 50-65 or 49-65 (H2) and 93-102, 94-102, or 95-102 (H3) in the VH. As used herein, the terms “HVR” and “CDR” are used interchangeably.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

The term “constant region” or “constant domain” refers to a carboxyl terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

An “affinity matured” antibody is one with one or more alterations (e.g., amino acid sequence variations, including changes, additions and/or deletions) in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. For review, see Hudson and Souriau, Nat. Med. 2003, 9, 129-134; Hoogenboom, Nat. Biotechnol. 2005, 23, 1105-1116; Quiroz and Sinclair, Revista Ingenieria Biomedica 2010, 4, 39-51.

A “blocking” antibody or an “antagonist” antibody is one which inhibits or reduces the binding of the antigen. In certain embodiments, blocking antibodies or antagonist antibodies substantially or completely block the binding of the antigen. In certain embodiments, a “blocking” antibody or an “antagonist” antibody is one which inhibits or reduces the biological activity of the antigen it binds. In other embodiments, the blocking antibodies or antagonist antibodies substantially or completely inhibit the biological activity of the antigen. For example, a blocking anti-SIRP antibody substantially or completely prevents the interaction between SIPRα and CD47.

A “non-blocking” antibody is one which does not inhibit or reduce the binding of the antigen. In certain embodiments, a “non-blocking” antibody is one which does not inhibit or reduce the biological activity of the antigen it binds. In other embodiments, a non-blocking antibody binds to distinct and non-overlapping epitope to which the antigen binds. In some embodiments, a non-blocking antibody is an agonist antibody.

An “agonist antibody” is an antibody that triggers a response, e.g., one that mimics at least one of the functional activities of a polypeptide of interest. An agonist antibody includes an antibody that is a ligand mimetic, for example, wherein a ligand binds to a cell surface receptor and the binding induces cell signaling or activities via an intercellular cell signaling pathway and wherein the antibody induces a similar cell signaling or activation.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

The terms “treat,” “treating,” and “treatment” are meant to include alleviating or abrogating a condition, disorder, or disease, or one or more of the symptoms associated with the condition, disorder, or disease; or alleviating or eradicating the cause(s) of the condition, disorder, or disease itself.

The terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject's risk of acquiring a condition, disorder, or disease.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” are meant to include the amount of a compound that, when administered, is sufficient to prevent development of, or alleviate to some extent, one or more of the symptoms of the condition, disorder, or disease being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 22nd ed.; Allen Ed.: Philadelphia, Pa., 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “CpG-Ab immunoconjugate” or “CpG-Ab” as used herein refers to the linkage of an antibody (Ab) or an antigen binding fragment thereof with a CpG-containing immunostimulating polynucleotide as described herein.

The term “T-cell agonist” as used herein refers to any agent that selectively stimulates the proliferation, differentiation, and/or survival of T cells from a mixed starting population of cells. Thus, the resulting cell population is enriched with an increased number of T cells compared with the starting population of cells. T cell agonists finding use in the present disclosure include but are not limited to antigen molecules specifically binding to T cell receptors (TCRs), as well as T cell co-stimulatory molecules. Examples of T cell co-stimulatory molecules includes but are not limited to OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 and CD83 ligand. In particular embodiments, the T-cell agonist is an antibody against a T cell co-stimulatory molecule. In particular embodiments, the T cell agonist is a tumor associated antigen (TAA). In particular embodiments, the T cell agonist is a pathogenic antigen.

As used herein, an “immune checkpoint” or “immune checkpoint molecule” is a molecule in the immune system that modulates a signal. An immune checkpoint molecule can be a stimulatory checkpoint molecule, i.e., turn up a signal, or inhibitory checkpoint molecule, i.e., turn down a signal. In specific embodiments, immune checkpoint is a protein expressed either by T cells or by antigen presenting cells (APC). Certain types of cancer cells express immune checkpoint proteins to evade immune clearance. Use of immune checkpoint modulators to inhibit the interaction between the immune checkpoint protein expressed by cancer cells and the immune checkpoint protein expressed by T cells has proved effective in certain cancer treatment.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, PD-1, PD-L1, PD-L2, TIM-3, LAG-3, CEACAM-1, CEACAM-5, VISTA, BTLA, TIGIT, LAIR1, CD160, CD47, 2B4, and TGFR. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1 or an anti-PD-L1 antibody.

The term “targeted delivery” or the verb form “target” as used herein refers to the process that promotes the arrival of a delivered agent (such as an immunostimulating polynucleotide) at a specific organ, tissue, cell and/or intracellular compartment (referred to as the targeted location) more than any other organ, tissue, cell or intracellular compartment (referred to as the non-target location). Targeted delivery can be detected using methods known in the art, for example, by comparing the concentration of the delivered agent in a targeted cell population with the concentration of the delivered agent at a non-target cell population after systemic administration. As provided herein, targeted delivery results in at least 2 fold higher concentration at a targeted location as compared to a non-target location. Targeted delivery may be achieved by specific binding of the targeting moiety to a receiving moiety associated with a targeted cell. As used herein, a receiving moiety associated with a targeted cell may be located on the surface or within the cytosol of the targeted cell. In some embodiments, the receiving moiety is an antigen associated with the targeted cell.

The term “DAR” refers to a drug-antibody ratio of an immunomodulating polynucleotide antibody conjugate, more specifically an immunomodulating polynucleotide-antibody ratio.

### Immunomodulating Polynucleotides

In one embodiment, provided herein is an immunomodulating (e.g., immunostimulating) polynucleotide.

In certain embodiments, the immunomodulating polynucleotide comprises a 5-modified uridine or 5-modified cytidine. In certain embodiments, the inclusion of 5-modified uridine (e.g., 5-ethynyl-uridine) at the 5′-terminus of the immunomodulating polynucleotide (e.g., among the two 5′-terminal nucleosides) enhances the immunomodulating properties of the polynucleotide. In certain embodiments, the immunomodulating polynucleotide is shorter (e.g., comprising a total of from about 6 to about 16 nucleotides or from about 12 to about 14 nucleotides) than a typical CpG ODN, which is from 18 to 28 nucleotides in length. In certain embodiments, the shorter immunomodulating polynucleotide (e.g., those comprising a total of from about 6 to about 16 nucleotides or from about 12 to about 14 nucleotides) retains the immunomodulating activity of a longer, typical CpG ODN; or exhibits higher immunomodulating activity (e.g., as measured by NFκB activation or by the changes in the expression levels of at least one cytokine (e.g., IL-6 or IL-10), as compared to the longer CpG ODN. In certain embodiments, the immunomodulating polynucleotide comprises an abasic spacer. In certain embodiments, the immunomodulating polynucleotide comprises an internucleoside phosphotriester. Exemplary descriptions of immunomodulating polypeptides can be found in WO2018189382.

In certain embodiments, the immunomodulating polynucleotide provided herein exhibits stability (e.g., stability against nucleases) that is superior to that of a CpG ODN containing mostly internucleoside phosphate (e.g., more than 50% of internucleoside phosphates) without substantially sacrificing its immunostimulating activity. This effect can be achieved, e.g., by incorporating at least 50% (e.g., at least 70%) internucleoside phosphorothioates or phosphorodithioates or through the inclusion of internucleoside phosphotriesters and/or internucleoside abasic spacers. Phosphotriesters and abasic spacers are also convenient for conjugation to a targeting moiety. Phosphate-based phosphotriesters and abasic spacers can also be used for reduction of off-target activity, relative to polynucleotides with fully phosphorothioate backbones. Without wishing to be bound by theory, this effect may be achieved by reducing self-delivery without disrupting targeting moiety-mediated delivery to target cells. Accordingly, a polynucleotide provided herein can include about 15 or fewer, about 14 or fewer, about 13 or fewer, about 12 or fewer, about 11 or fewer, or about 10 or fewer contiguous internucleoside phosphorothioates. For example, an immunostimulating polynucleotide comprising a total of from about 12 to about 16 nucleosides can contain about 10 or fewer contiguous internucleoside phosphorothioates.

The immunostimulating polynucleotide provided herein can contain a total of about 50 or fewer, about 30 or fewer, about 28 or fewer, or about 16 or fewer nucleosides. The immunostimulating polynucleotide can contain a total of at least 6, about 10 or more, or about 12 or more nucleosides. For example, the immunostimulating polynucleotide can contain a total of from about 6 to about 30, from about 6 to about 28, from about 6 to about 20, from about 6 to about 16, from about 10 to about 20, from about 10 to about 16, from about 12 to about 28, from about 12 to about 20, or from about 12 to about 16 nucleosides.

In certain embodiments, the immunostimulating polynucleotide comprises one or more phosphotriesters (e.g., internucleoside phosphotriesters) and/or phosphorothioates (e.g., from about 1 to about 6 or from about 1 to about 4), e.g., at one or both termini (e.g., within the six 5′-terminal nucleosides or the six 3′-terminal nucleosides). The inclusion of one or more internucleoside phosphotriesters and/or phosphorothioates can enhance the stability of the polynucleotide by reducing the rate of exonuclease-mediated degradation.

In certain embodiments, the immunostimulating polynucleotide comprises a phosphotriester or a terminal phosphodiester, where the phosphotriester or the terminal phosphodiester comprises a linker bonded to a targeting moiety or a conjugating group and optionally to one or more (e.g., from about 1 to about 6) auxiliary moieties. In certain embodiments, the immunostimulating polynucleotide comprises only one linker. In certain embodiments, the immunostimulating polynucleotide comprises only one conjugating group.

The polynucleotide provided herein can be a hybridized polynucleotide including a strand and its partial or whole complement. The hybridized polynucleotides can have at least 6 complementary base pairings (e.g., about 6, about 7, about 8, about 9, about 10, about 11, about 12, about 13, about 14, about 15, about 16, about 17, about 18, about 19, about 20, about 21, about 22, or about 23), up to the total number of the nucleotides present in the included shorter strand. For example, the hybridized portion of the hybridized polynucleotide can contain about 6, about 7, about 8, about 9, about 10, about 11, about 12, about 13, about 14, about 15, about 16, about 17, about 18, about 19, about 20, about 21, about 22, or about 23 base pairs.

In one embodiment, provided herein is an immunostimulating polynucleotide of Formula (A):

X⁵—(X^{N})_{b}—Y^{P}—(X^{N})_{c}—X^{3′} (A)

or a stereoisomer, a mixture of two or more diastereomers, a tautomer, or a mixture of two or more tautomers thereof; or a pharmaceutically acceptable salt, solvate, or hydrate thereof; wherein:

each X^{N} is independently a nucleotide;

X^{3′} is a 3′ terminal nucleotide;

X^{5′} is a 5′ terminal nucleotide;

Y^{P} is an internucleoside phosphotriester; and

b and c are each an integer ranging from about 0 to about 25; with the proviso that their sum is no less than 5.

In certain embodiments, the immunostimulating polynucleotide comprises a nucleotide with a modified nucleobase

In certain embodiments, b is an integer ranging from about 1 to about 15. In certain embodiments, b is an integer of about 1, about 2, about 3, about 4, about 5, about 6, about 7, about 8, about 9, about 10, about 11, about 12, about 13, about 14, or about 15. In certain embodiments, b is an integer of about 3, about 4, about 11, or about 14. In certain embodiments, b is an integer of about 3. In certain embodiments, b is an integer of about 4. In certain embodiments, b is an integer of about 11. In certain embodiments, b is an integer of about 14.

In certain embodiments, c is an integer ranging from about 0 to about 10. In certain embodiments, c is an integer of about 0, about 1, about 2, about 3, about 4, about 5, about 6, about 7, about 8, about 9, or about 10. In certain embodiments, c is an integer of about 0 or about 8. In certain embodiments, c is an integer of about 0. In certain embodiments, c is an integer of about 8.

In certain embodiments, b is an integer of about 3 and c is an integer of about 8. In certain embodiments, b is an integer of about 4 and c is an integer of about 8. In certain embodiments, b is an integer of about 11 and c is an integer of about 0. In certain embodiments, b is an integer of about 14 and c is an integer of about 0.

In certain embodiments, b and c together in total are ranging from about 5 to about 20. In certain embodiments, b and c together in total are ranging from about 5 to about 15. In certain embodiments, b and c together in total are about 5, about 6, about 7, about 8, about 9, about 10, about 11, about 12, about 13, about 14, or about 15. In certain embodiments, b and c together in total are about 8, about 9, about 10, about 11, about 12, about 13, or about 14. In certain embodiments, b and c together in total are about 11. In certain embodiments, b and c together in total are about 12. In certain embodiments, b and c together in total are about 14.

In certain embodiments, each X^{N} is independently a 2′-deoxyribonucleotide or a 2′-modified ribonucleotide. In certain embodiments, each X^{N} is independently 2′-deoxyadenosine (A), 2′-deoxyguanosine (G), 2′-deoxycytidine (C), a 5-halo-2′-deoxycytidine, 2′-deoxythymidine (T), 2′-deoxyuridine (U), a 5-halo-2′-deoxyuridine, a 2′-fluororibonucleotide, a 2′-methoxyribonucleotide, or a 2′-(2-methoxyethoxy)ribonucleotide. In certain embodiments, each X^{N} is independently a 2′-deoxyribonucleotide. In certain embodiments, each X^{N} is independently 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, each X^{N} is independently 2′-deoxyadenosine, 2′-deoxyguanosine, 2′-deoxycytidine, 2′-deoxythymidine, 5-bromo-2′-deoxyuridine, or 5-iodo-2′-deoxyuridine.

In certain embodiments, X^{3′} is a 2′-deoxyribonucleotide or a 2′-modified ribonucleotide. In certain embodiments, X^{3′} is a 2′-deoxyribonucleotide. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleotide, a 2′-methoxyribonucleotide, or a 2′-(2-methoxyethoxy)ribonucleotide. In certain embodiments, X3′ is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X3′ is 2′-deoxythymidine. In certain embodiments, X^{3′} is a 2′-deoxyribonucleotide with a substituted pyrimidine base. In certain embodiments, X^{3′} is a 2′-deoxyribonucleotide with a 5-substituted pyrimidine base. In certain embodiments, X^{3′} is 2′-deoxythymidine, a 5-halo-2′-deoxycytidine, or a 5-halo-2′-deoxyuridine. In certain embodiments, X^{3′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{3′} is 2′-deoxythymidine, 5-bromo-2′-deoxyuridine, or 5-iodo-2′-deoxyuridine. In certain embodiments, X3′ is a terminal nucleotide comprising a 3′ capping group. In certain embodiments, the 3′ capping group is a terminal phosphoester. In certain embodiments, the 3′ capping group is 3-hydroxyl-propylphosphoryl (i.e., —P(O₂)—OCH₂CH₂CH₂OH).

In certain embodiments, X^{5′} is a 2′-deoxyribonucleotide or a 2′-modified ribonucleotide. In certain embodiments, X^{5′} is a 2′-deoxyribonucleotide. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleotide, a 2′-methoxyribonucleotide, or a 2′-(2-methoxyethoxy)ribonucleotide. In certain embodiments, X5′ is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X5′ is a 2′-deoxyribonucleotide with a substituted pyrimidine base. In certain embodiments, X5′ is a 2′-deoxyribonucleotide with a 5-substituted pyrimidine base. In certain embodiments, X^{5′} is 2′-deoxythymidine, a 5-halo-2′-deoxycytidine, or a 5-halo-2′-deoxyuridine. In certain embodiments, X^{5′} is a 5-halo-2′-deoxycytidine. In certain embodiments, X^{5′} is a 5-halo-2′-deoxyuridine. In certain embodiments, X^{5′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{5′} is 2′-deoxythymidine, 5-bromo-2′-deoxyuridine, or 5-iodo-2′-deoxyuridine. In certain embodiments, X^{5′} is 5-bromo-2′-deoxyuridine. In certain embodiments, X^{5′} is 5-iodo-2′-deoxyuridine. In certain embodiments, X^{5′} has a 3′-phosphorothioate group. In certain embodiments, X^{5′} has a 3′-phosphorothioate group with a chirality of Rp. In certain embodiments, X^{5′} has a 3′-phosphorothioate group with a chirality of Sp.

In certain embodiments, Y^{P} is an internucleoside phosphothiotriester.

In certain embodiments, Y^{P} is:

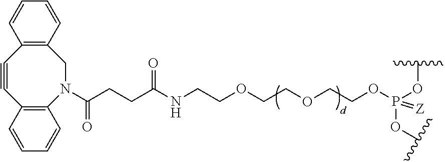

wherein Z is O or S; and d is an integer ranging from about 0 to about 50. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In certain embodiments, Y^{P} is:

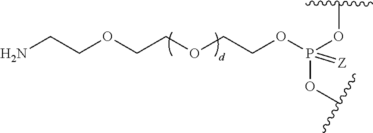

wherein Z is O or S; and d is an integer ranging from about 0 to about 50. In certain embodiments, Y^{P} is:

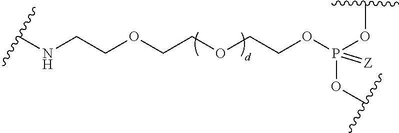

wherein Z is O or S; and d is an integer ranging from about 0 to about 50. In certain embodiments, Y^{P} is:

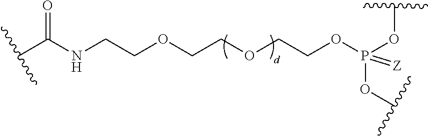

wherein Z is O or S; and d is an integer ranging from about 0 to about 50. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In certain embodiments, the immunostimulating polynucleotide of Formula (A) comprises one additional internucleoside phosphotriester. In one embodiment, the additional internucleoside phosphotriester is a C₁₋₆ alkylphosphotriester. In another embodiment, the additional internucleoside phosphotriester is ethylphosphotriester.

In certain embodiments, the immunostimulating polynucleotide of Formula (A) comprises one 5-halo-2′-deoxyuridine. In one embodiment, the 5-halo-2′-deoxyuridine is 5-fluoro-2′-deoxyuridine, 5-bromo-2′-deoxyuridine, or 5-iodo-2′-deoxyuridine. In another embodiment, the 5-halo-2′-deoxyuridine is 5-bromo-2′-deoxyuridine or 5-iodo-2′-deoxyuridine. In yet another embodiment, the 5-halo-2′-deoxyuridine is 5-fluoro-2′-deoxyuridine. In yet another embodiment, the 5-halo-2′-deoxyuridine is 5-bromo-2′-deoxyuridine. In still another embodiment, the 5-halo-2′-deoxyuridine is 5-iodo-2′-deoxyuridine.

In certain embodiments, the immunostimulating polynucleotide of Formula (A) comprises three or more 2′-deoxycytidines. In certain embodiments, the immunostimulating polynucleotide of Formula (A) comprises three 2′-deoxycytidines.

In certain embodiments, the immunostimulating polynucleotide of Formula (A) comprises four or more 2′-deoxyguanosines. In certain embodiments, the immunostimulating polynucleotide of Formula (A) comprises four 2′-deoxyguanosines.

In certain embodiments, the immunostimulating polynucleotide of Formula (A) comprises three 2′-deoxycytidines and four 2′-deoxyguanosines. In certain embodiments, the immunostimulating polynucleotide of Formula (A) comprises one, two, or three CG dinucleotides. In certain embodiments, the immunostimulating polynucleotide of Formula (A) comprises three CG dinucleotides.

In certain embodiments, the immunostimulating polynucleotide of Formula (A) comprises three or more 2′-deoxythymidines. In certain embodiments, the immunostimulating polynucleotide of Formula (A) comprises three, four, five, six, seven, or eight 2′-deoxythymidines. In certain embodiments, the immunostimulating polynucleotide of Formula (A) comprises three, four, five, or eight 2′-deoxythymidines.

In certain embodiments, the immunostimulating polynucleotide of Formula (A) does not comprise a 2′-deoxyadenosine. In certain embodiments, the immunostimulating polynucleotide of Formula (A) comprises one or two 2′-deoxyadenosines.

In certain embodiments, the immunostimulating polynucleotide of Formula (A) has a length ranging from about 5 to about 20 or from about 6 to about 15. In certain embodiments, the immunostimulating polynucleotide of Formula (A) has a length of about 6, about 7, about 8, about 9, about 10, about 11, about 12, about 13, about 14, or about 15. In certain embodiments, the immunostimulating polynucleotide of Formula (A) has a length of about 10, about 11, about 12, about 13, about 14, or about 15.

In certain embodiments, the immunostimulating polynucleotide of Formula (A) comprises one or more internucleoside phosphorothioates. In certain embodiments, all the internucleoside phosphoesters in the immunostimulating polynucleotide of Formula (A) are internucleoside phosphorothioates. In certain embodiments, the immunostimulating polynucleotide of Formula (A) comprises one or more chiral internucleoside phosphorothioates.

In certain embodiments, the immunostimulating polynucleotide of Formula (A) is p275, p276, p313, or p347. In certain embodiments, the immunostimulating polynucleotide of Formula (A) is p236, p238, p243, p246, p308, p361, p362, or p425. In certain embodiments, the immunostimulating polynucleotide of Formula (A) is p236, p238, p243, p246, p275, p276, p308, p313, p347, p361, p362, p425, p433, p434, p435, p436, p437, p438, p477, p478, p479, p480, p481, p482, p483, p484, p485, p486, p487, p488, or p489.

In one embodiment, provided herein is an immunostimulating polynucleotide having a sequence of N¹N²CGN³CG(T)ₓGN⁴CGN⁵T (SEQ ID NO:586), or a stereoisomer, a mixture of two or more diastereomers, a tautomer, or a mixture of two or more tautomers thereof; or a pharmaceutically acceptable salt, solvate, or hydrate thereof; wherein:

x is an integer ranging from about 1 to about 4;

N¹ is absent or 2′-deoxythymidine;

N² is a 2′-deoxyribonucleotide with a modified nucleobase;

N³ is 2′-deoxyadenosine or 2′-deoxythymidine, each optionally comprising a 3′-phosphotriester;

N⁴ is 2′-deoxyadenosine or 2′-deoxythymidine;

N⁵ is 2′-deoxythymidine optionally comprising a 3′-phosphotriester; and

C is 2′-deoxycytidine and G is 2′-deoxyguanosine.

In certain embodiments, in N¹N²CGN³CG(T)ₓGN⁴CGN⁵T (SEQ ID NO:586), x is an integer of about 1, about 2, about 3, or about 4. In certain embodiments, in N¹N²CGN³CG(T)ₓGN⁴CGN⁵T (SEQ ID NO:586), x is an integer of about 1. In certain embodiments, in N¹N²CGN³CG(T)ₓGN⁴CGN⁵T (SEQ ID NO:586), x is an integer of about 4.

In certain embodiments, in N¹N²CGN³CG(T)ₓGN⁴CGN⁵T (SEQ ID NO:586), N¹ is absent. In certain embodiments, in N¹N²CGN³CG(T)ₓGN⁴CGN⁵T (SEQ ID NO:586), N¹ is 2′-deoxythymidine.

In certain embodiments, in N¹N²CGN³CG(T)ₓGN⁴CGN⁵T (SEQ ID NO:586), N² is a 2′-deoxyribonucleotide with a substituted pyrimidine base. In certain embodiments, in N¹N²CGN³CG(T)ₓGN⁴CGN⁵T (SEQ ID NO:586), N² is a 2′-deoxyribonucleotide with a 5-substituted pyrimidine base. In certain embodiments, in N¹N²CGN³CG(T)ₓGN⁴CGN⁵T (SEQ ID NO:586), N² is a 5-halo-2′-deoxycytidine or a 5-halo-2′-deoxyuridine. In certain embodiments, in N¹N²CGN³CG(T)ₓGN⁴CGN⁵T (SEQ ID NO:586), N² is 5-bromo-2′-deoxyuridine or 5-iodo-2′-deoxyuridine.

In certain embodiments, in N¹N²CGN³CG(T)ₓGN⁴CGN⁵T (SEQ ID NO:586), N³ is 2′-deoxyadenosine comprising a 3′-phosphotriester. In certain embodiments, in N¹N²CGN³CG(T)ₓGN⁴CGN⁵T (SEQ ID NO:586), N³ is 2′-deoxythymidine. In certain embodiments, in N¹N²CGN³CG(T)ₓGN⁴CGN⁵T (SEQ ID NO:586), N³ is 2′-deoxythymidine comprising a 3′-phosphotriester.

In certain embodiments, in N¹N²CGN³CG(T)ₓGN⁴CGN⁵T (SEQ ID NO:586), N⁴ is 2′-deoxyadenosine. In certain embodiments, in N¹N²CGN³CG(T)ₓGN⁴CGN⁵T (SEQ ID NO:586), N⁴ is 2′-deoxythymidine.

In certain embodiments, in N¹N²CGN³CG(T)ₓGN⁴CGN⁵T (SEQ ID NO:586), N⁵ is 2′-deoxythymidine. In certain embodiments, in N¹N²CGN³CG(T)ₓGN⁴CGN⁵T (SEQ ID NO:586), N⁵ is 2′-deoxythymidine comprising a 3′-phosphotriester.

In certain embodiments, the immunostimulating polynucleotide of N¹N²CGN³CG(T)ₓGN⁴CGN⁵T (SEQ ID NO:586) comprises one or more internucleoside phosphorothioates. In certain embodiments, the immunostimulating polynucleotide of N¹N²CGN³CG(T)ₓGN⁴CGN⁵T (SEQ ID NO:586) comprises at least one chiral internucleoside phosphorothioates.

In certain embodiments, the immunostimulating polynucleotide of N¹N²CGN³CG(T)ₓGN⁴CGN⁵T is p275, p276, or p313. In certain embodiments, the immunostimulating polynucleotide of N¹N²CGN³CG(T)ₓGN⁴CGN⁵T (SEQ ID NO:586) is p236, p238, p243, p246, p308, p361, p362, or p425. In certain embodiments, the immunostimulating polynucleotide of N¹N²CGN³CG(T)ₓGN⁴CGN⁵T is p236, p238, p243, p246, p275, p276, p308, p313, p347, p361, p362, p425, p433, p434, p435, p436, p437, p438, p477, p478, p479, p480, p481, p482, p483, p484, p485, p486, p487, p488, or p489.

In certain embodiments, the immunostimulating polynucleotide provided herein is an immunostimulating polynucleotide. In certain embodiments, the immunostimulating polynucleotide provided herein functions as a PAMS. In certain embodiments, the immunostimulating polynucleotide provided herein activates innate immune response or stimulates the adaptive immune response by triggering TLR9 signaling. In certain embodiments, the immunostimulating polynucleotide provided herein is a TLR9 agonist.

In certain embodiments, the immunostimulating polynucleotide provided herein is a class B CpG polynucleotide, or its modification including 5-halouridine or 5-alkynyluridine, or a truncated version thereof (e.g., those comprising a total of about 6 to about 16 nucleosides). In certain embodiments, the truncated immunostimulating polynucleotide provided herein comprises a truncated class B CpG polynucleotide sequence (e.g., a class B CpG polynucleotide sequence, from which one or more 3′-terminal nucleotides are eliminated or one or more of the intra-sequence nucleotides excised).

In certain embodiments, the immunostimulating polynucleotide provided herein comprises at least one immunostimulating sequence (ISS). In certain embodiments, the immunostimulating polynucleotide provided herein comprises about 1, about 2, about 3, or about 4 ISS. The ISS in immunostimulating polynucleotides is dependent on the targeted organism. The common feature of the ISS used in the immunostimulating polynucleotide provided herein is the cytidine-p-guanosine sequence, in which p is an internucleoside phosphodiester (e.g., phosphate or phosphorothioate) or an internucleoside phosphotriester. In certain embodiments, cytidine and guanosine in the ISS each independently comprises 2′-deoxyribose. In certain embodiments, the immunostimulating polynucleotide provided herein comprises about 1, about 2, or about 3 human ISSs. In certain embodiments, the human ISS is CGor NCG, where Nis uridine, cytidine, or thymidine, or a modified uridine or cytidine; and G is guanosine or a modified guanosine. In certain embodiments, the modified uridine or cytidine is a 5-halouridine (e.g., 5-iodouridine or 5-bromouridine), a 5-alkynyluridine (e.g., 5-ethynyluridine or 5-propynyluridine), 5-heteroaryluridine, or 5-halocytidine. In certain embodiments, the modified guanosine is 7-deazaguanosine. In certain embodiments, the human ISS is NCG, in one embodiment, Nis 5-halouridine. In certain embodiments, the human ISS is UCG, in one embodiment, Uis 5-alkynyluridine, and in another embodiment, U is 5-ethynyluridine. In certain embodiments, the immunostimulating polynucleotide provided herein targeting humans comprises an ISS within four contiguous nucleotides that include a 5′-terminal nucleotide. In certain embodiments, the immunostimulating polynucleotide provided herein targeting humans comprises a 5′-terminal ISS. In certain embodiments, the immunostimulating polynucleotide provided herein comprises a murine ISS. In certain embodiments, the murine ISS is a hexameric nucleotide sequence: Pu—Pu-CG-Py-Py, where each Pu is independently a purine nucleotide, and each Py is independently a pyrimidine nucleotide.

In certain embodiments, the 5′-flanking nucleotides relative to CpG in the immunostimulating polynucleotide provided herein does not contain 2′-alkoxyriboses. In certain embodiments, the 5′-flanking nucleotides relative to CpG in the immunostimulating polynucleotide provided herein comprises only 2′-deoxyriboses as sugars.

In certain embodiments, the immunostimulating polynucleotide provided herein has (1) a high content of phosphorothioates (e.g., at least 50%, at least 60%, at least 70%, or at least 80% of nucleosides may be linked by phosphorothioates); (2) absence of poly-G tails; (3) nucleosides in the immunostimulating polynucleotide comprises 2′-deoxyriboses or 2′-modified riboses (e.g., 2′-halo (e.g., 2′-fluoro) or optionally substituted 2′-alkoxy (e.g., 2′-methoxy)); and/or (4) the inclusion of 5′-terminal ISS that is NCG, in which Nis uridine, cytidine, or thymidine, or a modified uridine or cytidine, and G is guanosine or a modified guanosine.

In certain embodiments, the immunomodulating polynucleotide provided herein suppresses the adaptive immune response by reducing activation of TLR9 signaling (e.g., through TLR9 antagonism). In certain embodiments, the immunosuppressive polynucleotide provided herein comprises at least two 2′-alkoxynucleotides that are 5′-flanking relative to CpG as described by the formula of N¹—N²—CG, where N¹ and N² are each independently a nucleotide containing 2′-alkoxyribose (e.g., 2′-methoxyribose).

### Structural Features of Immunomodulating Polynucleotides

Abasic Spacers

In certain embodiments, the immunomodulating polynucleotides provided herein comprises one or more, in one embodiment, one or two abasic spacers, each of which is independently an internucleoside abasic spacer or terminal abasic spacer. When the immunomodulating polynucleotide includes two or more of the abasic spacers, the structures of the abasic spacers can be same or different.

In one embodiment, the abasic spacer is of formula (I):

R¹-L¹-[-L²-(L¹)ₙ₁-]ₙ₂-R², (I)

wherein:

n1 is an integer of about 0 or about 1,

n2 is an integer from about 1 to about 6,

R¹ is a bond to a nucleoside in the immunomodulating polynucleotide,

R² is a bond to a nucleoside in the immunomodulating polynucleotide or to a capping group,

each L¹ is independently a phosphodiester or a phosphotriester, and

each L² is a sugar analogue.

In certain embodiments, if the abasic spacer is an internucleoside abasic spacer, n1 is about 1, and R² is a bond to a nucleoside; and if the abasic spacer is a terminal abasic spacer, n1 is about 0 or about 1, and R² is a bond to a capping group.

In certain embodiments, the abasic spacer is an internucleoside abasic spacer. In certain embodiments, the abasic spacer is a 3′-terminal abasic spacer. In certain embodiments, each two contiguous L² groups are separated by L¹ groups (e.g., n1 is 1 for L¹ disposed between two contiguous L² groups).

In certain embodiments, the immunostimulating polynucleotide provided herein comprises an ISS disposed within four contiguous nucleotides that include a 5′-terminal nucleotide of the immunostimulating polynucleotide, where the ISS is NCG, where Nis uridine, cytidine, or thymidine, or a modified uridine or cytidine, in one embodiment.., a 5-halouridine (e.g., 5-iodouridine or 5-bromouridine), a 5-alkynyluridine (e.g., 5-ethynyluridine or 5-propynyluridine), 5-heteroaryluridine, or 5-halocytidine; and where N and C are linked to each other through a phosphodiester or phosphotriester.

Sugar Analogues

In one embodiment, a sugar analogue is a divalent or trivalent group that is a C₃₋₆ monosaccharide or C₃₋₆ alditol (e.g., glycerol), which is modified to replace two hydroxyl groups with bonds (i) to an oxygen atom in one phosphoester and (ii) to an oxygen atom in another phosphoester or to a capping group. A sugar analogue is cyclic or acyclic. Further optional modifications included in a sugar analogue are: a replacement of one, two, or three of the remaining hydroxyl groups or carbon-bonded hydrogen atoms with H; optionally substituted C₁₋₆ alkyl; -LinkA(-T)ₚ, as defined herein; a conjugating group; —(CH₂)ₜ₁—OR^{Z}, where t1 is an integer from about 1 to about 6, and R^{Z} is optionally substituted C₁₋₆ alkyl, optionally substituted C₂₋₆ alkenyl, optionally substituted C₂₋₆ alkynyl, optionally substituted C₆₋₁₄ aryl, optionally substituted C₃₋₈ cycloalkyl, optionally substituted (C₁₋₉ heterocyclyl)-C₁₋₆-alkyl, optionally substituted (C₆₋₁₀ aryl)-C₁₋₆-alkyl, or optionally substituted (C₃₋₈ cycloalkyl)-C₁₋₆-alkyl; introduction of one or two unsaturation(s) (e.g., one or two double bonds); and replacement of one, two, or three hydrogens or hydroxyl groups with substituents as defined for alkyl, alkenyl, cycloalkyl, cycloalkenyl, or heterocyclyl. In some embodiments, R^{Z} is optionally substituted C₁₋₆ aminoalkyl (e.g., optionally substituted C₁₋₆ amino alkyl containing —NH₂).

Non-limiting examples of sugar analogues are optionally substituted C₂₋₆ alkylene, optionally substituted C₂₋₆ alkenylene, optionally substituted C₅ cycloalkane-1,3-diyl, optionally substituted C₅ cycloalkene-1,3-diyl, optionally substituted heterocycle-1,3-diyl (e.g., optionally substituted pyrrolidine-2,5-diyl, optionally substituted tetrahydrofuran-2,5-diyl, or optionally substituted tetrahydrothiophene-2,5-diyl), or optionally substituted (C₁₋₄ alkyl)-(C₃₋₈ cycloalkylene) (e.g., optionally substituted (C₁ alkyl)-(C₃ cycloalkylene)). Non-limiting examples of sugar analogues are:

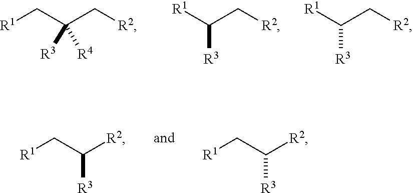

wherein:

each of R¹ and R² is independently a bond to an oxygen atom in a phosphoester;

each of R³ and R⁴ is independently H; optionally substituted C₁₋₆ alkyl; —(CH₂)ₜ₁—OR^{Z}; or -LinkA-RT;

where t1 is an integer from about 1 to about 6;

R^{Z} is optionally substituted C₁₋₆ alkyl, optionally substituted C₂₋₆ alkenyl, optionally substituted C₂₋₆ alkynyl, optionally substituted C₆₋₁₄ aryl, optionally substituted C₃₋₈ cycloalkyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl;

LinkA is a linker; and

R^{T} is a bond to a targeting moiety; a conjugation moiety; optionally substituted C₁₋₆ alkyl, optionally substituted C₂₋₆ alkenyl, optionally substituted C₂₋₆ alkynyl, optionally substituted C₆₋₁₄ aryl, optionally substituted C₃₋₈ cycloalkyl, optionally substituted (C₁₋₉ heterocyclyl)-C₁₋₆-alkyl, optionally substituted (C₆₋₁₀ aryl)-C₁₋₆-alkyl, or optionally substituted (C₃₋₈ cycloalkyl)-C₁₋₆-alkyl.

In certain embodiments, R^{Z} is optionally substituted C₁₋₆ aminoalkyl (e.g., optionally substituted C₁₋₆ amino alkyl containing —NH₂).

Phosphoesters

In certain embodiments, the immunomodulating polynucleotide provided herein comprises one or more internucleoside phosphotriesters and/or one or two terminal phosphodiesters and/or phosphotriesters. In certain embodiments, a phosphotriester comprises a phosphate, phosphorothioate, or phosphorodithioate, in which one or two valencies are substituted with nucleosides and/or abasic spacers, and the remaining valencies are bonded to a bioreversible group, a non-bioreversible group, a linker bonded to a targeting moiety, or a conjugating group. In certain embodiments, an internucleoside phosphotriester is bonded to two nucleosides and/or abasic spacers, and the remaining valency is bonded to a bioreversible group, a non-bioreversible group, a linker bonded to a targeting moiety, or a conjugating group. In certain embodiments, an internucleoside phosphodiester is bonded to two nucleosides and/or abasic spacers. In certain embodiments, a terminal phosphodiester comprises a phosphate, phosphorothioate, or phosphorodithioate at the 5′- or 3′-terminus of the immunomodulating polynucleotide, where one of the two remaining valencies is bonded to a bioreversible group, a non-bioreversible group, a linker bonded to a targeting moiety, or a conjugating group.

Linkers and Conjugation Moieties

In certain embodiments, the immunomodulating polynucleotide provided herein comprises a linker bonded to a targeting moiety and optionally one or more auxiliary moieties. In certain embodiments, the linker is a multivalent group, in which the first valency is bonded to an internucleoside or terminal phosphate, an internucleoside or terminal phosphorothioate, an internucleoside or terminal phosphorodithioate, an abasic spacer, a capping group, or a nucleobase, and a second valency is bonded to a targeting moiety. In certain embodiments, the linker further include one or more valencies, each of which is independently bonded to an auxiliary moiety. In certain embodiments (e.g., when the targeting moiety is a small molecule), the immunomodulating polynucleotide provided herein comprises multiple linkers to multiple targeting moieties. In certain embodiments (e.g., when the targeting moiety is an antibody or an antigen-binding fragment thereof), the immunomodulating polynucleotide provided herein comprises one linker to a targeting moiety.

In certain embodiments, the immunomodulating polynucleotide provided herein comprises a conjugating group. A conjugating group is a functional group that is capable of undergoing a conjugation reaction, e.g., a cycloaddition reaction (e.g., dipolar cycloaddition), amidation reaction, or nucleophilic aromatic substitution. Upon reaction with a complementary reactive group, the conjugating group produces the linker in the immunomodulating polynucleotide provided herein.

In certain embodiments, the linker bonded to a targeting moiety is a part of an internucleoside phosphotriester. In certain embodiments, the linker bonded to a targeting moiety is a part of an abasic spacer.

In certain embodiments, the linker or a conjugating group is of formula (II):

—Z¹-Q^{A1}-Z²—(-Q²-Z³—)ₖ-RT, (II)

wherein:

Z¹ is a divalent group, a trivalent group, a tetravalent group, or a pentavalent group, in which one of valency is bonded to Q^{A1}, the second valency is open or, if formula (II) is for the linker, is bonded to R^{T}, and each of the remaining valencies, when present, is independently bonded to an auxiliary moiety;

Z² is absent, a divalent group, a trivalent group, a tetravalent group, or a pentavalent group, in which one of valency is bonded to Q^{A1}, the second valency is bonded to Q^{A2} or R^{T}, and each of the remaining valencies, when present, is independently bonded to an auxiliary moiety;

Z³ is absent, a divalent group, a trivalent group, a tetravalent group, or a pentavalent group, in which one of valency is bonded to Q^{A2}, the second valency is bonded to R^{T}, and each of the remaining valencies, when present, is independently bonded to an auxiliary moiety;

R^{T} is absent or a bond to a targeting moiety;

k is an integer of about 0 or about 1.

If formula (II) is for the linker,

Q^{A1} and Q^{A2} is independently absent, optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

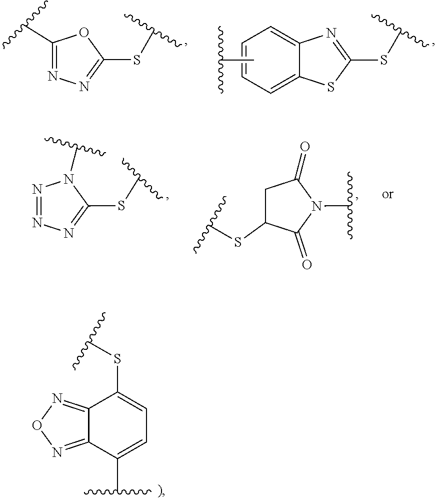

optionally substituted C₁₋₁₂ heterocyclylene (e.g., 1,2,3-triazole-1,4-diyl or

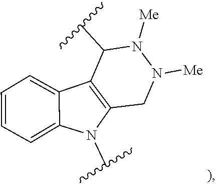 [SMILES: *C1c2c(n(*)c3ccccc23)CN(C)N1C]

cyclobut-3-ene-1,2-dione-3,4-diyl, pyrid-2-yl hydrazone, optionally substituted C₆₋₁₆ triazoloheterocyclylene (e.g.,

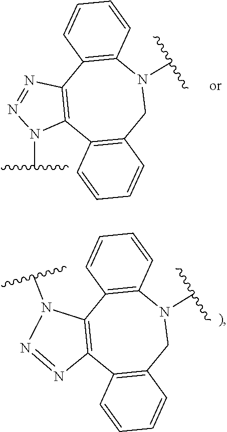

optionally substituted C₈₋₁₆ triazolocycloalkenylene (e.g.,

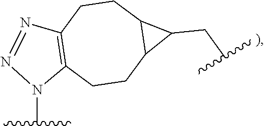 [SMILES: *CC1C2CCc3nnn(*)c3CCC12]

or a dihydropyridazine group (e.g.,

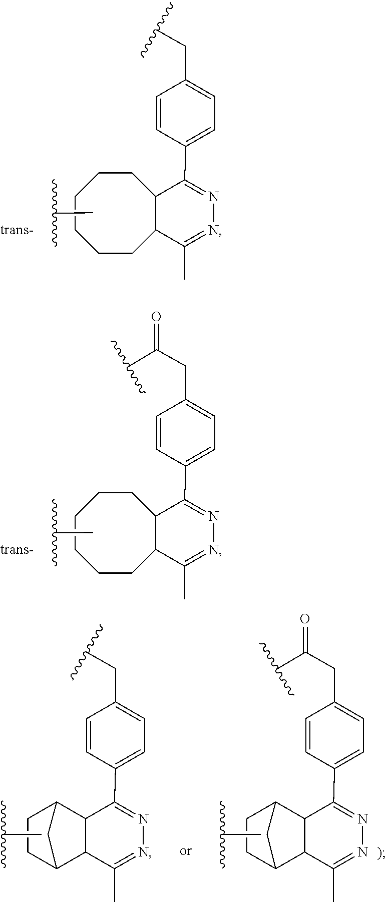

and

R^{T} is a bond to a targeting moiety;

provided that at least one of Q^{A1} and Q^{A2} is present.

If formula (II) is for a conjugating group,

either

(i) Q^{A2} is absent, and Q^{A1} is a conjugation moiety, e.g., optionally substituted C₂₋₁₂ alkynyl, optionally substituted N-protected amino, azido, N-maleimido, S-protected thiol,

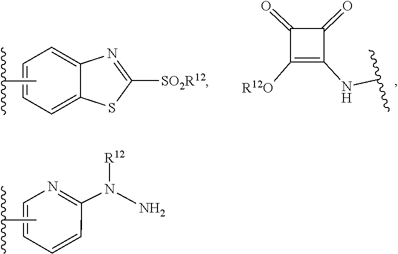

or an N-protected version thereof,

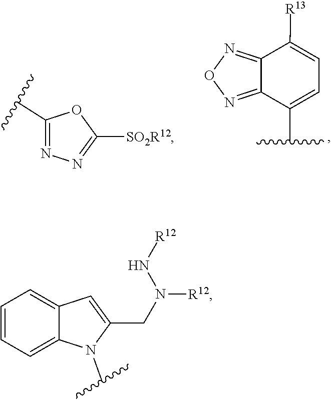

optionally substituted C₆₋₁₆ heterocyclyl containing an endocyclic carbon-carbon triple bond (e.g.,

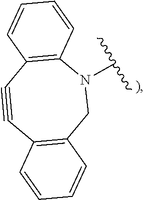 [SMILES: *N1Cc2ccccc2C#Cc2ccccc21]

1,2,4,5-tetrazine group (e.g.,

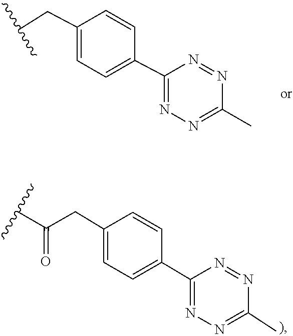 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

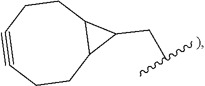 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO; and

k is an integer of about 0;

or

(ii) Q^{A1} is as defined for the linker, and Q^{A2} is a conjugation moiety, e.g., optionally substituted C₂₋₁₂ alkynyl, optionally substituted N-protected amino, azido, N-maleimido, S-protected thiol,

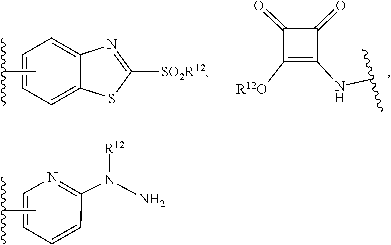

or an N-protected version thereof,

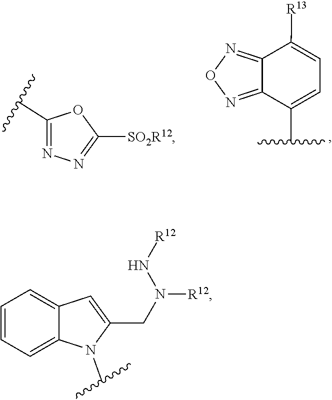

optionally substituted C₆₋₁₆ heterocyclyl containing an endocyclic carbon-carbon triple bond (e.g.,

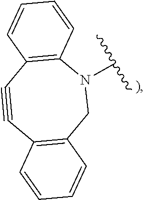 [SMILES: *N1Cc2ccccc2C#Cc2ccccc21]

1,2,4,5-tetrazine group (e.g.,

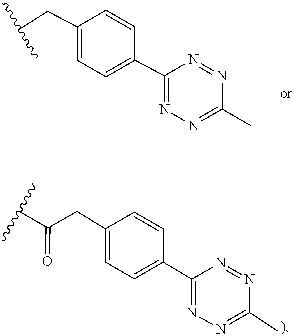 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

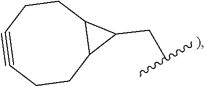 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO; and

k is an integer of about 1;

where:

R^{N1} is H, N-protecting group, or optionally substituted C₁₋₆ alkyl;

each R¹² is independently H or optionally substituted C₁₋₆ alkyl;

R¹³ is halogen or F; and

Z³ and R^{T} are absent.

In certain embodiments, Z¹ has a branching group and two divalent segments, where the branching group is bonded to each of the two divalent segments,

wherein:

one of the divalent segments is bonded to an internucleoside or terminal phosphate, an internucleoside or terminal phosphorothioate, an internucleoside or terminal phosphorodithioate, an abasic spacer, or a nucleobase, and the remaining divalent segment is bonded to Q^{A1};

the branching group is optionally substituted C₁₋₁₂ alkane-triyl or optionally substituted C₂₋₁₂ heteroalkane-triyl, in which two valencies are substituted with the divalent segments, and the remaining valency is substituted with

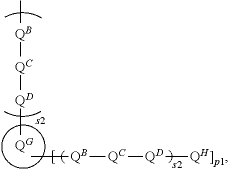

wherein:
p1 is an integer of about 1, about 2, or about 3;
each s2 is independently an integer from about 0 to about 10;
each Q^{B} and Q^{D} are independently absent, —CO—, —NH—, —O—, —S—, —SO₂—, —OC(O)—COO—, —NHC(O)—, —C(O)NH—, —CH₂—, —CH₂NH—, —NHCH₂—, —CH₂O—, or —OCH₂—;
each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₁₋₉ heterocyclylene, or —P(Z)(OH)—, where Z is O or S;
each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each Q^{H} is independently R^{M1} or -Q^{G}[(Q^{B}-Q^{C}-Q^{D})ₛ₂—R^{M1}]ₚ₁, where each R^{M1} is independently a bond to an auxiliary moiety.

In certain embodiments, Z² has a branching group and two divalent segments, where the branching group is bonded to each of the two divalent segments,

wherein:

one of the divalent segments is bonded to a targeting moiety or Q^{A2}, and the remaining divalent segment is bonded to Q^{A1};

the branching group is optionally substituted C₁₋₁₂ alkane-triyl or optionally substituted C₂₋₁₂ heteroalkane-triyl, in which two valencies are substituted with the divalent segments, and the remaining valency is substituted with

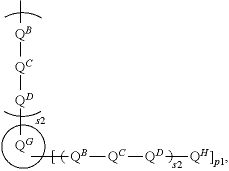

wherein:
p1 is an integer of about 1, about 2, or about 3;
each s2 is independently an integer from about 0 to about 10;
each Q^{B} and Q^{D} are independently absent, —CO—, —NH—, —O—, —S—, —SO₂—, —OC(O)—COO—, —NHC(O)—, —C(O)NH—, —CH₂—, —CH₂NH—, —NHCH₂—, —CH₂O—, or —OCH₂—;
each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₁₋₉ heterocyclylene, or —P(Z)(OH)—, where Z is O or S;
each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each Q^{H} is independently R^{M1} or -Q^{G}[(Q^{B}-Q^{C}-Q^{D})ₛ₂—R^{M1}]ₚ₁, where each R^{M1} is independently a bond to an auxiliary moiety.

In certain embodiments, Z³ has a branching group and two divalent segments, where the branching group is bonded to each of the two divalent segments, wherein:

one of the divalent segments is bonded to a targeting moiety, and the remaining divalent segment is bonded to Q^{A2};

the branching group is optionally substituted C₁₋₁₂ alkane-triyl or optionally substituted C₂₋₁₂ heteroalkane-triyl, in which two valencies are substituted with the divalent segments, and the remaining valency is substituted with

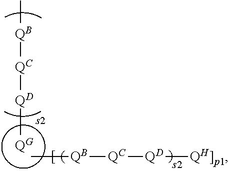

wherein:
p1 is an integer of about 1, about 2, or about 3;
each s2 is independently an integer from about 0 to about 10;
each Q^{B} and Q^{D} are independently absent, —CO—, —NH—, —O—, —S—, —SO₂—, —OC(O)—COO—, —NHC(O)—, —C(O)NH—, —CH₂—, —CH₂NH—, —NHCH₂—, —CH₂O—, or —OCH₂—;
each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₁₋₉ heterocyclylene, or —P(Z)(OH)—, where Z is O or S;
each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each Q^{H} is independently R^{M1} or -Q^{G}[(Q^{B}-Q^{C}-Q^{D})ₛ₂—R^{M1}]ₚ₁, where each R^{M1} is independently a bond to an auxiliary moiety.

In certain embodiments, the divalent segment in Z¹, Z², or Z³ is -(-Q^{B}-Q^{C}-Q^{D}-)ₛ₁- wherein:

each s1 is independently an integer from about 1 to about 50 or from about 1 to about 30;

each Q^{B} and Q^{D} are independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—; and

each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene;

provided that at least one of Q^{B}, Q^{C}, and Q^{D} is present.

In certain embodiments, at least one Q^{C} is present in the divalent segment. In certain embodiments, Q^{C} is present in each monomeric unit of the divalent segment. In certain embodiments, Z¹ is bonded through a Q^{C} that is present. In certain embodiments, at least one of Q^{B} and Q^{D} is present in each monomeric unit of Z¹. In certain embodiments, at least one of Q^{B} and Q^{D} is present in each monomeric unit of Z². In certain embodiments, only one of Z¹, Z², and Z³, when present, contains a branching group.

In certain embodiments, one, two, or three of Z¹, Z², and Z³ are independently

-(-Q^{B}-Q^{C}-Q^{D}-)ₛ₁-Q^{E}-(-Q^{B}-Q^{C}-Q^{D}-)ₛ₁-, (III)

wherein:

each s1 is independently an integer from about 1 to about 50 or from about 1 to about 30;

each Q^{B} and Q^{D} are independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;

each Q^{c} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₁₋₉ heterocyclylene, or —P(Z)(OH)—, where Z is 0 or S; and

Q^{E} is absent or a branching group of formula (IV):

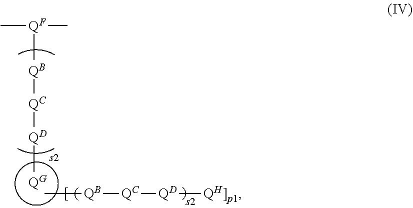

wherein:
p1 is an integer of about 1, about 2, or about 3;
each s2 is independently an integer from about 0 to about 10;
Q^{F} is optionally substituted C₁₋₁₂ alkane-triyl or optionally substituted C₂₋₁₂ heteroalkane-triyl; and
each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each Q^{H} is independently R^{M1} or -Q^{G}[(-Q^{B}-Q^{C}-Q^{D})₂—R^{M1}]ₚ₁, where each R^{M1} is independently a bond to an auxiliary moiety.

In formula (IV), Q^{G} is absent, if p1 is about 1; and at least one Q^{G} is present, if p1 is 2 or 3.

In certain embodiments, Z¹ is bonded to an internucleoside or terminal phosphate, an internucleoside or terminal phosphorothioate, an internucleoside or terminal phosphorodithioate, an abasic spacer, a capping group, or a nucleobase through a Q^{c} that is present.

In certain embodiments, at least one of Q^{B}, Q^{C}, Q^{D}, and Q^{E} is present (e.g., at least one Q^{C} is present, Q^{E} is present, or Q^{E} is absent) in the divalent segment. In certain embodiments, each Q^{B} and Q^{D} are independently absent, —CO—, —NH—, —O—, —S—, —SO₂—, —NHC(O)—, —C(O)NH—, —CH₂—, —CH₂NH—, —NHCH₂—, —CH₂O—, or —OCH₂—.

In certain embodiments, -(-Q^{B}-Q^{C}-Q^{D}-)ₛ₁-combine to form a group:

-Q^{B}-(CH₂)_{g1}—(CH₂OCH₂)_{g2}—(CH₂)_{g3}-Q^{D}-,

wherein:

g2 is an integer from about 1 to about 50;

g1 is an integer of about 1 and Q^{B} is —NHCO—, —CONH—, or —O—; or g1 is an integer of about 0 and Q^{D} is —NHCO—; and

g3 is an integer of about 1 and Q^{B} is —NHCO—, —CONH—, or —O—; or g3 is an integer of about 0 and Q^{D} is —CONH—.

The conjugation moiety may be protected until an auxiliary moiety is conjugated to the polynucleotide. For example, a conjugation moiety that is protected may include —COOR^{PGO} or —NHR^{PGN}, where R^{PGO} is an O-protecting group (e.g., a carboxyl protecting group), and R^{PGN} is an N-protecting group.

In certain embodiments, Link A is:

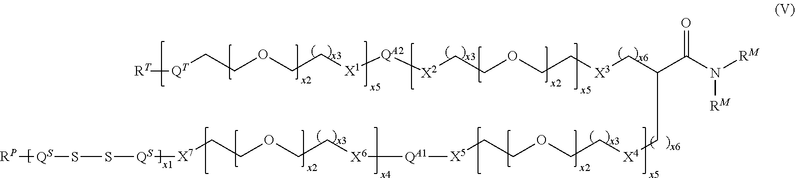

wherein:

Q^{A1} and Q^{A2} are each independently absent, optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

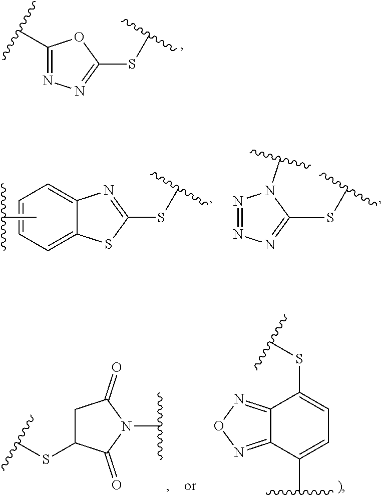

optionally substituted C₁₋₁₂ heterocyclylene (e.g., 1,2,3-triazole-1,4-diyl or

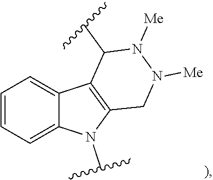 [SMILES: *C1c2c(n(*)c3ccccc23)CN(C)N1C]

cyclobut-3-ene-1,2-dione-3,4-diyl, pyrid-2-yl hydrazone, optionally substituted C₆₋₁₆ triazoloheterocyclylene (e.g.,

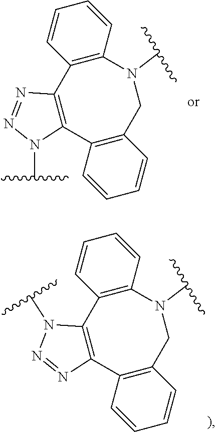

optionally substituted C₈₋₁₆ triazolocycloalkenylene (e.g.,

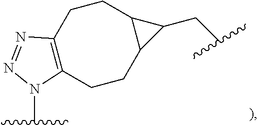 [SMILES: *CC1C2CCc3nnn(*)c3CCC12]

or a dihydropyridazine group (e.g.,

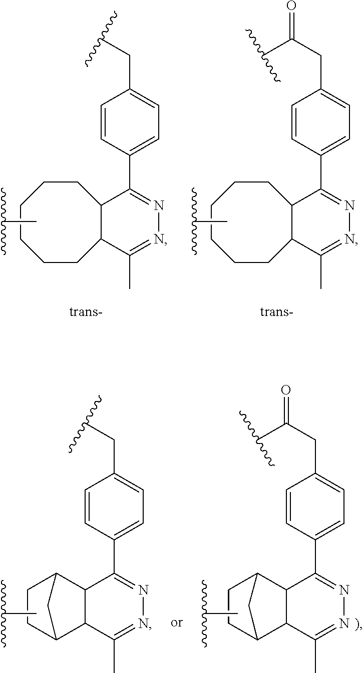

provided that at least one of Q^{A1} and Q^{A2} is present;

R^{T} is a bond to a targeting moiety;

R^{P} is a bond to an internucleoside bridging group, a nucleobase, a capping group, or an abasic spacer;

each Q^{T} is independently —CO—, —NH—, —NH—CH₂—, or —CO—CH₂—;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each R^{M} is independently H, auxiliary moiety, —(CH₂)_{q7}—CO—N(R^{M1})₂, or —C[—CH₂O—(CH₂)_{q7}—CO—N(R^{M1})₂]₃, where each q7 is independently an integer from about 1 to about 5, and each R^{M}1 is independently H or an auxiliary moiety;

each X¹, X³, and X⁵ are independently absent, —O—, —NH—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

X⁷ is absent, —O—, —O—P(O)(OH)—O—, —O—P(S)(OH)—O—, —NH—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

each of X², X⁴, and X⁶ is independently absent, —O—, —NH—, —C(O)—, —C(O)—NH—, —NH—C(O)—, —NH—C(O)—NH—, —O—C(O)—NH—, or —NH—C(O)—O—;

x1 and each x5 are independently an integer of about 0 or about 1;

each x2 is independently an integer from about 0 to about 50, from about 1 to about 40, or from about 1 to about 30;

each x3 is independently an integer from about 1 to about 11;

x4 is an integer of about 0, about 1, or about 2; and

each x6 is independently an integer from about 0 to about 10 or from about 1 to about 6, provided that the sum of both x6 is about 12 or less.

In certain embodiments, LinkA is:

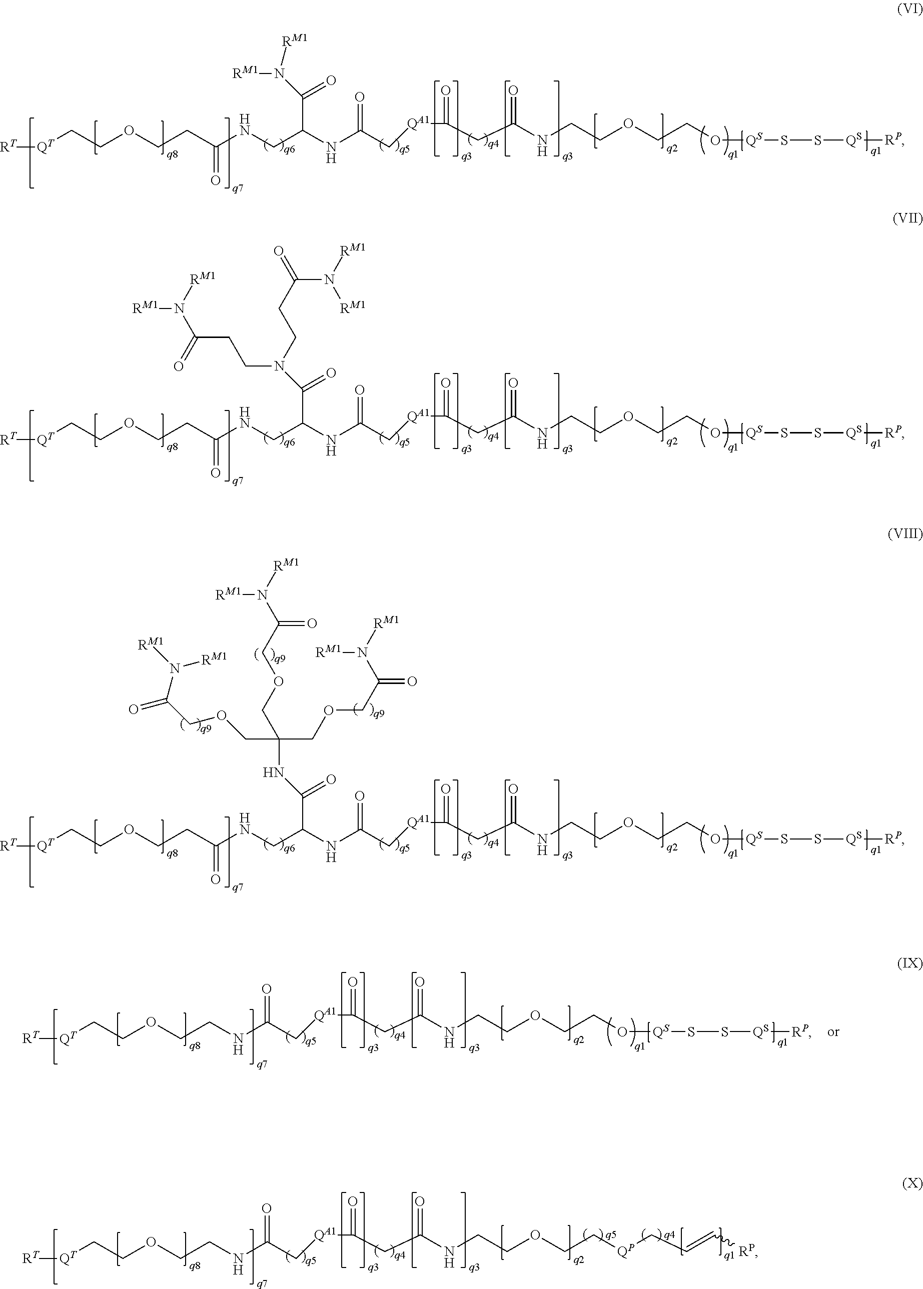

wherein:

Q^{A1} is optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

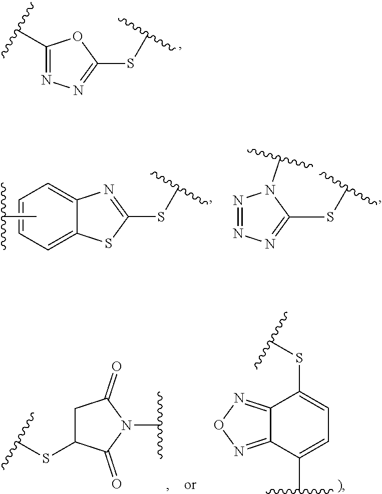

optionally substituted C₁₋₁₂ heterocyclylene (e.g., 1,2,3-triazole-1,4-diyl or

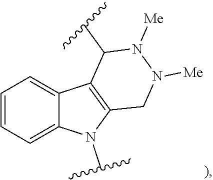 [SMILES: *C1c2c(n(*)c3ccccc23)CN(C)N1C]

cyclobut-3-ene-1,2-dione-3,4-diyl, or pyrid-2-yl hydrazone), optionally substituted C₆₋₁₆ triazoloheterocyclylene (e.g.,

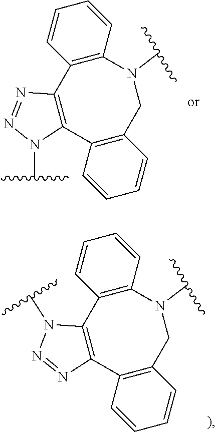

optionally substituted C₈₋₁₆ triazolocycloalkene (e.g.,

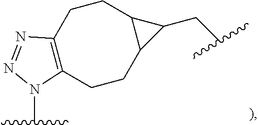 [SMILES: *CC1C2CCc3nnn(*)c3CCC12]

or a dihydropyridazine group (e.g.,

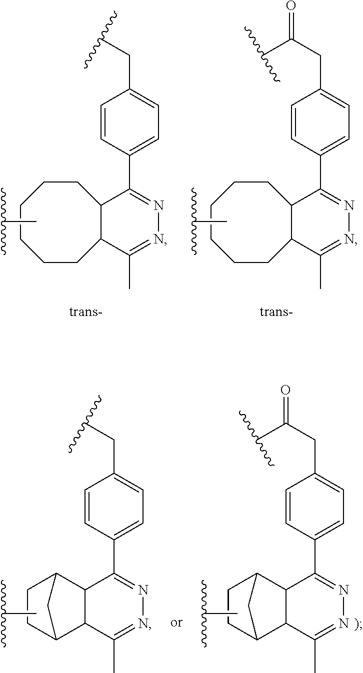

each R^{M1} is independently H or an auxiliary moiety;

each R^{T} is independently a bond to a targeting moiety;

each R^{P} is independently a bond to an internucleoside bridging group, a nucleobase, a capping group, or an abasic spacer;

each Q^{T} is independently —CO—, —NH—, —NH—CH₂—, or —CO—CH₂—;

each Q^{P} is independently —C(O)—N(H)—, —N(H)—C(O)—, —S(O)₂—N(H)—, or —N(H)—S(O)₂—;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each q1, q3, and q7 are independently an integer of about 0 or about 1;

each q2 and q8 are independently an integer from about 0 to about 50, from about 1 to about 40, or from about 1 to about 30;

each q4 is independently an integer from about 0 to about 10;

each q5 and q6 are independently an integer from about 1 to about 10 or from about 1 to about 6; and

each q9 is independently an integer from about 1 to about 10.

In certain embodiments, LinkA is:

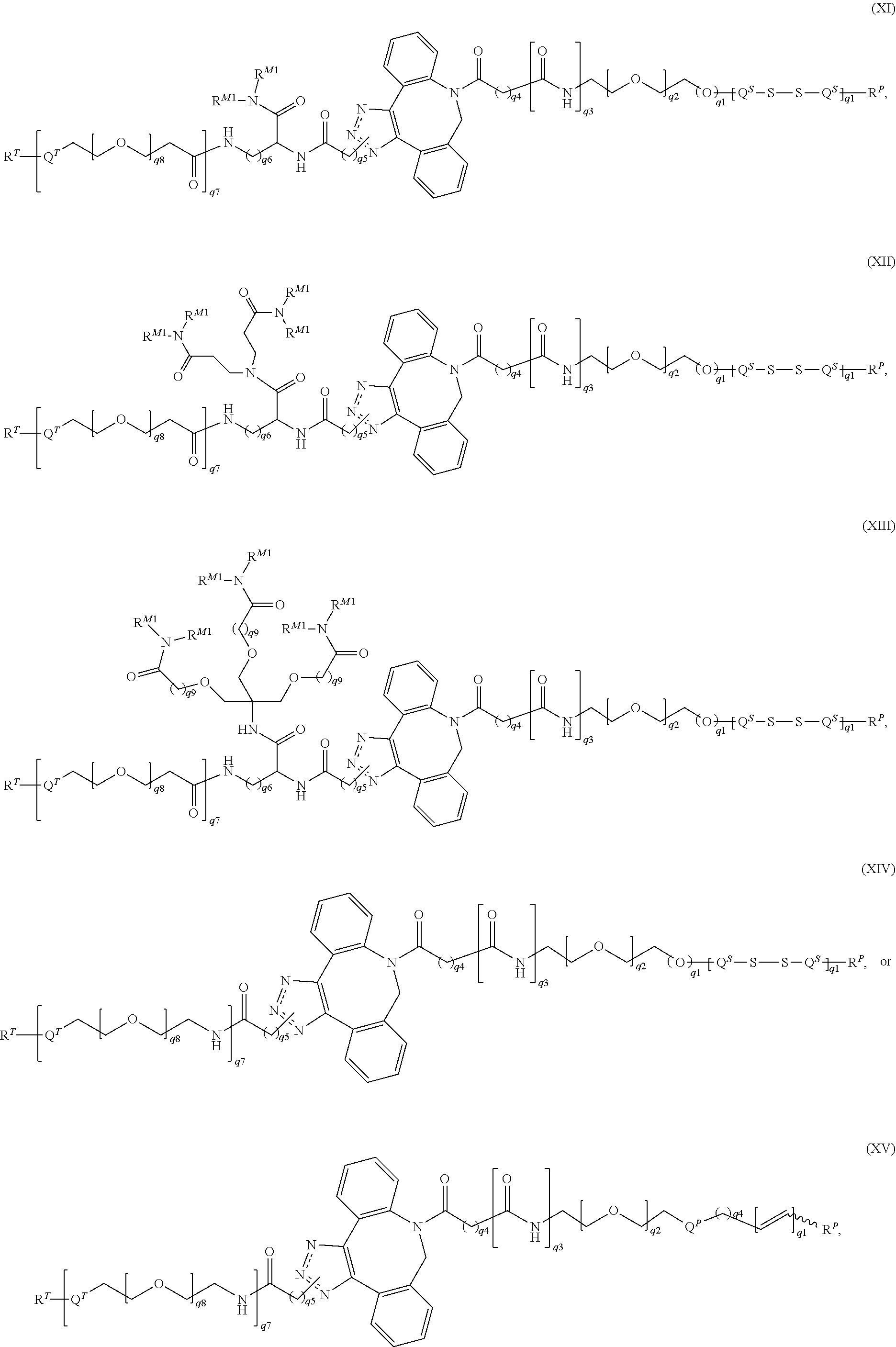

wherein:

in each structural formula, one [symbol] represents a single bond, and the other [symbol] represents a double bond;

[symbol]

each R^{M1} is independently H or an auxiliary moiety;

[symbol]

each R^{T} is independently a bond to a targeting moiety;

each R^{P} is independently a bond to an internucleoside bridging group, a nucleobase, a capping group, or an abasic spacer;

each Q^{T} is independently —CO—, —CO—CH₂—, —NH—, or —NH—CH₂—;

each Q^{P} is independently —C(O)—N(H)—, —N(H)—C(O)—, —S(O)₂—N(H)—, or —N(H)—S(O)₂—;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each q1, q3, and q7 are independently an integer of about 0 or about 1;

each q2 and q8 are independently an integer from about 0 to about 50, from about 1 to about 40, from about 1 to about 30;

each q4 is independently an integer from about 0 to about 10;

each q5 and q6 are independently an integer from about 1 to about 10 or from about 1 to about 6; and

each q9 is independently an integer from about 1 to about 10.

In certain embodiments, q5 is 0. In certain embodiments, q5 is an integer from about 2 to about 6.

In certain embodiments, a conjugating group is:

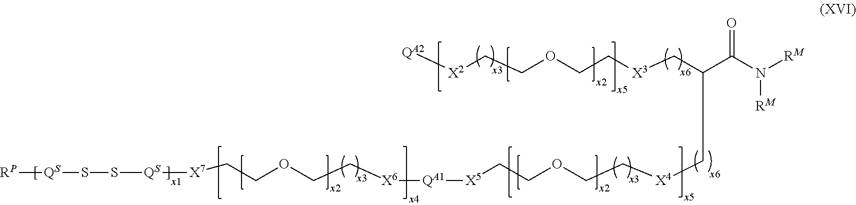

wherein:

Q^{A1} is independently optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

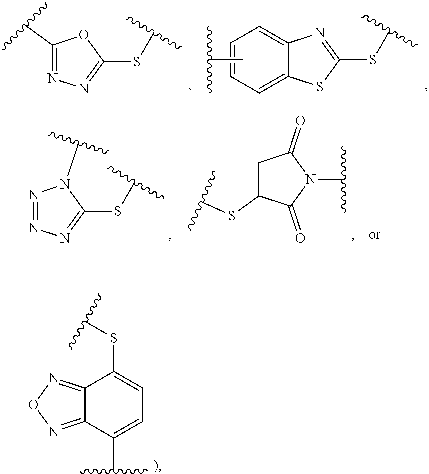

optionally substituted C₁₋₁₂ heterocyclylene (e.g., 1,2,3-triazole-1,4-diyl or

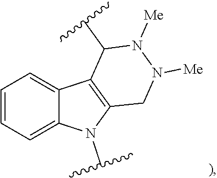 [SMILES: *C1c2c(n(*)c3ccccc23)CN(C)N1C]

cyclobut-3-ene-1,2-dione-3,4-diyl, pyrid-2-yl hydrazone, optionally substituted C₆₋₁₆ triazoloheterocyclylene (e.g.,

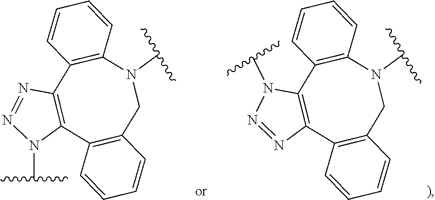 [SMILES: *N1Cc2ccccc2-c2c(nnn2*)-c2ccccc21 ; *N1Cc2ccccc2-c2nnn(*)c2-c2ccccc21]

optionally substituted C₈₋₁₆ triazolocycloalkenylene (e.g.,

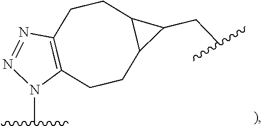 [SMILES: *CC1C2CCc3nnn(*)c3CCC12]

or a dihydropyridazine group (e.g.,

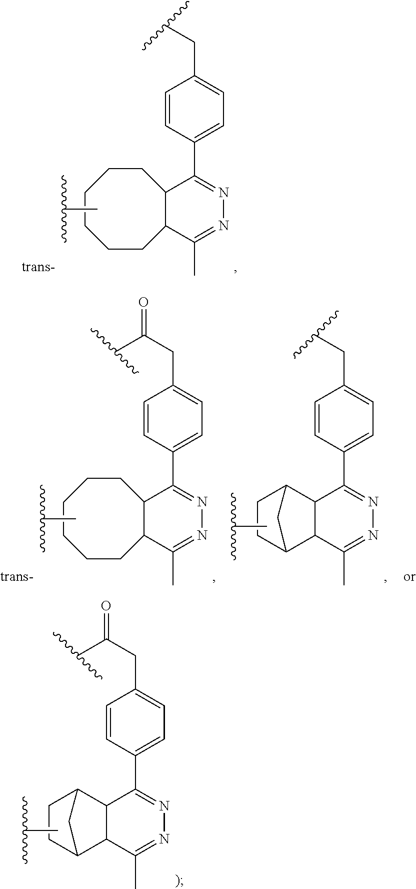

Q^{A2} is optionally substituted C₂₋₁₂ alkynyl, optionally substituted N-protected amino, azido, N-maleimido, S-protected thiol,

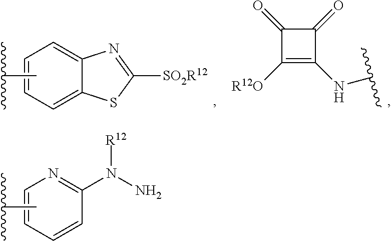

or an N-protected version thereof,

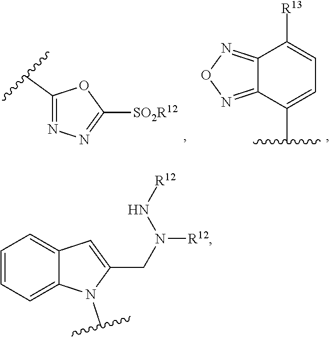

optionally substituted C₆₋₁₆ heterocyclyl containing an endocyclic carbon-carbon triple bond (e.g.,

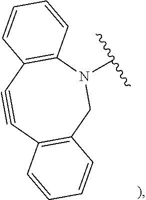 [SMILES: *N1Cc2ccccc2C#Cc2ccccc21]

1,2,4,5-tetrazine group (e.g.,

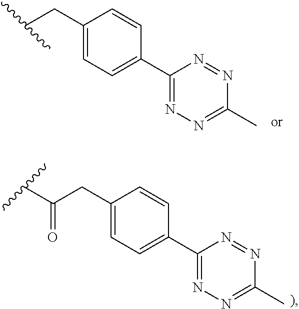 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

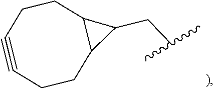 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;

R^{N1} is H, an N-protecting group, or optionally substituted C₁₋₆ alkyl;

each R¹² is independently H or optionally substituted C₁₋₆ alkyl;

R¹³ is halogen or F;

R^{P} is a bond to an internucleoside bridging group, a nucleobase, a capping group, or an abasic spacer;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each R^{M} is independently H, auxiliary moiety, —(CH₂)_{q7}—CO—N(R^{M1})₂, or —C[—CH₂O—(CH₂)_{q7}—CO—N(R^{M1})₂]₃, where each q7 is independently an integer from about 1 to about 5, and each R^{M1} is independently H or auxiliary moiety;

each X³ and X⁵ are independently absent, —O—, —NH—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

X⁷ is absent, —O—, —O—P(O)(OH)—O—, —O—P(S)(OH)—O—, —NH—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

each X², X⁴, and X⁶ are independently absent, —O—, —NH—, —O—, —C(O)—, —C(O)—NH—, —NH—C(O)—, —NH—C(O)—NH—, —O—C(O)—NH—, or —NH—C(O)—O—;

x1 and each x5 are independently an integer of about 0 or about 1;

each x2 is independently an integer from 0 to 50 (e.g., from 1 to 40 or from 1 to 30);

each x3 is independently an integer from 1 to 11;

x4 is 0, 1, or 2; and

each x6 is independently an integer from 0 to 10 (e.g., from 1 to 6), provided that the sum of both x6 is 12 or less.

In some embodiments, a conjugating group is:

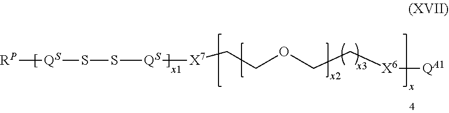

where:

Q^{A1} is optionally substituted C₂₋₁₂ alkynyl, optionally substituted N-protected amino azido, N-maleimido, S-protected thiol,

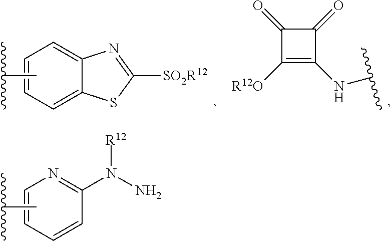

or N-protected version thereof,

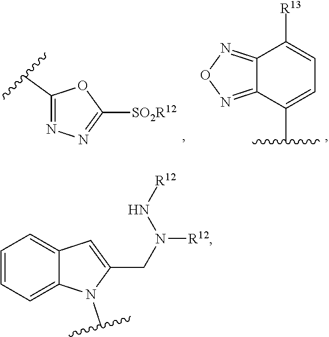

optionally substituted C₆₋₁₆ heterocyclyl containing an endocyclic carbon-carbon triple bond (e.g.,

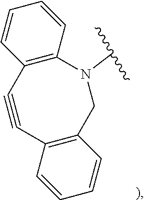 [SMILES: *N1Cc2ccccc2C#Cc2ccccc21]

1,2,4,5-tetrazine group (e.g.,

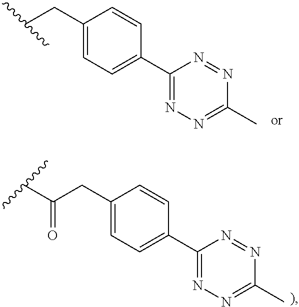 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

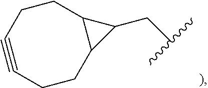 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;

R^{N1} is H, N-protecting group, or optionally substituted C₁₋₆ alkyl;

each R¹² is independently H or optionally substituted C₁₋₆ alkyl;

R¹³ is halogen (e.g., F);

R^{P} is a bond to an internucleoside bridging group, a nucleobase, a capping group, or an abasic spacer;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

X⁷ is absent, —O—, —NH—, —O—P(O)(OH)—O—, —O—P(S)(OH)—O—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

X⁶ is absent, —O—, —NH—, —O—, —C(O)—, —C(O)—NH—, —NH—C(O)—, —NH—C(O)—NH—, —O—C(O)—NH—, or —NH—C(O)—O—;

x1 is independently 0 or 1;

each x2 is independently an integer from 0 to 50, from 1 to 40, or from 1 to 30;

each x3 is independently an integer from 1 to 11; and

x4 is 0, 1, or 2.

In certain embodiments, a conjugating group is:

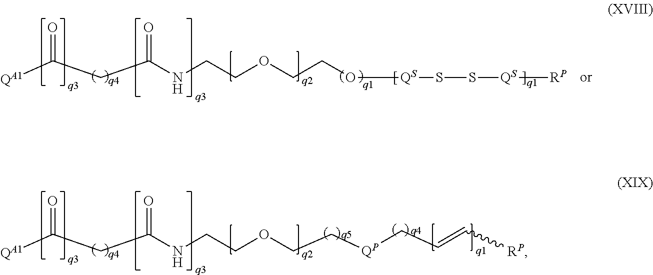

where:

Q^{A1} is optionally substituted C₂₋₁₂ alkynyl, optionally substituted N-protected amino, azido, N-maleimido, S-protected thiol,

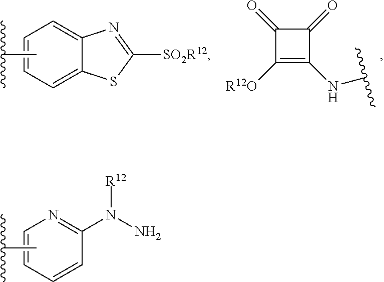

or N-protected version thereof,

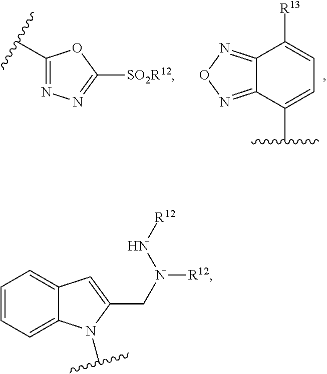

optionally substituted C₆₋₁₆ heterocyclyl containing an endocyclic carbon-carbon triple bond (e.g.,

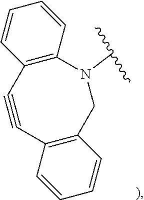 [SMILES: *N1Cc2ccccc2C#Cc2ccccc21]

1,2,4,5-tetrazine group (e.g.,

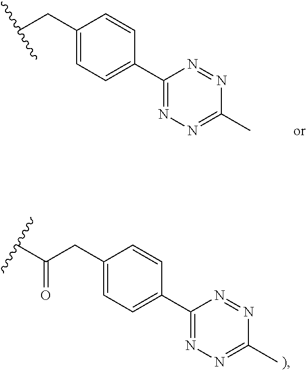 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

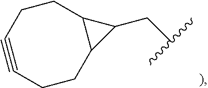 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;

R^{N1} is H, N-protecting group, or optionally substituted C₁₋₆ alkyl;

each R¹² is independently H or optionally substituted C₁₋₆ alkyl;

R¹³ is halogen (e.g., F);

R^{P} is a bond to an internucleoside bridging group, a nucleobase, a capping group, or an abasic spacer;

Q^{P} is —C(O)—N(H)—, —N(H)—C(O)—, —S(O)₂—N(H)—, or —N(H)—S(O)₂—;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each of q1 and q3 is independently 0 or 1;

q2 is an integer from 0 to 50, from 1 to 40, or from 1 to 30;

q4 is an integer from 0 to 10; and

q5 is an integer from 1 to 10 or from 1 to 6.

In yet further embodiments, the conjugating group is:

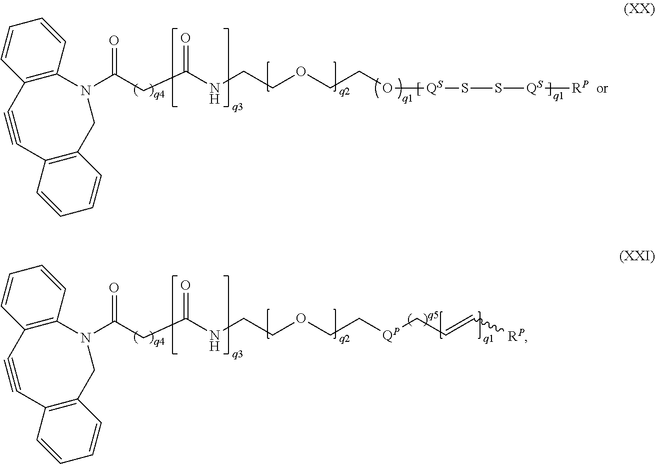

where:

R^{P} is a bond to an internucleoside bridging group, a nucleobase, a capping group, or an abasic spacer;

Q^{P} is —C(O)—N(H)—, —N(H)—C(O)—, —S(O)₂—N(H)—, or —N(H)—S(O)₂—;

each Q^{S} is independently optionally substituted C₂₋₁₂ alkylene, optionally substituted C₂₋₁₂ alkenylene, optionally substituted C₂₋₁₂ alkynylene, or optionally substituted (C₆₋₁₀ aryl)-C₁₋₆-alkylene;

each of q1 and q3 is independently 0 or 1;

q2 is an integer from 0 to 50, from 1 to 40, or from 1 to 30;

q4 is an integer from 0 to 10; and

q5 is an integer from 1 to 10 or from 1 to 6.

In certain exemplary embodiments, a conjugating group is:

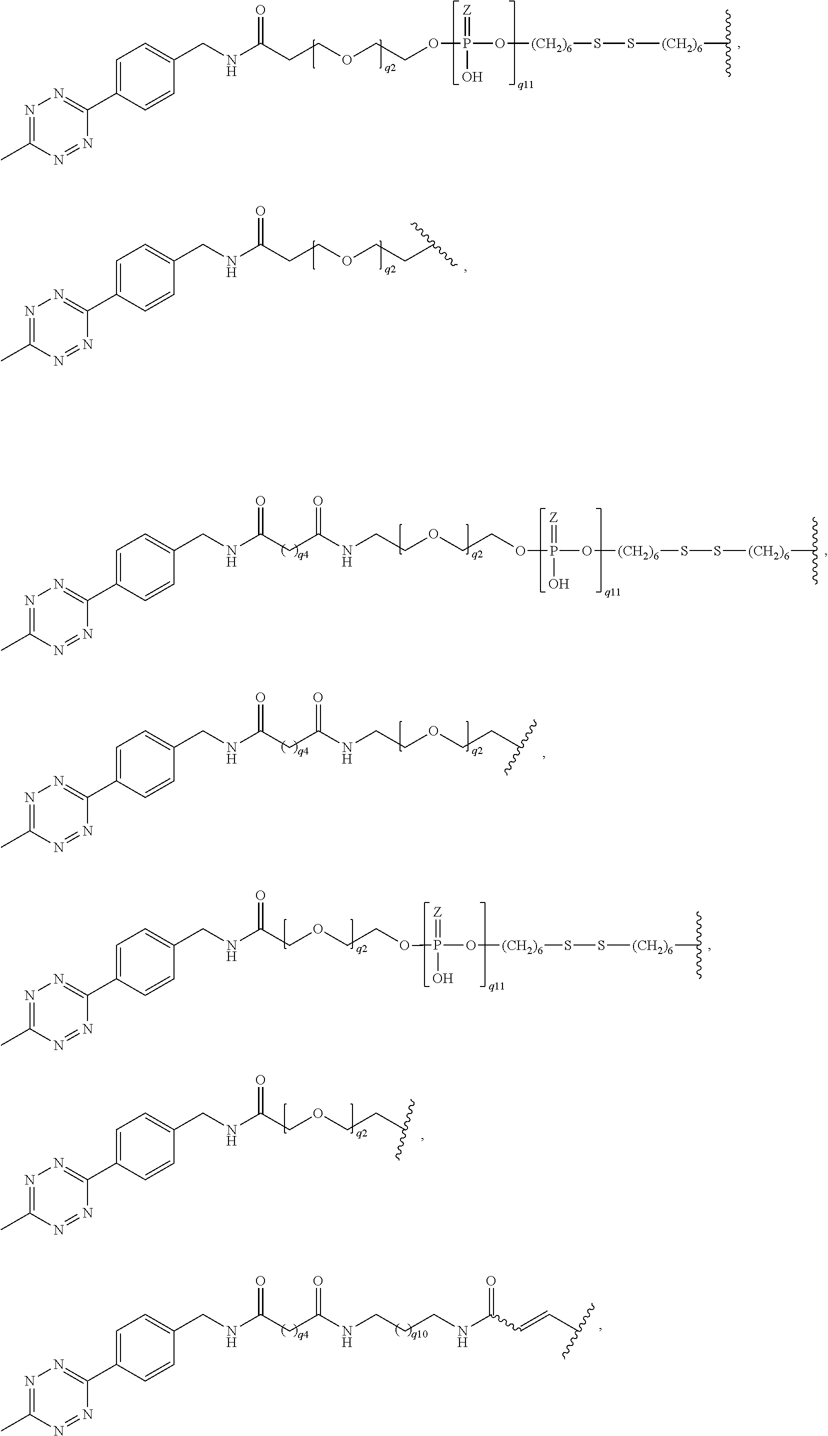

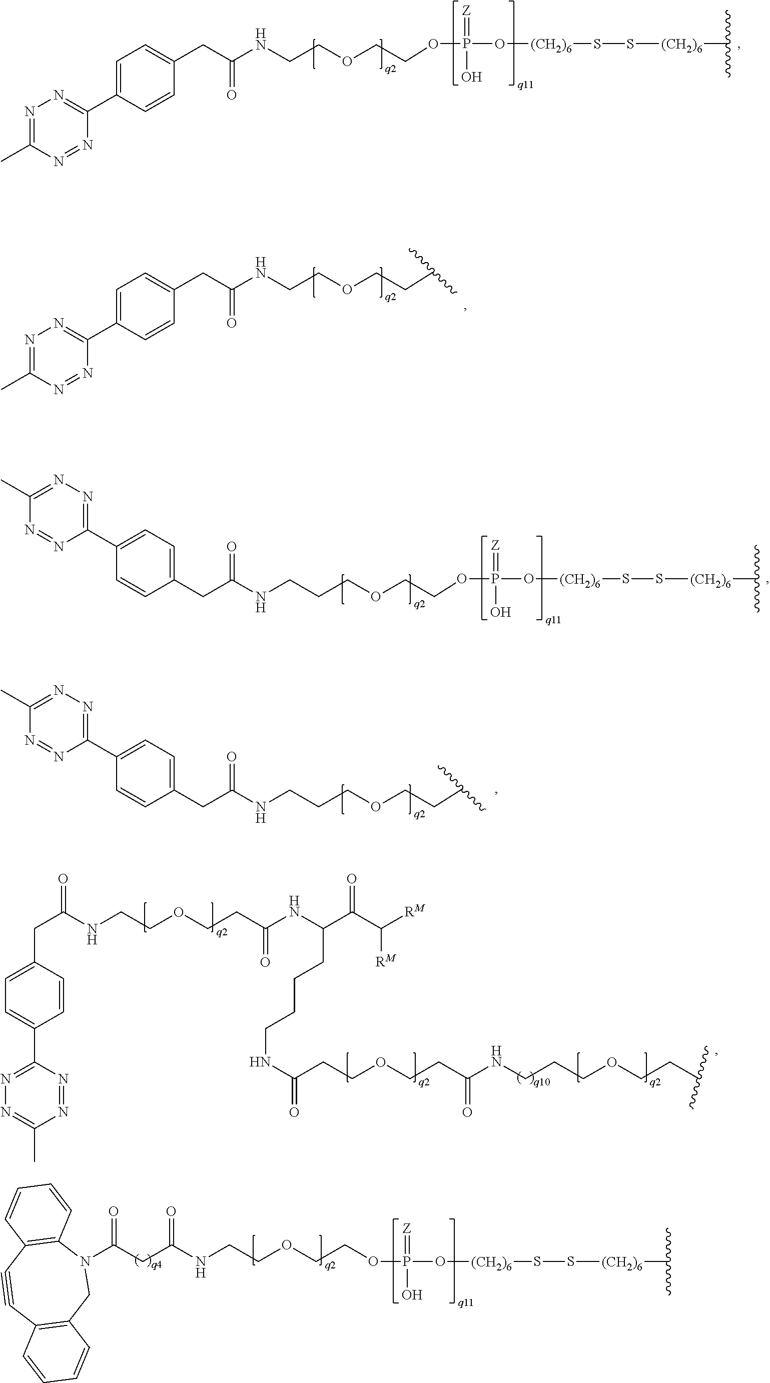

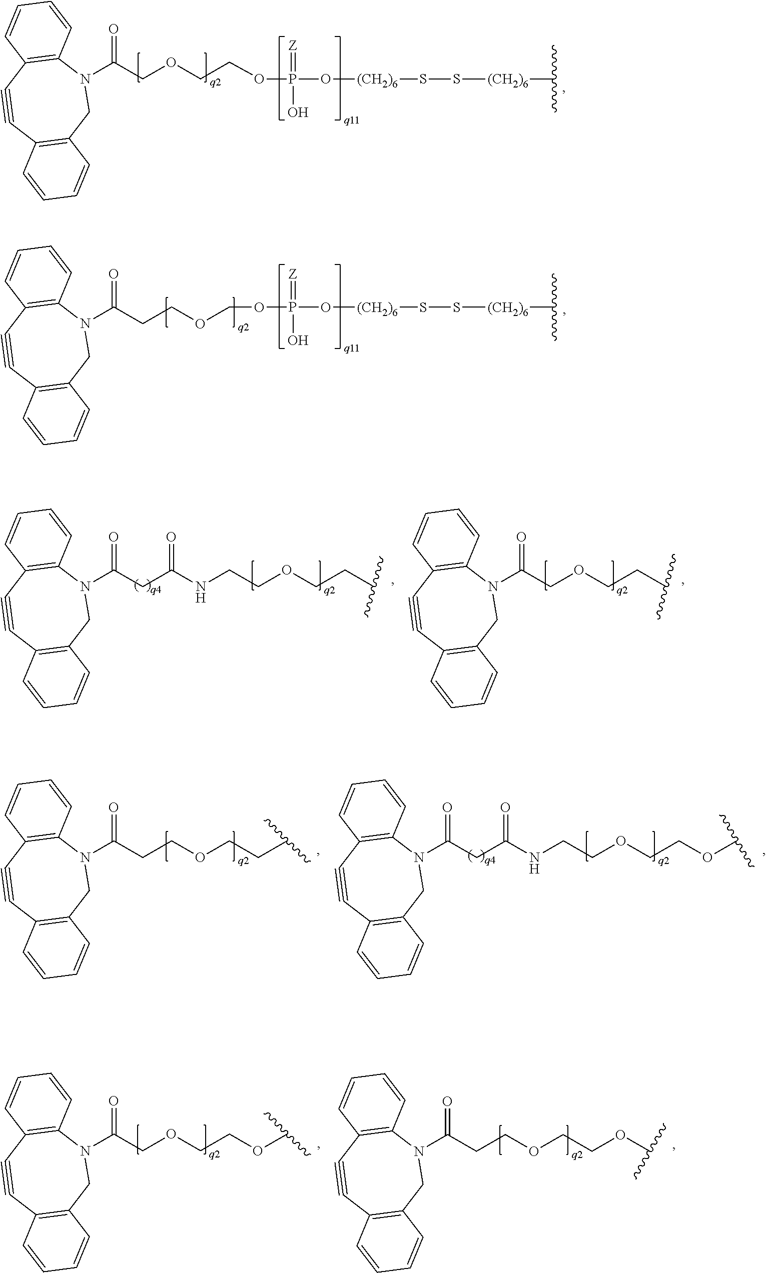

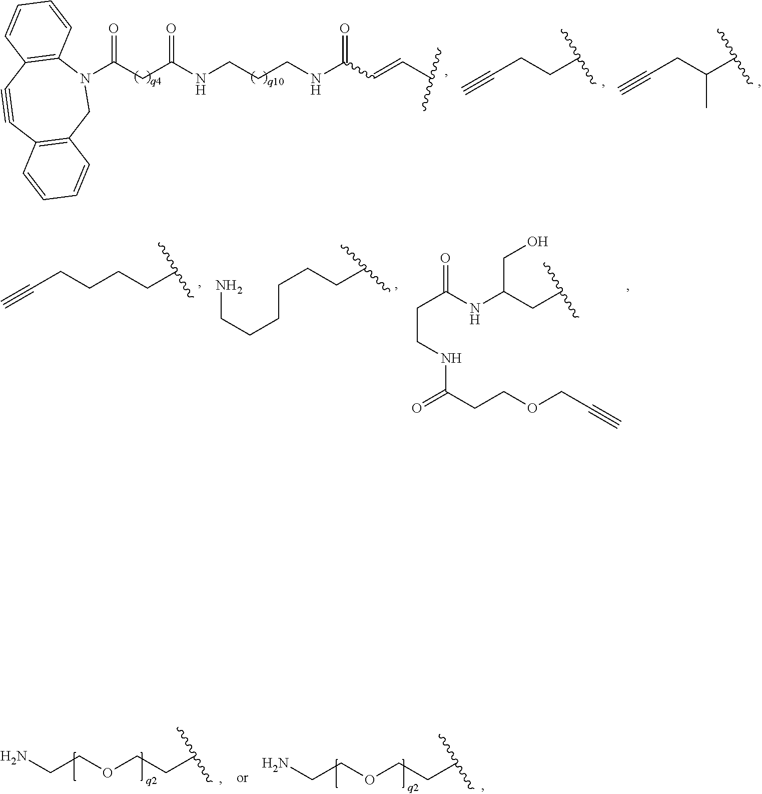

wherein: q2 is an integer from about 1 to about 50 (e.g., an integer from about 1 to about 24 or from about 1 to about 8 (e.g., about 2 or about 3)), q4 is an integer from about 0 to about 10 (e.g., about 0, about 1, about 2, about 3, about 4, about 5, about 6, about 7, or about 8), q10 is an integer from about 0 to about 8 (e.g., about 1, about 2, about 3, about 4, about 5, or about 6), q11 is about 0 or about 1, Z is O or S, and each R^{M} is independently H, an auxiliary moiety, —(CH₂)_{q7}—CO—N(R^{M1})₂, or —C[—CH₂O—(CH₂)_{q7}—CO—N(R^{M1})₂]₃, where each q7 is independently an integer from about 1 to about 5, and each R^{M1} is independently H or an auxiliary moiety.

In certain embodiments, the conjugating group for conjugation to a targeting moiety through a metal-catalyzed cycloaddition is:

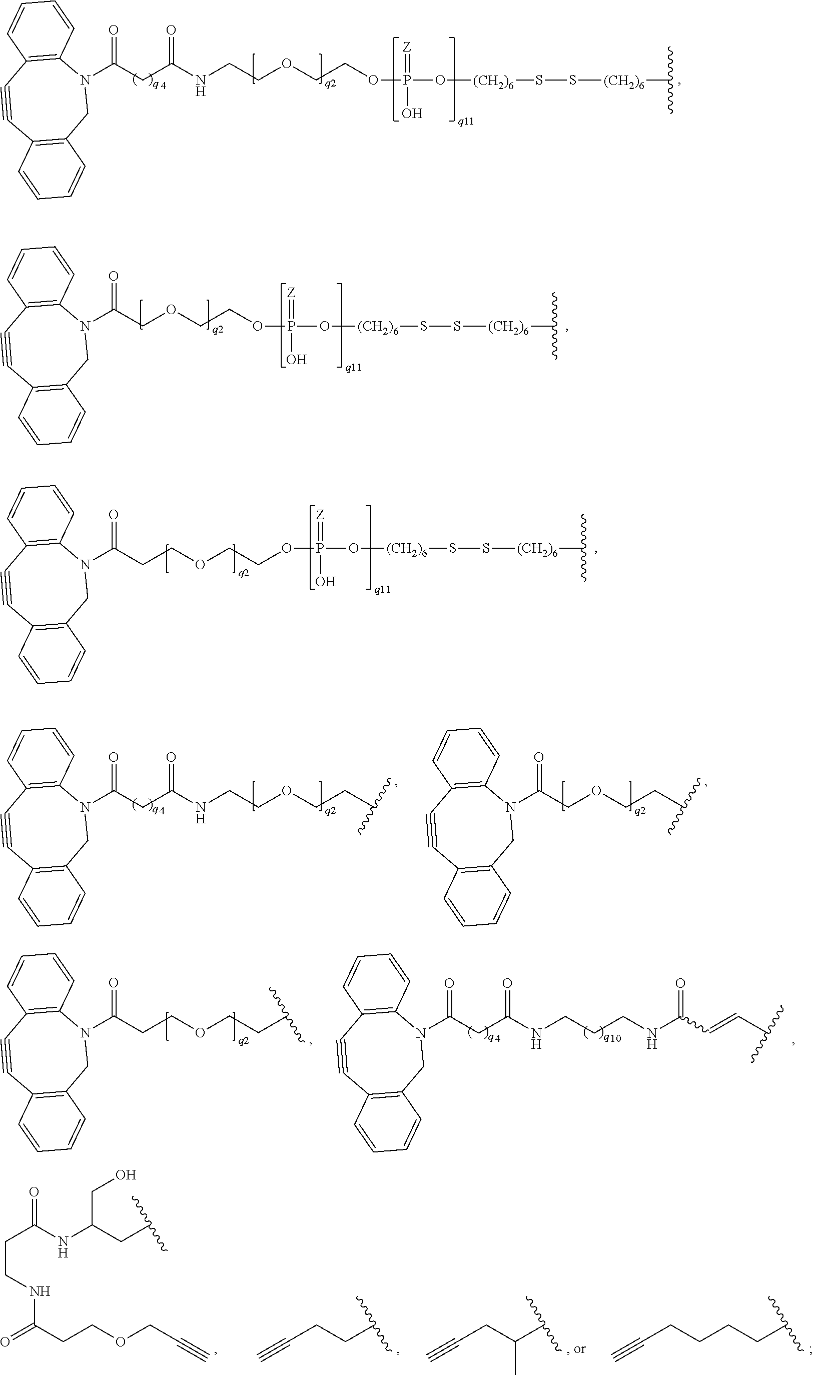

where q2 is an integer from about 1 to about 50 (e.g., an integer from about 1 to about 24 or from about 1 to about 8 (e.g., about 2 or about 3)), q4 is an integer from about 0 to about 10 (e.g., about 0, about 1, about 2, about 3, about 4, about 5, about 6, about 7, or about 8), q10 is an integer from about 0 to about 8 (e.g., about 1, about 2, about 3, about 4, about 5, or about 6), q11 is about 0 or about 1, and Z is 0 or S.

In certain embodiments, the conjugating group for conjugation to a targeting moiety through a metal-free cycloaddition is:

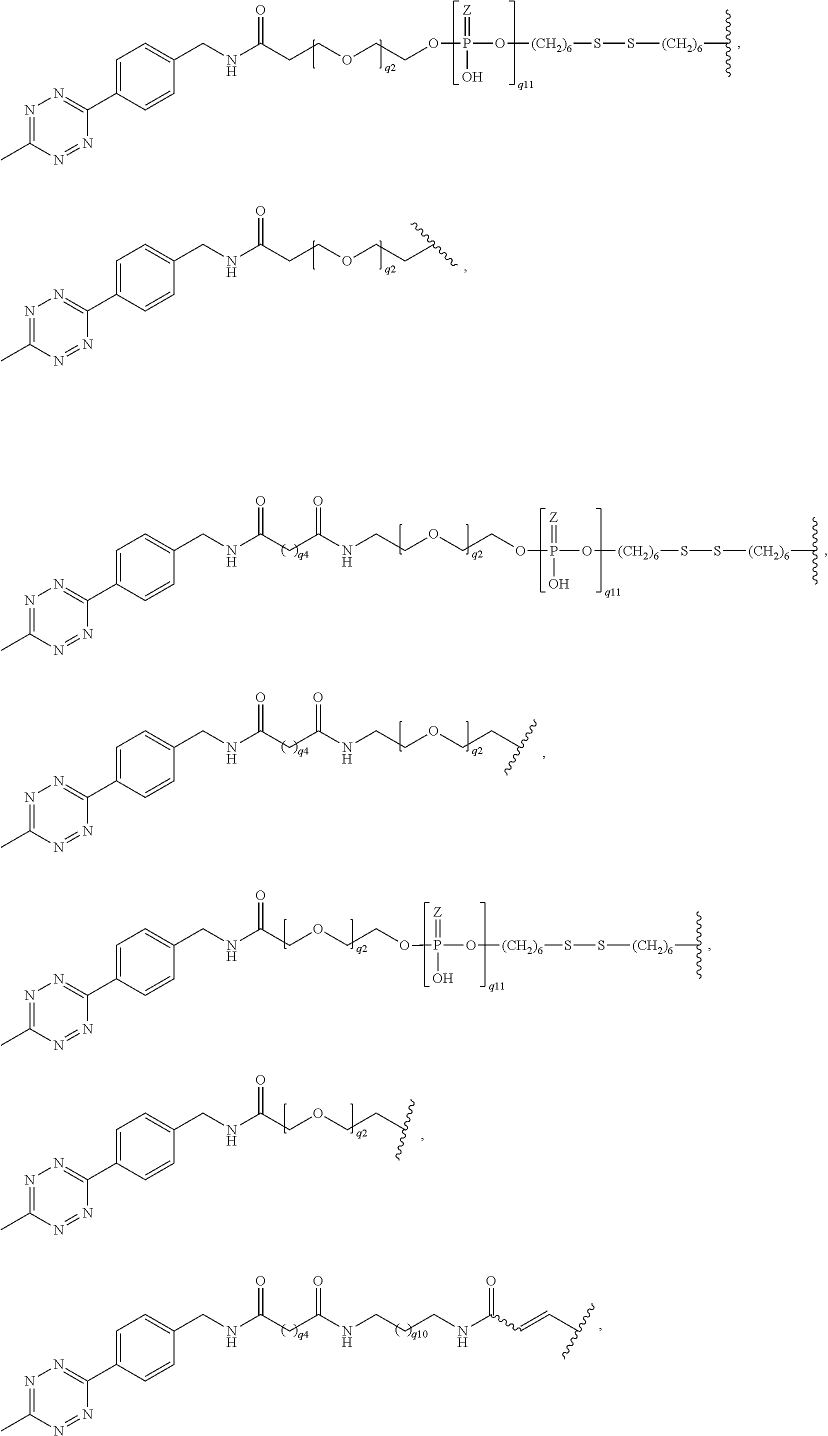

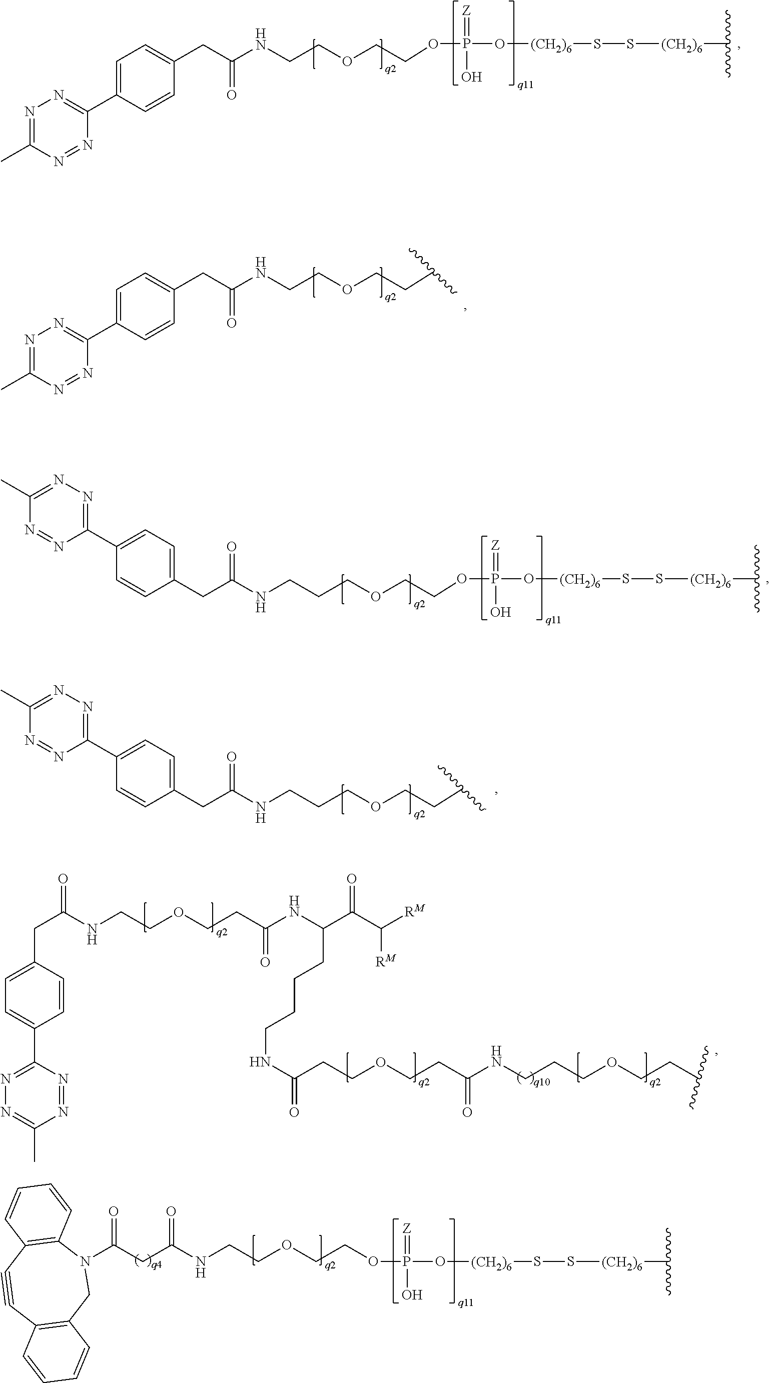

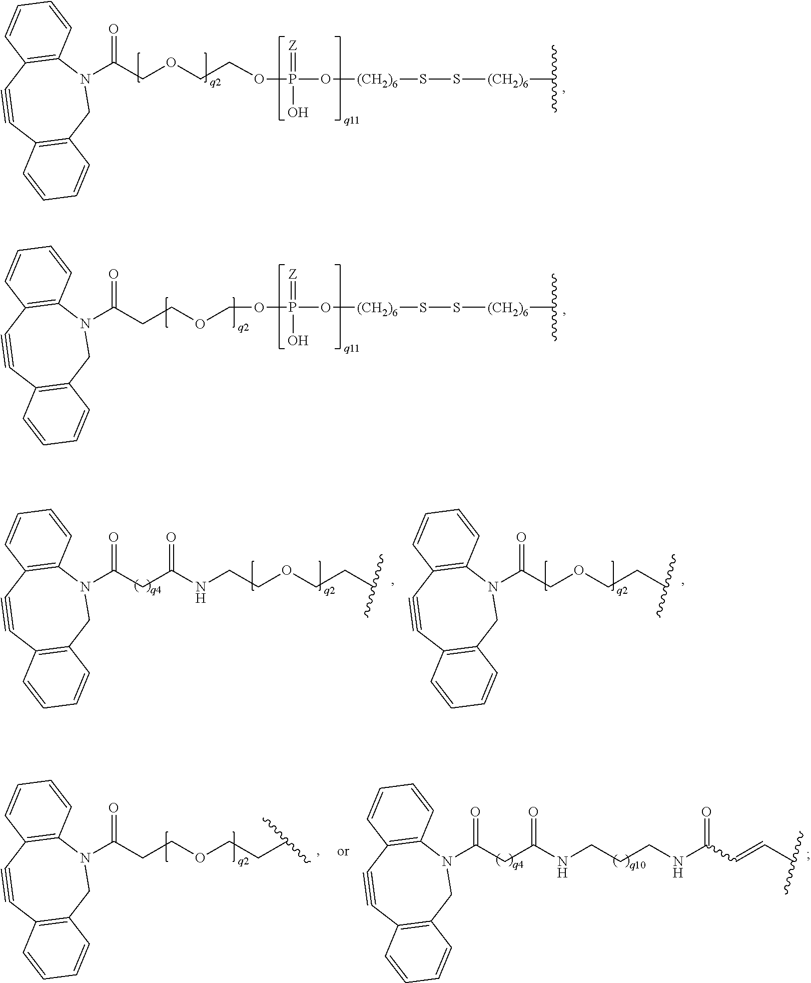

wherein: q2 is an integer from about 1 to about 50 (e.g., an integer from about 1 to about 24 or from about 1 to about 8 (e.g., about 2 or about 3)), q4 is an integer from about 0 to about 10 (e.g., about 0, about 1, about 2, about 3, about 4, about 5, about 6, about 7, or about 8), q10 is an integer from about 0 to about 8 (e.g., about 1, about 2, about 3, about 4, about 5, about or about 6), q11 is about 0 or about 1, Z is 0 or S, and each R^{M} is independently H, an auxiliary moiety, —(CH₂)_{q7}—CO—N(R^{M}1)₂, or —C[—CH₂O—(CH₂)_{q7}—CO—N(R^{M1})₂]₃, where each q7 is independently an integer from about 1 to about 5, and each R^{M1} is independently H or an auxiliary moiety.

In certain embodiments, the conjugating group for conjugation to a targeting moiety through amide formation is:

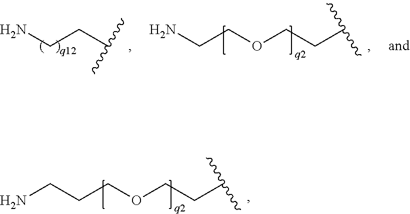

wherein q2 is an integer from about 0 to about 50 (e.g., an integer from about 1 to about 8 (e.g., about 2 or about 3)), and q12 is an integer from about 1 to about 11 (e.g., an integer from about 1 to about 5 (e.g., about 1, about 2, about 3, about 4, or about 5).

Bioreversible Groups

In certain embodiments, a bioreversible group comprises a disulfide (—S—S—). In certain embodiments, the bioreversible group is cleavable intracellularly under physiological conditions.

In certain embodiments, a bioreversible group is of formula (XXII):

R⁵—S—S-(LinkB)-, (XXII)

wherein:

LinkB is a divalent group containing an sp³-hybridized carbon atom bonded to phosphate, phosphorothioate, or phosphorodithioate, and a carbon atom bonded to —S—S—, and R⁵ is optionally substituted C₁₋₆ alkyl, optionally substituted C₆₋₁₀ aryl, or -LinkC(—R^{M})ᵣ, or LinkB is a trivalent linker containing an sp³-hybridized carbon atom bonded to phosphate, phosphorothioate, or phosphorodithioate, and a carbon atom bonded to —S—S—, in which the third valency of LinkB combines with —S—S— and R⁵ to form optionally substituted C₃₋₉ heterocyclylene;

LinkC is a multivalent group;

each R^{M} is independently H, an auxiliary moiety, or -Q^{G}[-Q^{B}-Q^{C}-Q^{D})ₛ₂—R^{M1}]ₚ₁,

where:
each R^{M1} is independently H or an auxiliary moiety,
each Q^{B} and each Q^{D} is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—,
each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene,
each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl,
each s2 is independently an integer from 0 to 10, and
p1 is 2 or 3;

and

r is an integer from 1 to 6 (e.g., 1, 2, or 3).

In certain embodiments, LinkB and/or R⁵ includes a bulky group attached to —S—S—. The inclusion of a bulky group attached to —S—S— may enhance the stability of the sulfur-sulfur bond, e.g., during the polynucleotide synthesis.

In further embodiments, LinkB consists of 1, 2, or 3 groups, each of the groups being independently selected from the group consisting of optionally substituted C₁₋₁₂ alkylene, optionally substituted C₂₋₁₂ alkenylene, optionally substituted C₂₋₁₂ alkynylene, optionally substituted C₆₋₁₀ arylene, optionally substituted C₂₋₁₂ heteroalkylene, and optionally substituted C₁₋₉ heterocyclylene.

In particular embodiments, LinkB and —S—S— combine to form a structure selected from the group consisting of:

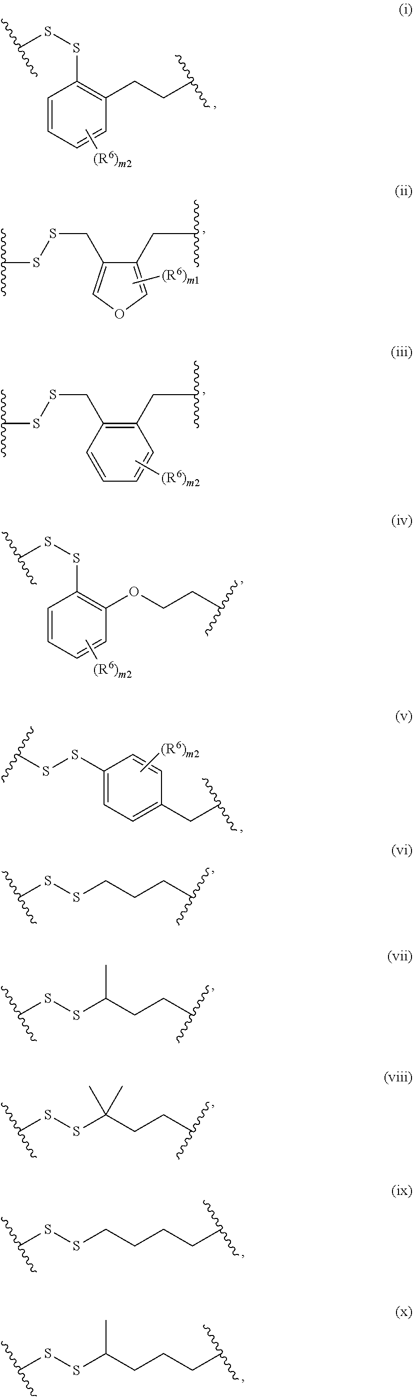

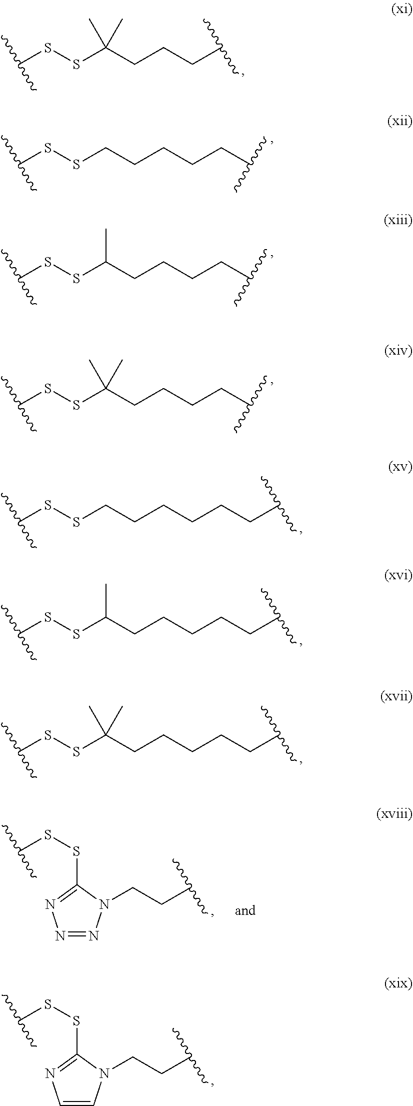

where:

each R⁶ is independently C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{c} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; or —S(O)R^{H} where R^{H} is selected from the group consisting of hydrogen, C₁-C₆ alkyl, C₆₋₁₀ aryl, and (C₆₋₁₀ aryl)-C₁₋₄-alkyl; or two adjacent R⁶ groups, together with the atoms to which each of the R⁶ groups is attached combine to form a cyclic group selected from the group consisting of C₆ aryl, C₂₋₅ heterocyclyl, or C₂₋₅ heteroaryl, wherein the cyclic group is optionally substituted with 1, 2, or 3 substituents selected from the group consisting of C₂₋₇ alkanoyl; C₁₋₆ alkyl; C₂₋₆ alkenyl; C₂₋₆ alkynyl; C₁₋₆ alkylsulfinyl; C₆₋₁₀ aryl; amino; (C₆₋₁₀ aryl)-C₁₋₄-alkyl; C₃₋₈ cycloalkyl; (C₃₋₈ cycloalkyl)-C₁₋₄-alkyl; C₃₋₈ cycloalkenyl; (C₃₋₈ cycloalkenyl)-C₁₋₄-alkyl; halo; C₁₋₉ heterocyclyl; C₁₋₉ heteroaryl; (C₁₋₉ heterocyclyl)oxy; (C₁₋₉ heterocyclyl)aza; hydroxy; C₁₋₆ thioalkoxy; —(CH₂)_{q}CO₂R^{A}, where q is an integer from zero to four, and R^{A} is selected from the group consisting of C₁₋₆ alkyl, C₆₋₁₀ aryl, and (C₆₋₁₀ aryl)-C₁₋₄-alkyl; —(CH₂)_{q}CONR^{B}R^{C}, where q is an integer from zero to four and where R^{B} and R^{C} are independently selected from the group consisting of hydrogen, C₁₋₆ alkyl, C₆₋₁₀ aryl, and (C₆₋₁₀ aryl)-C₁₋₄-alkyl; —(CH₂)_{q}SO₂R^{D}, where q is an integer from zero to four and where R^{D} is selected from the group consisting of C₁₋₆ alkyl, C₆₋₁₀ aryl, and (C₆₋₁₀ aryl)-C₁₋₄-alkyl; —(CH₂)_{q}SO₂NR^{E}R^{F}, where q is an integer from zero to four and where each of R^{E} and R^{F} is, independently, selected from the group consisting of hydrogen, alkyl, aryl, and (C₆₋₁₀ aryl)-C₁₋₄-alkyl; thiol; aryloxy; cycloalkoxy; arylalkoxy; (C₁₋₉ heterocyclyl)-C₁₋₄-alkyl; (C₁₋₉ heteroaryl)-C₁₋₄-alkyl; C₃₋₁₂ silyl; cyano; and —S(O)R^{H} where R^{H} is selected from the group consisting of hydrogen, C₁-C₆ alkyl, C₆₋₁₀ aryl, and (C₆₋₁₀ aryl)-C₁₋₄-alkyl;

m1 is 0, 1, or 2; and

m2 is 0, 1, 2, 3, or 4;

or LinkB, —S—S—, and R⁵ combine to form a group containing

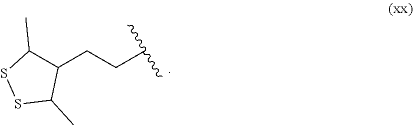 [SMILES: *CCC1C(C)SSC1C]

In yet further embodiments, LinkC can include from 0 to 3 multivalent monomers (e.g., optionally substituted C₁₋₆ alkane-triyl, optionally substituted C₁₋₆ alkane-tetrayl, or trivalent nitrogen atom) and one or more divalent monomers (e.g., from 1 to 40), where each divalent monomer is independently optionally substituted C₁₋₆ alkylene; optionally substituted C₂₋₆ alkenylene; optionally substituted C₂₋₆ alkynylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, wherein m is 0, 1, or 2. In some embodiments, each monomer is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; imino; optionally substituted N; O; or S(O)ₘ, where m is 0, 1, or 2 (e.g., m is 2). In certain embodiments, each monomer is independently optionally substituted C₁₋₆ alkylene; optionally substituted C₃₋₈ cycloalkylene; optionally substituted C₃₋₈ cycloalkenylene; optionally substituted C₆₋₁₄ arylene; optionally substituted C₁₋₉ heteroarylene having 1 to 4 heteroatoms selected from N, O, and S; optionally substituted C₁₋₉ heterocyclylene having 1 to 4 heteroatoms selected from N, O, and S; optionally substituted N; O; or S(O)ₘ, where m is 0, 1, or 2 (e.g., m is 2). The non-bioreversible linker connecting the auxiliary moiety to the conjugation moiety or to the reaction product thereof can include from 2 to 500 (e.g., 2 to 300, 2 to 200, 2 to 100, or 2 to 50) of such monomers. LinkC may include one or more polyethylene glycols (e.g., the polyethylene glycols may have a molecular weight of from 88 Da to 1 kDa (e.g., from 88 Da to 500 Da).

Compounds that may be used in the preparation of group -LinkC(—R^{M})ᵣ in formula (IIa) are described herein as well as in WO 2015/188197. Non-limiting examples of -LinkC(—R^{M})ᵣ include:

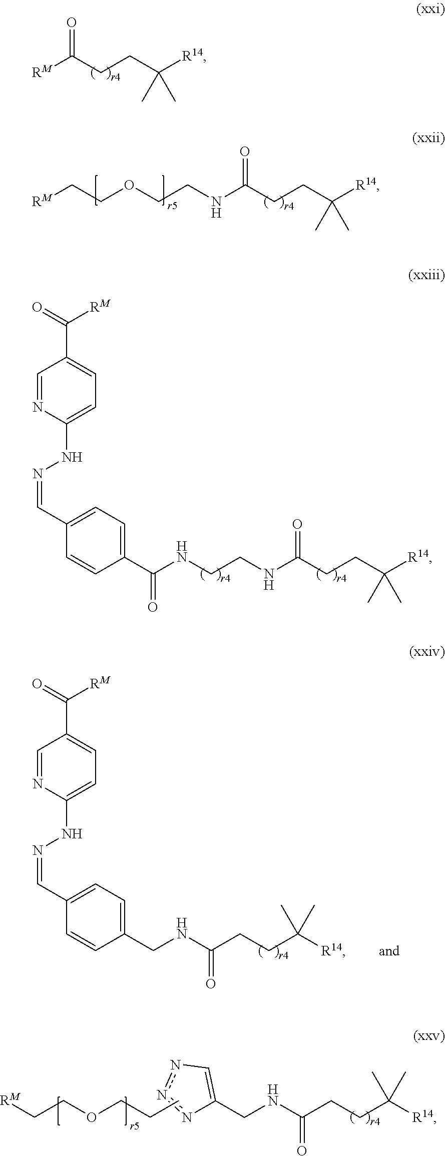

where:

R¹⁴ is a bond to —S—S—,

R^{M} is an auxiliary moiety or -Q^{G}[(-Q^{B}-Q^{C}-Q^{D})ₛ₂—R^{M1}]ₚ₁,
where:
   each R^{M1} is independently H or an auxiliary moiety,
   each Q^{B} and each Q^{D} is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—,
   each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene;
   each Q^{G} is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl,
   each s2 is independently an integer from 0 to 10, and
   p1 is 2 or 3;

each r4 is independently an integer from 1 to 6; and

each r5 is independently an integer from 0 to 10.

In certain embodiments, R^{M} is an auxiliary moiety. In some embodiments, at least one R^{M1} is an auxiliary moiety.

In certain embodiments, the bioreversible linker group is

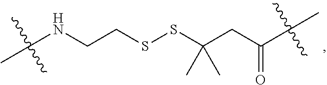 [SMILES: *NCCSSC(C)(C)CC(*)=O]

wherein one end of the group is connected to a polynucleotide and the other end is connected to a target moiety (in one embodiment, an antibody).

Non-Bioreversible Groups

A non-bioreversible group is a monovalent substituent that does not contain bonds cleavable under physiologic conditions in serum or in an endosome (e.g., esters, thioesters, or disulfides). The non-bioreversible group may be optionally substituted C₂₋₁₆ alkyl; optionally substituted C₃₋₁₆ alkenyl; optionally substituted C₃₋₁₆ alkynyl; optionally substituted C₃₋₈ cycloalkyl; optionally substituted C₃₋₈ cycloalkenyl; optionally substituted (C₃₋₈ cycloalkyl)-C₁₋₄-alkyl; optionally substituted (C₃₋₈ cycloalkenyl)-C₁₋₄-alkyl; optionally substituted C₆₋₁₄ aryl; optionally substituted (C₆₋₁₄ aryl)-C₁₋₄-alkyl; optionally substituted C₁₋₉ heteroaryl having 1 to 4 heteroatoms selected from N, O, and S; optionally substituted (C₁₋₉ heteroaryl)-C₁₋₄-alkyl having 1 to 4 heteroatoms selected from N, O, and S; optionally substituted C₂₋₉ heterocyclyl having 1 to 4 heteroatoms selected from N, O, and S, where the heterocyclyl does not contain an S—S bond; optionally substituted (C₂₋₉ heterocyclyl)-C₁₋₄-alkyl having 1 to 4 heteroatoms selected from N, O, and S, where the heterocyclyl does not contain an S—S bond; or a group of formula (XXIII):

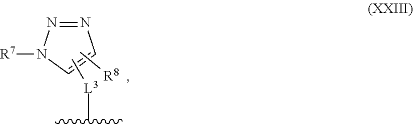

where:
L³ is C₂₋₆ alkylene;
R⁷ is optionally substituted C₂₋₆ alkyl; optionally substituted C₆₋₁₄ aryl; optionally substituted (C₆₋₁₄ aryl)-C₁₋₄-alkyl; optionally substituted C₃₋₈ cycloalkyl; optionally substituted (C₃₋₈ cycloalkyl)-C₁₋₄-alkyl; optionally substituted C₁₋₉ heteroaryl having 1 to 4 heteroatoms selected from the group consisting of N, O, and S; optionally substituted (C₁₋₉ heteroaryl)-C₁₋₄-alkyl having 1 to 4 heteroatoms selected from the group consisting of N, O, and S; optionally substituted C₂₋₉ heterocyclyl having 1 to 4 heteroatoms selected from the group consisting of N, O, and S, wherein the heterocyclyl does not contain an S—S bond; optionally substituted (C₂₋₉ heterocyclyl)-C₁₋₄-alkyl having 1 to 4 heteroatoms selected from N, O, and S, wherein the heterocyclyl does not contain an S—S bond; and a poly(ethylene glycol) terminated with —OH, C₁₋₆ alkoxy, or —COOH; and
R⁸ is H or C₁₋₆ alkyl.

A non-bioreversible phosphotriester may be a phosphate or a phosphorothioate substituted with a substituent that is a conjugating group, C₂₋₁₆ alkyl,

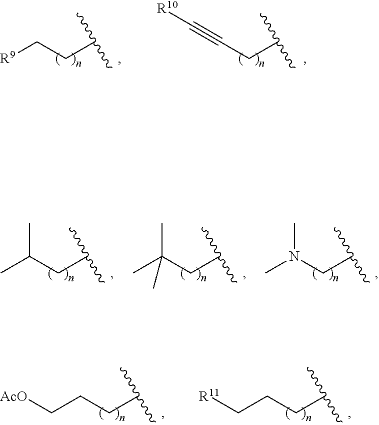

or a group formed by cycloaddition reaction of

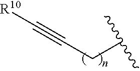

with an azido-containing substrate,
where:

n is an integer from 1 to 6;

R⁹ is optionally substituted C₆ aryl; optionally substituted C₄₋₅ heteroaryl that is a six member ring containing 1 or 2 nitrogen atoms; or optionally substituted C₄₋₅ heterocyclyl that is a six member ring containing 1 or 2 nitrogen atoms;

R¹⁰ is H or C₁₋₆ alkyl;

R¹¹ is a halogen, —COOR^{11A}, or —CON(R^{11B})₂, where each of R^{11A} and R^{11B} is independently H, optionally substituted C₁₋₆ alkyl, optionally substituted C₆₋₁₄ aryl, optionally substituted C₁₋₉ heteroaryl, or optionally substituted C₂₋₉ heterocyclyl; and

the azido-containing substrate is

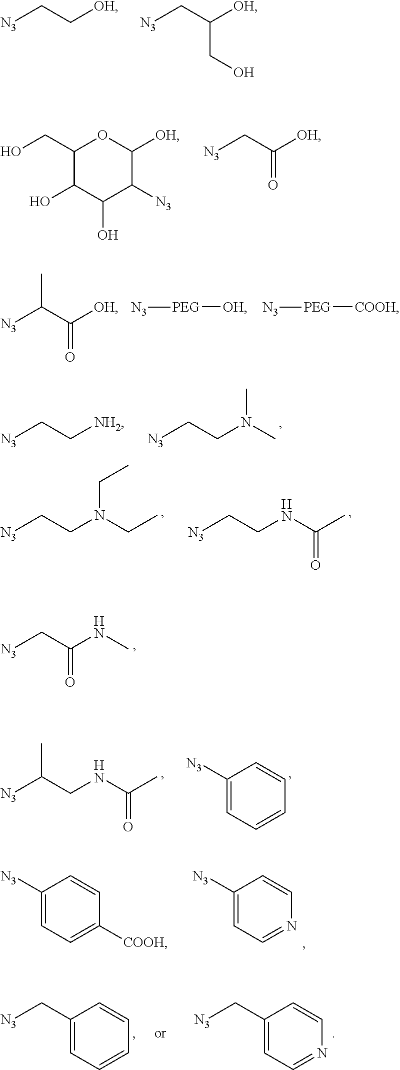

In some embodiments, a non-bioreversible group is -LinkD(-R^{M1})ᵣ₁, where LinkD is a multivalent linker, each R^{M1} is independently H or an auxiliary moiety, and r1 is an integer from 1 to 6.

In some instances, -LinkD(-R^{M1})ᵣ₁ is of formula (XXIV):

-Q^{R}-Q³([-Q⁴-Q⁵-Q⁶]ᵣ₂-Q⁷-R^{M1})ᵣ₁, (XXIV)

where:

r1 is an integer from 1 to 6;

each r2 is independently an integer from 0 to 50 (e.g., from 0 to 30), where the repeating units are same or different;

Q^{R} is [-Q⁴-Q⁵-Q⁶]ᵣ₂-Q^{L}-, where Q^{L} is optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

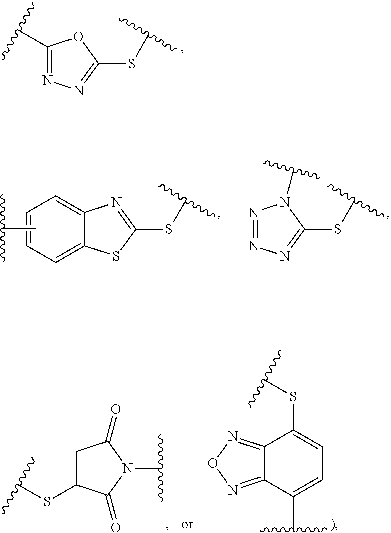

optionally substituted C₁₋₁₂ heterocyclylene (e.g., 1,2,3-triazole-1,4-dylyl or

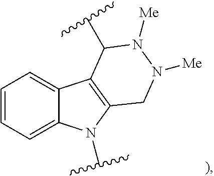 [SMILES: *C1c2c(n(*)c3ccccc23)CN(C)N1C]

cyclobut-3-ene-1,2-dione-3,4-diyl, pyrid-2-yl hydrazone, optionally substituted C₆₋₁₆ triazoloheterocyclylene (e.g.,

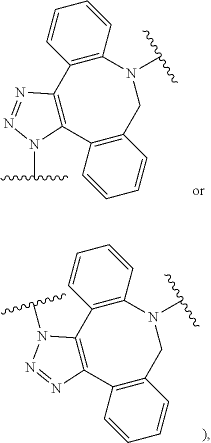

optionally substituted C₈₋₁₆ triazolocycloalkenylene (e.g.,

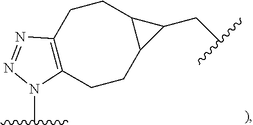 [SMILES: *CC1C2CCc3nnn(*)c3CCC12]

or a dihydropyridazine group (e.g.,

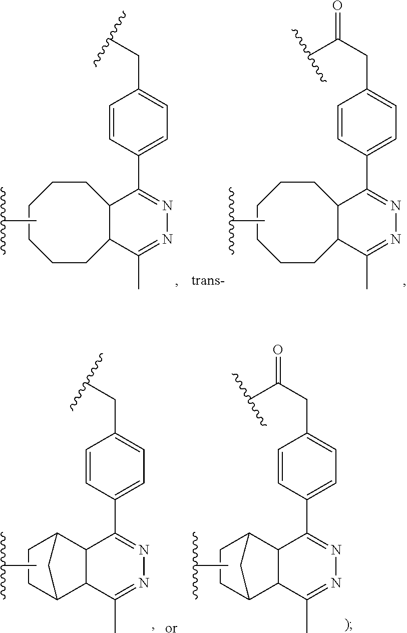

Q³ is a linear group (e.g., [-Q⁴-Q⁵-Q⁶]ᵣ₂-), if r1 is 1, or a branched group (e.g., [-Q⁴-Q⁵-Q⁶]-Q⁸([-Q⁴-Q⁵-Q⁶]ᵣ₂-(Q⁸)ᵣ₃)ᵣ₄, where r3 is 0 or 1, r4 is 0, 1, 2, or 3), if r1 is an integer from 2 to 6; each r2 is independently an integer from 0 to 50 (e.g., from 0 to 30), where the repeating units are the same or different;

each Q⁴ and each Q⁶ is independently absent —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;

each Q^{S} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene;

each Q⁷ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, or —C(O)—CH(R^{a})—NH—;

each Q⁸ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and

each R^{a} is independently H or an amino acid side chain; and

each R^{M1} is independently H or an auxiliary moiety.

In formula (XXIV), at least one of Q⁴, Q⁵, and Q⁶ is present. In formula (XXIV), LinkD may include a single branching point, if each r3 is 0, or multiple branching points, if at least one r3 is 1. In formula (XXIV), Q^{R} may be -Q⁵-Q⁴-Q^{L}-, where Q^{S} is optionally substituted C₂₋₁₂ heteroalkylene or optionally substituted C₁₋₁₂ alkylene, and Q⁴ is —CO—, —NH—, or —O—. In formula (XXIV), Q^{L} may be:

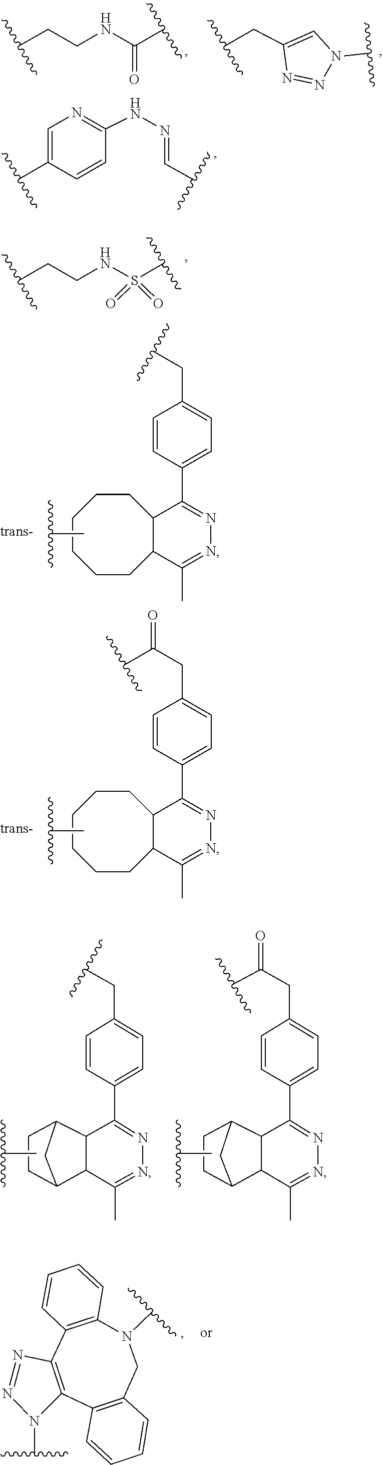

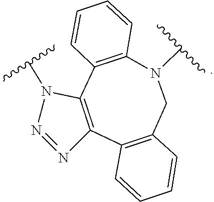 [SMILES: *N1Cc2ccccc2-c2nnn(*)c2-c2ccccc21]

In formula (XXIV), Q³ may be a linear group of formula [-Q⁴-Q⁵-Q⁶]ᵣ₂-, where Q⁴, Q⁵, and Q⁶ are as defined for formula (XXIV). Alternatively, Q³ may be a branched group [-Q⁴-Q⁵-Q⁶]ᵣ₂-Q⁸([-Q⁴-Q⁵-Q⁶]ᵣ₂-(Q⁸)ᵣ₃)ᵣ₄, where each Q⁸ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;

where:

each r2 is independently an integer from 0 to 50 (e.g., from 0 to 30), where the repeating units are the same or different;

r3 is 0 or 1;

r4 is 0, 1, 2, or 3;

where,
when r3 is 0, LinkD is a trivalent or tetravalent group, and,
when r3 is 1, LinkD is a tetravalent, pentavalent, or hexavalent group.

In certain embodiments, r3 is 0.

In some embodiments, Q⁸ is:

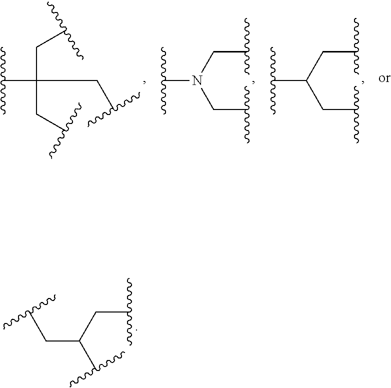

Compounds that may be used in the preparation of group -LinkD(-R^{M1})ₚ in formula (I) are described herein as well as in WO 2015/188197.

In certain embodiments, the non-bioreversible linker group is

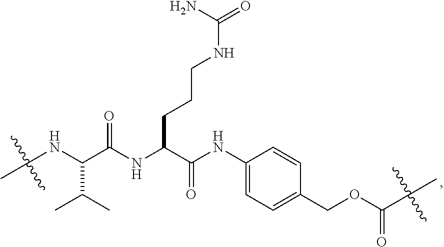 [SMILES: *N[C@H](C(=O)N[C@@H](CCCNC(N)=O)C(=O)Nc1ccc(COC(*)=O)cc1)C(C)C]

wherein one end of the group is connected to a polynucleotide and the other end is connected to a target moiety (in one embodiment, an antibody).

Auxiliary Moieties

An auxiliary moiety is a monovalent group containing a dye or a hydrophilic group or a combination thereof (e.g., a hydrophilic polymer (e.g., poly(ethylene glycol) (PEG)), a positively charged polymer (e.g., poly(ethylene imine)), or a sugar alcohol (e.g., glucitol)). An auxiliary moiety may have a theoretical molecular weight of from 100 Da to 2.5 kDa (e.g., from 350 Da to 2.5 kDa, from 100 Da to 1,200 Da, or from 1 kDa to 2.5 kDa).

Dyes may be included in the phosphoester groups for the purpose of visualization of uptake or monitoring the movement of the conjugates of the invention inside a cell (e.g., using Fluorescence Recovery After Photobleaching (FRAP)). Dyes known in the art may be included as an auxiliary moiety linked to the polynucleotide via a phosphate or phosphorothioate at the 5′- or 3′-terminus or via a phosphate or phosphorothioate bonding two consecutive nucleosides together. Non-limiting examples of useful structures that can be used as dyes include FITC, RD1, allophycocyanin (APC), aCFTM dye (Biotium, Hayward, Calif.), BODIPY (Invitrogen™ 10 of Life Technologies, Carlsbad, Calif.), AlexaFluor® (Invitrogen™ of Life Technologies, Carlsbad, Calif.), DyLight Fluor (Thermo Scientific Pierce Protein Biology Products, Rockford, Ill.), ATTO (ATTO-TEC GmbH, Siegen, Germany), FluoProbe (Interchim SA, Motluçon, France), and Abberior Probes (Abberior GmbH, Göttingen, Germany).

Hydrophilic polymers and positively charged polymers that may be used as auxiliary moieties in the immunomodulating polynucleotides of the invention and in the conjugates of the invention are known in the art. A non-limiting example of a hydrophilic polymer is poly(ethylene glycol). A non-limiting example of a positively charged polymer is poly(ethylene imine).

A sugar alcohol-based auxiliary moiety may be, e.g., amino-terminated glucitol or a glucitol cluster. The amino-terminated glucitol auxiliary moiety is:

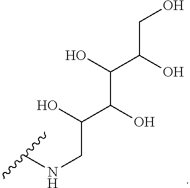 [SMILES: *NCC(O)C(O)C(O)C(O)CO]

Non-limiting examples of glucitol clusters are:

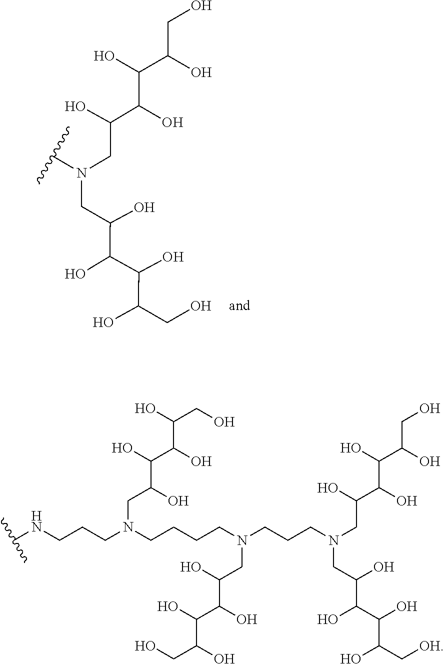 [SMILES: *N(CC(O)C(O)C(O)C(O)CO)CC(O)C(O)C(O)C(O)CO ; *NCCCN(CCCCN(CCCN(CC(O)C(O)C(O)C(O)CO)CC(O)C(O)C(O)C(O)CO)CC(O)C(O)C(O)C(O)CO)CC(O)C(O)C(O)C(O)CO]

In one embodiment, provided herein is a compound of Formula (B):

R^{x}-L^{N}-(Q)ₑ (B)

or a stereoisomer, a mixture of two or more diastereomers, a tautomer, or a mixture of two or more tautomers; or a pharmaceutically acceptable salt, solvate, or hydrate thereof; wherein:

R^{x} is a conjugating group;

L^{N} is a linker;

each Q is independently a polynucleotide comprising a phosphotriester; and

e is an integer of 1, 2, 3, or 4.

In certain embodiments, in Formula (B), R^{x} is

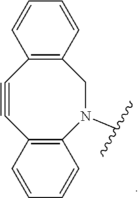 [SMILES: *N1Cc2ccccc2C#Cc2ccccc21]

In certain embodiments, in Formula (B), L^{N} is a linker comprising a polyethylene glycol.

In certain embodiments, in Formula (B), L^{N} is

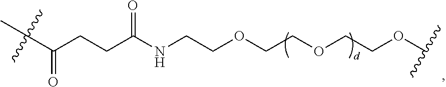

wherein d is an integer ranging from about 0 to about 50.

In certain embodiments, d is an integer ranging from about 0 to about 10. In certain embodiments, d is an integer ranging from about 0 to about 5. In certain embodiments, d is an integer of about 0, about 1, or about 3.

In certain embodiments, in Formula (B), e is an integer of 1.

In certain embodiments, in Formula (B), each Q independently has the structure of Formula (D):

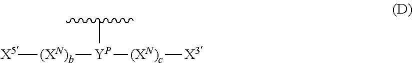

wherein X^{N}, X^{3′}, X^{5′}, Y^{P}, b, and c are each as defined herein.

### Targeting Moieties

The targeting moiety used in the conjugate provided herein is to a target specific cell and tissue in a body for targeted delivery of a conjugated payload polynucleotide. In certain embodiments, the cell targeted by the conjugate provided herein is a natural killer cell. In certain embodiments, the cell targeted by the conjugate provided herein is myeloid cell. In certain embodiments, the cell targeted by the conjugate provided herein is a neutrophil. In certain embodiments, the cell targeted by the conjugate provided herein is a monocyte. In certain embodiments, the cell targeted by the conjugate provided herein is a macrophage. In certain embodiments, the cell targeted by the conjugate provided herein is a dendritic cell (DC). In certain embodiments, the cell targeted by the conjugate provided herein is a mast cell. In certain embodiments, the cell targeted by the conjugate provided herein is a tumor-associated macrophage (TAM). In certain embodiments, the cell targeted by the conjugate provided herein is a myeloid-derived suppressor cell (MDSC).

In certain embodiments, the targeting moiety is an antigen-binding moiety. In certain embodiments, the targeting moiety is an antibody or antigen-binding fragment thereof.

In certain embodiments, the antigen-binding moiety in the conjugate provided herein is an antibody or an antigen-binding fragment thereof (e.g., F(ab)₂ or Fab) or an engineered derivative thereof (e.g., Fcab or a fusion protein (e.g., scFv)). In certain embodiments, the antigen-binding moiety in the conjugate provided herein is a human or chimeric (e.g., humanized) antibody.

The antigen-binding moiety targets the cell having the surface antigen that is recognized by the antigen-binding moiety.

In certain embodiments, the targeting moiety is an antibody binding to an antigen expressed by an NK cell. Exemplary antigens expressed by a NK cell and can be targeted by the conjugated provided herein include, but are not limited to, CD11 b, CD11c, CD16/32, CD49b, CD56 (NCAM), CD57, CD69, CD94, CD122, CD158 (Kir), CD161 (NK-1.1), CD244 (2B4), CD314 (NKG2D), CD319 (CRACC), CD328 (Siglec-7), CD335 (NKp46), Ly49, Ly108, Va24-Ja18 TCR (iNKT), granulysin, granzyme, perforin, SIRP-α, LAIR1, SIGLEC-3 (CD33), SIGLEC-7, SIGLEC-9, LIR1 (ILT2, LILRB1), NKR-P1 A (KLRB1), CD94-NKG2 A, KLRG1, KIR2DL5 A, KIR2DL5B, KIR2DL1, KIR2DL2, KIR2DL3, KIR2DS2, KIR2DS3, KIR2DS4, KIR2DS5, KIR3DS1, KIR2DS1, CD94-NKG2C/E, NKG2D, CD160 (BY55), CD16 (FcγRIIIA), NKp46 (NCR1), NKp30 (NCR3), NKp44 (NCR2), DNAM1(CD226), CRTAM, CD2, CD7, CD11 a, CD18, CD25, CD27, CD28, NTB-A (SLAMF6), PSGL1, CD96 (Tactile), CD100 (SEMA4D), NKp80 (KLRF1, CLEC5C), SLAMF7 (CRACC, CS1, CD319), and CD244 (2B4, SLAMF4).

In certain embodiments, the targeting moiety is an antibody binding to an antigen expressed by a myeloid cell. Exemplary antigens expressed by a myeloid cell and can be targeted by the conjugated provided herein include, but are not limited to, siglec-3, siglec 7, siglec 9, siglec 15, CD200, CD200R, LILRB1, LILRB2, LILRB3, LILRB4, LILRB5, M-CSF, CSF-1 R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1R, CD31, SIRPα, SIRPβ, PD-L1, CEACAM-8/CD66b, CD103, BDCA-1, BDCA2. BDCA-4, CD123, and ILT-7.

In certain embodiments, the targeting moiety is an antibody binding to an antigen expressed by an MDSC. Exemplary antigens expressed by an MDSC and can be targeted by the conjugated provided herein include, but are not limited to, siglec-3, Siglec 7, siglec 9, siglec 15, CD200, CD200R, LILRB1, LILRB2, LILRB3, LILRB4, LILRB5, M-CSF, CSF-1 R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1R, CD31, SIRPα, SIRPβ, PD-L1, CEACAM-8/CD66b, CD103, BDCA-1, BDCA2. BDCA-4, CD123, and ILT-7.

In certain embodiments, the targeting moiety is an antibody binding to an antigen expressed by a TAM. Exemplary antigens expressed by a TAM and can be targeted by the conjugated provided herein include, but are not limited to, siglec-3, Siglec 7, siglec 9, siglec 15, CD200, CD200R, LILRB1, LILRB2, LILRB3, LILRB4, LILRB5, M-CSF, CSF-1 R, GM-CSF R, IL4 R, arginase, IDO, TDO, MPO, EP2, COX-2, CCR2, CCR-7, CXCR1, CX3CR1, CXCR2, CXCR3, CXCR4, CXCR7, c-Kit, CD244, L-selectin/CD62L, CD11b, CD11c, CD68, CD163, CD204, DEC205, IL-1 R, CD31, SIRPα, SIRPβ, PD-L1, CEACAM-8/CD66b, CD103, BDCA-1, BDCA2. BDCA-4, CD123, and ILT-7.

In certain embodiments, the targeting moiety is an antibody binding to an antigen specific to a NK cell. In certain embodiments, an NK cell is targeted by an anti-CD56 antibody. In certain embodiments, the targeting moiety is an anti-CD56 antibody. In certain embodiments, the antibody is a monoclonal anti-CD56 antibody. In certain embodiments, the antibody is a murine anti-CD56 antibody. In certain embodiments, the murine anti-CD56 antibody is clone 5.1 H11 (BioLegend, Cat No: 362502). In certain embodiments, the murine anti-CD56 antibody is clone MEM-188 (BioLegend, 304601). In certain embodiments, the murine anti-CD56 antibody is clone QA17 A16 (BioLegend, Cat No: 392402). In certain embodiments, the antibody is a humanized anti-CD56 antibody. In certain embodiments, the antibody is a human anti-CD56 antibody. In certain embodiments, the antibody is a humanized anti-CD56 antibody

In certain embodiments, the targeting moiety is an antibody binding to an antigen specific to a myeloid cell. In certain embodiments, a myeloid cell is targeted by an anti-SIRPα antibody. In certain embodiments, the targeting moiety is an anti-SIRPα antibody. In certain embodiments, the antibody is a monoclonal anti-SIRPα antibody. In certain embodiments, the antibody is a murine anti-SIRPα antibody. In certain embodiments, the antibody is a humanized anti-SIRPα antibody. In certain embodiments, the antibody is a human anti-SIRPα antibody.

In certain embodiments, the anti-SIRPα antibody(119 or 119 germline mutants) is a human antibody comprising a VH and VL, wherein the VH is independently selected from the sequences listed below:

| Antibody | | | SEQ ID | |
|---|---|---|---|---|
| Name | Source | Domain | NO: | Sequence |
| 119 | Human | VH | 490 | EVQLLESGGGVVQPGGSLRLSCAASGFS |
| | | | | FSNFAMTWVRQAPGEGLEWVSTIGSGD |
| | | | | TYYADSVKGRFTISRDNSKNTLYLQMNSL |
| | | | | RAEDTAVYYCAKDSTVSWSGDFFDYWG |
| | | | | LGTLVTVSS |
| AB119 mut | Human | VH | 491 | EVQLLESGGGVVQPGGSLRLSCAASGFSF |
| | (D1E, | | | SNFAVTWVRQAPGKGLEWVSTIGSGDTYY |
| | E43K, | | | ADSVKGRFTISRDNSKNTLYLQMNSLRAED |
| | L112Q, | | | TAVYYCAKDSTVSWSGDFFDYWGQGTLV |
| | and M34V | | | TVSS |
| | germline | | | |
| | mutants) | | | |
| 119 VH | Human | VH | 492 | EVQLLESGGGVVQPGGSLRLSCAASGFSF |
| MutAll_ | (D1E, | | | SNFAMTWVRQAPGKGLEWVSTIGSGDTYY |
| V34M | E43K, and | | | ADSVKGRFTISRDNSKNTLYLQMNSLRAED |
| | L112Q | | | TAVYYCAKDSTVSWSGDFFDYWGQGTLV |
| | germline | | | TVSS |
| | mutants) | | | |
| 119_VH_ | Human | VH | 493 | EVQLLESGGGVVQPGGSLRLSCAASGFSF |
| MutAll_ | (germline | | | SNFALTWVRQAPGKGLEWVSTI |
| V34L | mutants) | | | GSGDTYYADSVKGRFTISRDNSKNTLYLQ |
| | | | | MNSLRAEDTAVYYCAKDSTVSWSGDFFDY |
| | | | | WGQGTLVTVSS |
| 119_M34L | Human | VH | 494 | DVQLLESGGGVVQPGGSLRLSCAASGFSF |
| | (germline | | | SNFALTWVRQAPGEGLEWVSTIGSGDTYY |
| | mutants) | | | ADSVKGRFTISR |
| | | | | DNSKNTLYLQMNSLRAEDTAVYYCAKDST |
| | | | | VSWSGDFFDYWGLGTLVTVSS |
| 119_M34V | Human | VH | 495 | DVQLLESGGGVVQPGGSLRLSCAASGFSF |
| | (germline | | | SNFAVTWVRQAPGEGLEWVSTIGSGDTYY |
| | mutants) | | | ADSVKGRFTISR |
| | | | | DNSKNTLYLQMNSLRAEDTAVYYCAKDST |
| | | | | VSWSGDFFDYWGLGTLVTVSS |

and the VL is independently selected from the sequences listed below:

| Antibody | | | SEQ | |
|---|---|---|---|---|
| Name | Source | Domain | ID NO | Sequence |
| 119 | Human | VL | 496 | EIVLTQSPATLSVSPGERATFSCRASQNVKN |
| | | | | DLAWYQQRPGQAPRLLIYAARIRETGIPERFS |
| | | | | GSGSGTEFTLTITSLQSEDFAVYYCQQYYDW |
| | | | | PPFTFGGGTKVEIK |
| 119 mut all | Human | VL | 497 | EIVLTQSPATLSVSPGERATLSCRASQNVKN |
| | (F21L, | | | DLAWYQQKPGQAPRLLIYAA |
| | R39K, | | | RIRETGIPARFSGSGSGTEFTLTISSLQSEDF |
| | E60A, and | | | AVYYCQQYYDWPPFTFGGGTKVEIK |
| | T76S | | | |
| | germline | | | |
| | mutants) | | | |

In certain embodiments, the andi-SIRPα antibody (119 or 119 germline mutants) is a human antibody comprising a HVR-H1, HVR-H2, HVR-H3, HVR-1L1, HVR-1L2, and HVR-1L3, each of which is independently selected from the table below.

| | | SEQ ID | |
|---|---|---|---|
| Antibody | Domain | NO: | Sequence |
| 119 | HVR-H1 | 498 | GFSFSNFAMT or |
| | | 499 | GFSFSNFAVT or |
| | | 500 | GFSFSNFALT |
| 119 | HVR-H2 | 501 | TIGSGDTYYADSVKG |
| 119 | HVR-H3 | 502 | DSTVSWSGDFFDY |
| 119 | HVR-L1 | 503 | RASQNVKNDLA |
| 119 | HVR-L2 | 504 | AARIRET |
| 119 | HVR-L3 | 505 | QQYYDWPPFT |

119 human antibodies are CD47-blockers, which are described in Table P of WO 2018/057669 A1, the disclosure of which is incorporated herein by reference in its entirety.

In certain embodiments, the anti-SIRPα antibody (135 or 135 germline mutants) is a human antibody comprising a VH and VL, wherein the VH is independently selected from the sequences listed below:

| Antibody | | | SEQ | |
|---|---|---|---|---|
| Name | Source | Domain | ID NO: | Sequence |
| 135 | Human | VH | 506 | DVQLVESGGGVVRPGESLRLSCAASGFS |
| | | | | FSIYAMSWVRQAPGEGLEWVSTIGADDT |
| | | | | YYADSVKGRFTISRDNSKNTLYLQMNSLR |
| | | | | AEDTAVYYCAKDSTVGWSGDFFDYWGL |
| | | | | GTLVTVSS |
| AB135 | Human | VH | 507 | EVQLVESGGGVVQPGGSLRLSCAASGFSF |
| mut | D1E, R130, | | | SIYAVSWVRQAPGKGLEWVSTIGADDTYYA |
| | E16G, E43K, | | | DSVKGRFTISRDNSKNTLYLQMNSLRAEDT |
| | L112Q, and | | | AVYYCAKDSTVGWSGDFFDYWGQGTLVTV |
| | M34V | | | SS |
| | germline | | | |
| | mutations | | | |
| 135 VH | Human | VH | 508 | EVQLVESGGGVVQPGGSLRLSCAASGFSF |
| MutAll_ | D1E, R13Q, | | | SIYAMSWVRQAPGKGLEWVSTIGADDTYYA |
| V34M | E16G, E43K, | | | DSVKGRFTISRDNSKNTLYLQMNSLRAEDT |
| | and L112Q | | | AVYYCAKDSTVGWSGDFFDYWGQGTLVTV |
| | germline | | | SS |
| | mutations | | | |
| 135_VH_ | Human | VH | 509 | EVQLVESGGGVVQPGGSLRLSCAASGFSF |
| MutAll_ | Germline back- | | | SIYALSWVRQAPGKGLEWVSTI |
| V34L | mutations and | | | GADDTYYADSVKGRFTISRDNSKNTLYLQM |
| | liability | | | NSLRAEDTAVYYCAKDSTVGWSGDFFDYW |
| | mutation | | | GQGTLVTVSS |

and the VL s independently selected from the sequences listed below:

| Antibody | | | SEQ ID | |
|---|---|---|---|---|
| Name | Source | Domain | NO: | Sequence |
| 135 | Human | VL | 510 | EIVLTQSPATLSVSPGERVTFSCRASQNV |
| | | | | RSDIAWYQQKPGQAPRLLIYAASSRDTGI |
| | | | | PDRFSGSGSGTDFTLTISSLQSEDFGVYY |
| | | | | CQQYYDWPPFTFGGGTKVEIK |
| 135 | Human | VL | 511 | EIVLTQSPATLSVSPGERVTLSCRASQNV |
| | F21L and | | | RSDIAWYQQKPGQAPRLLIYAASSRDTGI |
| | D60A | | | PARFSGSGSGTDFTLTISSLQSEDFGVYY |
| | germline | | | CQQYYDWPPFTFGGGTKVEIK |
| | mutations | | | |

In certain embodiments, the anti-SIRPα antibody (135 or 135 germline mutants) is a human antibody comprising a HVR-H1, HVR-H2, HVR-H3, HVR-1L1, HVR-1L2, and HVR-1L3, each of which is independently selected from the table below.

| Antibody | | SEQ ID | |
|---|---|---|---|
| Name/Source | Domain | NO: | Sequence |
| 135/Human | HVR-H1 | 512 | GFSFSIYAMS or |
| | | 513 | GFSFSIYAVS or |
| | | 514 | GFSFSIYALS |
| 135/Human | HVR-H2 | 515 | TIGADDTYYADSVKG |
| 135/Human | HVR-H3 | 516 | DSTVGWSGDFFDY |
| 135/Human | HVR-L1 | 517 | RASQNVRSDIA |
| 135/Human | HVR-L2 | 518 | AASSRDT |
| 135/Human | HVR-L3 | 519 | QQYYDWPPFT |

135 human antibodies are CD47-blockers, which are described in Table P of WO 2018/057669 A1, the disclosure of which is incorporated herein by reference in its entirety.

In certain embodiments, the anti-SIRPα antibody (AB21, AB21 germline mutants or humanized version of AB21) is an antibody comprising a VH and VL, wherein the VH is independently selected from the sequences listed below:

| Antibody | | | SEQ ID | |
|---|---|---|---|---|
| Name | Source | Domain | NO: | Sequence |
| AB21 | Human | VH | 520 | DVQLVESGGGVVRPGESLRLSCAASGFTF |
| | | | | SSNAMSWVRQAPGKGLEWLAGISAGGSDT |
| | | | | YYPASVKGRFTISRDNSKNTLYLQMNTLTA |
| | | | | EDTAVYYCARETWNHLFDYWGLGTLVTVS |
| | | | | S |
| AB21 | Human with | VH | 521 | EVQLVESGGGVVQPGGSLRLSCAASGFTF |
| Mut All | germline | | | SSNAMSWVRQAPGKGLEWVAGISAGGSDT |
| | back- | | | YYPASVKGRFTISRDNSKNTLYLQMNSLRA |
| | mutations | | | EDTAVYYCARETWNHLFDYWGQGTLVTVS |
| | | | | S |
| AB21 | Human with | VH | 522 | EVQLVESGGGVVQPGGSLRLSCAASGFTF |
| Mut All | germline | | | SSNAVSWVRQAPGKGLEWVAGISAGGSDT |
| M34V | back- | | | YYPASVKGRFTISRDNSKNTLYLQMNSLRA |
| | mutations | | | EDTAVYYCARETWNHLFDYWGQGTLVTVS |
| | and liability | | | S |
| | mutation | | | |
| AB21_HC_ | Human with | VH | 523 | EVQLVESGGGVVQPGGSLRLSCAASGFTF |
| MutAll_ | germline | | | SSNALSWVRQAPGKGLEWVAGISAGGSDT |
| M34L | back- | | | YYPASVKGRFTISRDNSKNTLYLQMNSLRA |
| | mutations | | | EDTAVYYCARETWNHLFDYWGQGTLVTVS |
| | and liability | | | S |
| | mutation | | | |

and the VL is independently selected from the sequences listed below:

| Antibody | | | SEQ ID | |
|---|---|---|---|---|
| Name | Source | Domain | NO: | Sequence |
| AB21 | Chicken | VL | 524 | ALTQPASVSANPGETVKIACSGGDYYS |
| | | | | YYYGWYQQKAPGSALVTVIYSDDKRPS |
| | | | | DIPSRFSGSASGSTATLTITGVRAEDEA |
| | | | | VYYCGGYDYSTYANAFGAGTTLTVL |
| Hum1 | Humanized | VL | 525 | SYELTQPPSVSVSPGQTARITCSGGSY |
| | | | | SSYYYAWYQQKPGQAPVTLIYSDDKRP |
| | | | | SNIPERFSGSSSGTTVTLTISGVQAEDE |
| | | | | ADYYCGGYDQSSYTNPFGGGTKLTVL |
| Hum2 | Humanized | VL | 526 | QSVLTQPPSVSAAPGQKVTISCSGGSY |
| | | | | SSYYYAWYQQLPGTAPKTLIYSDDKRP |
| | | | | SNIPDRFSGSKSGTSATLGITGLQTGDE |
| | | | | ADYYCGGYDQSSYTNPFGTGTKVTVL |
| Hum3 | Humanized | VL | 527 | SYELTQPPSVSVSPGQTARITCSGGDY |
| | | | | YSTYYAWYQQKPGQAPVTVIHSDDKRP |
| | | | | SDIPERFSGSSSGTTVTLTISGVQAEDE |
| | | | | ADYYCGGYDGRTYINTFGGGTKLTVL |
| Hum4 | Humanized | VL | 528 | QSVLTQPPSVSAAPGQKVTISCSGGDY |
| | | | | YSTYYAWYQQLPGTAPKTVIHSDDKRP |
| | | | | SDIPDRFSGSKSGTSATLGITGLQTGDE |
| | | | | ADYYCGGYDGRTYINTFGTGTKVTVL |
| Hum5 | Humanized | VL | 529 | QSALTQPASVSGSPGQSITISCTGTSSD |
| | | | | VGSYSSYYYAWYQQHPGKAPKTLIYSD |
| | | | | DKRPSNVSNRFSGSKSGNTASLTISGL |
| | | | | QAEDEADYYCGGYDQSSYTNPFGGGT |
| | | | | KLTVL |
| Hum6 | Humanized | VL | 530 | QSVLTQPPSVSAAPGQKVTISCSGGDY |
| | | | | YSYYYGWYQQLPGTAPKTVIYSDDKRP |
| | | | | SDIPDRFSGSKSGTSATLGITGLQTGDE |
| | | | | ADYYCGGYDYSTYANAFGTGTKVTVL |
| Hum8 | Humanized | VL | 531 | SYELTQPPSVSVSPGQTARITCSGGAYSS |
| | | | | YYYAWYQQKPGQAPVLVIYSDSKRPSGIP |
| | | | | ERFSGSSSGTTVTLTISGVQAEDEADYYC |
| | | | | GGYDQSSYTNPFGGGTKLTVL |
| Hum9 | Humanized | VL | 532 | SYELTQPPSVSVSPGQTARITCSGGAYSS |
| | | | | YYYAWYQQKPGQAPVLVIYSDDKRPSGIP |
| | | | | ERFSGSSSGTTVTLTISGVQAEDEADYYC |
| | | | | GGYDQSSYTNPFGGGTKLTVL |

In certain embodiments, the anti-SIRPα antibody (AB21, AB21 germline mutants or humanized version of AB21) is a humanized antibody comprising a HVR-H1, HVR-H2, HVR-H3, HVR-L1, HVR-L2, and HVR-L3, each of which is independently selected from the table below.

| Name/Source | Domain | SEQ ID NO: | Sequence |
|---|---|---|---|
| AB21/Human | HVR-H1 | 533 | GFTFSSNALS or |
| | | 534 | GFTFSSNAMS or |
| | | 535 | GFTFSSNAVS |
| AB21/Human | HVR-H2 | 536 | ISAGGSDT |
| AB21/Human | HVR-H3 | 537 | ARETWNHLFDY |
| AB21/Chicken | HVR-L1 | 538 | SGGDYYSYYYG or |
| or humanized | | 539 | SGGSYSSYYYA or |
| | | 540 | SGGDYYSTYYA or |
| | | 541 | GSYSSYYYA or |
| | | 542 | SGGAYSSYYYA |
| AB21/Chicken | HVR-L2 | 543 | SDDKRPS |
| or humanized | | | |
| AB21/Chicken | HVR-L3 | 544 | GGYDYSTYANA or |
| or humanized | | 545 | GGYDQSSYTNP or |
| | | 546 | GGYDGRTYINT |

AB21 humanized antibodies are CD47-blockers, which are described in Table P of WO 2018/057669 A1, the disclosure of which is incorporated herein by reference in its entirety.

In certain embodiments, the anti-SIRPα antibody (136 or 136 germline mutants) is a human antibody comprising a VH and VL, wherein the VH is independently selected from the sequences listed below:

| Antibody | | | SEQ ID | |
|---|---|---|---|---|
| Name | Source | Domain | NO: | Sequence |
| 136 | Human | VH | 547 | DVQLVESGGGVVRPGESLRLSCAASGFTFS |
| | | | | SYDMNWVRQAPGEGLEWVSLISGSGEIIYY |
| | | | | ADSVKGRFTISRDNSKNTLYLQMNSLRAED |
| | | | | TAVYYCAKENNRYRFFDDWGLGTLVTVSS |
| 136 mut | Human with | VH | 548 | EVQLVESGGGVVQPGRSLRLSCAASGFTFSS |
| all | D1E, R130, | | | YDVNWVRQAPGKGLEWVSLISGSGEIIYYADS |
| | E16R, E43K, | | | VKGRFTISRDNSKNTLYLQMNSLRAEDTAVYY |
| | L111Q, and | | | CAKENNRYRFFDDWGQGTLVTVSS |
| | M34V | | | |
| | germline | | | |
| | mutations | | | |
| 136_VH_ | Human with | VH | 549 | EVQLVESGGGVVQPGRSLRLSCAASGFTFSS |
| Mutall_ | germline | | | YDLNWVRQAPGKGLEWVSLI |
| V34L | back- | | | SGSGEIIYYADSVKGRFTISRDNSKNTLYLQMN |
| | mutations | | | SLRAEDTAVYYCAKENNRYRFFDDWGQGTLV |
| | and liability | | | TVSS |
| | mutation | | | |
| 136 VH | Human with | VH | 550 | EVQLVESGGGVVQPGRSLRLSCAASGFTFSS |
| MutAll_ | D1E, R13Q, | | | YDMNWVRQAPGKGLEWVSLISGSGEIIYYADS |
| V34M | E16R, E43K, | | | VKGRFTISRDNSKNTLYLQMNSLRAEDTAVYY |
| | and L111Q | | | CAKENNRYRFFDDWGQGTLVTVSS |
| | germline | | | |
| | mutations | | | |

and the VL is independently selected from the sequences listed below:

| Antibody | | | SEQ ID | Sequence |
|---|---|---|---|---|
| Name | Source | Domain | NO: | |
| 136 | Human | VL | 551 | ETVLTQSPGTLTLSPGERATLTCRASQSVYTY |
| | | | | LAWYQEKPGQAPRLLIYGASSRATGIPDRFSG |
| | | | | SGSGTEFTLTISSLQSEDFAVYYCQQYYDRPP |
| | | | | LTFGGGTKVEIK |
| 136 mut | Human with | VL | 552 | EIVLTQSPGTLSLSPGERATLSCRASQSVYTYL |
| all | T2I, T12S, | | | AWYQQKPGQAPRLLIYGASSRATGIPDRFSG |
| | T22S, and | | | SGSGTEFTLTISSLQSEDFAVYYCQQYYDRPP |
| | E38Q | | | LTFGGGTKVEIK |
| | germline | | | |
| | mutations | | | |
| 136 mut | Human with | VL | 553 | ETVLTQSPGTLSLSPGERATLSCRASQSVYTY |
| all_I2T | T12S, T22S, | | | LAWYQQKPGQAPRLLIYGASSRATGIPDRFSG |
| | and E380 | | | VSGSGTEFTLTISSLQSEDFAVYYCQQYYDRPP |
| | germline | | | LTFGGGTKVEIK |
| | mutations | | | |

In certain embodiments, the anti-SIRPα antibody (136 or 136 germline mutants) is a human antibody comprising a HVR-H1, HVR-H2, HVR-H3, HVR-1L1, HVR-L-2, and HVR-L-3, each of which is independently selected from the table below.

| Antibody | | SEQ | |
|---|---|---|---|
| Name/Source | Domain | ID NO: | Sequence |
| 136/Human | HVR-H1 | 554 | GFTFSSYDMN or |
| | | 555 | GFTFSSYDVN or |
| | | 556 | GFTFSSYDLN |
| 136/Human | HVR-H2 | 557 | LISGSGEIIYYADSVKG |
| 136/Human | HVR-H3 | 558 | ENNRYRFFDD |
| 136/Human | HVR-L1 | 559 | RASQSVYTYLA |
| 136/Human | HVR-L2 | 560 | GASSRAT |
| 136/Human | HVR-L3 | 561 | QQYYDRPPLT |

136 human antibodies are non-blockers, which are described in Table P of WO 2018/057669 A1, the disclosure of which is incorporated herein by reference in its entirety.

In certain embodiments, the anti-SIRPα antibody (218 or humanized 218) is an antibody comprising a VH and VL, wherein the VH has the sequence of DVQLVESGGGVVRPGESLTLSCTASGFTFTSSTMNWVRQAPGEGLDWVSSISTSGVITYYADSVKG RATISRDNSKNTLYLRLFSLRADDTAIYYCATDTFDHWGPGTLVTVSS (SEQ ID NO: 584); and the VL is independently selected from the sequences listed below:

| Antibody | | | SEQ ID | |
|---|---|---|---|---|
| Name | Source | Domain | NO: | Sequence |
| 218 | Chicken | VL | 585 | ALTQPASVSANPGETVKITCFGSSGNYGWF |
| | | | | QQKSPGSAPVTVIHYNNKRPSDIPSRFSGS |
| | | | | KSGSTGTLTITGVRAEDEAVYFCGAWETGS |
| | | | | ATFGAGTTLTVL |
| 218_Hum13 | Humanized | VL | 562 | QSALTQPASVSGSPGQSITISCFGSSGNYG |
| (218 VL with | | | | LVSWYQQHPGKAPKLMIYYNNKRPSGVSN |
| human | | | | RFSGSKSGNTASLTISGLQAEDEADYYCGA |
| IGLV2) | | | | WETGSATFGGGTKLTVL |
| 218_Hum14 | Humanized | VL | 563 | SYELTQPPSVSVSPGQTASITCFGSSGNYG |
| (218 VL with | | | | WYQQKPGQSPVLVIYYNNKRPSGIPERFSG |
| human | | | | SNSGNTATLTISGTQAMDEADYYCGAWET |
| IGLV3) | | | | GSATFGGGTKLTVL |

218 human antibodies are non-blockers, which are described in Table P of WO 2018/057669 A1, the disclosure of which is incorporated herein by reference in its entirety.

In some embodiments, an anti-SIRPα antibody comprises a heavy chain variable (VH) domain comprising an HVR-H1 comprising a sequence selected from the group consisting of SEQ ID NOs:498-500, an HVR-H2 comprising the sequence of SEQ ID NO:501, and an HVR-H3 comprising the sequence of SEQ ID NO:502; and/or a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505. In some embodiments, an anti-SIRPα antibody comprises a heavy chain variable (VH) domain comprising an HVR-H1 comprising the sequence of SEQ ID NO:498, an HVR-H2 comprising the sequence of SEQ ID NO:501, and an HVR-H3 comprising the sequence of SEQ ID NO:502; and/or a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505. In some embodiments, an anti-SIRPα antibody comprises a heavy chain variable (VH) domain comprising an HVR-H1 comprising the sequence of SEQ ID NO:499, an HVR-H2 comprising the sequence of SEQ ID NO:501, and an HVR-H3 comprising the sequence of SEQ ID NO:502; and/or a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505. In some embodiments, an anti-SIRPα antibody comprises a heavy chain variable (VH) domain comprising an HVR-H1 comprising the sequence of SEQ ID NO:500, an HVR-H2 comprising the sequence of SEQ ID NO:501, and an HVR-H3 comprising the sequence of SEQ ID NO:502; and/or a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505.

In some embodiments, an anti-SIRPα antibody comprises a VH domain comprising a sequence selected from the group consisting of SEQ ID NOs:490-495 and/or a VL domain comprising the sequence of SEQ ID NO:496 or 497. In some embodiments, an anti-SIRPα antibody comprises a VH domain comprising the sequence of SEQ ID NO:490 and/or a VL domain comprising the sequence of SEQ ID NO:496. In some embodiments, an anti-SIRPα antibody comprises a VH domain comprising the sequence of SEQ ID NO:491 and/or a VL domain comprising the sequence of SEQ ID NO:496. In some embodiments, an anti-SIRPα antibody comprises a VH domain comprising the sequence of SEQ ID NO:492 and/or a VL domain comprising the sequence of SEQ ID NO:496. In some embodiments, an anti-SIRPα antibody comprises a VH domain comprising the sequence of SEQ ID NO:493 and/or a VL domain comprising the sequence of SEQ ID NO:496. In some embodiments, an anti-SIRPα antibody comprises a VH domain comprising the sequence of SEQ ID NO:494 and/or a VL domain comprising the sequence of SEQ ID NO:496. In some embodiments, an anti-SIRPα antibody comprises a VH domain comprising the sequence of SEQ ID NO:495 and/or a VL domain comprising the sequence of SEQ ID NO:496. In some embodiments, an anti-SIRPα antibody comprises a VH domain comprising the sequence of SEQ ID NO:490 and/or a VL domain comprising the sequence of SEQ ID NO:497. In some embodiments, an anti-SIRPα antibody comprises a VH domain comprising the sequence of SEQ ID NO:491 and/or a VL domain comprising the sequence of SEQ ID NO: 497. In some embodiments, an anti-SIRPα antibody comprises a VH domain comprising the sequence of SEQ ID NO:492 and/or a VL domain comprising the sequence of SEQ ID NO: 497. In some embodiments, an anti-SIRPα antibody comprises a VH domain comprising the sequence of SEQ ID NO:493 and/or a VL domain comprising the sequence of SEQ ID NO: 497. In some embodiments, an anti-SIRPα antibody comprises a VH domain comprising the sequence of SEQ ID NO:494 and/or a VL domain comprising the sequence of SEQ ID NO: 497. In some embodiments, an anti-SIRPα antibody comprises a VH domain comprising the sequence of SEQ ID NO:495 and/or a VL domain comprising the sequence of SEQ ID NO: 497.

In some embodiments, an anti-SIRPα antibody comprises a heavy chain variable (VH) domain comprising an HVR-H1 comprising a sequence selected from the group consisting of SEQ ID NOs:512-514, an HVR-H2 comprising the sequence of SEQ ID NO:515, and an HVR-H3 comprising the sequence of SEQ ID NO:516; and/or a light chain variable (VL) domain comprising an HVR-L1 comprising the sequence of SEQ ID NO:517, an HVR-L2 comprising the sequence of SEQ ID NO:518, and an HVR-L3 comprising the sequence of SEQ ID NO:519. In some embodiments, an anti-SIRPα antibody comprises a heavy chain variable (VH) domain comprising an HVR-H1 comprising the sequence of SEQ ID NO:512, an HVR-H2 comprising the sequence of SEQ ID NO:515, and an HVR-H3 comprising the sequence of SEQ ID NO:516; and/or a light chain variable (VL) domain comprising an HVR-L1 comprising the sequence of SEQ ID NO:517, an HVR-L2 comprising the sequence of SEQ ID NO:518, and an HVR-L3 comprising the sequence of SEQ ID NO:519. In some embodiments, an anti-SIRPα antibody comprises a heavy chain variable (VH) domain comprising an HVR-H1 comprising the sequence of SEQ ID NO:513, an HVR-H2 comprising the sequence of SEQ ID NO:515, and an HVR-H3 comprising the sequence of SEQ ID NO:516; and/or a light chain variable (VL) domain comprising an HVR-L1 comprising the sequence of SEQ ID NO:517, an HVR-L2 comprising the sequence of SEQ ID NO:518, and an HVR-L3 comprising the sequence of SEQ ID NO:519. In some embodiments, an anti-SIRPα antibody comprises a heavy chain variable (VH) domain comprising an HVR-H1 comprising the sequence of SEQ ID NO:514, an HVR-H2 comprising the sequence of SEQ ID NO:515, and an HVR-H3 comprising the sequence of SEQ ID NO:516; and/or a light chain variable (VL) domain comprising an HVR-L1 comprising the sequence of SEQ ID NO:517, an HVR-L2 comprising the sequence of SEQ ID NO:518, and an HVR-L3 comprising the sequence of SEQ ID NO:519.

In some embodiments, an anti-SIRPα antibody comprises a VH domain comprising a sequence selected from the group consisting of SEQ ID NOs:506-509 and/or a VL domain comprising the sequence of SEQ ID NO:510 or 511. In some embodiments, an anti-SIRPα antibody comprises a VH domain comprising the sequence of SEQ ID NO:506 and/or a VL domain comprising the sequence of SEQ ID NO:510. In some embodiments, an anti-SIRPα antibody comprises a VH domain comprising the sequence of SEQ ID NO:507 and/or a VL domain comprising the sequence of SEQ ID NO:510. In some embodiments, an anti-SIRPα antibody comprises a VH domain comprising the sequence of SEQ ID NO:508 and/or a VL domain comprising the sequence of SEQ ID NO:510. In some embodiments, an anti-SIRPα antibody comprises a VH domain comprising the sequence of SEQ ID NO:509 and/or a VL domain comprising the sequence of SEQ ID NO:510. In some embodiments, an anti-SIRPα antibody comprises a VH domain comprising the sequence of SEQ ID NO:506 and/or a VL domain comprising the sequence of SEQ ID NO:511. In some embodiments, an anti-SIRPα antibody comprises a VH domain comprising the sequence of SEQ ID NO:507 and/or a VL domain comprising the sequence of SEQ ID NO:511. In some embodiments, an anti-SIRPα antibody comprises a VH domain comprising the sequence of SEQ ID NO:508 and/or a VL domain comprising the sequence of SEQ ID NO:511. In some embodiments, an anti-SIRPα antibody comprises a VH domain comprising the sequence of SEQ ID NO:509 and/or a VL domain comprising the sequence of SEQ ID NO:511.

In some embodiments, an anti-SIRPα antibody comprises a heavy chain variable (VH) domain comprising an HVR-H1 comprising a sequence selected from the group consisting of SEQ ID NOs:533-535, an HVR-H2 comprising the sequence of SEQ ID NO:536, and an HVR-H3 comprising the sequence of SEQ ID NO:537; and/or a light chain variable (VL) domain comprising an HVR-L1 comprising a sequence selected from the group consisting of SEQ ID NOs:538-542, an HVR-L2 comprising the sequence of SEQ ID NO:543, and an HVR-L3 comprising a sequence selected from the group consisting of SEQ ID NOs:544-546. In some embodiments, an anti-SIRPα antibody comprises a heavy chain variable (VH) domain comprising an HVR-H1 comprising the sequence of SEQ ID NO:534, an HVR-H2 comprising the sequence of SEQ ID NO:536, and an HVR-H3 comprising the sequence of SEQ ID NO:537; and/or a light chain variable (VL) domain comprising an HVR-L1 comprising the sequence of SEQ ID NO:539, an HVR-L2 comprising the sequence of SEQ ID NO:543, and an HVR-L3 comprising the sequence of SEQ ID NO:545. In some embodiments, an anti-SIRPα antibody comprises a heavy chain variable (VH) domain comprising an HVR-H1 comprising the sequence of SEQ ID NO:533, an HVR-H2 comprising the sequence of SEQ ID NO:536, and an HVR-H3 comprising the sequence of SEQ ID NO:537; and/or a light chain variable (VL) domain comprising an HVR-L1 comprising the sequence of SEQ ID NO:542, an HVR-L2 comprising the sequence of SEQ ID NO:543, and an HVR-L3 comprising the sequence of SEQ ID NO:546. In some embodiments, an anti-SIRPα antibody comprises a heavy chain variable (VH) domain comprising an HVR-H1 comprising the sequence of SEQ ID NO:498, an HVR-H2 comprising the sequence of SEQ ID NO:501, and an HVR-H3 comprising the sequence of SEQ ID NO:502; and/or a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505. In some embodiments, an anti-SIRPα antibody comprises a heavy chain variable (VH) domain comprising an HVR-H1 comprising the sequence of SEQ ID NO:554, an HVR-H2 comprising the sequence of SEQ ID NO:557, and an HVR-H3 comprising the sequence of SEQ ID NO:558; and/or a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561.

In some embodiments, an anti-SIRPα antibody comprises a VH domain comprising a sequence selected from the group consisting of SEQ ID NOs:520-523 and/or a VL domain comprising a sequence selected from the group consisting of SEQ ID NOs:525-532.

In some embodiments, an anti-SIRPα antibody comprises a heavy chain variable (VH) domain comprising an HVR-H1 comprising a sequence selected from the group consisting of SEQ ID NOs:554-556, an HVR-H2 comprising the sequence of SEQ ID NO:557, and an HVR-H3 comprising the sequence of SEQ ID NO:558; and/or a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561. In some embodiments, an anti-SIRPα antibody comprises a heavy chain variable (VH) domain comprising an HVR-H1 comprising the sequence of SEQ ID NO:554, an HVR-H2 comprising the sequence of SEQ ID NO:557, and an HVR-H3 comprising the sequence of SEQ ID NO:558; and/or a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561. In some embodiments, an anti-SIRPα antibody comprises a heavy chain variable (VH) domain comprising an HVR-H1 comprising the sequence of SEQ ID NO:555, an HVR-H2 comprising the sequence of SEQ ID NO:557, and an HVR-H3 comprising the sequence of SEQ ID NO:558; and/or a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561. In some embodiments, an anti-SIRPα antibody comprises a heavy chain variable (VH) domain comprising an HVR-H1 comprising the sequence of SEQ ID NO:556, an HVR-H2 comprising the sequence of SEQ ID NO:557, and an HVR-H3 comprising the sequence of SEQ ID NO:558; and/or a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561.

In some embodiments, an anti-SIRPα antibody comprises a VH domain comprising a sequence selected from the group consisting of SEQ ID NOs:547-550 and/or a VL domain comprising a sequence selected from the group consisting of SEQ ID NOs:551-553. In some embodiments, an anti-SIRPα antibody comprises a VH domain comprising the sequence of SEQ ID NO:547 and/or a VL domain comprising the sequence of SEQ ID NO:551. In some embodiments, an anti-SIRPα antibody comprises a VH domain comprising the sequence of SEQ ID NO:548 and/or a VL domain comprising the sequence of SEQ ID NO:551. In some embodiments, an anti-SIRPα antibody comprises a VH domain comprising the sequence of SEQ ID NO:549 and/or a VL domain comprising the sequence of SEQ ID NO:551. In some embodiments, an anti-SIRPα antibody comprises a VH domain comprising the sequence of SEQ ID NO:550 and/or a VL domain comprising the sequence of SEQ ID NO:551. In some embodiments, an anti-SIRPα antibody comprises a VH domain comprising the sequence of SEQ ID NO:547 and/or a VL domain comprising the sequence of SEQ ID NO:552. In some embodiments, an anti-SIRPα antibody comprises a VH domain comprising the sequence of SEQ ID NO:548 and/or a VL domain comprising the sequence of SEQ ID NO:552. In some embodiments, an anti-SIRPα antibody comprises a VH domain comprising the sequence of SEQ ID NO:549 and/or a VL domain comprising the sequence of SEQ ID NO:552. In some embodiments, an anti-SIRPα antibody comprises a VH domain comprising the sequence of SEQ ID NO:550 and/or a VL domain comprising the sequence of SEQ ID NO:552. In some embodiments, an anti-SIRPα antibody comprises a VH domain comprising the sequence of SEQ ID NO:547 and/or a VL domain comprising the sequence of SEQ ID NO:553. In some embodiments, an anti-SIRPα antibody comprises a VH domain comprising the sequence of SEQ ID NO:548 and/or a VL domain comprising the sequence of SEQ ID NO:553. In some embodiments, an anti-SIRPα antibody comprises a VH domain comprising the sequence of SEQ ID NO:549 and/or a VL domain comprising the sequence of SEQ ID NO:553. In some embodiments, an anti-SIRPα antibody comprises a VH domain comprising the sequence of SEQ ID NO:550 and/or a VL domain comprising the sequence of SEQ ID NO:553.

In some embodiments, an anti-SIRPα antibody comprises a VH domain comprising the sequence of SEQ ID NO:584 and/or a VL domain comprising a sequence selected from the group consisting of SEQ ID NOs:585, 562, and 563. In some embodiments, an anti-SIRPα antibody comprises a VH domain comprising the sequence of SEQ ID NO:584 and/or a VL domain comprising the sequence of SEQ ID NO:585. In some embodiments, an anti-SIRPα antibody comprises a VH domain comprising the sequence of SEQ ID NO:584 and/or a VL domain comprising the sequence of SEQ ID NO:562. In some embodiments, an anti-SIRPα antibody comprises a VH domain comprising the sequence of SEQ ID NO:584 and/or a VL domain comprising the sequence of SEQ ID NO:563. In some embodiments, an anti-SIRPα antibody comprises a VH domain comprising three HVRs of the sequence of SEQ ID NO:584 and/or a VL domain comprising three HVRs of the sequence of SEQ ID NO:585. In some embodiments, an anti-SIRPα antibody comprises a VH domain comprising three HVRs of the sequence of SEQ ID NO:584 and/or a VL domain comprising three HVRs of the sequence of SEQ ID NO:562. In some embodiments, an anti-SIRPα antibody comprises a VH domain comprising three HVRs of the sequence of SEQ ID NO:584 and/or a VL domain comprising three HVRs of the sequence of SEQ ID NO:563.

Additional anti-SIRP antibodies are disclosed in US 2018/0037652 A1; WO 2016/205042 A1; WO 2017/178653 A2; WO 2018/107058 A1; and WO 2018/057669 A1; the disclosure of each of which is incorporated herein by reference in its entirety.

In some embodiments, an antibody provided herein comprises a human Fc region, e.g., a human IgG1, IgG2, or IgG4 Fc region.

In some embodiments, the Fc region of the antibody provided herein includes one or more mutations that influence one or more antibody properties, such as stability, pattern of glycosylation or other modifications, effector cell function, pharmacokinetics, and so forth. In some embodiments, an antibody provided herein has reduced or minimal glycosylation. In some embodiments, an antibody provided herein has ablated or reduced effector function. Exemplary Fc mutations include without limitation (i) a human IgG1 Fc region mutations L234 A, L235 A, G237 A, and N297 A; (ii) a human IgG2 Fc region mutations A330 S, P331 S and N297 A; and (iii) a human IgG4 Fc region mutations S228P, E233P, F234V, L235 A, delG236, and N297 A (EU numbering). In some embodiments, the human IgG2 Fc region comprises A330 S and P331 S mutations. In some embodiments, the human IgG4 Fc region comprises an S288P mutation. In some embodiments, the human IgG4 Fc region comprises S288P and L235E mutations.

In some embodiments, an antibody provided herein comprises a human IgG1 Fc region comprising L234 A, L235 A, and G237 A mutations, according to EU numbering. In some embodiments, an antibody provided herein comprises a human IgG1 Fc region comprising L234 A, L235 A, G237 A, and N297 A mutations, according to EU numbering. In some embodiments, an antibody provided herein comprises a human IgG1 Fc region comprising an N297 A mutation, according to EU numbering. In some embodiments, an antibody provided herein comprises a human IgG1 Fc region comprising a D265 A mutation, according to EU numbering. In some embodiments, an antibody provided herein comprises a human IgG1 Fc region comprising D265 A and N297 A mutations, according to EU numbering. In some embodiments, an antibody provided herein comprises a human IgG2 Fc region comprising A330 S and P331 S mutations, according to EU numbering. In some embodiments, an antibody provided herein comprises a human IgG2 Fc region comprising A330 S, P331 S, and N297 A mutations, according to EU numbering. In some embodiments, an antibody provided herein comprises a human IgG2 Fc region comprising an N297 A mutation, according to EU numbering. In some embodiments, an antibody provided herein comprises a human IgG4 Fc region comprising an S228P mutation, according to EU numbering. In some embodiments, an antibody provided herein comprises a human IgG4 Fc region comprising S228P and D265 A mutations, according to EU numbering. In some embodiments, an antibody provided herein comprises a human IgG4 Fc region comprising S228P and L235E mutations, according to EU numbering. In some embodiments, an antibody provided herein comprises a human IgG4 Fc region comprising S228P and N297 A mutations, according to EU numbering. In some embodiments, an antibody provided herein comprises a human IgG4 Fc region comprising S228P, E233P, F234V, L235 A, delG236, and N297 A mutations, according to EU numbering. In some embodiments, an antibody provided herein comprises an Fc region that comprises a sequence selected from the group consisting of SEQ ID NOs:564-578.

In some embodiments, an antibody provided herein comprises a human kappa light chain constant domain, e.g., an Fc region comprising the sequence of SEQ ID NO:579. In some embodiments, an antibody provided herein comprises a human lambda light chain constant domain, e.g., IGLC1 or IGLC2 (such as the exemplary Fc region sequences shown in SEQ ID Nos:580 and 581, respectively).

Antibodies that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and, release of inflammatory mediators, placental transfer, and control of immunoglobulin production. Additionally, binding of the C1 component of complement to antibodies can activate the complement system. Activation of complement can be important for the lysis of cellular pathogens. However, the activation of complement can also stimulate the inflammatory response and can also be involved in autoimmune hypersensitivity or other immunological disorders. Variant Fc regions with reduced or ablated ability to bind certain Fc receptors are useful for developing therapeutic antibodies and Fc-fusion polypeptide constructs which act by targeting, activating, or neutralizing ligand functions while not damaging or destroying local cells or tissues.

In some embodiments, an Fc domain monomer refers to a polypeptide chain that includes second and third antibody constant domains (e.g., CH₂ and CH₃). In some embodiments, an Fc domain monomer also includes a hinge domain. In some embodiments, the Fc domain monomer is of any immunoglobulin antibody isotype, including IgG, IgE, IgM, IgA, and IgD. Additionally, in some embodiments, an Fc domain monomer is of any IgG subtype (e.g., IgG1, IgG2, IgG2a, IgG2b, IgG2c, IgG3, and IgG4). In some embodiments, Fc domain monomers include as many as ten changes from a wild-type Fc domain monomer sequence (e.g., 1-10, 1-8, 1-6, 1-4 amino acid substitutions, additions or insertions, deletions, or combinations thereof) that alter the interaction between an Fc domain and an Fc receptor.

In some embodiments, an Fc domain monomer of an immunoglobulin or a fragment of an Fc domain monomer is capable of forming an Fc domain with another Fc domain monomer. In some embodiments, an Fc domain monomer of an immunoglobulin or a fragment of an Fc domain monomer is not capable of forming an Fc domain with another Fc domain monomer. In some embodiments, an Fc domain monomer or a fragment of an Fc domain is fused to a polypeptide of the disclosure to increase serum half-life of the polypeptide. In some embodiments, an Fc domain monomer or a fragment of an Fc domain monomer fused to a polypeptide of the disclosure dimerizes with a second Fc domain monomer to form an Fc domain which binds an Fc receptor, or alternatively, an Fc domain monomer binds to an Fc receptor. In some embodiments, an Fc domain or a fragment of the Fc domain fused to a polypeptide to increase serum half-life of the polypeptide does not induce any immune system-related response. An Fc domain includes two Fc domain monomers that are dimerized by the interaction between the CH₃ antibody constant domains.

A wild-type Fc domain forms the minimum structure that binds to an Fc receptor, e.g., FcγRI, FcγRIIa, FcγRIIb, FcγRIIIa, FcγRIIIb, and FcγRIV. In some embodiments, the Fc domain in an antibody of the present disclosure comprises one or more amino acid substitutions, additions or insertions, deletions, or any combinations thereof that lead to decreased effector function such as decreased antibody-dependent cell-mediated cytotoxicity (ADCC), decreased complement-dependent cytolysis (CDC), decreased antibody-dependent cell-mediated phagocytosis (ADCP), or any combinations thereof. For example, an antibody of the present disclosure can exhibit decreased binding (e.g., minimal binding or absence of binding) to a human Fc receptor and decreased binding (e.g., minimal binding or absence of binding) to complement protein C1q; decreased binding (e.g., minimal binding or absence of binding) to human FcγRI, FcγRIIA, FcγRIIB, FcγRIIIB, FcγRIIIB, or any combinations thereof, and C1q; altered or reduced antibody-dependent effector function, such as ADCC, CDC, ADCP, or any combinations thereof; and so forth. Exemplary mutations include without limitation one or more amino acid substitutions at E233, L234, L235, G236, G237, D265, D270, N297, E318, K320, K322, A327, A330, P331, or P329 (numbering according to the EU index of Kabat (Sequences of Proteins of Immunological Interest, 5th Ed. Public Health Service, National Institutes of Health, Bethesda, Md. (1991)).

In some embodiments, an antibody of the present disclosure has reduced or ablated binding to CD16a, CD32a, CD32b, CD32c, and CD64 Fcγ receptors. In some embodiments, an antibody with a non-native Fc region described herein exhibits at least a 5%, 10%, 15%, 20%, 30%, 40%, 50%, 60%, 70%, 80%, 90% or greater reduction in C1q binding compared to an antibody comprising a wild-type Fc region. In some embodiments, an antibody with a non-native Fc region as described herein exhibit at least a 5%, 10%, 15%, 20%, 30%, 40%, 50%, 60%, 70%, 80%, 90% or greater reduction in CDC compared to an antibody comprising a wild-type Fc region.

In some embodiments, the Fc variants herein are minimally glycosylated or have reduced glycosylation relative to a wild-type sequence. In some embodiments, deglycosylation is accomplished with a mutation of N297 A, or by mutating N297 to any amino acid which is not N.

In some embodiments, variants of antibody IgG constant regions (e.g., Fc variants) possess a reduced capacity to specifically bind Fcγ receptors or have a reduced capacity to induce phagocytosis. In some embodiments, variants of antibody IgG constant regions (e.g., Fc variants) possess a reduced capacity to specifically bind Fcγ receptors and have a reduced capacity to induce phagocytosis. For example, in some embodiments, an Fc domain is mutated to lack effector functions, typical of a “dead” Fc domain. For example, in some embodiments, an Fc domain includes specific amino acid substitutions that are known to minimize the interaction between the Fc domain and an Fcγ receptor. In some embodiments, an Fc domain monomer is from an IgG1 antibody and includes one or more of amino acid substitutions L234 A, L235 A, G237 A, and N297 A (as designated according to the EU numbering system per Kabat et al., 1991). In some embodiments, an Fc domain monomer is from an IgG1 antibody and includes one or more of amino acid substitutions L234 A, L235 A and G237 A (as designated according to the EU numbering system per Kabat et al., 1991). In some embodiments, an Fc domain monomer is from an IgG1 antibody and includes N297 A (as designated according to the EU numbering system per Kabat et al., 1991). In some embodiments, an Fc domain monomer is from an IgG1 antibody and includes D265 A (as designated according to the EU numbering system per Kabat et al., 1991). In some embodiments, an Fc domain monomer is from an IgG1 antibody and includes one or more of amino acid substitutions D265 A and N297 A (as designated according to the EU numbering system per Kabat et al., 1991). In some embodiments, one or more additional mutations are included in such IgG1 Fc variant. Non-limiting examples of such additional mutations for human IgG1 Fc variants include E318 A and K322 A. In some instances, a human IgG1 Fc variant has up to 12, 11, 10, 9, 8, 7, 6, 5 or 4 or fewer mutations in total as compared to wild-type human IgG1 sequence. In some embodiments, one or more additional deletions are included in such IgG1 Fc variant. For example, in some embodiments, the C-terminal lysine of the Fc IgG1 heavy chain constant region is deleted, for example to increase the homogeneity of the polypeptide when the polypeptide is produced in bacterial or mammalian cells. In some instances, a human IgG1 Fc variant has up to 12, 11, 10, 9, 8, 7, 6, 5 or 4 or fewer deletions in total as compared to wild-type human IgG1 sequence.

In some embodiments, an Fc domain monomer is from an IgG2 antibody and includes amino acid substitutions A330 S, P331 S, or both A330 S and P331 S. The aforementioned amino acid positions are defined according to Kabat, et al. (1991). The Kabat numbering of amino acid residues can be determined for a given antibody by alignment at regions of homology of the sequence of the antibody with a “standard” Kabat numbered sequence. In some embodiments, the Fc variant comprises a human IgG2 Fc sequence comprising one or more of A330 S, P331 S and N297 A amino acid substitutions (as designated according to the EU numbering system per Kabat, et al. (1991). In some embodiments, the Fc variant comprises a human IgG2 Fc sequence comprising one or more of D265 A and N297 A amino acid substitutions (as designated according to the EU numbering system per Kabat, et al. (1991). In some embodiments, the Fc variant comprises a human IgG2 Fc sequence comprising N297 A amino acid substitutions (as designated according to the EU numbering system per Kabat, et al. (1991). In some embodiments, one or more additional mutations are included in such IgG2 Fc variants. Non-limiting examples of such additional mutations for human IgG2 Fc variant include V234 A, G237 A, P238 S, V309L and H268 A (as designated according to the EU numbering system per Kabat et al. (1991)). In some instances, a human IgG2 Fc variant has up to 12, 11, 10, 9, 8, 7, 6, 5, 4, 3 or fewer mutations in total as compared to wild-type human IgG2 sequence. In some embodiments, one or more additional deletions are included in such IgG2 Fc variant.

When the Fc variant is an IgG4 Fc variant, in some embodiments, such Fc variant comprises a S228P, E233P, F234V, L235 A, L235E, or delG236 mutation (as designated according to Kabat, et al. (1991)). In other instances, such Fc variant comprises a S228P and L235E mutation (as designated according to Kabat, et al. (1991)). In some instances, a human IgG4 Fc variant has up to 12, 11, 10, 9, 8, 7, 6, 5, 4, 3, 2 or 1 mutation(s) in total as compared to wild-type human IgG4 sequence.

In some embodiments, the Fc variant exhibits reduced binding to an Fc receptor of the subject compared to the wild-type human IgG Fc region. In some embodiments, the Fc variant exhibits ablated binding to an Fc receptor of the subject compared to the wild-type human IgG Fc region. In some embodiments, the Fc variant exhibits a reduction of phagocytosis compared to the wild-type human IgG Fc region. In some embodiments, the Fc variant exhibits ablated phagocytosis compared to the wild-type human IgG Fc region.

Antibody-dependent cell-mediated cytotoxicity, which is also referred to herein as ADCC, refers to a form of cytotoxicity in which secreted Ig bound onto Fc receptors (FcRs) present on certain cytotoxic cells (e.g., Natural Killer (NK) cells and neutrophils) enabling these cytotoxic effector cells to bind specifically to an antigen-bearing target cell and subsequently kill the target cell. Antibody-dependent cell-mediated phagocytosis, which is also referred to herein as ADCP, refers to a form of cytotoxicity in which secreted Ig bound onto Fc receptors (FcRs) present on certain phagocytic cells (e.g., macrophages) enabling these phagocytic effector cells to bind specifically to an antigen-bearing target cell and subsequently engulf and digest the target cell. Ligand-specific high-affinity IgG antibodies directed to the surface of target cells can stimulate the cytotoxic or phagocytic cells and can be used for such killing. In some embodiments, polypeptide constructs comprising an Fc variant as described herein exhibit reduced ADCC or ADCP as compared to a polypeptide construct comprising a wild-type Fc region. In some embodiments, polypeptide constructs comprising an Fc variant as described herein exhibit at least a 5%, 10%, 15%, 20%, 30%, 40%, 50%, 60%, 70%, 80%, 90% or greater reduction in ADCC or ADCP compared to a polypeptide construct comprising a wild-type Fc region. In some embodiments, antibodies comprising an Fc variant as described herein exhibit ablated ADCC or ADCP as compared to a polypeptide construct comprising a wild-type Fc region.

Complement-directed cytotoxicity, which is also referred to herein as CDC, refers to a form of cytotoxicity in which the complement cascade is activated by the complement component C1q binding to antibody Fc. In some embodiments, polypeptide constructs comprising an Fc variant as described herein exhibit at least a 5%, 10%, 15%, 20%, 30%, 40%, 50%, 60%, 70%, 80%, 90% or greater reduction in C1q binding compared to a polypeptide construct comprising a wild-type Fc region. In some cases, polypeptide constructs comprising an Fc variant as described herein exhibit reduced CDC as compared to a polypeptide construct comprising a wild-type Fc region. In some embodiments, polypeptide constructs comprising an Fc variant as described herein exhibit at least a 5%, 10%, 15%, 20%, 30%, 40%, 50%, 60%, 70%, 80%, 90% or greater reduction in CDC compared to a polypeptide construct comprising a wild-type Fc region. In some cases, antibodies comprising an Fc variant as described herein exhibit negligible CDC as compared to a polypeptide construct comprising a wild-type Fc region.

Fc variants herein include those that exhibit reduced binding to an Fcγ receptor compared to the wild-type human IgG Fc region. For example, in some embodiments, an Fc variant exhibits binding to an Fcγ receptor that is less than the binding exhibited by a wild-type human IgG Fc region to an Fcγ receptor. In some instances, an Fc variant has reduced binding to an Fcγ receptor by a factor of 10%, 20% 30%, 40%, 50%, 60%, 70%, 80%, 90%, 95%, 96%, 97%, 98%, 99% or 100% (fully ablated effector function). In some embodiments, the reduced binding is for any one or more Fcγ receptors, e.g., CD16a, CD32a, CD32b, CD32c, or CD64.

In some instances, the Fc variants disclosed herein exhibit a reduction of phagocytosis compared to its wild-type human IgG Fc region. Such Fc variants exhibit a reduction in phagocytosis compared to its wild-type human IgG Fc region, wherein the reduction of phagocytosis activity is, e.g., by a factor of 10%, 20%, 30%, 40%, 50%, 60%, 70%, 80%, 90%, 95%, 96%, 97%, 98%, 99% or 100%. In some instances, an Fc variant exhibits ablated phagocytosis compared to its wild-type human IgG Fc region.

In some embodiments, the Fc variants disclosed herein are coupled to one or more fusion partners. In some cases the fusion partner is a therapeutic moiety, such as a cytotoxic agent of the present disclosure. In some cases, the fusion partner is selected to enable targeting of an expressed protein, purification, screening, display, and the like. In some embodiments, the fusion partner also affects the degree of binding to Fc receptors or the degree of phagocytosis reduction.

In certain embodiments, the targeting moiety is a bispecific antibody. In certain embodiments, the bispecific antibody comprises a first antigen binding domain that binds an extracellular domain of a human CD56 polypeptide and a second antigen binding domain that binds an antigen expressed by a cancer cell. In certain embodiments, the bispecific antibody comprises a first antigen binding domain that binds an extracellular domain of a human SIRP-α polypeptide and a second antigen binding domain that binds an antigen expressed by a cancer cell. In certain embodiments, the antigen expressed by the cancer cell is selected from the group consisting of CD19, CD20, CD22, CD30, CD33, CD38, CD52, CD56, CD70, CD74, CD79b, CD123, CD138, CS1/SLAMF7, Trop-2, 5T4, EphA4, BCMA, Mucin 1, Mucin 16, PD-L1, PTK7, STEAP1, Endothelin B Receptor, mesothelin, EGFRvIII, ENPP3, SLC44 A4, GNMB, nectin 4, NaPi2b, LIV-1 A, Guanylyl cyclase C, DLL3, EGFR, HER2, VEGF, VEGFR, integrin αVβ3, integrin α5β1, MET, IGF1 R, TRAILR1, TRAILR2, RANKL, FAP, Tenascin, Ley, EpCAM, CEA, gpA33, PSMA, TAG72, a mucin, CAIX, EPHA3, folate receptor α, GD2, GD3, and an MHC/peptide complex comprising a peptide from NY-ESO-1/LAGE, SSX-2, a MAGE family protein, MAGE-A3, gp100/pmel17, Melan-A/MART-1, gp75/TRP1, tyrosinase, TRP2, CEA, PSA, TAG-72, immature laminin receptor, MOK/RAGE-1, WT-1, SAP-1, BING-4, EpCAM, MUC1, PRAME, survivin, BRCA1, BRCA2, CDK4, CML66, MART-2, p53, Ras, β-catenin, TGF-βRII, HPV E6, or HPV E7. In certain embodiments, the antibody comprises a first antigen binding domain that binds an extracellular domain of a human CD56 polypeptide and a second antigen binding domain that binds an antigen expressed by an immune cell. In certain embodiments, the antibody comprises a first antigen binding domain that binds an extracellular domain of a human SIRP-α polypeptide and a second antigen binding domain that binds an antigen expressed by an immune cell. In some embodiments, the antigen expressed by the immune cell is selected from the group consisting of BDCA2, BDCA4, ILT7, LILRB1, LILRB2, LILRB3, LILRB4, CSF-1R, CD40, CD40L, CD163, CD206, DEC205, CD47, CD123, IDO, TDO, 41BB, CTLA4, PD1, PD-L1, PD-L-2, TIM-3, BTLA, VISTA, LAG-3, CD28, OX40, GITR, CD137, CD27, HVEM, CCR4, CD25, CD103, KIrg1, Nrp1, CD278, Gpr83, TIGIT, CD154, CD160, PVRIG, DNAM, and ICOS.

In certain embodiments, the antibody comprises a constant region sequence selected from the table below.

| SEQ ID | | |
|---|---|---|
| NO: | Name | Sequence |
| IgG1 wildtype | 564 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | REE |
| | | MTKNQVSLTCLVKGFYPSDIAVEWESNGQPENNYKTTPP |
| | | VLDSDGSFFLYSKLTVDKSRW |
| | | QQGNVFSCSVMHEALHNHYTQKSLSLSPGK |
| IgG1_AAA_N297A | 565 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPEAAG |
| | | APSVFLFPPKPKDTLMISRTPEVTCVVVDVSHEDPEVKFN |
| | | WYVDGVEVHNAKTKPREEQYASTYRVVSVLTVLHQDWL |
| | | NGKEYKCKVSNKALPAPIEKTISKAKGQPREPQVYTLPPS |
| | | REEMTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| IgG1_AAA | 566 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPEAAG |
| | | APSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | REEMTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| IgG1_N297A | 567 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYASTYRVVSVLTVLHQDWL |
| | | NGKEYKCKVSNKALPAPIEKTISKAKGQPREPQVYTLPPS |
| | | REE |
| | | MTKNQVSLTCLVKGFYPSDIAVEWESNGQPENNYKTTPP |
| | | VLDSDGSFFLYSKLTVDKSRW |
| | | QQGNVFSCSVMHEALHNHYTQKSLSLSPGK |
| IgG1_D265A | 568 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVAVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | REE |
| | | MTKNQVSLTCLVKGFYPSDIAVEWESNGQPENNYKTTPP |
| | | VLDSDGSFFLYSKLTVDKSRW |
| | | QQGNVFSCSVMHEALHNHYTQKSLSLSPGK |
| IgG1_N297A/D265A | 569 | ASTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKKVEPKSCDKTHTCPPCPAPELLG |
| | | GPSVFLFPPKPKDTLMISRTPEVTCVVVAVSHEDPEVKFN |
| | | WYVDGVEVHNAKTKPREEQYASTYRVVSVLTVLHQDWL |
| | | NGKEYKCKVSNKALPAPIEKTISKAKGQPREPQVYTLPPS |
| | | REE |
| | | MTKNQVSLTCLVKGFYPSDIAVEWESNGQPENNYKTTPP |
| | | VLDSDGSFFLYSKLTVDKSRW |
| | | QQGNVFSCSVMHEALHNHYTQKSLSLSPGK |
| IgG2 | 570 | ASTKGPSVFPLAPCSRSTSESTAALGCLVKDYFPEPVTVS |
| | | WNSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDG |
| | | VEVHNAKTKPREEQFNSTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSD |
| | | GSFFLYSKLTVDKSRWQQGNV |
| | | FSCSVMHEALHNHYTQKSLSLSPGK |
| IgG2Da | 571 | ASTKGPSVFPLAPCSRSTSESTAALGCLVKDYFPEPVTVS |
| | | WNSGALTSGVHTFPAVLQSSGLYSLSSVVTVPSSNFGTQ |
| | | TYTCNVDHKPSNTKVDKTVERKCCVECPPCPAPPVAGPS |
| | | VFLFPPKPKDTLMISRTPEVTCVVVDVSHEDPEVQFNWYV |
| | | DGVEVHNAKTKPREEQFNSTFRVVSVLTVVHQDWLNGKE |
| | | YKCKVSNKGLPSSIEKTISKTKGQPREPQVYTLPPSREEM |
| | | TKNQVSLTCLVKGFYPSDIAVEWESNGQPENNYKTTPPM |
| | | LDSDGSFFLYSKLTVDKSRWQQGNVFSCSVMHEALHNHY |
| | | TQKSLSLSPGK |
| IgG2Da_N297A | 572 | ASTKGPSVFPLAPCSRSTSESTAALGCLVKDYFPEPVTVS |
| | | WNSGALTSGVHTFPAVLQSSGLYSLSSVVTVPSSNFGTQ |
| | | TYTCNVDHKPSNTKVDKTVERKCCVECPPCPAPPVAGPS |
| | | VFLFPPKPKDTLMISRTPEVTCVVVDVSHEDPEVQFNWYV |
| | | DGVEVHNAKTKPREEQFASTFRVVSVLTVVHQDWLNGKE |
| | | YKCKVSNKGLPSSIEKTISKTKGQPREPQVYTLPPSREEM |
| | | TKNQVSLTCLVKGFYPSDIAVEWESNGQPENNYKTTPPM |
| | | LDSDGSFFLYSKLTVDKSRWQQGNVFSCSVMHEALHNHY |
| | | TQKSLSLSPGK |
| IgG2_N297A | 573 | ASTKGPSVFPLAPCSRSTSESTAALGCLVKDYFPEPVTVS |
| | | WNSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDG |
| | | VEVHNAKTKPREEQFASTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSD |
| | | GSFFLYSKLTVDKSRWQQGNV |
| | | FSCSVMHEALHNHYTQKSLSLSPGK |
| IgG2Da_D265A | 574 | ASTKGPSVFPLAPCSRSTSESTAALGCLVKDYFPEPVTVS |
| | | WNSGALTSGVHTFPAVLQSSGLYSLSSVVTVPSSNFGTQ |
| | | TYTCNVDHKPSNTKVDKTVERKCCVECPPCPAPPVAGPS |
| | | VFLFPPKPKDTLMISRTPEVTCVVVAVSHEDPEVQFNWYV |
| | | DGVEVHNAKTKPREEQFNSTFRVVSVLTVVHQDWLNGKE |
| | | YKCKVSNKGLPSSIEKTISKTKGQPREPQVYTLPPSREEM |
| | | TKNQVSLTCLVKGFYPSDIAVEWESNGQPENNYKTTPPM |
| | | LDSDGSFFLYSKLTVDKSRWQQGNVFSCSVMHEALHNHY |
| | | TQKSLSLSPGK |
| IgG4_S228P | 575 | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVDVSQEDPEVQFNWYV |
| | | DGVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPVL |
| | | DSDGSFFLYSRLTVDKSRWQEGNVFSCSVMHEALHNHYT |
| | | QKSLSLSLGK |
| IgG4_S228P_D265A | 576 | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVAVSQEDPEVQFNWYV |
| | | DGVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPVL |
| | | DSDGSFFLYSRLTVDKSRWQEGNVFSCSVMHEALHNHYT |
| | | QKSLSLSLGK |
| IgG4_S228P, L235E | 577 | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFEGGPS |
| | | VFLFPPKPKDTLMISRTPEVTCVVVDVSQEDPEVQFNWYV |
| | | DGVEVHNAKTKPREEQFNSTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPVL |
| | | DSDGSFFLYSRLTVDKSRWQEGNVFSCSVMHEALHNHYT |
| | | QKSLSLSLGK |
| IgG4 S228P, N297A | 578 | ASTKGPSVFPLAPCSRSTSESTAALGCLVKDYFPEPVTVS |
| | | WNSGALTSGVHTFPAVLQSSGLYSLSSVVTVPSSSLGTKT |
| | | YTCNVDHKPSNTKVDKRVESKYGPPCPPCPAPEFLGGPS |
| | | VFLFPPKPKDTLMISRTPEVTCVVVDVSQEDPEVQFNWYV |
| | | DGVEVHNAKTKPREEQFASTYRVVSVLTVLHQDWLNGKE |
| | | YKCKVSNKGLPSSIEKTISKAKGQPREPQVYTLPPSQEEM |
| | | TKNQVSLTCLVKGFYPSDIAVEWESNGQPENNYKTTPPVL |
| | | DSDGSFFLYSRLTVDKSRWQEGNVFSCSVMHEALHNHYT |
| | | QKSLSLSLGK |
| Human Kappa | 579 | RTVAAPSVFIFPPSDEQLKSGTASVVCLLNNFYPREAKVQ |
| | | WKVDNALQSGNSQESVTEQDSKDSTYSLSSTLTLSKADY |
| | | EKHKVYACEVTHQGLSSPVTKSFNRGEC |
| Human Lambda IGLC1 | 580 | GQPKANPTVTLFPPSSEELQANKATLVCLISDFYPGAVTV |
| | | AWKADGSPVKAGVETTKPSKQSNNKYAASSYLSLTPEQW |
| | | KSHRSYSCQVTHEGSTVEKTVAPTECS |
| Human Lambda IGLC2 | 581 | GQPKAAPSVTLFPPSSEELQANKATLVCLISDFYPGAVTV |
| | | AWKADSSPVKAGVETTTPSKQSNNKYAASSYLSLTPEQW |
| | | KSHRSYSCQVTHEGSTVEKTVAPTECS |

In certain embodiments, the targeting moiety is a polypeptide. In certain embodiments, the targeting moiety is a RGD peptide, a rabies virus glycoprotein (RVG), or a DC3 peptide. In certain embodiments, the targeting moiety is an aptamer. In certain embodiments, the targeting moiety comprises a small molecule. In certain embodiments, the targeting moiety comprises folate, mannose, or a PSMA ligand.

### Conjugates

In one embodiment, a conjugate provided herein comprise a targeting moiety and one or more immunomodulating polynucleotides, in certain embodiments, from about 1 to about 6 or from about 1 to about 4, about 1, or about 2 immunomodulating polynucleotides. In certain embodiments, the conjugate comprises a linker that links the targeting moiety covalently to the immunomodulating polynucleotides. In certain embodiments, the linker is bonded to a nucleobase, abasic spacer, phosphate, phosphorothioate, or phosphorodithioate in the immunomodulating polynucleotide.

In one embodiment, provided herein is a conjugate of Formula (C):

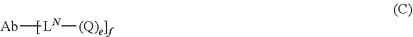

or a stereoisomer, a mixture of two or more diastereomers, a tautomer, or a mixture of two or more tautomers thereof; or a pharmaceutically acceptable salt, solvate, or hydrate thereof; wherein Ab is a targeting moiety; f is an integer of 1, 2, 3, or 4; and L^{N}, Q, and e are each as defined herein.

In certain embodiments, in Formula (C), Ab is an antibody. In certain embodiments, in Formula (C), Ab is a monoclonal antibody.

In certain embodiments, in Formula (C), f is an integer of 1 or 2. In certain embodiments, in Formula (C), f is an integer of 1.

In certain embodiments, in Formula (C), both e and f are each an integer of 1.

In one embodiment, the CpG antibody conjugate has a DAR ranging from about 1 to about of about 20, from about 1 to about 10, from about 1 to about 8, from about 1 to about 4, or from about 1 to about 2. In another embodiment, the CpG antibody conjugate has a DAR of about 1, about 2, about 3, about 4, about 5, about 6, about 7, or about 8.

### Preparation of Conjugates

Conjugation

Reactions useful for conjugating a targeting moiety to an immunomodulating polynucleotide are known in the art, including, but not limited to Huisgen cycloaddition (metal-catalyzed or metal-free) between an azido and an alkyne-based conjugating group (e.g., optionally substituted C₆₋₁₆ heterocyclylene containing an endocyclic carbon-carbon triple bond or optionally substituted C₈₋₁₆ cycloalkynyl) to form a triazole moiety; the Diels-Alder reaction between a dienophile and a diene/hetero-diene; bond formation via pericyclic reactions such as the ene reaction; amide or thioamide bond formation; sulfonamide bond formation (e.g., with azido compounds); alcohol or phenol alkylation (e.g., Williamson alkylation), condensation reactions to form oxime, hydrazone, or semicarbazide group; conjugate addition reactions by nucleophiles (e.g., amines and thiols); disulfide bond formation; and nucleophilic substitution (e.g., by an amine, thiol, or hydroxyl nucleophile) at a carbonyl (e.g., at an activated carboxylic acid ester, such as pentafluorophenyl (PFP) ester or tetrafluorophenyl (TFP) ester) or at an electrophilic arene (e.g., S_{N}Ar at an oligofluorinated arene, a fluorobenzonitrile group, or fluoronitrobenzene group).

In certain embodiments, the conjugation reaction is a dipolar cycloaddition, and the conjugation moiety includes azido, optionally substituted C₆₋₁₆ heterocyclylene containing an endocyclic carbon-carbon triple bond, or optionally substituted C₈₋₁₆ cycloalkynyl. The complementary reactive group and the conjugating group are selected for their mutual complementarity. For example, an azide is used in one of the conjugating group and the complementary reactive group, while an alkyne is used in the other of the conjugating group and the complementary reactive group.

### Preparation of Immunomodulating Polynucleotides

The immunomodulating polynucleotide provided herein can be prepared according to methods known in the art of chemical synthesis of polynucleotides, e.g., from nucleoside phosphoramidites. The phosphoramidite can include a conjugating group covalently linked to the phosphorus atom of the phosphoramidite.

### Preparation of a Targeting Moiety Portion

A targeting moiety can be conjugated to an immunomodulating polynucleotide by forming a bond between a conjugating group in the immunomodulating polynucleotide and a complementary reactive group bonded to the targeting moiety. In certain embodiments, the targeting moiety intrinsically possess a complementary reactive group (e.g., a Q-tag (e.g., LLQGG (SEQ ID NO:582) or GGGLLQGG (SEQ ID NO:583)) in an antibody or antigen-binding fragment or an engineered derivative thereof). In certain embodiments, the targeting moiety, is modified to include a complementary reactive group (e.g., by attaching a complementary reactive group to a Q-tag). Methods of introducing such complementary reactive groups into a targeting moiety is known in the art.

In certain embodiments, the complementary reactive group is optionally substituted C₂₋₁₂ alkynyl, optionally substituted N-protected amino, azido, N-maleimido, S-protected thiol,

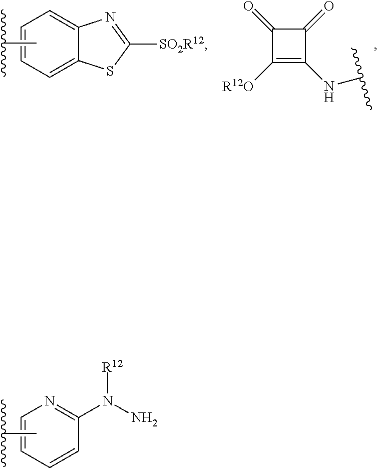

or a N-protected moiety thereof,

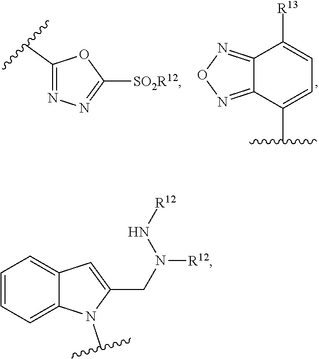

optionally substituted C₆₋₁₆ heterocyclyl containing an endocyclic carbon-carbon triple bond (e.g.,

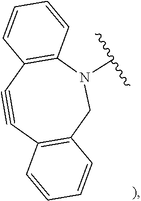 [SMILES: *N1Cc2ccccc2C#Cc2ccccc21]

1,2,4,5-tetrazine group (e.g.,

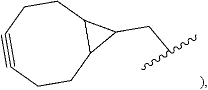 [SMILES: *CC1C2CCC#CCCC12]

optionally substituted C₈₋₁₆ cycloalkynyl (e.g.,

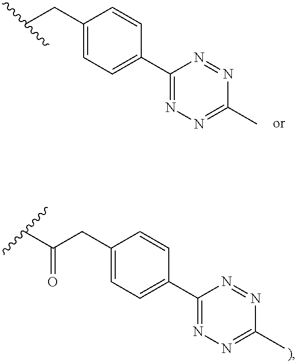 [SMILES: *Cc1ccc(-c2nnc(C)nn2)cc1 ; *C(=O)Cc1ccc(-c2nnc(C)nn2)cc1]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;
wherein:

R^{N1} is H, N-protecting group, or optionally substituted C₁₋₆ alkyl;

each R¹² is independently H, optionally substituted C₁₋₆ alkyl, or O-protecting group (e.g., a carboxyl protecting group); and

R¹³ is halogen (e.g., F).

In certain embodiments, the complementary reactive group is protected until the conjugation reaction. For example, a complementary reactive group that is protected can include —COOR^{PGO} or —NHR^{PGN}, where R^{PGO} is an O-protecting group (e.g., a carboxyl protecting group), and R^{PGN} is an N-protecting group.

In certain embodiments, a complementary reactive group is —Z³-Q^{A3}, wherein:

Z³ is a divalent, trivalent, tetravalent, or pentavalent group, in which one of the valencies is substituted with Q^{A3}, one of the valencies is open, and each of the remaining valencies, if present, is independently substituted with an auxiliary moiety;

Q^{A3} is optionally substituted C₂₋₁₂ alkynyl, optionally substituted N-protected amino, azido, N-maleimido, S-protected thiol,

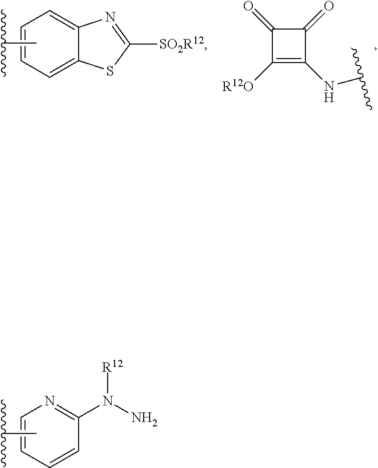

or N-protected version thereof,

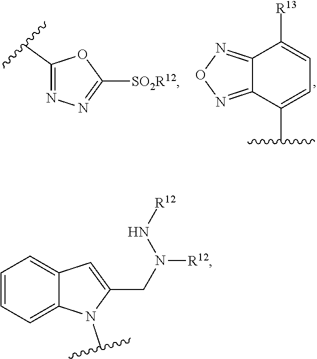

optionally substituted C₆₋₁₆ heterocyclyl containing an endocyclic carbon-carbon triple bond (e.g.,

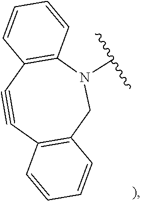 [SMILES: *N1Cc2ccccc2C#Cc2ccccc21]

1,2,4,5-tetrazine group (e.g.,

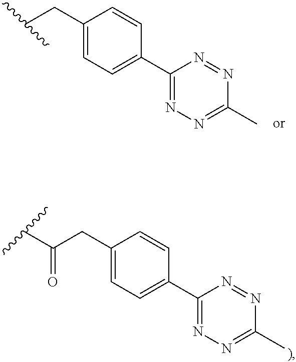 [SMILES: *Cc1ccc(-c2nnc(C)nn2)cc1 ; *C(=O)Cc1ccc(-c2nnc(C)nn2)cc1]

optionally substituted C₈₋₁₆ cycloalkynyl (e.g.,

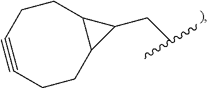 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;
wherein:

R^{N1} is H, N-protecting group, or optionally substituted C₁₋₆ alkyl;

each R¹² is independently H, optionally substituted C₁₋₆ alkyl, 0-protecting group, or a carboxyl protecting group; and

R¹³ is halogen or F.

In certain embodiments, Z³ comprises a branching group and two divalent segments, wherein the branching group is bonded to each of the two divalent segments, wherein one of the divalent segments has an open valency, and the remaining divalent segment is bonded to Q^{A3}; and the branching group comprises one or two monomers independently selected from the group consisting of optionally substituted C₁₋₁₂ alkane-triyl, optionally substituted C₁₋₁₂ alkane-tetrayl, optionally substituted C₂₋₁₂ heteroalkane-triyl, and optionally substituted C₂₋₁₂ heteroalkane-tetrayl, where two valencies of the branching group are bonded to the two divalent segments, and each of the remaining valencies is independently substituted with an auxiliary moiety.

In certain embodiments, the divalent segment in Z³ is -(-Q^{B}-Q^{C}-Q^{D}-)ₛ₁, wherein:

s1 is an integer from about 1 to about 50 or from about 1 to about 30;

each Q^{B} and Q^{D} are independently absent, —CO—, —NH—, —O—, —S—, —SO₂—, —OC(O)—, —COO—,

—NHC(O)—, —C(O)NH—, —CH₂—, —CH₂NH—, —NHCH₂—, —CH₂O—, or —OCH₂—; and

each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene.

In certain embodiments, at least one of Q^{B} and Q^{D} is present in each monomeric unit of Z³.

In certain embodiments, —Z³-Q^{A3} is

—(-Q^{B}-Q^{C}-Q^{D}-)ₛ₁-Q^{E}-(-Q^{B}-Q^{C}-Q^{D}-)ₛ₁-Q^{A3}, (Vb)

wherein:

each s1 is independently an integer from about 1 to about 50 or from about 1 to about 30;

Q^{A3} is as described herein;

each Q^{B} and Q^{D} are independently absent, —CO—, —NH—, —O—, —S—, —SO₂—, —OC(O)—, —COO—,

—NHC(O)—, —C(O)NH—, —CH₂—, —CH₂NH—, —NHCH₂—, —CH₂O—, or —OCH₂—; and

each Q^{C} is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene; and

Q^{E} is absent or a branching group of formula (IV) as described herein.

In certain embodiments, -(-Q^{B}-Q_{C}-Q^{D}-)ₛ₁— is a group:

-Q^{B}-(CH₂)_{g1}—(CH₂OCH₂)_{g2}—(CH₂)_{g3}-Q^{D}-,

wherein:
(i) g2 is an integer from about 1 to about 50, from about 1 to about 40, or from about 1 to about 30;
(ii) g1 is 1 and Q^{B} is —NHCO—, —CONH—, or —O—; or g1 is 0 and Q^{D} is —NHCO—; and
(iii) g3 is 1 and Q^{B} is —NHCO—, —CONH—, or —O—; or g3 is 0 and Q^{D} is —CONH—.

In certain embodiments, the complementary reactive group is:

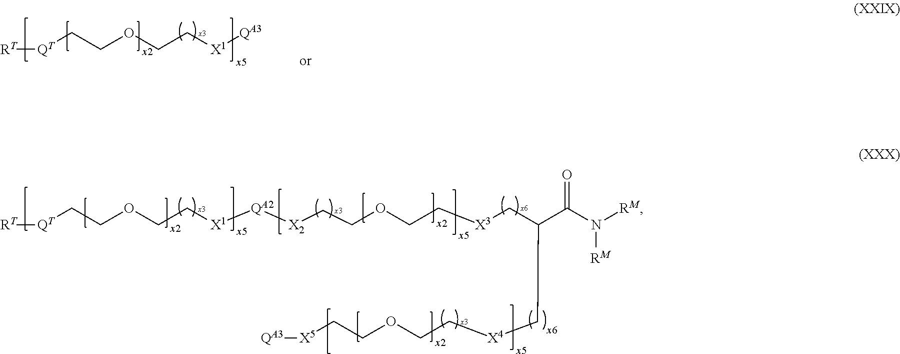

wherein:

Q^{A2} is absent, optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

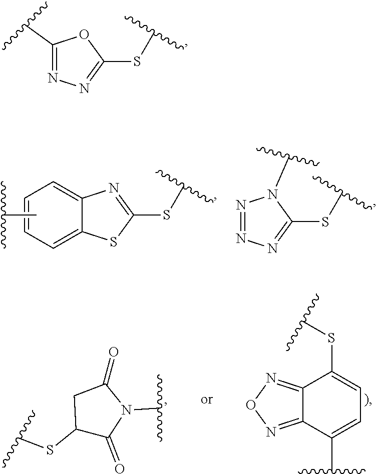

optionally substituted C₁₋₁₂ heterocyclylene (e.g., 1,2,3-triazole-1,4-diyl or

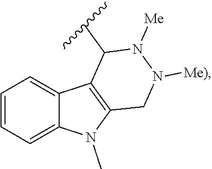

cyclobut-3-ene-1,2-dione-3,4-diyl, pyrid-2-yl hydrazone, optionally substituted C₆₋₁₆ triazoloheterocyclylene (e.g.,

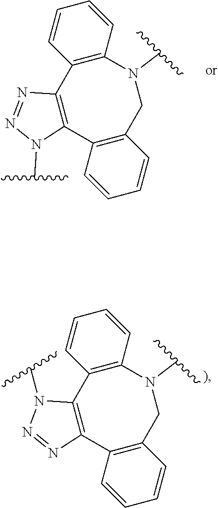 [SMILES: *N1Cc2ccccc2-c2c(nnn2*)-c2ccccc21 ; *N1Cc2ccccc2-c2nnn(*)c2-c2ccccc21]

optionally substituted C₈₋₁₆ triazolocycloalkenylene (e.g.,

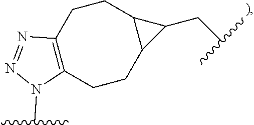 [SMILES: *CC1C2CCc3nnn(*)c3CCC12]

or a dihydropyridazine group (e.g.,

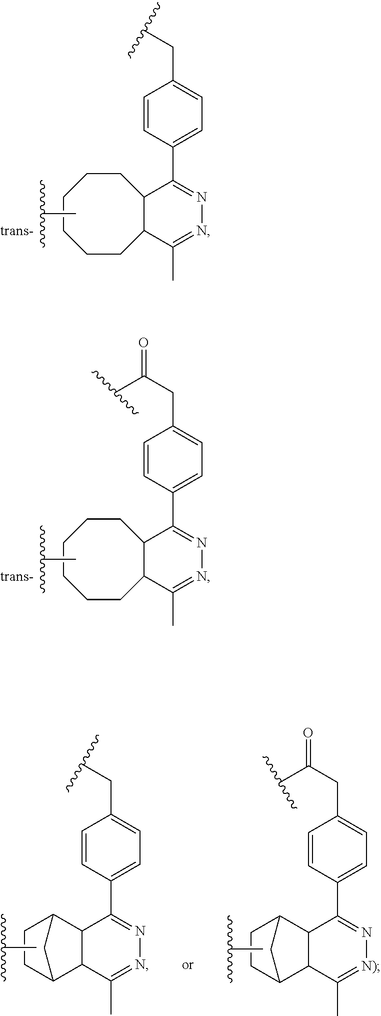

each Q^{A3} is independently optionally substituted C₂₋₁₂ alkynyl, optionally substituted N-protected amino, azido, N-maleimido, S-protected thiol,

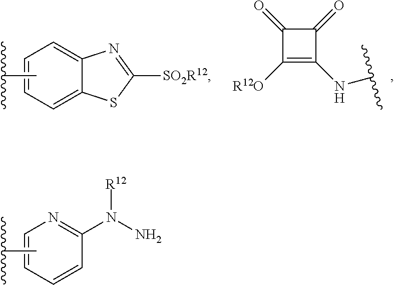

or an N-protected version thereof,

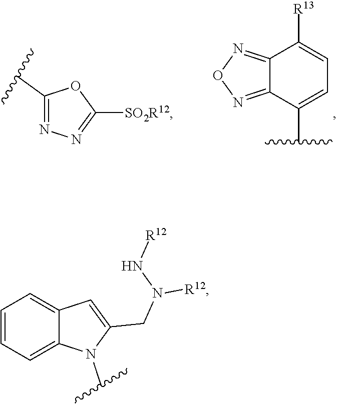

optionally substituted C₆₋₁₆ heterocyclyl containing an endocyclic carbon-carbon triple bond (e.g.,

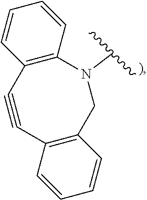 [SMILES: *N1Cc2ccccc2C#Cc2ccccc21]

1,2,4,5-tetrazine group (e.g.,

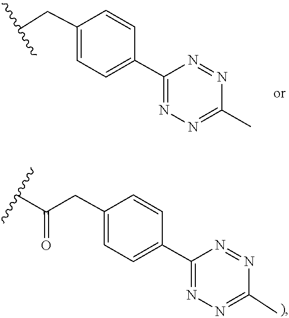 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

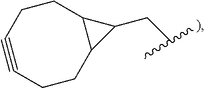

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;

R^{N1} is H, N-protecting group, or optionally substituted C₁₋₆ alkyl;

each R¹² is independently H or optionally substituted C₁₋₆ alkyl;

R¹³ is halogen or F;

each R^{T} is independently a bond to a targeting moiety;

each Q^{T} is independently —CO—, —NH—, —NH—CH₂—, or —CO—CH₂—;

each X¹, X³, and X⁵ are independently absent, —O—, —NH—, —CH₂—NH—, —C(O)—, —C(O)—NH—, —NH—C(O)—, —NH—C(O)—NH—, —O—C(O)—NH—, —NH—C(O)—O—, —CH₂—NH—C(O)—NH—, —CH₂—O—C(O)—NH—, or —CH₂—NH—C(O)—O—;

each X² and X⁴ are independently absent, —O—, —NH—, —C(O)—, —C(O)—NH—, —NH—C(O)—, —NH—C(O)—NH—, —O—C(O)—NH—, or —NH—C(O)—O—;

each x2 is independently an integer from about 0 to about 50, from about 1 to about 40, or from about 1 to about 30;

each x3 is independently an integer from about 1 to about 11; and

each x5 is independently an integer of about 0 or about 1; and

each x6 is independently an integer from about 0 to about 10 or from about 1 to about 6, provided that the sum of both x6 is about 12 or less.

In certain embodiments, the complementary reactive group is:

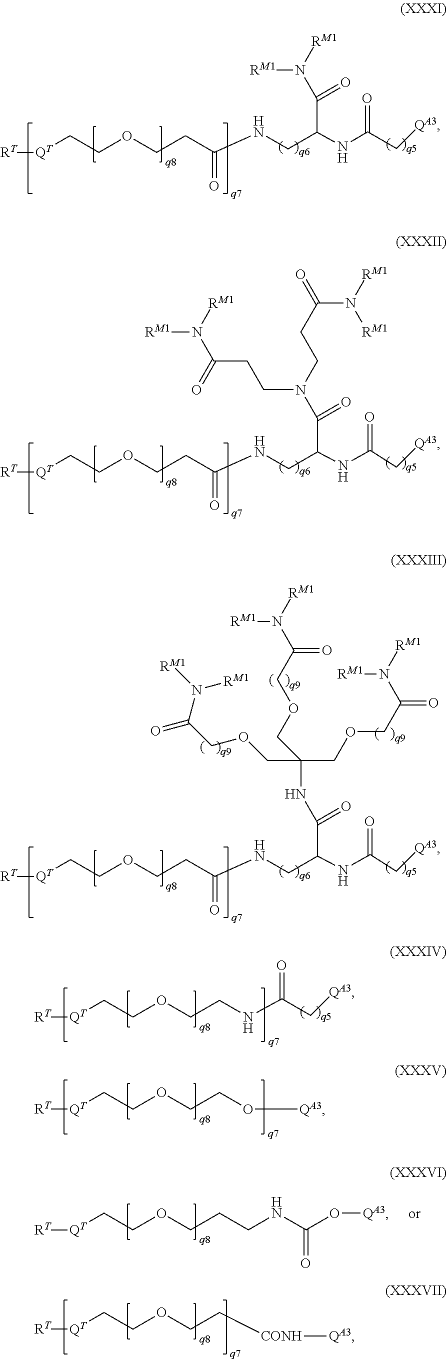

wherein:
each Q^{A3} is independently optionally substituted C₂₋₁₂ alkynyl, optionally substituted N-protected amino, azido, N-maleimido, S-protected thiol,

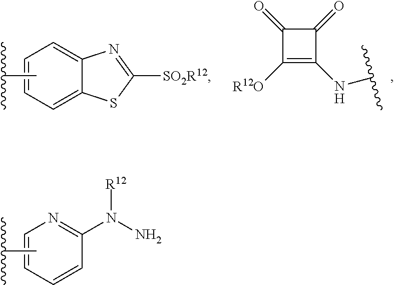

or an N-protected version thereof,

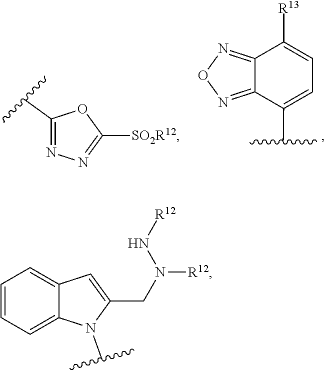

optionally substituted C₆₋₁₆ heterocyclyl containing an endocyclic carbon-carbon triple bond (e.g.,

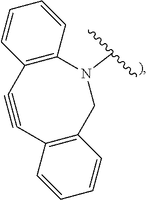 [SMILES: *N1Cc2ccccc2C#Cc2ccccc21]

1,2,4,5-tetrazine group (e.g.,

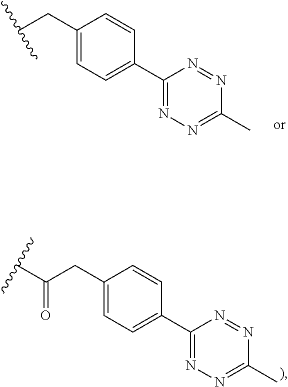 [SMILES: *Cc1ccc(-c2nnc(C)nn2)cc1 ; *C(=O)Cc1ccc(-c2nnc(C)nn2)cc1]

or optionally substituted C₈₋₁₆ cycloalkynyl (e.g.,

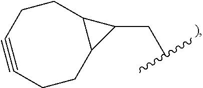 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;

each R^{M1} is independently H or an auxiliary moiety;

each R^{N1} is independently H, N-protecting group, or optionally substituted C₁₋₆ alkyl;

each R¹² is independently H or optionally substituted C₁₋₆ alkyl;

each R¹³ is independently halogen or F;

each Q^{T} is independently —CO—, —NH—, —NH—CH₂—, or —CO—CH₂—;

each R^{T} is independently a bond to a targeting moiety;

each q5 and q6 are independently an integer from about 1 to about 10 or from about 1 to about 6;

each q7 is independently an integer of about 0 or about 1;

each q8 is independently an integer from about 0 to about 50, from about 1 to about 40, or from about 1 to about 30; and

each q9 is independently an integer from about 1 to about 10.

In certain embodiments, the complementary reactive group is:

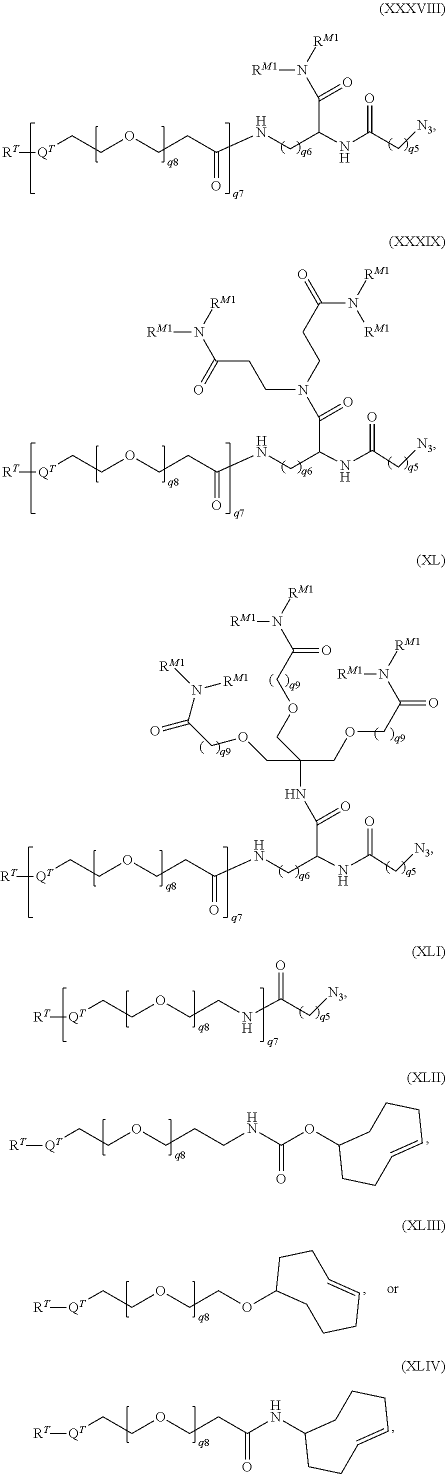

wherein:

each R^{M1} is independently H or an auxiliary moiety;

each Q^{T} is independently —CO—, —NH—, —NH—CH₂—, or —CO—CH₂—;

each R^{T} is independently a bond to a targeting moiety;

each q5 and q6 are independently an integer from about 1 to about or from about 1 to about 6;

each q7 is independently an integer of about 0 or about 1;

each q8 is independently an integer from about 0 to about 50, from about 1 to about 40, or from about 1 to about 30; and

each q9 is independently an integer from about 1 to about 10.

### Pharmaceutical Compositions

Delivery of a conjugate provided herein can be achieved by contacting a cell with the conjugate using a variety of methods known to those of skill in the art. In certain embodiments, the conjugate provided herein is formulated as a pharmaceutical composition including a pharmaceutically acceptable excipient. In certain embodiments, the pharmaceutical composition is a liquid or solid (e.g., lyophilized).

The conjugate provided herein can be administered alone or in admixture with a pharmaceutical acceptable excipient selected with regard to the intended route of administration and standard pharmaceutical practice. The pharmaceutical compositions for use thus can be formulated in a conventional manner using one or more physiologically acceptable carriers, excipients, and auxiliaries that facilitate processing the conjugate into preparations which can be used pharmaceutically.

Frequently used carriers or excipients include sugars (e.g., lactose, mannitol), milk protein, gelatin, starch, vitamins, cellulose and its derivatives, poly(ethylene glycol)s and solvents, such as sterile water, alcohols, glycerol, and polyhydric alcohols. Intravenous vehicles can include fluid and nutrient replenishers. Other pharmaceutically acceptable carriers include aqueous solutions, non-toxic excipients, including salts, preservatives, buffers and the like, as described, for instance, in Remington: The Science and Practice of Pharmacy, 21^{st} Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition can be adjusted in accordance with routine practices in the art. See Goodman and Gilman's, the Pharmacological Basis for Therapeutics.

In making the pharmaceutical compositions, the active ingredient is typically mixed with an excipient (e.g., in lyophilized formulations) or diluted by an excipient. When the excipient serves as a diluent, it can be a solid, semisolid, or liquid material (e.g., phosphate-buffered saline), which acts as a vehicle, carrier, or medium for the active ingredient. Thus, the compositions can be in the form of tablets, powders, elixirs, suspensions, emulsions, solutions, and syrups. As is known in the art, the type of diluent can vary depending upon the intended route of administration. The resulting compositions can include additional agents, e.g., preservatives. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009). Preservatives can include antimicrobial agents, anti-oxidants, chelating agents, and inert gases.

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions are well-known to those skilled in the art of pharmaceutical formulation. In preparing a formulation, a conjugate can be milled to provide the appropriate particle size prior to combining with the other ingredients.

Route of Administration

The pharmaceutical compositions can be administered locally or systemically. The therapeutically effective amounts will vary according to factors, such as the extent of the diseases progression in a subject, the age, sex, and weight of the individual. Dosage regimens can be adjusted to provide the optimum therapeutic response. For example, several divided doses can be administered daily or the dose can be proportionally reduced as indicated by the exigencies of the therapeutic situation.

The pharmaceutical compositions can be administered to a patient in a variety of forms depending on the selected route of administration, as will be understood by those skilled in the art. The conjugates used in the methods described herein can be administered, for example, by parenteral administration. Parenteral administration includes intramuscular, intravenous, intraarterial, intracranial, subcutaneous, intraorbital, intraventricular, intraspinal, intrathecal, intraperitoneal, rectal, and topical routes of administration. Topical route of administration includes transdermal, intradermal, buccal, and sublingual routes of administration. The pharmaceutical compositions are formulated according to the selected route of administration. Parenteral administration can be by continuous infusion over a selected period of time.

Formulations for Parenteral Administration

A conjugate provided herein can be administered to a patient in need thereof in a pharmaceutically acceptable parenteral (e.g., intravenous, intramuscular, or subcutaneous) formulation as described herein. The pharmaceutical formulation can also be administered parenterally (e.g., intravenously, intramuscularly, or subcutaneously) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the patient; and aqueous and non-aqueous sterile suspensions which may include suspending agents and thickening agents. For example, to prepare such a composition, a conjugate provided herein can be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer (e.g., phosphate buffered saline), 1,3-butanediol, Ringer's solution and isotonic sodium chloride solution. The aqueous formulation can also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation of the conjugate provided herein can be any one of the four general types of preparations identified by the USP-NF as suitable for parenteral administration:
(1) “Drug for Injection”: the drug substance (e.g., a conjugate provided herein) as a dry (e.g., lyophilized) solid that will be combined with the appropriate sterile vehicle for parenteral administration as a drug injection;
(2) “Drug Injectable Emulsion”: a liquid preparation of the drug substance (e.g., a conjugate provided herein) that is dissolved or dispersed in a suitable emulsion medium;
(3) “Drug Injectable Suspension”: a liquid preparation of the drug substance (e.g., a conjugate provided herein) suspended in a suitable liquid medium; and
(4) “Drug for Injectable Suspension”: the drug substance (e.g., a conjugate provided herein) as a dry solid that will be combined with the appropriate sterile vehicle for parenteral administration as a drug injectable suspension.

Exemplary formulations for parenteral administration include solutions of a conjugate provided herein prepared in water suitably mixed with a surfactant, e.g., hydroxypropylcellulose. Dispersions can also be prepared in glycerol, liquid poly(ethylene glycol)s, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

Biocompatible, biodegradable lactide polymer, lactide/glycolide copolymer, or polyoxyethylene-polyoxypropylene copolymers can be used to control the release of a conjugate provided herein. Other potentially useful parenteral delivery systems for a conjugate provided herein include ethylene-vinyl acetate copolymer particles, osmotic pumps or implantable infusion systems. The parenteral formulation can be formulated for prompt release or for sustained/extended release of the polynucleotides and/or conjugates. Exemplary formulations for parenteral release of a conjugate provided herein include: aqueous solutions, powders for reconstitution, cosolvent solutions, oil/water emulsions, suspensions, microspheres, and polymeric gels.

### Methods of Use

In one embodiment, provided herein is a method for treating, preventing, or ameliorating one or more symptoms of a proliferative disease in a subject, comprising administering to the subject a therapeutically effective amount of a conjugate disclosed herein.

In certain embodiments, the subject is a mammal. In certain embodiments, the subject is a human. In certain embodiments, the subject is a primate other than a human, a farm animal such as cattle, a sport animal, or a pet such as a horse, dog, or cat.

In certain embodiments, the proliferative disease is a tumor. In certain embodiments, the proliferative disease is a liquid or hematologic tumor. In certain embodiments, the proliferative disease is a solid tumor. In certain embodiments, the proliferative disease is a neoplastic disease.

In certain embodiments, the proliferative disease is cancer. In certain embodiments, the cancer is relapsed cancer. In certain embodiments, the cancer is drug-resistant cancer. In certain embodiments, the cancer is relapsed drug-resistant cancer. In certain embodiments, the cancer is multidrug-resistant cancer. In certain embodiments, the cancer is relapsed multidrug-resistant cancer.

In certain embodiments, the cancer treatable with a conjugate provided herein includes, but is not limited to, (1) leukemias, including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome or a symptom thereof (such as anemia, thrombocytopenia, neutropenia, bicytopenia or pancytopenia), refractory anemia (R^{A}), R^{A} with ringed sideroblasts (RARS), R^{A} with excess blasts (RAEB), RAEB in transformation (RAEB-T), preleukemia, and chronic myelomonocytic leukemia (CMML), (2) chronic leukemias, including, but not limited to, chronic myelocytic (granulocytic) leukemia, chronic lymphocytic leukemia, and hairy cell leukemia; (3) polycythemia vera; (4) lymphomas, including, but not limited to, Hodgkin's disease and non-Hodgkin's disease; (5) multiple myelomas, including, but not limited to, smoldering multiple myeloma, nonsecretory myeloma, osteosclerotic myeloma, plasma cell leukemia, solitary plasmacytoma, and extramedullary plasmacytoma; (6) Waldenström's macroglobulinemia; (7) monoclonal gammopathy of undetermined significance; (8) benign monoclonal gammopathy; (9) heavy chain disease; (10) bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma; (11) brain tumors, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; (12) breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; (13) adrenal cancer, including, but not limited to, pheochromocytom and adrenocortical carcinoma; (14) thyroid cancer, including, but not limited to, papillary or follicular thyroid cancer, medullary thyroid cancer, and anaplastic thyroid cancer; (15) pancreatic cancer, including, but not limited to, insulinoma, gastrinoma, glucagonoma, vipoma, somatostatin-secreting tumor, and carcinoid or islet cell tumor; (16) pituitary cancer, including, but limited to, Cushing's disease, prolactin-secreting tumor, acromegaly, and diabetes insipius; (17) eye cancer, including, but not limited, to ocular melanoma such as iris melanoma, choroidal melanoma, and cilliary body melanoma, and retinoblastoma; (18) vaginal cancer, including, but not limited to, squamous cell carcinoma, adenocarcinoma, and melanoma; (19) vulvar cancer, including, but not limited to, squamous cell carcinoma, melanoma, adenocarcinoma, basal cell carcinoma, sarcoma, and Paget's disease; (20) cervical cancers, including, but not limited to, squamous cell carcinoma, and adenocarcinoma; (21) uterine cancer, including, but not limited to, endometrial carcinoma and uterine sarcoma; (22) ovarian cancer, including, but not limited to, ovarian epithelial carcinoma, borderline tumor, germ cell tumor, and stromal tumor; (23) esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; (24) stomach cancer, including, but not limited to, adenocarcinoma, fungating (polypoid), ulcerating, superficial spreading, diffusely spreading, malignant lymphoma, liposarcoma, fibrosarcoma, and carcinosarcoma; (25) colon cancer; (26) rectal cancer; (27) liver cancer, including, but not limited to, hepatocellular carcinoma and hepatoblastoma; (28) gallbladder cancer, including, but not limited to, adenocarcinoma; (29) cholangiocarcinomas, including, but not limited to, pappillary, nodular, and diffuse; (30) lung cancer, including, but not limited to, non-small cell lung cancer, squamous cell carcinoma (epidermoid carcinoma), adenocarcinoma, large-cell carcinoma, and small-cell lung cancer; (31) testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor); (32) prostate cancer, including, but not limited to, adenocarcinoma, leiomyosarcoma, and rhabdomyosarcoma; (33) penal cancer; (34) oral cancer, including, but not limited to, squamous cell carcinoma; (35) basal cancer; (36) salivary gland cancer, including, but not limited to, adenocarcinoma, mucoepidermoid carcinoma, and adenoidcystic carcinoma; (37) pharynx cancer, including, but not limited to, squamous cell cancer and verrucous; (38) skin cancer, including, but not limited to, basal cell carcinoma, squamous cell carcinoma and melanoma, superficial spreading melanoma, nodular melanoma, lentigo malignant melanoma, and acral lentiginous melanoma; (39) kidney cancer, including, but not limited to, renal cell cancer, adenocarcinoma, hypernephroma, fibrosarcoma, and transitional cell cancer (renal pelvis and/or uterer); (40) Wilms' tumor; (41) bladder cancer, including, but not limited to, transitional cell carcinoma, squamous cell cancer, adenocarcinoma, and carcinosarcoma; and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas (See Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In certain embodiments, the cancer treatable with a conjugate provided herein include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal qammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, and cancer of hematologic tissues.

In certain embodiments, the cancer treatable with a conjugate provided herein include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In certain embodiments, the cancer is epithlelial in nature, including, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, and skin cancer. In certain embodiments, the cancer is breast cancer, prostate cancer, lung cancer, or colon cancer. In certain embodiments, the epithelial cancer is non-small-cell lung cancer, nonpapillary renal cell carcinoma, cervical carcinoma, ovarian carcinoma (e.g., serous ovarian carcinoma), or breast carcinoma.

In certain embodiments, the proliferative disease is an inflammatory disease. In certain embodiments, the proliferative disease is an immune disorder. In certain embodiments, the proliferative disease is an infectious disease. In certain embodiments, the proliferative disease is a viral infection.

In another embodiment, provided herein is a method of modulating a natural killer cell in a subject, comprising administering to the subject an effective amount of a conjugate disclosed herein.

In yet another embodiment, provided herein is a method of modulating a myeloid cell in a subject, comprising administering to the subject an effective amount of a conjugate disclosed herein.

Depending on the disorder, disease, or condition to be treated, and the subject's condition, the conjugate or pharmaceutical composition provided herein can be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration. Also provided is administration of the conjugate or pharmaceutical composition provided herein in a depot formulation, in which the active ingredient is released over a predefined time period.

In the treatment, prevention, or amelioration of one or more symptoms of the disorders, diseases, or conditions described herein, an appropriate dosage level generally is ranging from about 0.001 to 100 mg per kg subject body weight per day (mg/kg per day), from about 0.01 to about 75 mg/kg per day, from about 0.1 to about 50 mg/kg per day, from about 0.5 to about 25 mg/kg per day, or from about 1 to about 20 mg/kg per day, which can be administered in single or multiple doses. Within this range, the dosage can be ranging from about 0.005 to about 0.05, from about 0.05 to about 0.5, from about 0.5 to about 5.0, from about 1 to about 15, from about 1 to about 20, or from about 1 to about 50 mg/kg per day.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The conjugate provided herein can also be combined or used in combination with other agents or therapies useful in the treatment, prevention, or amelioration of one or more symptoms of the conditions, disorders, or diseases for which the conjugate provided herein is useful.

Suitable other therapeutic agents can also include, but are not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIlb/Illa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor Vila Inhibitors and Factor Xa Inhibitors; (26) farnesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

In certain embodiments, the other therapies that may be used in combination with the conjugate provided herein include, anticancer agents and cytotoxic agents, e.g., alkylating agents, such as nitrogen mustards, alkyl sulfonates, nitrosoureas, ethylenimines, and triazenes; e.g., alkylating agents, such as nitrogen mustards, alkyl sulfonates, nitrosoureas, ethylenimines, and triazenes.

In certain embodiments, the other therapies that may be used in combination with the conjugate provided herein include, but are not limited to, an immune checkpoint modulator. In certain embodiments, the immune checkpoint modulator is a PD-1 inhibitor. In certain embodiments, the immune checkpoint modulator is a PD-L1 inhibitor. In certain embodiments, the PD-1 inhibitor is an anti-PD-1 antibody or an antigen binding fragment thereof. In certain embodiments, the PD-L1 inhibitor is an anti-PD-L1 antibody or an antigen binding fragment thereof. In certain embodiments, the immune checkpoint modulator blocks interaction between PD-1 and PD-L1.

In certain embodiments, the other therapies that may be used in combination with the conjugate provided herein include, but are not limited to, a T cell costimulatory molecule and an immune checkpoint modulator. In certain embodiments, the T cell costimulatory molecule is OX40, CD2, CD27, CDS, ICAM-1, LFA-1/CD11a/CD18, ICOS/CD278, 4-1 BB/CD137, GITR, CD30, CD40, BAFFR, HVEM, CD7, LIGHT, NKG2C, SLAMF7, NKp80, CD160, B7-H3, or CD83, or a ligand thereof. In certain embodiments, the T cell costimulatory molecule is an anti-OX40 antibody, anti-ICOS/CD278 antibody, or anti-4-1 BB/CD137 antibody, or an antigen-binding fragment thereof. In certain embodiments, the immune checkpoint modulator is an inhibitor of immune checkpoint molecules selected from PD-1, PD-L1, PD-L2, TIM-3, LAG-3, CEACAM-1, CEACAM-5, CLTA-4, VISTA, BTLA, TIGIT, LAIR1, CD47, CD160, 2B4, CD172a, and TGFR. In certain embodiments, the immune checkpoint modulator is an anti-CD47 antibody, anti-PD-1 antibody, anti-PD-L1 antibody, or an antigen-binding fragment thereof.

In certain embodiments, the other therapies that may be used in combination with the conjugate provided herein include, but are not limited to, surgery, endocrine therapy, biologic response modifiers (e.g., interferons, interleukins, and tumor necrosis factor (TNF)), hyperthermia and cryotherapy, and agents to attenuate any adverse effects (e.g., antiemetics).

Such other agents or drugs can be administered by a route and in an amount commonly used therefor, simultaneously or sequentially with the conjugate provided herein. When a conjugate provided herein is used contemporaneously with one or more other drugs, a pharmaceutical composition containing such other drugs in addition to the conjugate provided herein can be utilized, but is not required. Accordingly, the pharmaceutical compositions provided herein include those that also contain one or more other active ingredients or therapeutic agents, in addition to a conjugate provided herein.

In certain embodiments, a conjugate provided herein is administered in combination with a second antibody, e.g., an antibody that binds an antigen expressed by the cancer (e.g., an effective amount of the second antibody. Exemplary antigens expressed by cancers are known in the art and include without limitation: CD19, CD20, CD22, CD30, CD33, CD38, CD52, CD56, CD70, CD74, CD79b, CD123, CD138, CS1/SLAMF7, Trop-2, 5T4, EphA4, BCMA, Mucin 1, Mucin 16, PTK7, PD-L1, STEAP1, Endothelin B Receptor, mesothelin, EGFRvIII, ENPP3, SLC44 A4, GNMB, nectin 4, NaPi2b, LIV-1 A, Guanylyl cyclase C, DLL3, EGFR, HER2, VEGF, VEGFR, integrin αVβ3, integrin α5β1, MET, IGF1 R, TRAILR1, TRAILR2, RANKL, FAP, Tenascin, Ley, EpCAM, CEA, gpA33, PSMA, TAG72, a mucin, CAIX, EPHA3, folate receptor α, GD2, GD3, and an MHC/peptide complex comprising a peptide from NY-ESO-1/LAGE, SSX-2, a MAGE family protein, MAGE-A3, gp100/pmel17, Melan-A/MART-1, gp75/TRP1, tyrosinase, TRP2, CEA, PSA, TAG-72, immature laminin receptor, MOK/RAGE-1, WT-1, SAP-1, BING-4, EpCAM, MUC1, PRAME, survivin, BRCA1, BRCA2, CDK4, CML66, MART-2, p53, Ras, p-catenin, TGF-βRII, HPV E6, or HPV E7. In certain embodiments, a conjugate provided herein is administered in combination with a monoclonal antibody that binds CD123 (also known as IL-3 receptor alpha), such as talacotuzumab (also known as CSL362 and JNJ-56022473). In certain embodiments, a conjugate provided herein is administered in combination with a monoclonal antibody that binds EGFR (such as cetuximab). In certain embodiments, the second antibody includes one or more effector functions, e.g., effector functions that are associated with Fc receptor (FcR) engagement on immune cells including without limitation ADCC or ADCP, and/or complement-dependent cytotoxicity (CDC). Without wishing to be bound to theory, it is thought that combining such an antibody with a conjugate provided herein is particularly advantageous, e.g., to direct FcR-expressing leukocytes to target a tumor cell to which the second antibody is bound while modulating the activities of NK or myeloid cells.

In certain embodiments, a conjugate provided herein is administered in combination with an immunotherapeutic agent (e.g., an effective amount of the immunotherapeutic agent. An immunotherapeutic agent may refer to any therapeutic that targets the immune system and promotes a therapeutic redirection of the immune system, such as a modulator of a costimulatory pathway, cancer vaccine, recombinantly modified immune cell, etc. Exemplary and non-limiting immunotherapeutic agents are described infra. Without wishing to be bound to theory, it is thought that a conjugate provided herein is suitable for use with immunotherapeutic agents due to complementary mechanisms of action, e.g., in activating both macrophages and other immune cells such as T_{effector} cells to target tumor cells.

In certain embodiments, the immunotherapeutic agent comprises an antibody. Exemplary antigens of immunotherapeutic antibodies are known in the art and include without limitation BDCA2, BDCA4, ILT7, LILRB1, LILRB2, LILRB3, LILRB4, CSF-1 R, CD40, CD40L, CD163, CD206, DEC205, CD47, CD123, IDO, TDO, 41BB, CTLA4, PD1, PD-L1, PD-L2, TIM-3, BTLA, VISTA, LAG-3, CD28, OX40, GITR, CD137, CD27, HVEM, CCR4, CD25, CD103, KIrg1, Nrp1, CD278, Gpr83, TIGIT, CD154, CD160, PVRIG, DNAM, and ICOS. Immunotherapeutic agents that are approved or in late-stage clinical testing include, without limitation, ipilimumab, pembrolizumab, nivolumab, atezolizumab, avelumab, durvalumab, and the like. In certain embodiments, an antibody of the present disclosure is administered in combination with an inhibitor of the PD-L1/PD-1 pathway, e.g., an anti-PD-L1 or anti-PD-1 antibody. As demonstrated herein, combined administration of an anti-SIRP-α antibody of the present disclosure and an inhibitor of the PD-L1/PD-1 pathway can result in synergistic anti-tumor activity.

In certain embodiments, the immunotherapeutic agent comprises a vaccine, oncolytic virus, adoptive cell therapy, cytokine, or small molecule immunotherapeutic agent. Examples of such immunotherapeutic agents are known in the art. For example, adoptive cell therapies and therapeutics can include without limitation chimeric antigen receptor T-cell therapy (CAR-T), tumor infiltrating lymphocytes (TILs), TCR engineered NK cell, and macrophage cell products. Vaccines can include without limitation polynucleotide vaccines, polypeptide vaccines, or cell-based (e.g., tumor or dendritic cell-based) vaccines. Various cytokines useful for the treatment of cancer are known and include without limitation IL-2, IL-15, IL-7, IL-10, and IFN. Small molecule immunotherapeutic agents can include without limitation IDO/TDO inhibitors, arginase inhibitors, A2a R inhibitors, TLR agonists, STING agonists, and Rig-1 agonists.

In certain embodiments, a conjugate provided herein is administered in combination with a therapeutic agent including and not limited to methotrexate (RHEUMATREX®, Amethopterin) cyclophosphamide (CYTOXAN®), thalidomide (THALIDOMID®), acridine carboxamide, Actimid®, actinomycin, 17-N-allylamino-17-demethoxygeldanamycin, aminopterin, amsacrine, anthra-cycline, antineoplastic, antineoplaston, 5-azacytidine, azathioprine, BL22, bendamustine, biricodar, bleomycin, bortezomib, b ostatin, busulfan, calyculin, camptothecin, capecitabine, carboplatin, cetuximab, chlorambucil, cispla-tin, cladribine, clofarabine, cytarabine, dacarbazine, dasatinib, daunorubicin, decitabine, dichloroacetic acid, discode olide, docetaxel, doxorubicin, epirubicin, epothilone, eribulin, estramustine, etoposide, exatecan, exisulind, ferruginol, floxuridine, fludarabine, fluorouracil, fosfestrol, fotemustine, ganciclovir, gemcitabine, hydroxyurea, IT-101, idarubicin, ifosfamide, imiquimod, irinotecan, irofulven, ixabepilone, laniquidar, lapatinib, lenalidomide, lomustine, lurtotecan, mafosfamide, masoprocol, mechlorethamine, melphalan, mercaptopurine, mitomycin, mitotane, mitoxan-trone, nelarabine, nilotinib, oblimersen, oxaliplatin, PAC-1, paclitaxel, pemetrexed, pentostatin, pipobroman, pixantrone, plicamycin, procarbazine, proteasome inhibitors (e.g., bortezomib), raltitrexed, rebeccamycin, Revlimid®, rubite-can, SN-38, salinosporamide A, satraplatin, streptozotocin, swainsonine, tariquidar, taxane, tegafur-uracil, temozolo-mide, testolactone, thioTEPA, tioguanine, topotecan, tra-bectedin, tretinoin, triplatin tetranitrate, tris(2-chloroethyl) amine, troxacitabine, uracil mustard, valrubicin, vinblastine, vincristine, vinorelbine, vorinostat, or zosuquidar.

In certain embodiments, a conjugate provided herein is administered in combination with a therapeutic agent including and not limited to 3F8, 8H9, Abagovomab, Abciximab, Abituzumab, Abrilumab, Actoxumab, Adalimumab, Adecatumumab, Aducanumab, Afelimomab, Afutuzumab, Alacizumab pegol, ALD518, Alemtuzumab, Alirocumab, Altumomab pentetate, Amatuximab, Anatumomab mafenatox, Anetumab ravtansine, Anifrolumab, kinzumab (IMA-638), Apolizumab, Arcitumomab, Ascrinvacumab, Aselizumab, Atezolizumab, Atinumab, Atlizumab (tocilizumab), Atorolimumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Blinatumomab, Blosozumab, Bococizumab, Brentuximab vedotin, Briakinumab, Brodalumab, Brolucizumab, Brontictuzumab, Canaki-numab, Cantuzumab mertansine, Cantuzumab ravtansine, Caplacizumab, Capromab pendetide, Carlumab, Catumaxomab, cBR96-doxorubicin immunoconjugate, CC49, Cedelizumab, Certolizumab pegol, Cetuximab, Ch. 14.18, Citatuzumab bogatox, Cixutumumab, Clazakizumab, Clenoliximab, Clivatuzumab tetraxetan, Codrituzumab, Coltuximab ravtansine, Conatumumab, Concizumab, Crenezumab, CR6261, Dacetuzumab, Daclizumab, Dalotuzumab, Dapi-rolizumab pegol, Daratumumab, Dectrekumab, Demcizumab, Denintuzumab mafodotin, Denosumab, Derlotuximab biotin, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Engumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Epratu-zumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Giren-tuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, In iximab, Intetumumab, Inolimomab, Inotuzumab ozogamicin, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Namatumab, Natalizumab, Nebacumab, Necitumumab, Nemolizumab, Nerelimomab, Nesvacumab, Nimotuzumab, Nivolumab, Nofetumomab me entan, Obiltoxaximab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Ravirumab, Ralpancizumab, Ramucirumab, Ranibizumab, Raxibacumab, Refanezumab, Regavirumab, Reslizumab, Rilotumumab, Rinucumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19 A, SGN-CD33 A, Sifalimumab, Siltuximab, Simtuzumab, Siplizumab, Sirukumab, Sotuzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Te bazumab, Telimomab aritox, Tenatumomab, Teneliximab, Teplizumab, Teprotumumab, Tesidolumab, TGN1412, Ticilimumab (tremelimumab), Tildrakizumab, Tigatuzumab, TNX650, Tocilizumab (atlizumab), Toralizumab, Tosatoxumab, Tositumomab, Tovetumab, Tralokinumab, Trastuzumab, TRBS07, Tregalizumab, Tremelimumab, Tucotuzumab celmoleukin, Tuvirumab, Ublituximab, Ulocuplumab, Urelumab, Urtoxazumab, Ustekinumab, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, or Zolimomab aritox.

Any cancer type known in the art may be included, such as but not limited to carcinoma, sarcoma, lymphoma, leukemia, lymphoma, and blastoma. More particular examples of such cancers include, but are not limited to, lung cancer, squamous cell cancer, brain tumors, glioblastoma, head and neck cancer, hepatocellular cancer, colorectal cancer (e.g., colon or rectal cancers), liver cancer, bladder cancer, gastric or stomach cancer, pancreatic cancer, cervical cancer, ovarian cancer, cancer of the urinary tract, breast cancer, peritoneal cancer, uterine cancer, salivary gland cancer, kidney or renal cancer, prostate cancer, vulval cancer, thyroid cancer, anal carcinoma, penile carcinoma, melanoma, multiple myeloma and B-cell lymphoma (including non-Hodgkin's lymphomas (NHL)); acute lymphoblastic leukemia (ALL); chronic lymphocytic leukemia (CLL); acute myeloid leukemia (AML); Merkel cell carcinoma; hairy cell leukemia; chronic myeloblastic leukemia (CML); and associated metastases.

The conjugate provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a conjugate provided herein.

In certain embodiments, the kit includes a container comprising a dosage form of a conjugate provided herein in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

### Example 1. Synthesis and Purification of the Nucleotides and Polynucleotides

Exemplary syntheses of immunomodulating polynucleotides and precursors therefor are described below.

### Precursors

Precursors useful in the preparation of the polynucleotides of the invention are provided in WO 2015/188197 (e.g., phosphoramidites, targeting moieties, and bioreversible groups containing PEG chains).

### Phosphoramidites and Other Monomers

Nucleoside-containing intermediates useful in the synthesis of polynucleotides of the invention are disclosed in WO 2015/188197 (e.g., compounds U1-U54, A1-A15, C1-9, and G1-G12 in WO 2015/188197).

Commercially available phosphoramidites were purchased from Glen Research (Sterling, Va.) or ChemGenes (Wilmington, Mass.). When required, other phosphoramidites were prepared from appropriately protected nucleosides using standard reaction conditions described here are elsewhere.

### Compound S61B

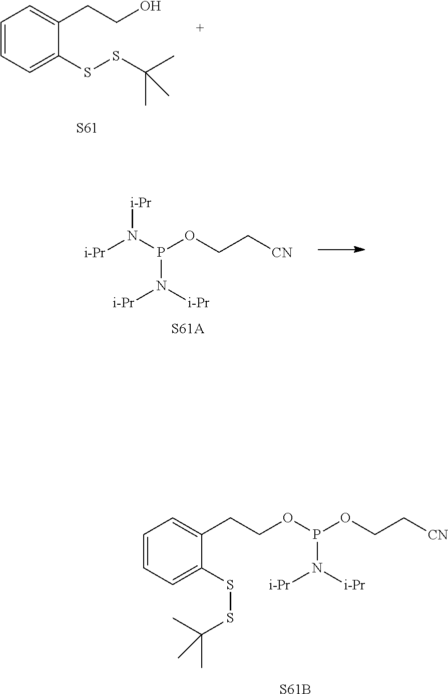 [SMILES: CC(C)(C)SSc1ccccc1CCO ; "S61" ; CC(C)N(C(C)C)P(OCCC#N)N(C(C)C)C(C)C ; CC(C)N(C(C)C)P(OCCC#N)OCCc1ccccc1SSC(C)(C)C ; "S61B"]

To a solution of S61 (0.48 g, 2.0 mmol) in DCM (5.0 mL) were added S61 A (0.60 g, 2.0 mmol) and ETT (0.25 M in acetonitrile, 4.8 mL, 1.2 mmol). The mixture was stirred for 2 h. Evaporation of the volatiles afforded a residue, which was subjected to flash silica gel column purification using ethylacete/hexane (0-30% gradient on Combi Flash Rf instrument) to give compound S61B as colorless oil (0.49 g, 55%). ³¹P NMR (202 MHz, CDCl₃; ppm): δ147.83 (s).

### Compound S108

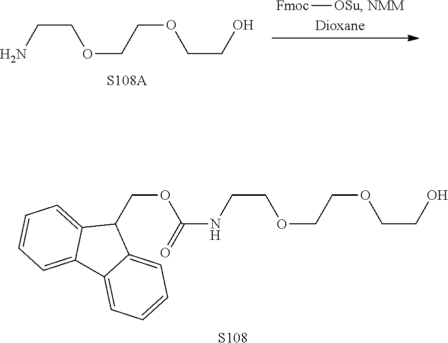

To a stirring mixture of 2-[2-(2-aminoethoxy)ethoxy]ethanol (S108 A, 25.0 g, 167 mmol) and N-methyl morpholine (21.0 mL, 191 mmol) in dioxane (100 mL) was added dropwise a solution of Fmoc-OSu (62.2 g, 184 mmol) in dioxane (50 mL). After stirring overnight, the reaction was concentrated in vacuo to afford a light yellow oil. The crude was re-dissolved in EtOAc and washed with sat. NaHCO₃ (aq.) and brine. The organic layer was removed in vacuo to afford an oil, which was purified by SiO₂ chromatography to provide the FmocNH-PEG2-OH (S108, 55 g, 88% yield). ESI+m/z calcd 371.4, found 372.2 [M+H]⁺.

X1 and X2 Abasic Spacer Synthesis—General Scheme:

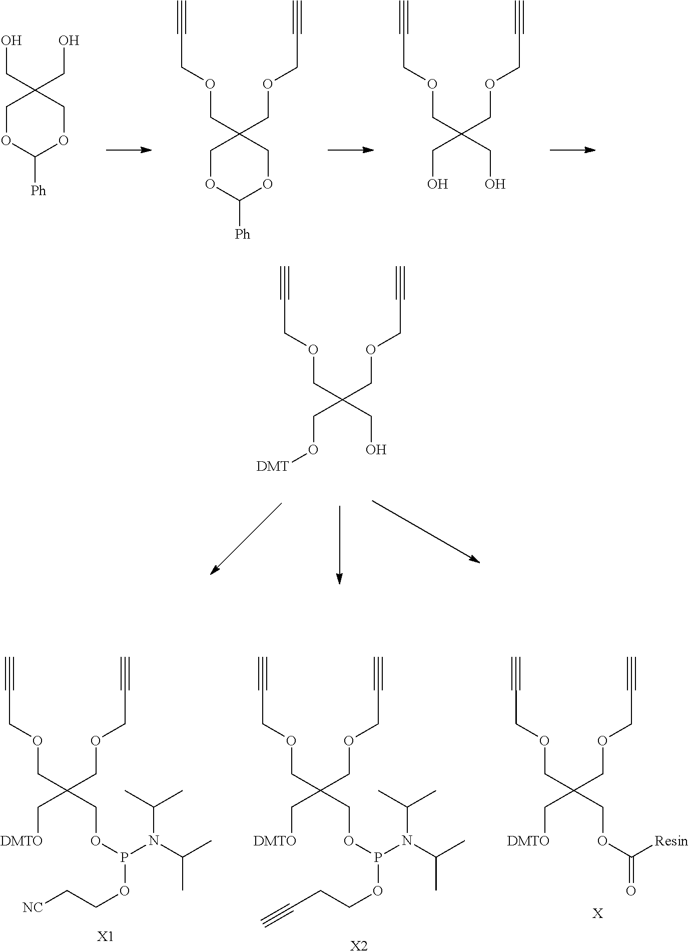

Compound S110

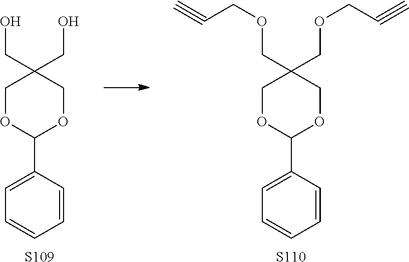 [SMILES: OCC1(CO)COC(c2ccccc2)OC1 ; C#CCOCC1(COCC#C)COC(c2ccccc2)OC1 ; "S109" ; "S110"]

To a suspension of NaH (13.2 g, 60% in mineral oil, 230.0 mmol) in THE (40 mL) under argon at 0° C. was added a solution of diol (S109, 4.92 g, 22.0 mmol) in THE (20 mL) dropwise; the resulting mixture was warmed to room temperature and stirred for 1 h. The reaction mixture was cooled to 0° C., a solution of propargyl bromide (18.6 g, 158.4 mmol) in THE (25 mL) was added slowly, and the resulting mixture was warmed to room temperature and stirred overnight at 40° C. After the product was consumed, as observed by TLC, the reaction was quenched by dropwise addition of water at 0° C., and the resulting mixture was extracted with dichloromethane (50 mL×2). The combined organic layers were washed with brine and dried over anhydrous Na₂SO₄, filtered, and evaporated to give a residue, which was purified by flash silica gel column using ISCO companion (hexane/ethyl acetate, 0-30%) to give 5.92 g (89.5%) of compound S110 as an oil. ¹H NMR (500 MHz, CDCl₃; ppm): δ7.49-7.47 (dd, J 8.0, 1.5 Hz, 2H), 7.38-7.34 (m, 3H), 5.43 (s, 1H), 4.21 (d, J 2.5 Hz, 2H), 4.12 (t, J 2.5 Hz, 4H), 4.10 (s, 1H), 3.91 (s, 1H), 3.89 (s, 1H), 3.37 (s, 2H); ESI MS for C₁₈H₂₀O₄ calculated 300.34, observed [M+H]⁺ 301.3.

### Compound S111

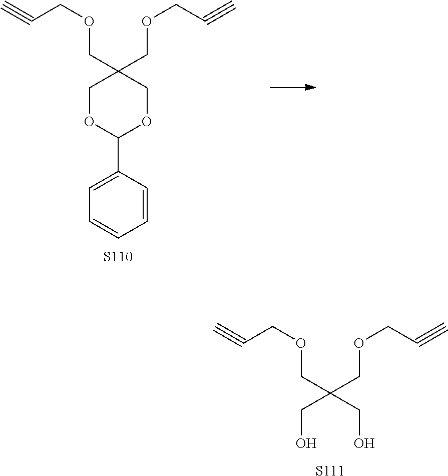 [SMILES: C#CCOCC1(COCC#C)COC(c2ccccc2)OC1 ; "S110" ; C#CCOCC(CO)(CO)COCC#C ; "S111"]

Bis-propargyl compound S110 (5.9 g, 19.64 mmol) was dissolved in acetic acid/water mixture (60 mL, 75:25), and the reaction was continued at 50° C. for 2 h. After completion of the reaction, the solution was evaporated and co-evaporated with toluene (2×20 mL). The residue was purified directly without any workup using the flash silica gel column using ISCO companion (hexane/ethyl acetate, 20-80%) to give 3.02 g (72.5%) of the compound S111 as an oil. ¹H NMR (500 MHz, CDCl₃; ppm): δ4.15 (d, J 2.5 Hz, 4H), 3.68 (s, 4H), 3.59 (s, 4H), 2.44 (t, J 2.5 Hz, 2H), 2.30-2.40 (br, 2H); ESI MS for C₁₁H₁₆O₄ calculated 212.24, observed [M+H]⁺ 213.2.

### Compound S112

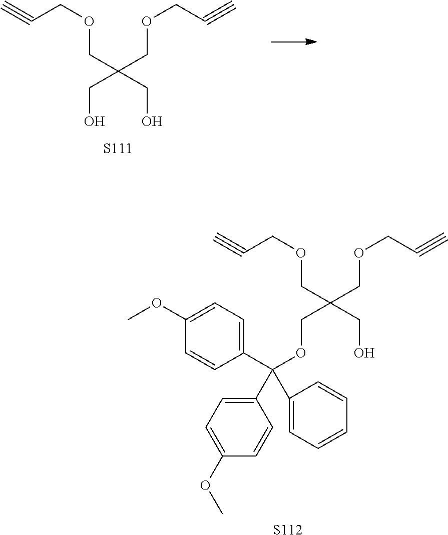 [SMILES: C#CCOCC(CO)(CO)COCC#C ; "S111" ; C#CCOCC(CO)(COCC#C)COC(c1ccccc1)(c1ccc(OC)cc1)c1ccc(OC)cc1 ; "S112"]

To a solution of diol S111 (3.0 g, 14.2 mmol), N,N-diisopropylethylamine (3.15 mL, 17.0 mmol), and DMAP (0.36 g, 2.83 mmol) in dichloromethane (25 mL) at 0° C. was added dropwise a solution of dimethoxytrityl chloride (4.8 g, 14.2 mmol) in dichloromethane (40 mL), and the reaction continued at room temperature overnight. The mixture was diluted with dichloromethane and washed with water followed by brine, and the organic layers were dried over anhydrous Na₂SO₄, filtered, and evaporated. The resulting residue was purified by flash silica gel column using ISCO companion (hexane/ethyl acetate, 0-40%) to give 5.29 g (73%) of the mono DMT protected compound S112 as white solid. ¹H NMR (500 MHz, CDCl₃; ppm): δ7.4-7.42 (m, 2H), 7.32-7.31 (m, 4H), 7.28-7.25 (m, 2H), 6.84-6.81 (m, 4H), 4.09 (d, J 2.5 Hz, 4H), 3.79 (s, 6H), 3.67 (d, J 6.0 Hz, 2H), 3.64-3.56 (m, 4H), 3.13 (s, 2H), 2.39 (t, J 2.5 Hz, 2H); ESI MS for C₃₂H₃₄O₆ calculated 514.6, observed [M+Na]⁺537.4,

### Compound S113

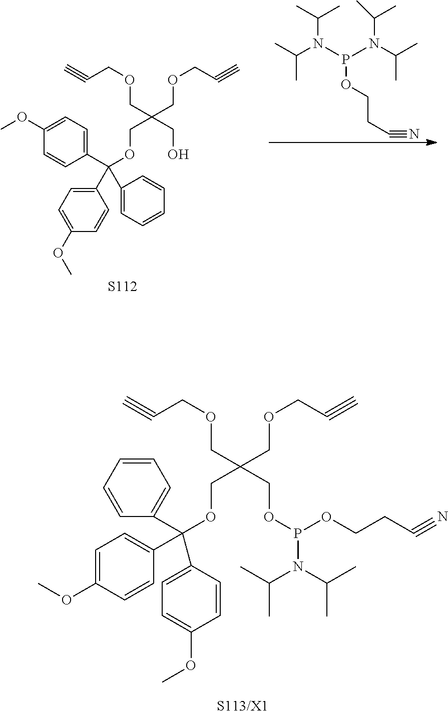 [SMILES: C#CCOCC(CO)(COCC#C)COC(c1ccccc1)(c1ccc(OC)cc1)c1ccc(OC)cc1 ; CC(C)N(C(C)C)P(OCCC#N)N(C(C)C)C(C)C ; "S112" ; C#CCOCC(COCC#C)(COP(OCCC#N)N(C(C)C)C(C)C)COC(c1ccccc1)(c1ccc(OC)cc1)c1ccc(OC)cc1 ; "S113/X1"]

To a solution of DMT-protected compound S112 (0.5 g, 0.98 mmol) in dichloromethane (4 mL) was added dropwise a solution of 2′-cyanoethyl-N,N,N′,N′-tetraisopropyl phosphoramidite (0.58 g, 1.95 mmol) in dichloromethane (3 mL) at room temperature followed by 5-benzylthio-1H-tetrazole (BTT; 0.25 M solution in acetonitrile, 0.78 mL, 0.18 mmol) under argon atmosphere. The reaction was continued until the starting material disappeared (2 h), and the crude mixture was diluted with 20 mL of dichloromethane, washed sequentially with saturated NaHCO₃ solution (10 mL) and brine (10 mL), and dried over anhydrous Na₂SO₄. The solvent was evaporated in vacuo, and the crude mixture was purified by silica gel column chromatography using ethyl acetate/hexane having 3% triethylamine as a co-solvent (0-30% gradient on Combi Flash Rf Instrument) to give 0.53 g of compound S113 (75%) as an oil. ESI MS for C₄₁H₅₁N₂O₇P Calculated 714.82, Observed 715.6 [M+H]⁺; ³¹P NMR (202 MHz, CDCl₃): δ147.89.

### Compound S114

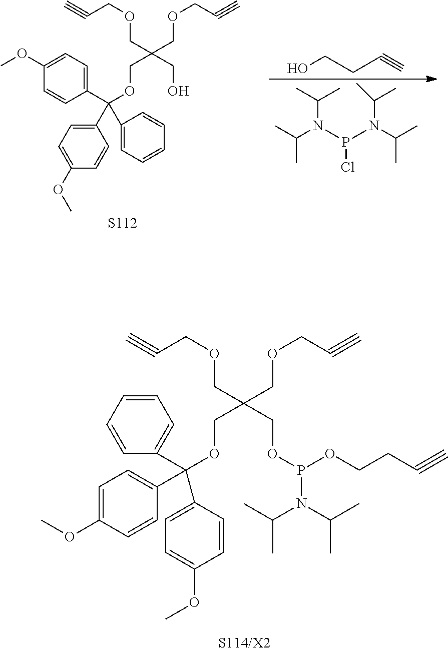

To a −78° C. solution of DMT-protected compound S112 (0.98 g, 1.9 mmol) and N,N-diisopropylethylamine (0.39 mL, 2.09 mmol) in 8.0 mL of dry dichloromethane under argon atmosphere was added dropwise a dichloromethane (4.0 mL) solution of bis-(N,N-diisopropylamino)-chlorophosphine (0.56 g, 2.09 mmol). The reaction mixture was allowed to warm to room temperature while stirring was maintained for 1 h. A solution of 3-butyne-1-ol (0.14 g, 1.9 mmol) in 2.0 mL of dry dichloromethane was added at room temperature; the resulting mixture was stirred for 10 minutes, at which time a 0.25M solution of ETT in acetonitrile (4.6 mL, 1.15 mmol) was added, and stirring continued for an additional 3 h. After completion of the reaction, as observed by the disappearance of the starting material by TLC, the crude mixture was diluted with 20 mL of dichloromethane and washed sequentially with saturated NaHCO₃ solution (10 mL) and brine (10 mL) and dried over anhydrous Na₂SO₄. The volatiles were evaporated in vacuo, and the crude mixture was purified by silica gel column chromatography using ethyl acetate/hexane with 3% triethylamine as solvent system (0-40% gradient on Combi Flash Rf Instrument) to give 0.33 g of compound S114 (25%) as an oil. ESI MS for C₄₂H₅₂NO₇P Calculated 713.83, Observed 714.7 [M+H]⁺; ³¹P NMR (202 MHz, CDCl₃): δ146.89.

### X3 and X4 Abasic Spacer Synthesis—General Scheme

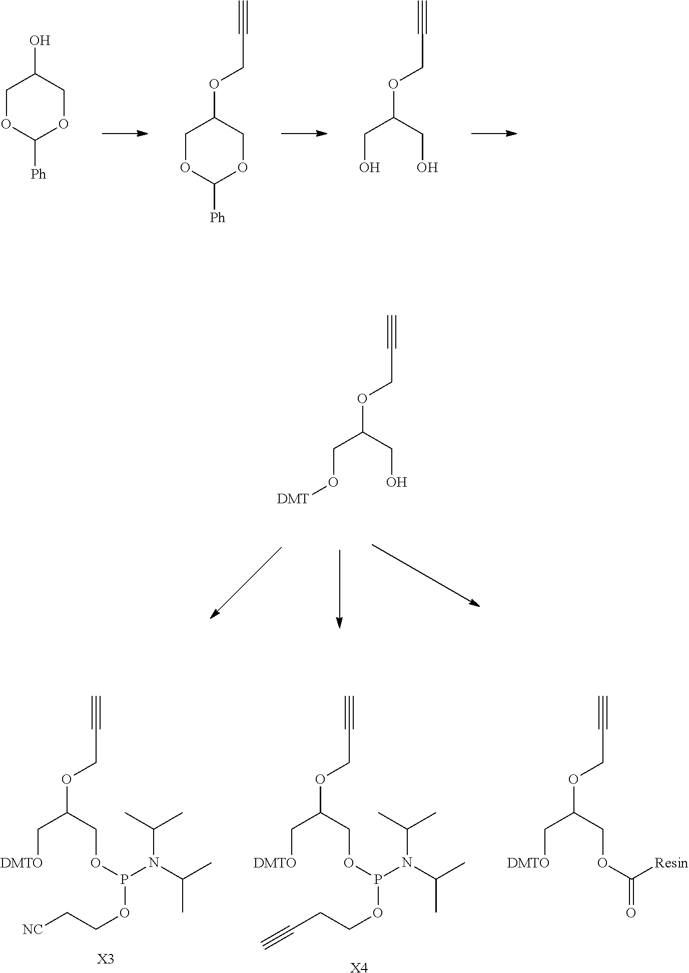

### Compound S116

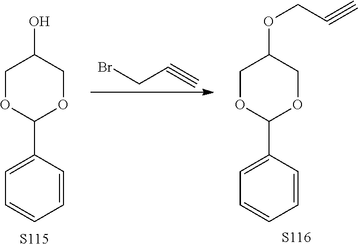

Compound S116 was prepared using the protocol described for compound S110 in 91% yield as oil. ¹H NMR (500 MHz, CDCl₃; ppm): δ7.51 (d, J 7.5 Hz, 2H), 7.37-7.32 (m, 3H), 5.56 (s, 1H), 3.37-3.35 (m, 4H), 4.10-4.07 (dd, J 13.0 Hz, J 2.5 Hz, 2H), 3.65-3.64 (m, 1H), 2.43-2.42 (t, J 6.5 Hz, 1H); ESI MS for C₁₃H₁₄O₃ calculated 218.24, observed [M+H]⁺ 219.2.

### Compound S117

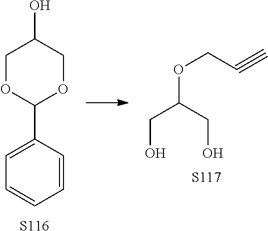

Compound S117 was prepared using the protocol described for compound S111 in 91% yield as oil. ¹H NMR (500 MHz, CDCl₃; ppm): δ4.33 (s, 2H), 3.83-3.70 (m, 5H), 2.48 (s, 1H), 2.04 (br, 2H); ESI MS for C₆H₁₀O₃ calculated 130.14, observed [M+Na]⁺153.0.

### Compound S118

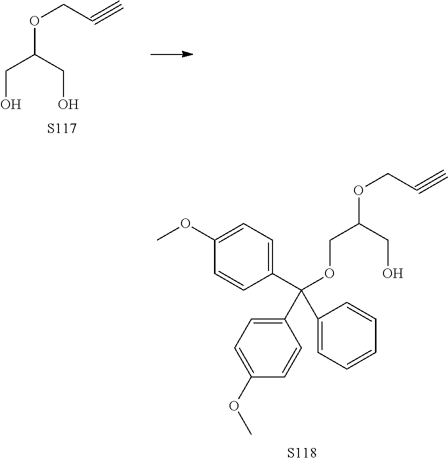

Compound S118 was prepared using the protocol described for compound S112 in 54% yield as a white solid. ¹H NMR (500 MHz, CDCl₃; ppm): δ7.43 (d, J 7.5 Hz, 2H), 7.37-7.27 (m, 5H), 7.23-7.16 (m, 2H), 6.83 (d, J 9.0 Hz, 3H), 6.78-6.76 (dd J 8.5 Hz, 1H), 4.35-4.22 (m, 2H), 3.77 (s, 6H) 3.76-3.72 (m, 2H), 3.71-3.64 (m, 1H), 3.27-3.19 (m, 2H), 2.48 (t, J 4.5 Hz, 1H), 2.03-1.96 (m, 1H); ESI MS for C₂₇H₂₈O₅ calculated 432.50, observed [M+Na]⁺455.4.

### Compound S119

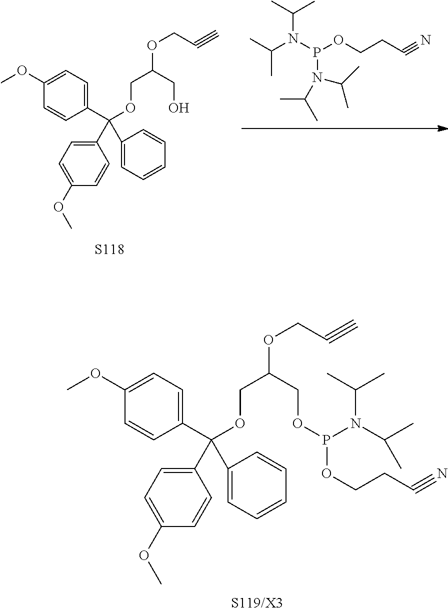

Compound S119 was prepared using the protocol described for compound S113 in 86% yield as oil. ESI MS for C₃₆H₄₅N₂O₆P Calculated 432.72, Observed 433.5 [M+H]⁺; ³¹P NMR (202 MHz, CDCl₃): δ149.05, 148.96.

### Compound S120

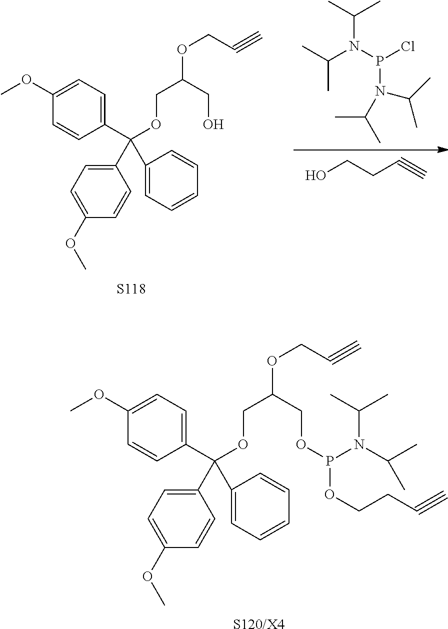

Compound S120 was prepared using the protocol described for compound S114 in 47% yield as oil. ESI MS for C₃₇H₄₆NO₆P Calculated 431.73, Observed 432.5 [M+H]⁺; ³¹P NMR (202 MHz, CDCl₃): δ147.80, 147.71.

### X5 and X6 Abasic Spacer Synthesis—General Scheme

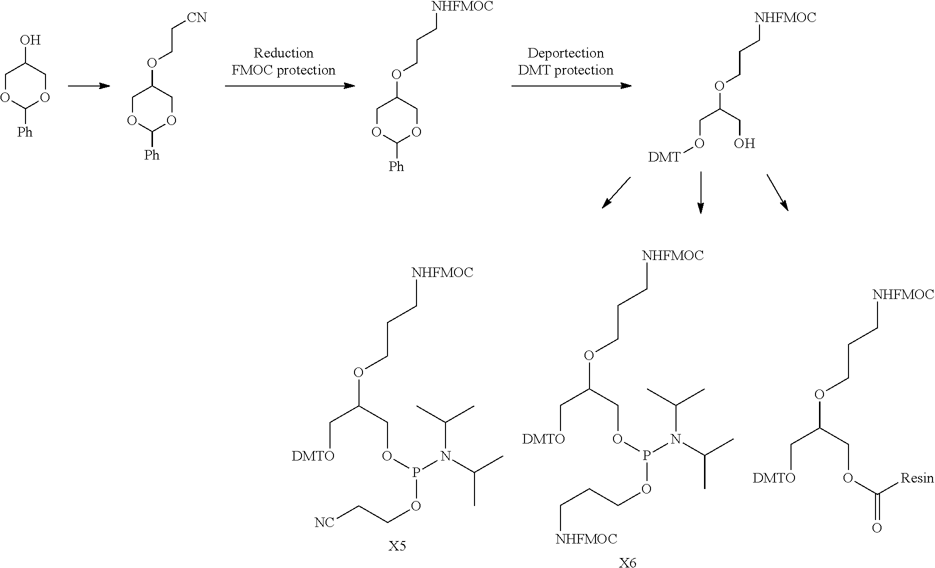

### Compound S121

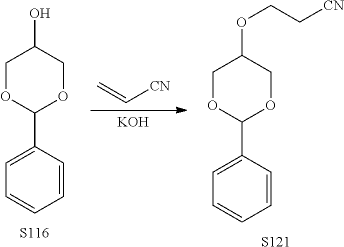

To a solution of S116 (4.0 g, 22.2 mmol) in dioxane (25 mL) was added a solution of KOH (0.12 g, 2.2 mmol) dissolved in minimum amount of water, and the resulting mixture was stirred for at least 30 minutes at room temperature. The mixture was cooled to 0° C., a solution of acrylonitrile (2.35 g, 44.4 mmol) in dioxane (15 mL) was added dropwise, and the resulting mixture was allowed to react at room temperature for overnight. Volatiles were evaporated in vacuo, the residue was diluted with water, and the pH was adjusted to near neutral. The crude product was extracted with ethyl acetate (2×50 mL), and the combined organic layers were washed with brine and dried over anhydrous Na₂SO₄, filtered, and evaporated to give a residue, which was purified by flash silica gel column using ISCO companion (dichloromethane/methanol, 0-5%) to give 3.1 g (60%) of the compound S121 as white solid. ¹H NMR (500 MHz, CDCl₃; ppm): δ7.49 (d, J 7.0 Hz, 2H), 7.36-7.34 (m, 3H), 5.56 (s, 1H), 3.36 (d, J 13.0 Hz 2H), 4.10-4.07 (dd, J 13.0 Hz, J 2.0 Hz, 2H), 3.84 (t, J 6.5 Hz, 2H), 3.42 (m, 1H), 3.69 (t, J 6.5 Hz, 2H); ESI MS for C₁₃H₁₅NO₃ calculated 233.2, observed [M+Na]⁺256.3.

### Compound S122

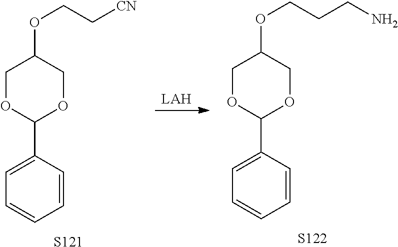 [SMILES: N#CCCOC1COC(c2ccccc2)OC1 ; "LAH" ; NCCCOC1COC(c2ccccc2)OC1 ; "S122" ; "S121"]

To a suspension of lithium aluminum hydride (0.83 g, 4.0 mmol) in THE (10 mL) at 0° C. was added dropwise a solution of compound S121 (1.28 g, 5.5 mmol) in THE (15 mL), the resulting mixture was warmed to room temperature, and stirring was continued for 3 h. After completion of the reaction, the reaction mixture was cooled to 0° C. and quenched by dropwise addition of water as required (ca. 2-3 mL). Additional ca. 8 mL of water were added, and the crude product was extracted into ethyl acetate (2×25 mL). The combined organic layers were washed with brine, dried over anhydrous Na₂SO₄, filtered, and evaporated to give compound S122, which was used in the subsequent step without further purification. ¹H NMR (500 MHz, CDCl₃; ppm): δ7.49 (d, J 7.0 Hz, 2H), 7.40-7.32 (m, 3H), 5.55 (d, J 5.0 Hz, 1H), 4.34 (d, J 13.0 Hz, 1H), 4.20-4.11 (dd, J 12.0 Hz 4H), 4.05-4.03 (d, J 13.0 Hz, J 2.0 Hz, 1H), 3.66-3.62 (m, 2H), 3.27 (m, 1H), 2.86 (t, J 6.5 Hz, 1H), 2.16 (br, 2H); ESI MS for C₁₃H₁₉NO₃ calculated 237.2, observed [M+H]⁺238.2.

### Compound S123

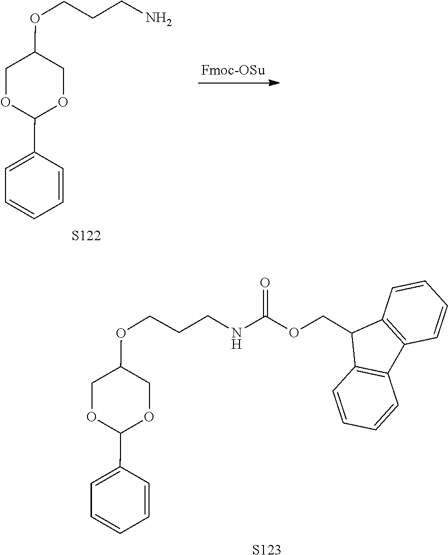 [SMILES: NCCCOC1COC(c2ccccc2)OC1 ; "S122" ; O=C(NCCCOC1COC(c2ccccc2)OC1)OCC1c2ccccc2-c2ccccc21 ; "S123"]

To compound S122 (1.0 g, 4.2 mmol) and N,N-diisopropylethylamine (2.3 mL, 12.6 mmol) in dichloromethane (8 mL) at 0° C. was added dropwise a solution of Fmoc-OSu (1.7 g, 5.0 mmol), and the resulting mixture was allowed to react at room temperature for 3 h. After completion, the reaction mixture was diluted with dichloromethane (10 mL) and washed with water followed by brine. The organic layer was separated, dried over anhydrous Na₂SO₄, filtered, and evaporated to give a residue. The residue was purified by flash silica gel column using ISCO companion (hexane/ethyl acetate, 0-50%) to give 0.65 g (35%) of the compound S123 as a white solid. ¹H NMR (500 MHz, CDCl₃; ppm): δ7.75 (d, J 7.5 Hz, 2H), 7.58 (d, J 7.5 Hz, 2H), 7.51 (d, J 7.5 Hz, 2H), 7.37 (t, J 7.5 Hz, 2H), 7.31-7.26 (m, 5H), 5.57 (s, 1H), 5.48 (br, 1H), 4.46-4.32 (m, 4H), 4.15 (d, J 7.0 Hz, 1H), 4.06 (t, J 12.5 Hz 2H), 3.67 (m, 2H), 3.54 (m, 2H), 3.41 (s, 1H), 1.88 (t, J 6.0 Hz, 2H); ESI MS for C₂₈H₂₉NO₅ calculated 459.5, observed [M+Na]⁺482.5.

### Compound S124

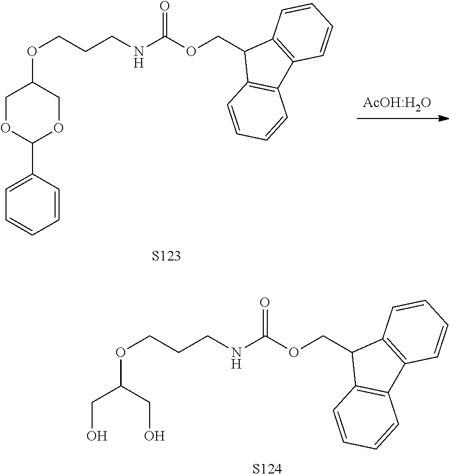

Compound S124 was prepared using the protocol described for compound S111 with quantitative yields as an oil. ¹H NMR (500 MHz, CDCl₃; ppm): δ7.76 (d, J 7.5 Hz, 2H), 7.58 (d, J 7.5 Hz, 2H), 7.39 (t, J 7.5 Hz, 2H), 7.32 (t, J 7.5 Hz, 2H), 5.18 (br, 1H), 4.44 (d, J 6.5 Hz, 2H), 4.21 (t, J 6.5 Hz, 1H), 4.76-4.73 (dd, J 11.5, 3.5 Hz 2H), 3.67-60 (m, 4H), 3.42 (m, 1H), 3.37 (br, 2H), 2.07 (m, 2H), 1.75 (br, 2H); ESI MS for C₂₁H₂₅NO₅ calculated 371.4, observed [M+Na]⁺394.3.

### Compound S125

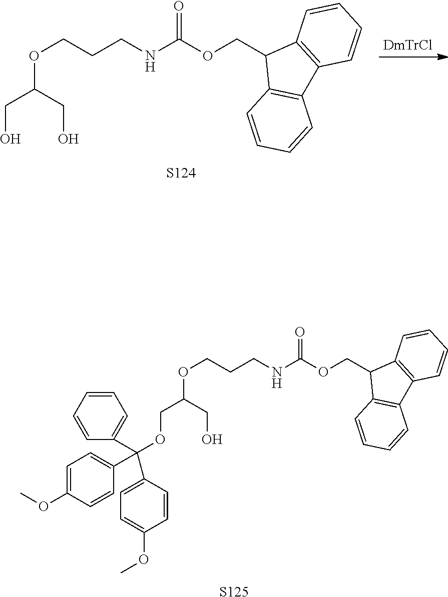 [SMILES: O=C(NCCCOC(CO)CO)OCC1c2ccccc2-c2ccccc21 ; "S124" ; COc1ccc(C(OCC(CO)OCCCNC(=O)OCC2c3ccccc3-c3ccccc32)(c2ccccc2)c2ccc(OC)cc2)cc1 ; "S125"]

Compound S125 was prepared using the protocol described for compound S112 with 48% of product (S125) yield as a white solid. ¹H NMR (500 MHz, CDCl₃; ppm): δ7.75 (t, J 7.5 Hz, 2H), 7.58 (t, J 7.5 Hz, 2H), 7.40-7.38 (m, 3H), 7.32-27 (m, 7H), 7.18-7.16 (m, 3H), 6.83 (t, J 7.0 Hz, 4H), 5.16 (br, 1H), 4.44 (d, J 6.5 Hz, 2H), 4.20 (m, 1H), 3.80 (s, 3H), 3.79 (m, 1H), 3.76 (s, 3H), 3.74 (m, 2H), 3.66-3.62 (m, 4H), 3.43-3.37 (m, 2H), 2.31 (br, 1H), 1.76 (br, 2H); ESI MS for C₄₂H₄₃NO₇ calculated 673.7, observed [M+Na]⁺696.7.

### Compound S126

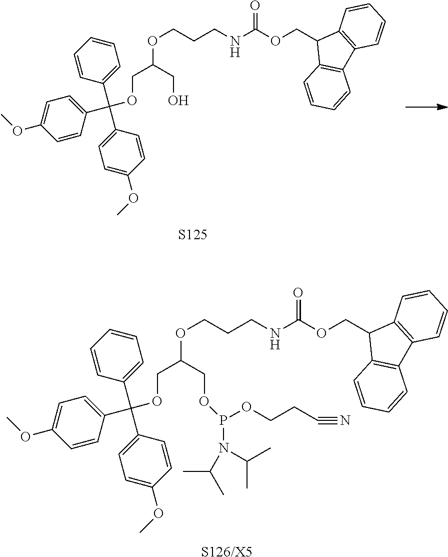 [SMILES: COc1ccc(C(OCC(CO)OCCCNC(=O)OCC2c3ccccc3-c3ccccc32)(c2ccccc2)c2ccc(OC)cc2)cc1 ; "S125" ; COc1ccc(C(OCC(COP(OCCC#N)N(C(C)C)C(C)C)OCCCNC(=O)OCC2c3ccccc3-c3ccccc32)(c2ccccc2)c2ccc(OC)cc2)cc1 ; "S126/X5"]

Compound S126 was prepared using the protocol described for compound S113 with 78% of product (S126) yield as an oil. ESI MS for C₅₁H₆₀N₃O₈P Calculated 874.0, Observed 896.9 [M+Na]⁺, 913.0 [M+K]+; ³¹P NMR (202 MHz, CDCl₃; ppm): δ148.90, 148.76.

### Synthesis of Abasic Spacer S131—General Scheme

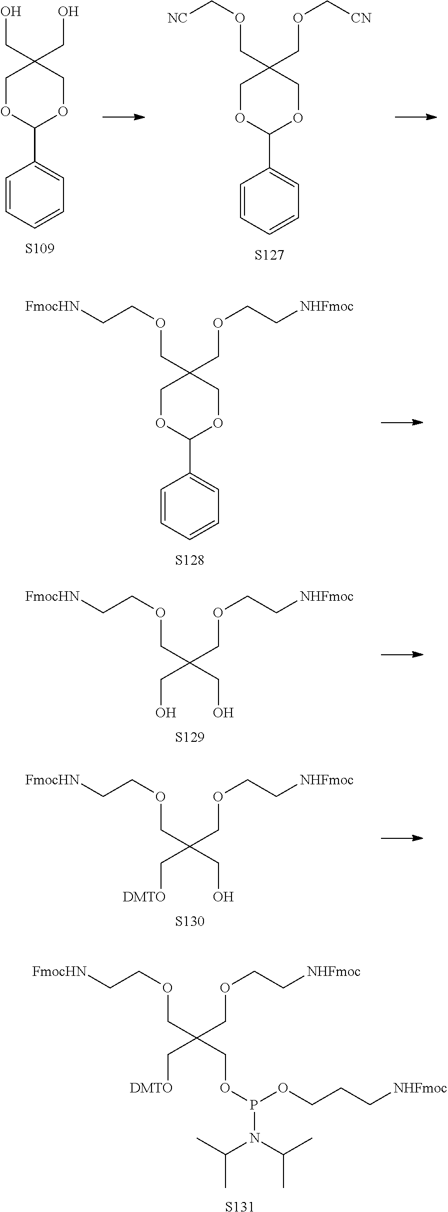

### Compound S127

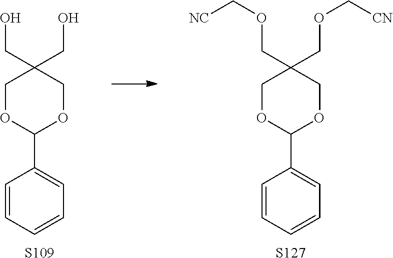 [SMILES: OCC1(CO)COC(c2ccccc2)OC1 ; N#CCOCC1(COCC#N)COC(c2ccccc2)OC1 ; "S127" ; "S109"]

To a solution of S109 (2.56 g, 11.4 mmol) in dichloromethane (50 mL) under argon were added bromoacetonitrile (3.01 g, 25.1 mmol), silver(I) oxide (5.28 g, 22.8 mmol), and tetrabutylammonium iodide (0.84 g, 2.28 mmol), and the resulting mixture was stirred overnight. The mixture was filtered over Celite®, and the filtrate was evaporated to give a black residue, which was subjected to flash silica gel column purification on ISCO companion (hexane/ethyl acetate, 15-90%) to give 1.34 g (39%) of the desired compound S127 as a viscous oil. ESI MS for C₁₆H₁₈N₂O₄ calculated 302.3, observed [M+H]⁺303.3.

### Compound S128

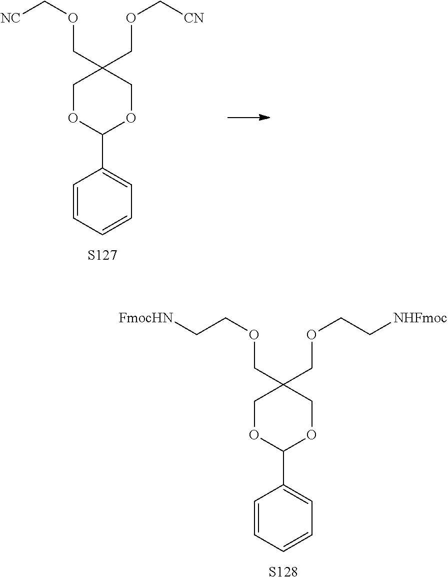

To a solution of compound S127 (1.34 g, 4.43 mmol) in THE (30 mL) was added a solution of LiAlH₄ in THE (2M, 8.9 mL, 17.7 mmol) under argon, and the mixture was heated to 55° C. for 4 h. Another portion of LiAlH₄ in THE (2M, 4 mL, 8.0 mmol) was added, and the stirring continued for 4 h. After completion of the reaction, the mixture was cooled to room temperature and quenched with Na₂SO₄.10H₂O. The solid was filtered off and washed with ethyl acetate. The filtrate was dried over anhydrous Na₂SO₄. The mixture was filtered and evaporated to give a residue, which was dissolved in dichloromethane (20 mL). To this solution were added Fmoc-OSu (1.5 g, 4.43 mmol) and DIEA (0.87 mL, 5.0 mmol). The mixture was stirred for 1 h, then evaporated to give a residue, which was subjected to flash silica gel column purification on a ISCO companion (hexane/ethyl acetate, 20-90%) to give 1.04 g (31%) of the compound S128 as a white foam. ¹H NMR (500 MHz, CDCl₃; ppm): δ7.75 (4H, dd, J 7.5, 4.5 Hz), 7.58 (4H, t, J 7.0 Hz), 7.48 (2H, d, J 7.0 Hz), 7.41-7.34 (7H, m), 7.32-7.26 (4H, m), 5.44 (1H, s), 5.15-5.05 (2H, m), 4.44 (2H, d, J 5.5 Hz), 4.38 (2H, d, J 6.0 Hz), 4.25-4.15 (2H, m), 4.10 (2H, d, J 11.5 Hz), 3.82 (2H, d, J 11.5 Hz), 3.78 (2H, s), 3.53 (2H, s), 3.42 (2H, s), 3.36-3.27 (4H, m), 3.25 (2H, s); ESI MS for C₄₆H₄₆N₂O₈ calculated 754.9, observed [M+H]⁺ 755.3.

### Compound S129

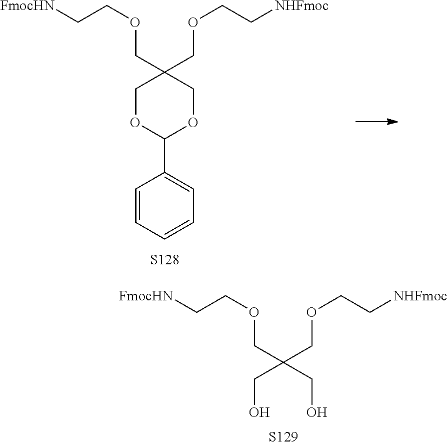

Compound S128 (1.1 g, 1.51 mmol) was dissolved in AcOH/H₂O mixture (10 mL, 3:1), and the reaction was continued at 55° C. for 5 h. After completion of the reaction, the volatiles were evaporated and co-evaporated with toluene (2×20 mL), and the residue was subjected to flash silica gel column purification on a ISCO companion (hexane/ethyl acetate, 30-100%) to give 0.54 g (54%) of the compound S129 as white foam. ¹H NMR (500 MHz, CDCl₃; ppm): δ7.75 (4H, d, J 7.5 Hz), 7.58 (4H, d, J 7.5 Hz), 7.39 (4H, t, J 7.5 Hz), 7.30 (4H, t, J 7.5 Hz), 5.20-5.05 (2H, m), 4.41 (4H, d, J 6.5 Hz), 4.21 (4H, t, J 6.5 Hz), 3.64 (4H, s), 3.48 (8H, s), 3.36 (4H, s); ESI MS for C₃₉H₄₂N₂O₈ calculated 666.7, observed [M+H]⁺667.3.

### Compound S130

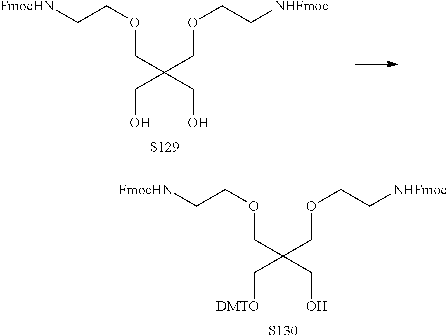

To a solution of diol S129 (0.73 g, 1.1 mmol), DIPEA (0.19 mL, 1.1 mmol) and DMAP (0.013 g, 0.11 mmol) in dichloromethane (6 mL) at 0° C. was added a solution of DMTrCl (0.34 g, 0.99 mmol) in dichloromethane (1 mL) dropwise. The resulting mixture was warmed to room temperature and stirred overnight. the mixture was evaporated to give a residue, which was subjected to flash silica gel column purification on a ISCO (hexane/ethyl acetate, 20-100%) to give 0.47 g (44%) of the mono dimethoxytrityl protected compound S130 as a white foam. ¹H NMR (500 MHz, CDCl₃; ppm): δ7.75 (4H, d, J 7.5 Hz), 7.58 (4H, d, J 7.5 Hz), 7.39 (4H, t, J 7.5 Hz), 7.32-7.25 (8H, m), 7.17 (4H, d, J 6.5 Hz), 6.83 (4H, d, J 6.5 Hz), 5.20-5.05 (2H, m), 4.41 (4H, d, J 6.5 Hz), 4.21 (4H, t, J 6.5 Hz), 3.82 (6H, s), 3.64 (4H, s), 3.48 (8H, s), 3.36 (4H, s); ESI MS for C₆₀H₆₀N₂O₁₀ calculated 969.1, observed [M+Na]⁺991.3.

### Compound S131

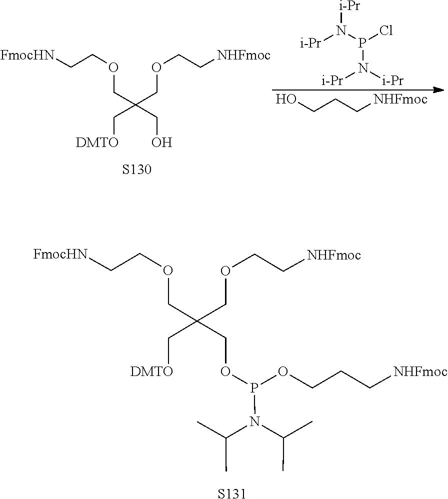

A solution of bis-(N,N-disiopropylamino)-chlorophosphine (0.085 g, 0.32 mmol) in dry CH₂Cl₂ (1.0 mL) were added dropwise to a solution of 3-Fmoc-amino-propan-1-ol (0.090 g, 0.30 mmol) and N,N-diisopropylethylamine (0.18 mL, 1.05 mmol) in dry CH₂Cl₂ (3.0 mL) at −78° C. The reaction mixture was warmed to room temperature and stirred for 1.5 h. A solution of compound S130 (0.30 g, 0.30 mmol) in 1.0 mL of dry CH₂Cl₂ was added, and the resulting mixture was stirred for 10 min. A solution of ETT (0.72 mL, 0.25M in acetonitrile, 0.18 mmol) was added to the reaction mixture, and the resulting mixture was stirred for 3 h. The mixture was diluted with CH₂Cl₂ (20 mL) and washed with saturated aqueous sodium bicarbonate (20 mL) and brine (20 mL). The organic layer was dried over anhydrous sodium sulfate, and the filtrate was evaporated in vacuum to afford a residue, which was subjected to flash silica gel column purification on a ISCO companion using ethyl acetate/hexane with 3% triethylamine as a co-solvent system (0-30% gradient) to give 0.12 g of product S131 (32%) as a white foam. ESI MS for C₈₄H₉₁N₄O₁₃P Calculated 1395.6, Observed 1395.7[M]⁺; ³¹P NMR (202 MHz, CDCl₃): δ146.41.

### Compound dT4

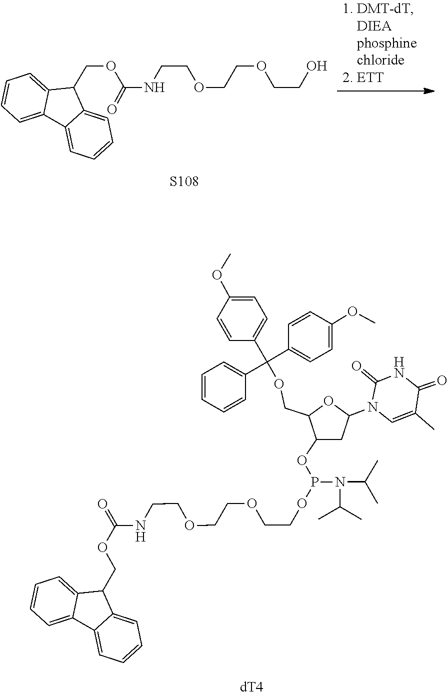 [SMILES: O=C(NCCOCCOCCO)OCC1c2ccccc2-c2ccccc21 ; "S108" ; COc1ccc(C(OCC2OC(n3cc(C)c(=O)[nH]c3=O)CC2OP(OCCOCCOCCNC(=O)OCC2c3ccccc3-c3ccccc32)N(C(C)C)C(C)C)(c2ccccc2)c2ccc(OC)cc2)cc1]

Synthesis of FmocNH-PEG2-hydroxyl-diisopropylamino-dT(5′-DMT) phosphoramidite (dT4). A stirring suspension of 5′-DMT-deoxythymidine (4.30 g, 7.89 mmol) and DIEA (1.51 mL, 8.68 mmol) in CH₂Cl₂ (40 mL) was cooled to −78° C. under argon. A solution of bis(diisopropylamino)chlorophosphine (2.32 g, 8.68 mmol) in CH₂Cl₂ (10 mL) was added dropwise. The mixture was removed from the cooling bath and stirred for 1 h. FmocNH-PGE2-OH (S108, 2.93 g, 7.89 mmol) in CH₂Cl₂ (15 mL) was added to the reaction mixture followed by a solution of ETT (0.25 M in acetonitrile, 18.9 mL). After stirring overnight, the mixture was concentrated in vacuo, re-dissolved in EtOAc, and washed with sat. NaHCO₃(aq.) and brine. The organic layer was removed in vacuo to afford a white foam. This crude material was purified by SiO₂ chromatography to provide the title phosphoramidite (dT4, 4.1 g, 50% yield).

Synthetic protocol described above was used for the synthesis of other phosphoramidite precursors of varying triesters.

### Compound dU6

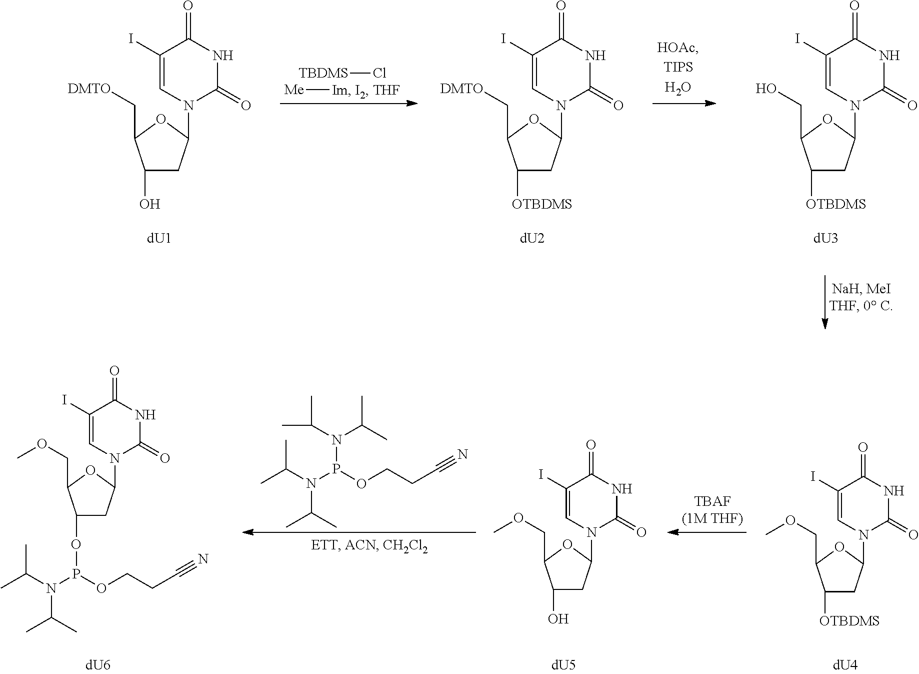

To a solution of dU1 (3.3 g, 5.0 mmol), 1-methylimidazole (1.2 mL, 15.0 mmol) and iodine (1.9 g, 15.0 mmol) in THF (10 mL) under Ar (g) at room temperature was added a solution of tert-butyldimethylsilyl chloride (0.8 g, 5.5 mmol) in THF (5 mL) dropwise with stirring. Reaction stirred at room temperature for 1 hour. TLC confirmed the completion of the reaction. Solvent was remove in vacuo, crude was dissolved in ethyl acetate and washed with aq. Na₂S₂O₃ (conc). Dried organic phase over Na₂SO₄, filtered and evaporated liquor. Crude was purified by flash silica gel column using an ISCO companion (hexanes/ethyl acetate, 0-50%) to give dU2 as a solid in quantitative yield. NMR consistent with published. Nucleic Acids Research, 2011, Vol. 39, No. 9, 3962-3971.

A solution of dU2 (3.9 g, 5.0 mmol) dissolved in an 80% aqueous acetic acid solution (40 mL) with triisopropylsilane (1.0 mL, 5.0 mmol) was stirred at room temperature for 1 hour. TLC confirmed the completion of the reaction. Remove solvent in vacuo. Crude was purified by a flash silica gel column using an ISCO companion (hexanes/ethyl acetate, 0-60%) to give 1 g (43%) of the desired compound dU3 as a solid. ESI MS for C₁₅H₂₅IN₂O₅Si calculated 468.4, observed [M+Na]⁺491.0.

To a solution of dU3 (1.0 g, 2.2 mmol) in THF (20 mL) under Ar (g) and cooled to 0° C. in an ice water bath was added sodium hydride (60% dispersion, 0.2 g, 4.7 mmol). The reaction was stirred for 30 minutes at 0° C. Iodomethane (0.7 mL, 10.8 mmol) was added dropwise and the reaction was stirred at 0° C. for 3 hours. RP—HPLC/MS confirmed the completion of the reaction. Reaction was quenched with 20 mL of methanol at 0° C. and warmed to room temperature. Aq. NaHCO₃ (sat.) was added and the mixture was extracted with CH₂Cl₂. Organic phase was dried over Na₂SO₄, filtered and liquor concentrated in vacuo. Purification by silica gel column chromatography (hexanes/ethyl acetate, 0-50%) gave solid dU4 (0.6 g, 58% yield). ESI MS for C₁₆H₂₇IN₂O₅Si calculated 482.4, observed [M+H]⁺ 483.1.

Tert-butylammonium fluoride (1 M THF, 3 mL, 3.0 mmol) was added dropwise with stirring to a cooled (0° C.) solution of dU4 (0.6 g, 1.3 mmol) dissolved in THF (20 mL) under Ar (g). The cooled solution was stirred for 30 minutes then warmed to room temperature. After 3.5 hours, RP—HPLC/MS confirmed the completion of the reaction. The crude product was purified by silica gel column chromatography (dichloromethane/methanol, 0-10%) to give solid dU5 (0.4 g, 92% yield). ESI MS for C₁₀H₁₃IN₂O₅ calculated 368.1, observed [M+H]⁺ 369.0.

To a solution dU5 (0.4 g, 1.2 mmol) in dichloromethane (5 mL) under Ar (g) at room temperature was added a solution of 2′-cyanoethyl-N, N, N′, N′tetraisopropyl phosphoramidite (0.4 mL, 1.3 mmol) in dichloromethane (5 mL) dropwise with stirring. Reaction stirred for 30 minutes at room temperature. Ethylthiotetrazole (0.25 M solution in ACN, 2.9 mL, 0.7 mmol) was then added and the reaction was continued overnight. TLC confirmed the completion of the reaction. Solvent removed in vacuo and the crude mixture was diluted with 20 mL of dichloromethane, washed sequentially with a saturated NaHCO₃ solution (10 mL) and brine (10 mL). Dried organic phase over Na₂SO₄, filtered and evaporated liquor. Crude mixture was dissolved in ethyl acetate and purified by silica gel column using an Isco companion (hexanes/ethyl acetate, 0-100%) to give 0.3 g (49.9%) of the desired compound dU6 as a solid. ESI MS for C₁₉H₃₀N₄O₆P Calculated 568.3, Observed 567.3 [M−H]⁻; ³¹P NMR (202 MHz, CDCl₃, ppm): δ149.25.

### Compound dU9

The title compound was prepared by reacting dU3 under standard reaction conditions shown below. ³¹P-NMR (202 mHz, CDCl₃, ppm): δ149.42, 149.31; MS ESI-m/z found 667.1 [M−H]. MS ESI+m/z found 669.2 [M+H], 691.3 [M+Na].

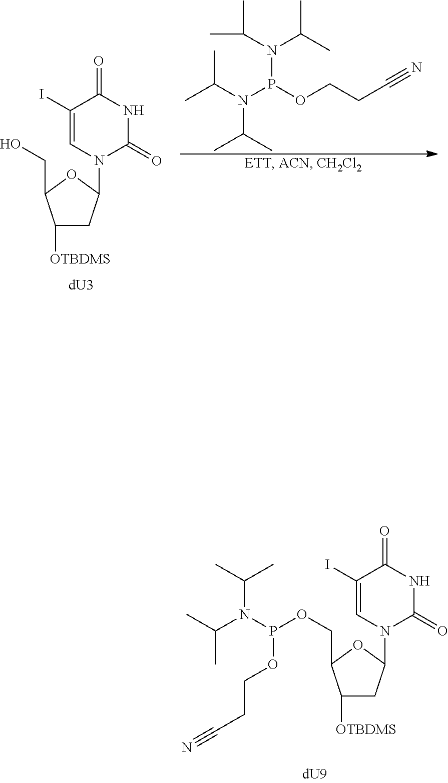

### Preparation of Linkers Bonded to Auxiliary Moieties

### Compounds PP2, PP3, and PP4

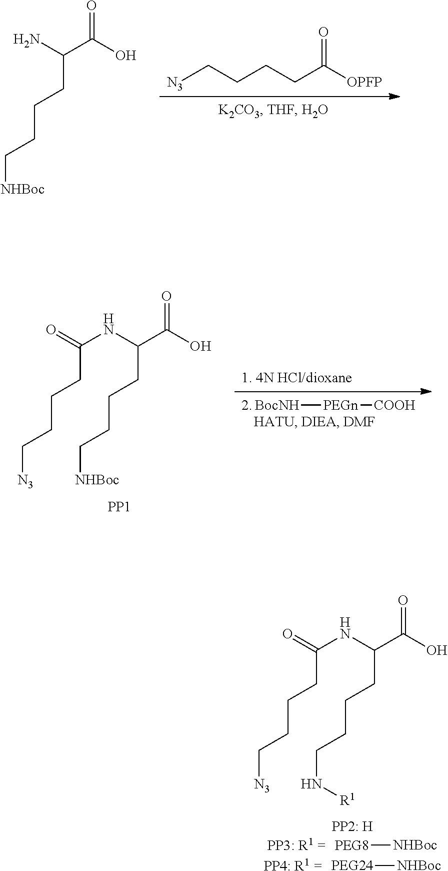

Preparation of (5-Azidovaleryl)-ε-N-Boc lysine (PP1). ε-N-Boc lysine (9.46 g, 38.4 mmol) and K₂CO₃ (2.67 g, 19.3 mmol) were dissolved in 1:1 THF:H₂O (60 mL). Pentafluorophenyl-5-azidovalerate (10.8 g, 34.9 mmol) in THE (10 mL) was added, and the reaction stirred overnight at room temperature. The desired product was observed by RP—HPLC-MS, 394.2 [M+Na]. The reaction was acidified to pH 5 by titration with 1 N HCl (aq.), and the product was extracted with EtOAc (3×100 mL). The organic layer was washed sequentially with H₂O (50 mL) and brine (50 mL). The organic layer was dried over MgSO₄ and concentrated in vacuo to a thick syrup. The crude product was purified by silica gel column chromatography to afford the desired product PP2 as white needles (8.1 g, 62% yield). ESI MS+ mass calculated C₁₆H₂₉N₅O₅: 371.4, found: 394.2 [M+Na]⁺.

General protocol for pegylation of PP1: preparation of (5-Azidovaleryl)-ε-N— (NH-Boc PEG24) lysine (PP4). PP1 (0.74 g, 2.0 mmol) was treated with HCl (2 mL, 4N in dioxane) for 4 h. HPLC-MS showed complete deprotection, 272.2 [M+H]⁺. The reaction was diluted with 1:1 H₂O:acetonitrile (10 mL), frozen, and lyophilized overnight to afford PP2 as a white solid in quantitative yield. NHBoc-PEG24 acid (1.1 g, 0.88 mmol) in DMF (3 mL) was activated with HATU (0.34 g, 0.88 mmol), HOBt (0.14 g, 0.88 mmol), and DIEA (0.7 mL, 4.0 mmol) then treated with PP2 (0.24 g, 0.8 mmol) for 2 hours. R^{P}-HPLCMS showed formation of the desired PP4. The crude was purified by R^{P}-HPLC to afford PP4 as a white solid (0.55 g, 46% yield). ESI MS+ mass calculated C₆₇H₁₃₀N₆O₃₀: 1499.77, found: 1499.9 [M+H]⁺, 1400.8 [M-Boc]⁺.

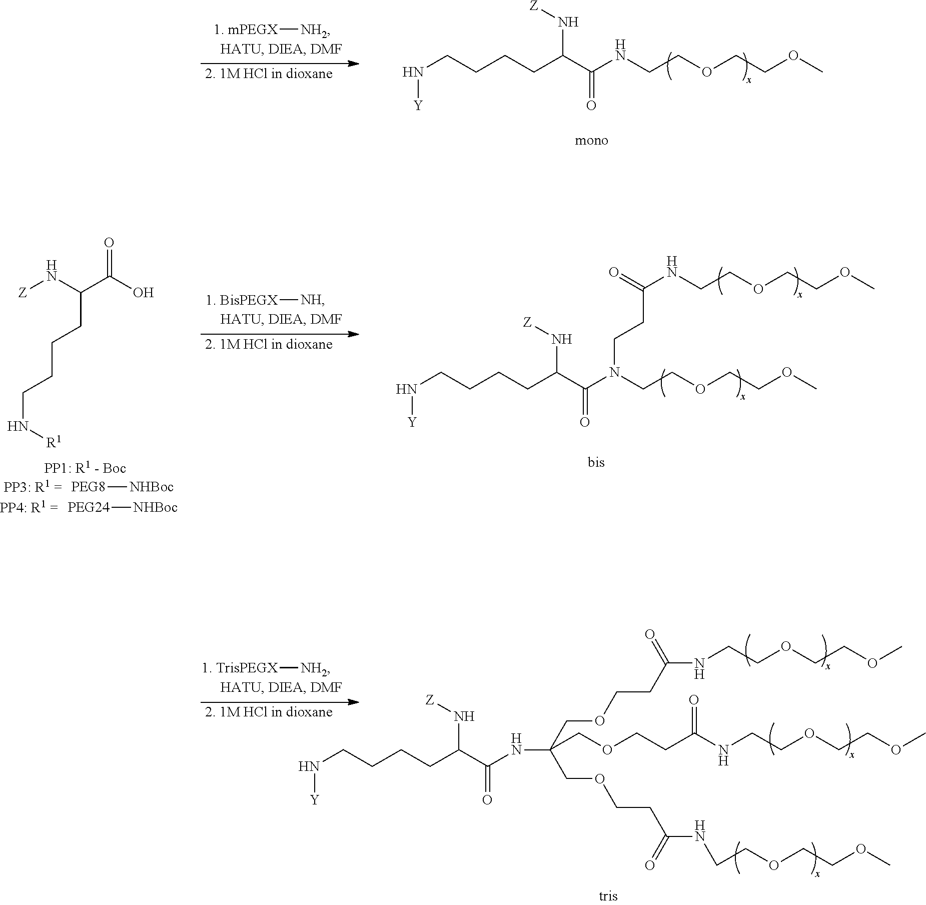

BisPegX-NH2 and TrisPegX-NH2 (where X=various PEG lengths) were prepared from commercially available starting materials using procedures described in WO2015/188197.

General protocol for pegylation of PP2, PP3, and PP4: Lysine PP1 (38 mg, 0.1 mmol) dissolved in DMF (1 mL) was treated with HATU (37 mg, 0.1 mmol), N,N-diisopropylethylamine (49 mL, 0.3 mmol), and mPEG48-NH₂ (200 mg, 0.09 mmol). RP—HPLC-MS showed complete PEG48 addition to PP1. The crude was purified by R^{P}-HPLC to afford NHBoc PP7 as a white solid (97 mg, 42% yield). ESI MS+ mass calculated C₁₁₃H₂₂₄N₆O₅₂: 2499.03, found: 833.7 [M+3H]³⁺, 625.6 [M+4H]⁴⁺. PP7 was deprotected with HCl (2 mL, 4N in dioxane) for 4 h. HPLC-MS showed complete deprotection, as observed by the disappearance of the peak having a mass of the starting material. The reaction was diluted with 1:1 H₂O:acetonitrile (10 mL), frozen, and lyophilized overnight to quantitatively afford a white solid PP8. ESI MS+ mass calculated C₁₀₈H₂₁₆N₆O₅₀: 2398.88, found: 1199.8 [M+2H]²⁺, 800.3 [M+3H]³⁺, 600.5 [M+4H]⁴⁺, 480.6 [M+5H]⁵⁺.

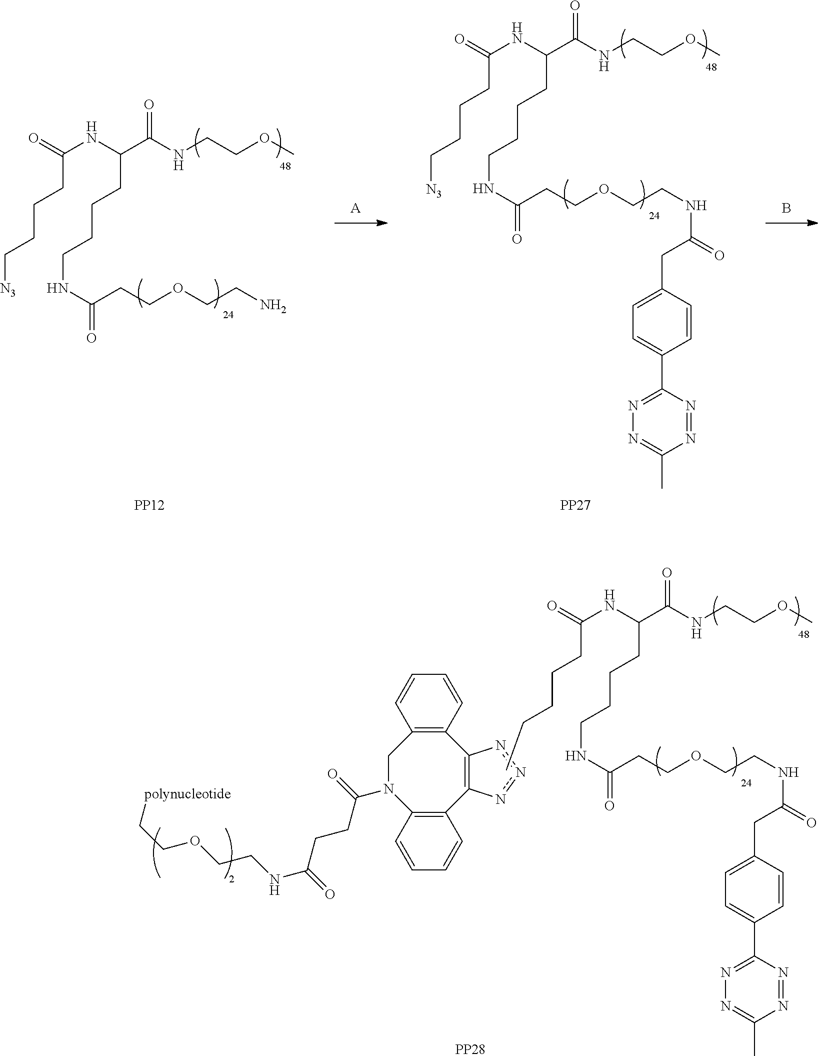

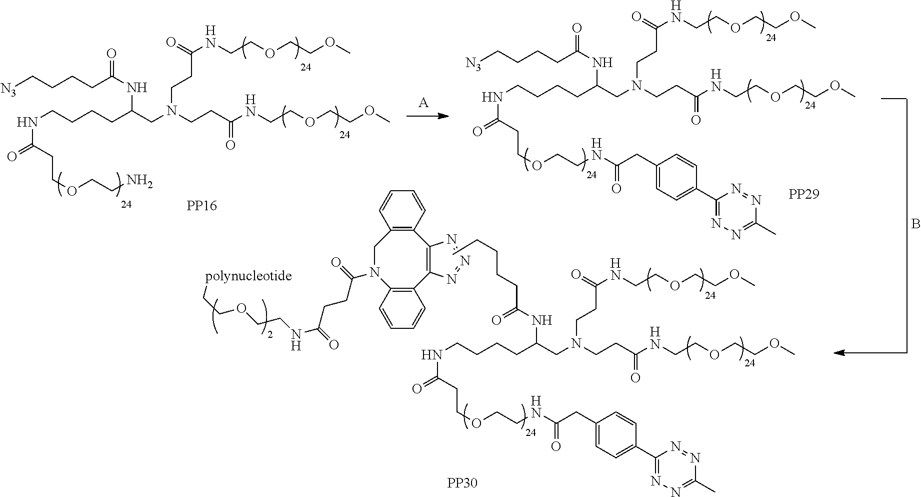

In this Scheme, conditions are:

A) 6-methytetrazine-OSu, HATU, Hunig's base, DMF; and

B) DBCO-CpG, acetonitrile/H₂O;

where 6-methyl tetrazine-OSu is of the following formula:

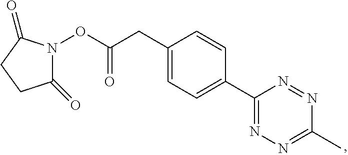 [SMILES: Cc1nnc(-c2ccc(CC(=O)ON3C(=O)CCC3=O)cc2)nn1]

and

DBCO-CpG is of the following formula:

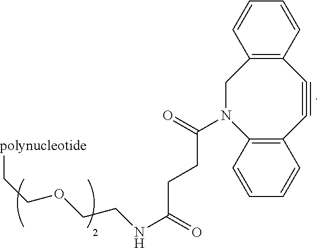

### General Protocol for Preparation of Linkers Loaded with Polynucleotides (PP28 and PP30)

Tetrazine-conjugation handle of PP12 and PP16: PP12 (43 mg, 0.12 mmol) was dissolved in DMF (0.5 mL), treated with HATU (4.6 mg, 0.12 mmol), DIEA (12.7 μL, 0.73 mmol), and, after 5 min, with 6-methyl-tetrazine-OSu (19.9 mg, 0.61 mmol). The crude reaction was stirred for 30 min at room temperature R^{P}-HPLCMS showed complete coupling of 6-methyl-tetrazine carboxylate to PP12. The crude was purified by R^{P}-HPLC, and the pooled fractions were lyophilized to afford PP27 as a purple solid (39 mg, 85% yield). ESI MS+ mass calculated C₁70H₃25N₁₁O76: 3739.47, found: 833.7 [M+3H]³⁺, 625.6 [M+4H]⁴. Pure PP27 was treated in DBCO-CpG in acetonitrile:water (1:1) and incubated at 37° C. for 1-2 hours and an additional 1 hour at room temperature to give PP28. PP28 was purified by preparative AEX (20 mM phosphate and 20 mM phosphate-1 M sodium bromide).

Alternative one-pot route to CpG loaded linkers PP28 and PP30. PP12 (400 nmol) is treated with DBCO-CpG (420 nmol) in acetonitrile:water (1:1) and incubated at 37° C. for 1-2 hours then an additional 1 hour at room temperature. Tetrazine-OSu (4000 nmol) in DMSO stock solution is added to crude PP12-DBCO-CpG solution and the purple solution is reacted for 3 hours at room temperature for 1-2 hours to afford PP28. The crude PP28 was purified by preparative R^{P}-HPLC (50 mM TEAA in water and 10% acetonitrile:water) or preparative AEX (20 mM phosphate and 20 mM phosphate-1 M sodium bromide).

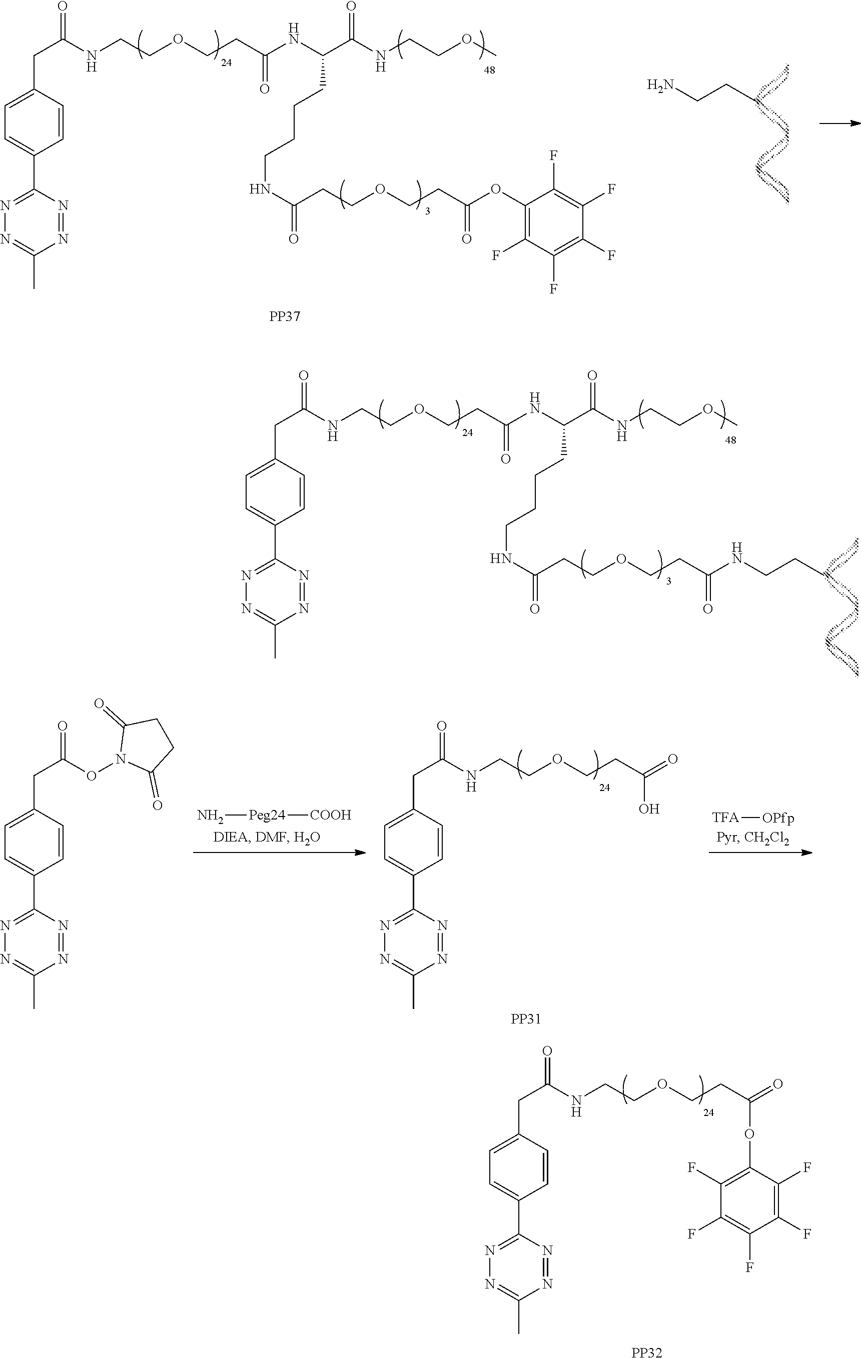

Preparation of Tetrazine-PEG24-OPFP (PP32). To a solution of amino-PEG24-carboxylic acid (1.0 g, 0.9 mmol) and diisopropylethylamine (0.8 mL, 4.4 mmol) in DMF/water (1:1, 12 mL) under Ar (g) was added methyltetrazinephenylacetyl succinimidyl ester (370 mg, 1.1 mmol) in DMF (3 mL) dropwise with stirring. Reaction stirred at room temperature for 2 hours. R^{P}-HPLC/MS indicated formation of product. Solvent was removed in vacuo and crude was purified by R^{P}-HPLC (TFA modifier) to provide PP31, 1.1 g (80%). ESI MS for C₆₂H₁₁₁N₅O₂₇ calculated 1358.56, observed [M+H]⁺1358.8. To a solution of PP31 (109 mg, 0.08 mmol) in dichloromethane (3 mL) under Ar (g) was added anhydrous pyridine (32 mg, 0.4 mmol) and pentafluorophenyl trifluoroacetate (67 mg, 0.24 mmol). Reaction stirred at room temperature overnight. Solvent was removed in vacuo. Crude product was redissolved in EtOAc and washed with aq. NaHCO₃ (5% w/v) (3×) and brine (1×). Organic phase was dried over Na₂SO₄, filtered, and concentrated in vacuo to give PP32 quantitatively. Used in next step without further purification. ESI MS for C₆₈H₁₁₀F₅N₅O₂₇ calculated 1524.61, observed [M+2H]²⁺763.0.

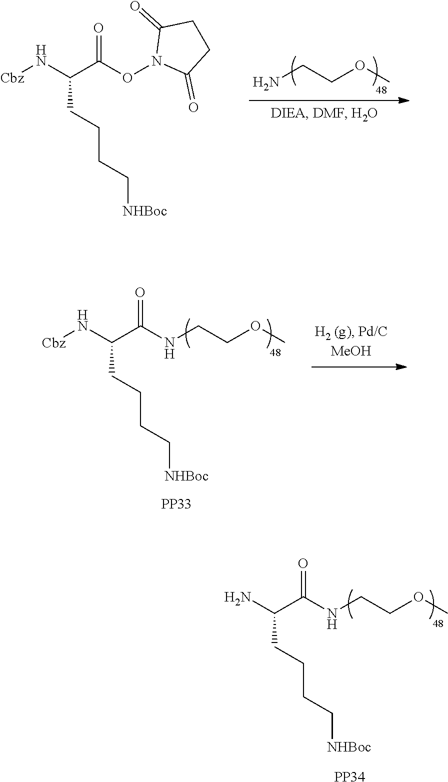

Preparation of PP34. To a solution of mPEG48-amine (2.15 g, 1.00 mmol) and diisopropylethylamine (0.87 mL, 5.00 mmol) in DMF/water (1:1, 10 mL) under Ar (g) was added Nα-Cbz-Nε-Boc-L-Lysine succinimidyl ester (570 mg, 1.2 mmol) in DMF (5 mL) dropwise with stirring. Reaction mixture was stirred at room temperature for 2 hours. R^{P}-HPLC/MS indicated formation of product, PP33. The reaction mixture was concentrated in vacuo and purified by silica gel chromatography (CH₂Cl₂:MeOH 0-10%). Recovered PP33 was used directly in next reaction. ESI MS for C₁₁₆H₂₂₃N₃O₅₃ calculated 2508.0, observed [M+3H]³⁺836.7, [M+4H]⁴⁺627.9. A solution of PP33 (1.00 mmol) in MeOH was flushed with nitrogen (g), and Palladium on activated carbon (10% wt, catalytic) was added. The solution was alternately evacuated and purged with hydrogen (g) (3×). RP—HPLC/MS after 2 hours showed formation of PP34. The heterogeneous mixture was filtered through a bed of Celite and washed with copious amounts of methanol. Removal of the solvent in vacuo, yielded PP34, (2.0 g, 84% yield, over 2 steps). ESI MS for C₁₀₈H₂₁₇N₃O₅₁ calculated 2373.87, observed [M+3H]³⁺792.0.

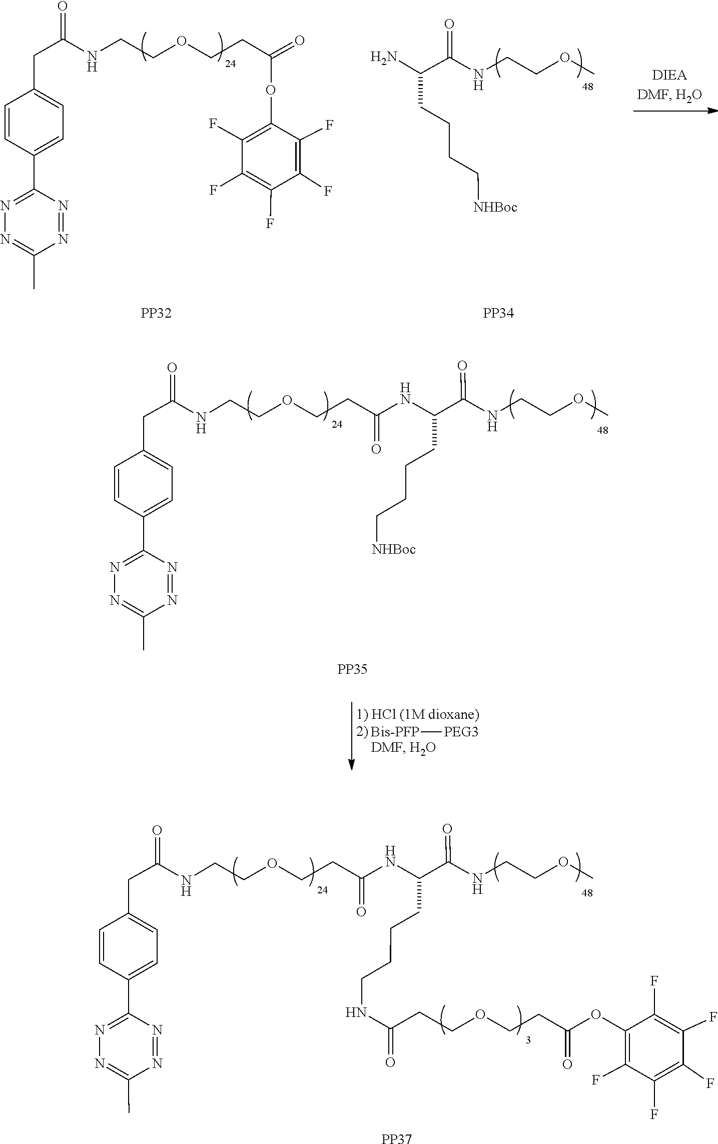

Preparation of PP37. To a solution of PP32 (124 mg, 0.08 mmol) and diisopropylethylamine (31 mg, 0.24 mmol) in DMF/water (1:1, 10 mL) under Ar (g) was added PP34 (230 mg, 0.1 mmol) in DMF/water (1:1, 10 mL) dropwise with stirring. The reaction was stirred at room temperature for 2 hours and R^{P}-HPLC/MS indicated formation of product PP35. Solvent was removed in vacuo and PP35 used in next step without further purification. ESI MS for C₁₇₀H₃₂₆N₈O₇₇ calculated 3714.4, observed [M+4H]⁴⁺929.5, [M+5H]⁵⁺743.8. Crude PP35 (0.08 mmol) treated with HCl (4 N in dioxane, 5 mL) under Ar (g). Reaction was stirred at room temperature for 2 hours and R^{P}-HPLC/MS indicated complete removal of Boc protecting group. The solvent was removed in vacuo and the amine was acylated with a solution of bis-Peg3-PFP ester (230 mg, 0.4 mmol) in DMF (5 mL) and diisopropylethylamine (140 uL, 0.8 mmol). After 2 hours, R^{P}-HPLC/MS indicated formation of product PP37 Solvent was removed in vacuo and crude was purified by R^{P}-HPLC (TFA modifier) to provide PP37 as a tetra-TFA salt, 31 mg in 8.7% yield. ESI MS for C₁₈₁H₃₃₃F₅N₈O₈₁ calculated 4012.56, observed [M+3H]³⁺1338.3, [M+4H]⁴⁺1004.0, [M+5H]⁵⁺803.4, [M+6H]⁶⁺669.

### List of the Linkers Containing Auxiliary Moieties:

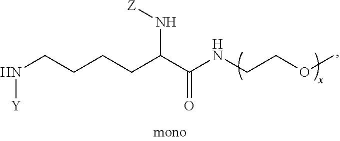

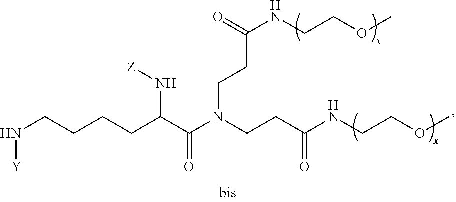

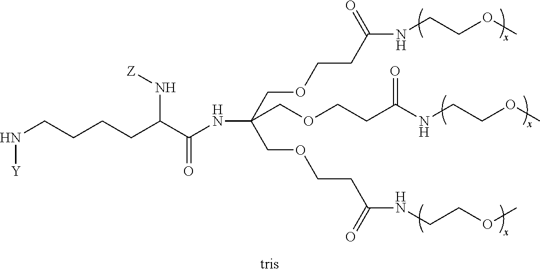

| | | | | Z | | MS | MS found |
|---|---|---|---|---|---|---|---|
| Linker | Valency | X | Y | (tether) | Formula | calc. | (ESI+) |
| PP6 | mono | 24 | H | N₃- | C₆₀H₁₂₀N₆O₂₆ | 1341.62 | 1341.7, 671.5 |
| | | | | valeramide | | | |
| PP8 | mono | 48 | H | N₃- | C₁₀₈H₂₁₆N₆O₅₀ | 2398.88 | 1199.8, |
| | | | | valeramide | | | 800.3, 600.5, |
| | | | | | | | 480.6 |
| PP10 | mono | 48 | CO-PEG08- | N₃- | C₁₂₇H₂₅₃N₇O₅₉ | 2822.38 | 1412.0, |
| | | | NH₂ | valeramide | | | 941.7, 706.5, |
| | | | | | | | 565.4, 471.4 |
| PP12 | mono | 48 | CO-PEG24- | N₃- | C₁₅₉H₃₁₇N₇O₇₅ | 3527.21 | 1176.5, |
| | | | NH₂ | valeramide | | | 882.6, 706.3 |
| PP14 | bis | 24 | CO-PEG08- | N₃- | C₁₃₄H₂₆₅N₉O₆₁ | 2978.56 | 1490.1, |
| | | | NH₂ | valeramide | | | 993.7, 745.6, |
| | | | | | | | 596.7, 497.4 |
| PP16 | bis | 24 | CO-PEG24- | N₃- | C₁₆₆H₃₂₉N₉O₇₇ | 3683.39 | 1228.6, |
| | | | NH₂ | valeramide | | | 921.7, 737.6, |
| | | | | | | | 615.0 |
| PP18 | bis | 48 | CO-PEG08- | N₃- | C₂₃₀H₄₅₇N₉O₁₀₉ | 5093.08 | 1247.2, |
| | | | NH₂ | valeramide | | | 1019.6, |
| | | | | | | | 849.8, 728.6, |
| | | | | | | | 637.7 |
| PP20 | bis | 48 | CO-PEG24- | N₃- | C₂₆₂H₅₂₁N₉O₁₂₅ | 5797.93 | 1450.3, |
| | | | NH₂ | valeramide | | | 1160.4, |
| | | | | | | | 967.1, 829.1, |
| | | | | | | | 725.8 |
| PP22 | tris | 24 | H | N₃- | C₁₇₁H₃₃₉N₉O₈₀ | 3801.52 | 1268.0, |
| | | | | valeramide | | | 951.2, 761.2 |
| PP24 | tris | 24 | CO-PEG08- | N₃- | C₁₉₀H₃₇₆N₁₀O₈₉ | 4225.02 | 1409.3, |
| | | | NH₂ | valeramide | | | 1057.0, |
| | | | | | | | 846.0, 705.2, |
| | | | | | | | 604.6 |
| PP26 | tris | 24 | CO-PEG24- | N₃- | C₂₂₂H₄₄₀N₁₀O₁₀₅ | 4929.87 | 1233.3, 968.8 |
| | | | NH₂ | valeramide | | | |
| PP27 | mono | 48 | CO-PEG24- | N₃- | C₁₇₀H₃₂₅N₁₁O₇₆ | 3739.47 | 1247.2, |
| | | | Tetrazine | valeramide | | | 935.7, 748.8, |
| | | | | | | | 624.1, 535.3 |
| PP28 | mono | 48 | CO-PEG24- | p313 + N₃- | | 8893.7 | 8891, |
| | | | Tetrazine | valeramide | | | deconvoluted |
| | | | | | | | ESI− |
| PP29 | bis | 24 | CO-PEG24- | N₃- | C₁₇₇H₃₃₇N₁₃O₇₈ | 3895.66 | 974.8, 780.0 |
| | | | Tetrazine | valeramide | | | |
| PP30 | bis | 24 | CO-PEG24- | p313 + N₃- | | 9049.9 | 9046, |
| | | | Tetrazine | valeramide | | | deconvoluted |
| | | | | | | | ESI− |
| PP37 | mono | 48 | PFP-PEG3 | CO- | C₁₈₁H₃₃₃F₅N₈O₈₁ | 4012.56 | 1338.3, |
| | | | | PEG24- | | | 1004.0, |
| | | | | Tetrazine | | | 803.4, 669.6 |
| PP38 | bis | 48 | CO-PEG24- | p313 + N₃- | | 11163.2 | 11159, |
| | | | Tetrazine | valeramide | | | deconvoluted |
| | | | | | | | ESI− |
| PP39 | tris | 24 | CO-PEG24- | p313 + N₃- | | 10281.1 | 10292, |
| | | | Tetrazine | valeramide | | | deconvoluted |
| | | | | | | | ESI− |

In the above table, groups identified as Y or Z have the following structures:

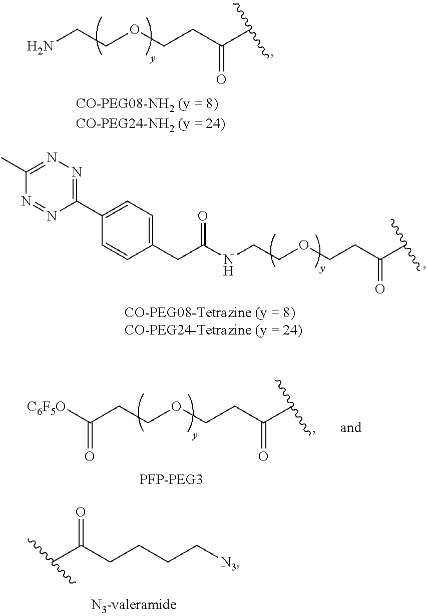

In the table above, group Z identified as “p313+N₃-valeramide” refers to a product of a cycloaddition reaction between p313 and a linker having N₃-valeramide as Z.

The phosphoramidite monomers shown in Table 1 were synthesized using the standard synthetic procedures described herein and in WO 2015/188197.

The bicyclic oxazaphospholidine monomers used in chiral phosphorothioate oligonucleotide synthesis were prepared using literature protocol as reported by Wada, J. Am. Chem. Soc. 130:16031-16037, 2008.

TABLE 1

| Com- | | | |
|---|---|---|---|
| pound | | ³¹P NMR (δ in ppm) | Yield |
| # | Structure | and/or ESI MS | (%) |
| dT1 | 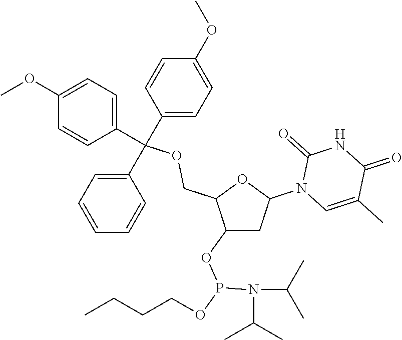 [SMILES: CCCCOP(OC1CC(n2cc(C)c(=O)[nH]c2=O)OC1COC(c1ccccc1)(c1ccc(OC)cc1)c1ccc(OC)cc1)N(C(C)C)C(C)C] | ESI MS calculated 747.8, observed 746.9 [M − H] ³¹P NMR (202 MHz, CDCl₃): δ147.50, 147.00 | |
| dC1 | 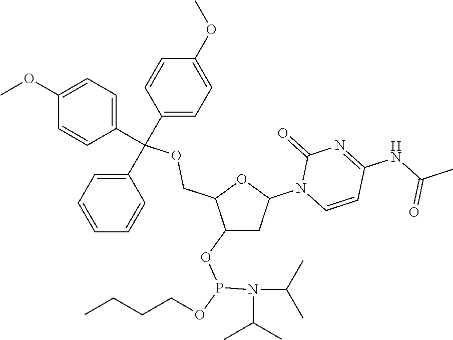 [SMILES: CCCCOP(OC1CC(n2ccc(NC(C)=O)nc2=O)OC1COC(c1ccccc1)(c1ccc(OC)cc1)c1ccc(OC)cc1)N(C(C)C)C(C)C] | — | |
| dA1 | 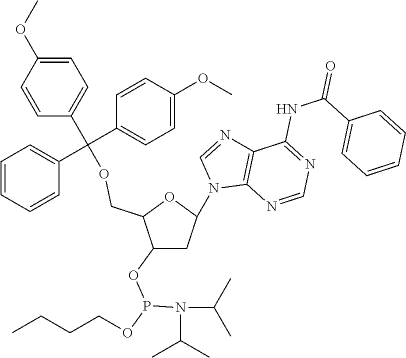 [SMILES: CCCCOP(OC1CC(n2cnc3c(NC(=O)c4ccccc4)ncnc32)OC1COC(c1ccccc1)(c1ccc(OC)cc1)c1ccc(OC)cc1)N(C(C)C)C(C)C] | ESI MS calculated 860.97, observed 859.9 [M − H], 862.0 [M + H] ³¹P NMR (202 MHz, CDCl₃): δ147.47, 147.35 | |
| dG1 | 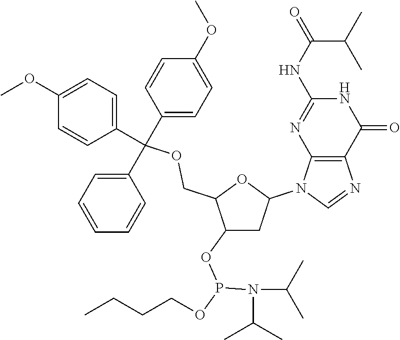 [SMILES: CCCCOP(OC1CC(n2cnc3c(=O)[nH]c(NC(=O)C(C)C)nc32)OC1COC(c1ccccc1)(c1ccc(OC)cc1)c1ccc(OC)cc1)N(C(C)C)C(C)C] | ESI MS calculated 842.96, observed 841.7 [M − H], 843.9 [M + H] ³¹P NMR (202 MHz, CDCl₃): δ147.05, 147.93 | |
| dT2 | 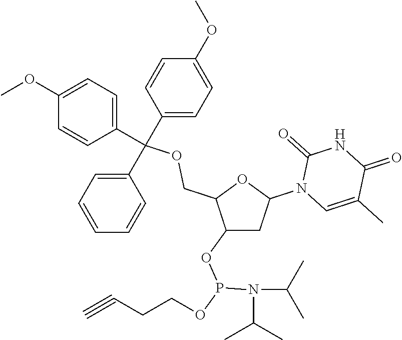 [SMILES: C#CCCOP(OC1CC(n2cc(C)c(=O)[nH]c2=O)OC1COC(c1ccccc1)(c1ccc(OC)cc1)c1ccc(OC)cc1)N(C(C)C)C(C)C] | ESI MS calculated 743.8, observed 742.8 [M − H], 744.7 [M + H] ³¹P NMR (202 MHz, CDCl₃): δ148.26, 147.77 | |
| dC2 | 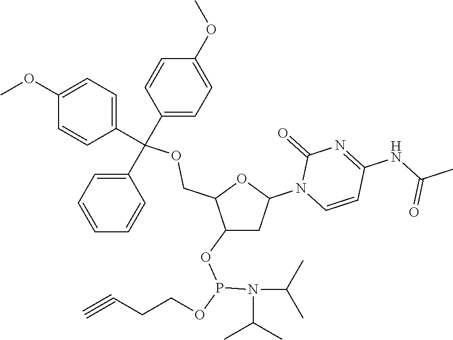 [SMILES: C#CCCOP(OC1CC(n2ccc(NC(C)=O)nc2=O)OC1COC(c1ccccc1)(c1ccc(OC)cc1)c1ccc(OC)cc1)N(C(C)C)C(C)C] | | |
| dA2 | 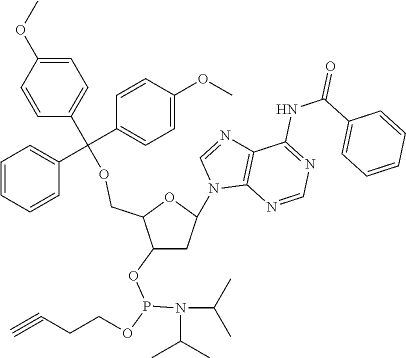 [SMILES: C#CCCOP(OC1CC(n2cnc3c(NC(=O)c4ccccc4)ncnc32)OC1COC(c1ccccc1)(c1ccc(OC)cc1)c1ccc(OC)cc1)N(C(C)C)C(C)C] | | |
| dG2 | 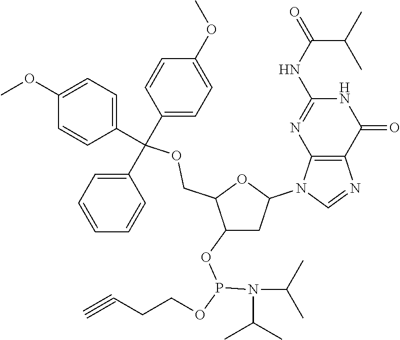 [SMILES: C#CCCOP(OC1CC(n2cnc3c(=O)[nH]c(NC(=O)C(C)C)nc32)OC1COC(c1ccccc1)(c1ccc(OC)cc1)c1ccc(OC)cc1)N(C(C)C)C(C)C] | | |
| dT3 | 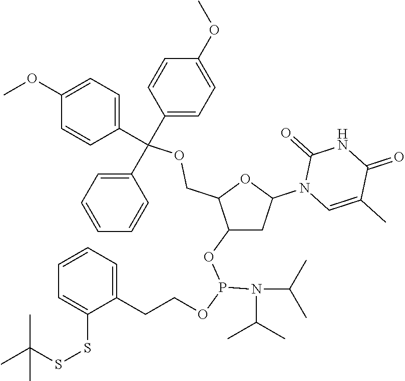 [SMILES: COc1ccc(C(OCC2OC(n3cc(C)c(=O)[nH]c3=O)CC2OP(OCCc2ccccc2SSC(C)(C)C)N(C(C)C)C(C)C)(c2ccccc2)c2ccc(OC)cc2)cc1] | ESI MS calculated 916.1, observed 915.6 [M − H] ³¹P NMR (202 MHz, CDCl₃): δ147.72, 147.21 | |
| dC3 | 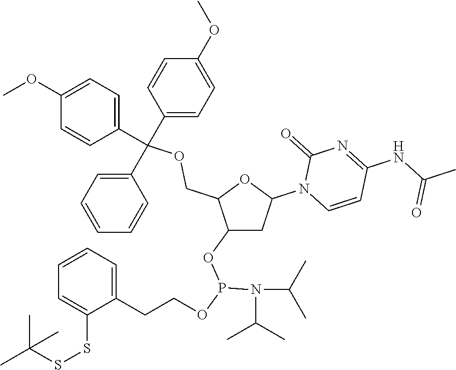 [SMILES: COc1ccc(C(OCC2OC(n3ccc(NC(C)=O)nc3=O)CC2OP(OCCc2ccccc2SSC(C)(C)C)N(C(C)C)C(C)C)(c2ccccc2)c2ccc(OC)cc2)cc1] | — | |
| dA3 | 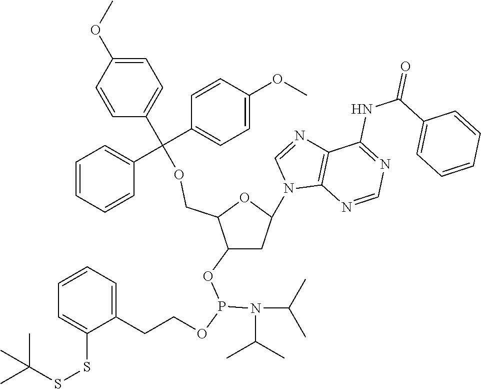 [SMILES: COc1ccc(C(OCC2OC(n3cnc4c(NC(=O)c5ccccc5)ncnc43)CC2OP(OCCc2ccccc2SSC(C)(C)C)N(C(C)C)C(C)C)(c2ccccc2)c2ccc(OC)cc2)cc1] | — | |
| dG3 | 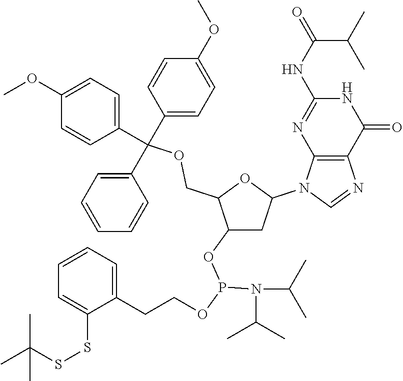 [SMILES: COc1ccc(C(OCC2OC(n3cnc4c(=O)[nH]c(NC(=O)C(C)C)nc43)CC2OP(OCCc2ccccc2SSC(C)(C)C)N(C(C)C)C(C)C)(c2ccccc2)c2ccc(OC)cc2)cc1] | — | |
| dT4 | 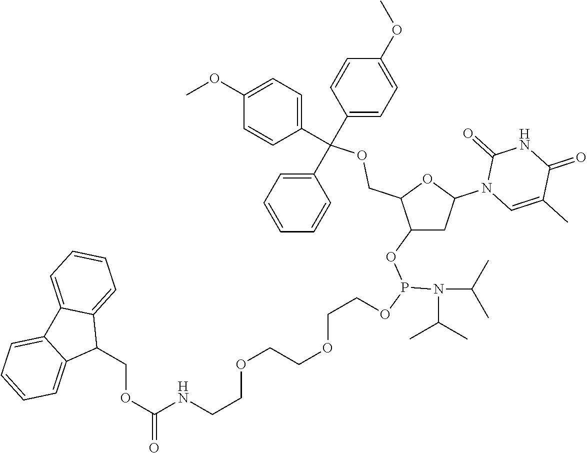 [SMILES: COc1ccc(C(OCC2OC(n3cc(C)c(=O)[nH]c3=O)CC2OP(OCCOCCOCCNC(=O)OCC2c3ccccc3-c3ccccc32)N(C(C)C)C(C)C)(c2ccccc2)c2ccc(OC)cc2)cc1] | ESI MS calculated 1045.2, observed 1046.3 [M + H] ³¹P NMR (202 MHz, CDCl₃): δ148.38, 148.27 | |
| dC4 | 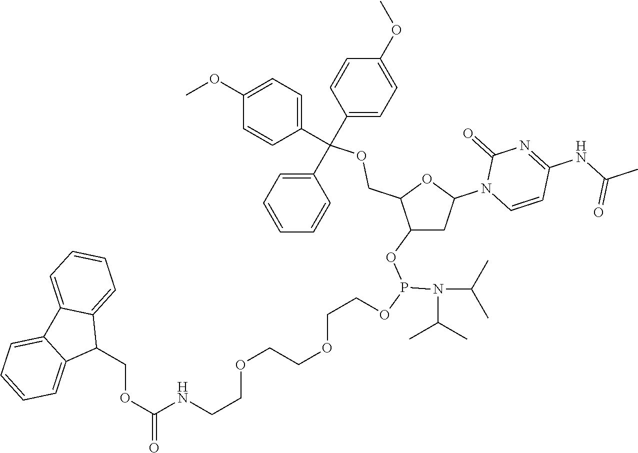 [SMILES: COc1ccc(C(OCC2OC(n3ccc(NC(C)=O)nc3=O)CC2OP(OCCOCCOCCNC(=O)OCC2c3ccccc3-c3ccccc32)N(C(C)C)C(C)C)(c2ccccc2)c2ccc(OC)cc2)cc1] | ESI MS calculated 1088.2, observed 1089.0 [M + H] ³¹P NMR (202 MHz, CDCl₃): δ149.29, 148.66 | |
| dA4 | 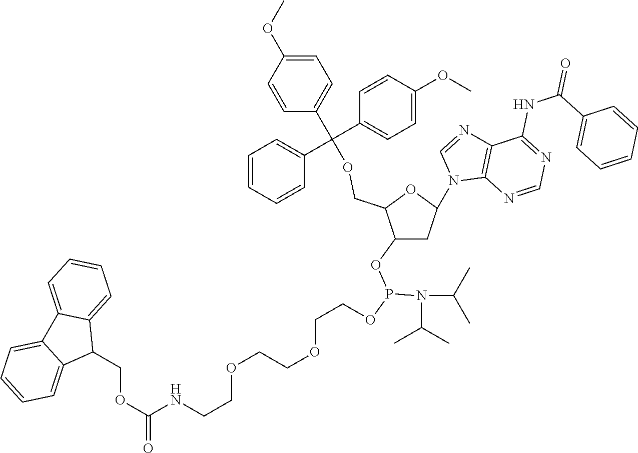 [SMILES: COc1ccc(C(OCC2OC(n3cnc4c(NC(=O)c5ccccc5)ncnc43)CC2OP(OCCOCCOCCNC(=O)OCC2c3ccccc3-c3ccccc32)N(C(C)C)C(C)C)(c2ccccc2)c2ccc(OC)cc2)cc1] | ESI MS calculated 1158.3, observed 1157.5 [M − H], 1159.0 [M + H] ³¹P NMR (202 MHz, CDCl₃): δ148.38 | |
| dG4 | 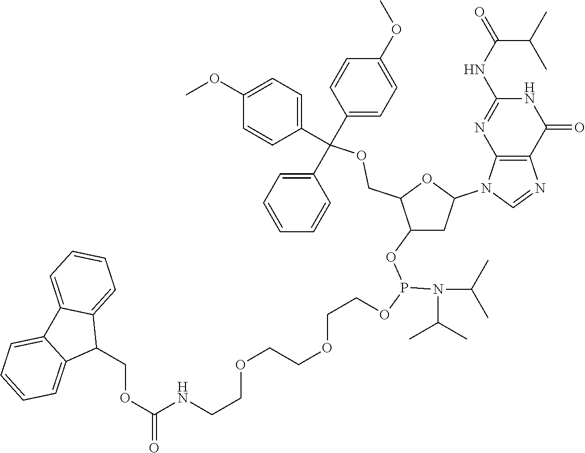 [SMILES: COc1ccc(C(OCC2OC(n3cnc4c(=O)[nH]c(NC(=O)C(C)C)nc43)CC2OP(OCCOCCOCCNC(=O)OCC2c3ccccc3-c3ccccc32)N(C(C)C)C(C)C)(c2ccccc2)c2ccc(OC)cc2)cc1] | ESI MS calculated 1140.3, observed 1139.1 [M − H], 1141.2 [M + H] ³¹P NMR (202 MHz, CDCl₃): δ147.76 | |
| dT5 | 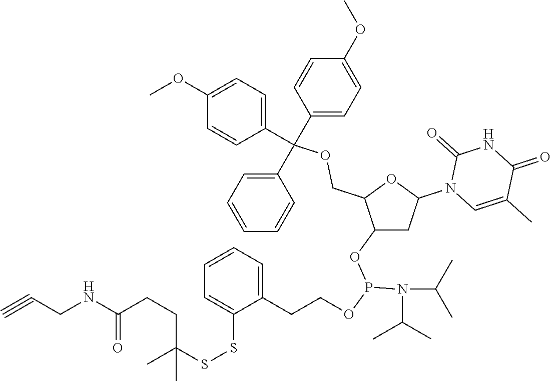 [SMILES: C#CCNC(=O)CCC(C)(C)SSc1ccccc1CCOP(OC1CC(n2cc(C)c(=O)[nH]c2=O)OC1COC(c1ccccc1)(c1ccc(OC)cc1)c1ccc(OC)cc1)N(C(C)C)C(C)C] | ESI MS calculated 1011.2, observed 978.6 [M − H] ³¹P NMR (202 MHz, CDCl₃): δ147.51, 147.32 | |
| dC5 | 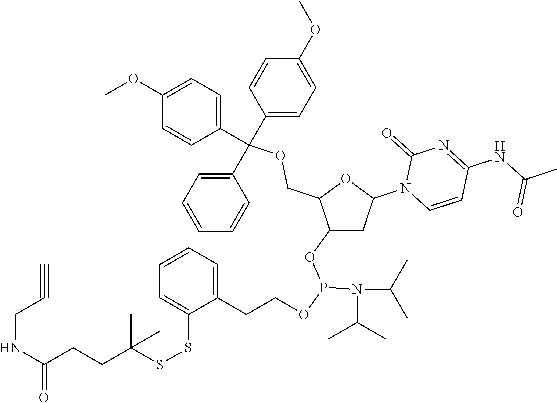 [SMILES: C#CCNC(=O)CCC(C)(C)SSc1ccccc1CCOP(OC1CC(n2ccc(NC(C)=O)nc2=O)OC1COC(c1ccccc1)(c1ccc(OC)cc1)c1ccc(OC)cc1)N(C(C)C)C(C)C] | — | |
| dA5 | 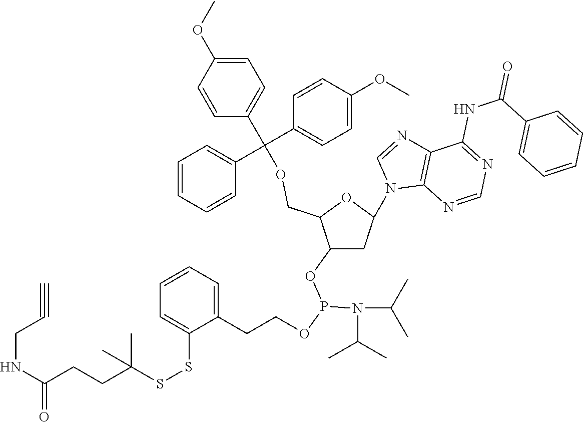 [SMILES: C#CCNC(=O)CCC(C)(C)SSc1ccccc1CCOP(OC1CC(n2cnc3c(NC(=O)c4ccccc4)ncnc32)OC1COC(c1ccccc1)(c1ccc(OC)cc1)c1ccc(OC)cc1)N(C(C)C)C(C)C] | — | |
| dG5 | 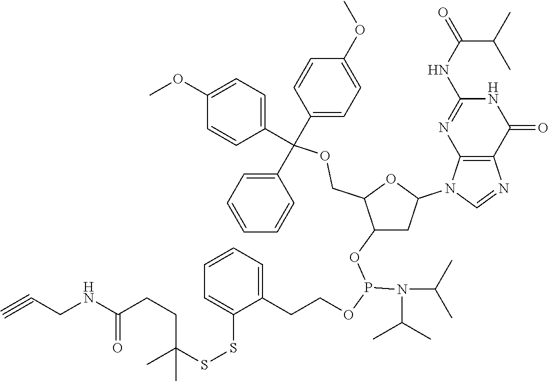 [SMILES: C#CCNC(=O)CCC(C)(C)SSc1ccccc1CCOP(OC1CC(n2cnc3c(=O)[nH]c(NC(=O)C(C)C)nc32)OC1COC(c1ccccc1)(c1ccc(OC)cc1)c1ccc(OC)cc1)N(C(C)C)C(C)C] | — | |
| dT6 | 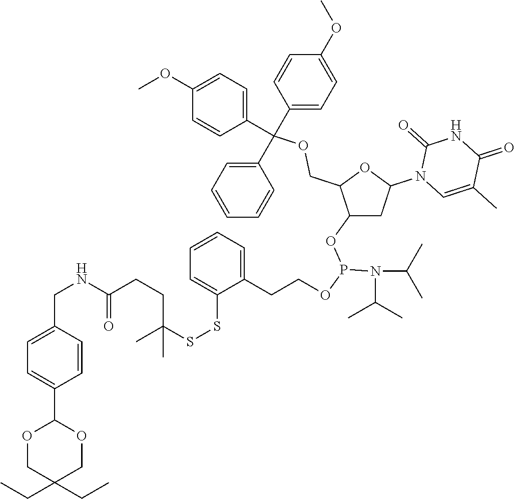 [SMILES: CCC1(CC)COC(c2ccc(CNC(=O)CCC(C)(C)SSc3ccccc3CCOP(OC3CC(n4cc(C)c(=O)[nH]c4=O)OC3COC(c3ccccc3)(c3ccc(OC)cc3)c3ccc(OC)cc3)N(C(C)C)C(C)C)cc2)OC1] | — | |
| dC6 | 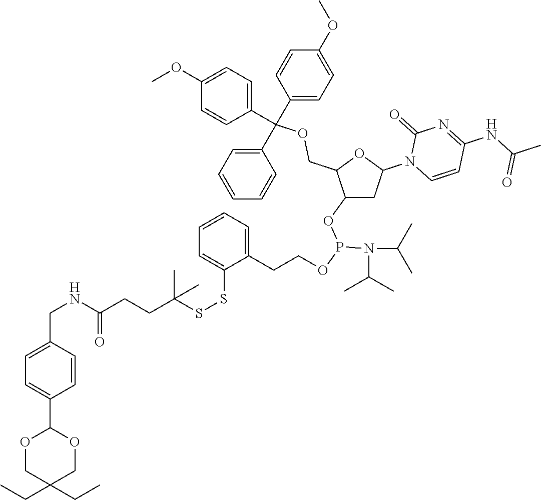 [SMILES: CCC1(CC)COC(c2ccc(CNC(=O)CCC(C)(C)SSc3ccccc3CCOP(OC3CC(n4ccc(NC(C)=O)nc4=O)OC3COC(c3ccccc3)(c3ccc(OC)cc3)c3ccc(OC)cc3)N(C(C)C)C(C)C)cc2)OC1] | — | |
| dA6 | 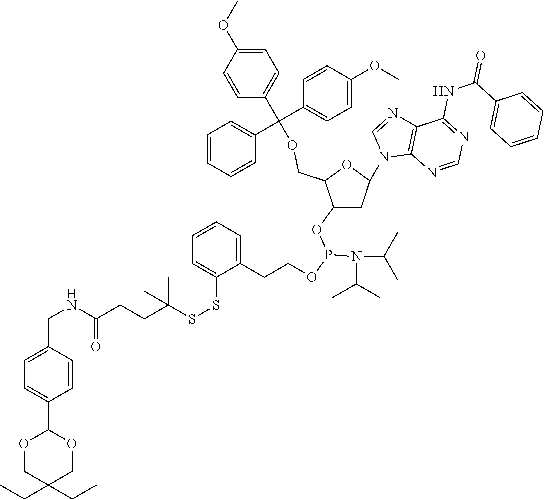 [SMILES: CCC1(CC)COC(c2ccc(CNC(=O)CCC(C)(C)SSc3ccccc3CCOP(OC3CC(n4cnc5c(NC(=O)c6ccccc6)ncnc54)OC3COC(c3ccccc3)(c3ccc(OC)cc3)c3ccc(OC)cc3)N(C(C)C)C(C)C)cc2)OC1] | — | |
| dG6 | 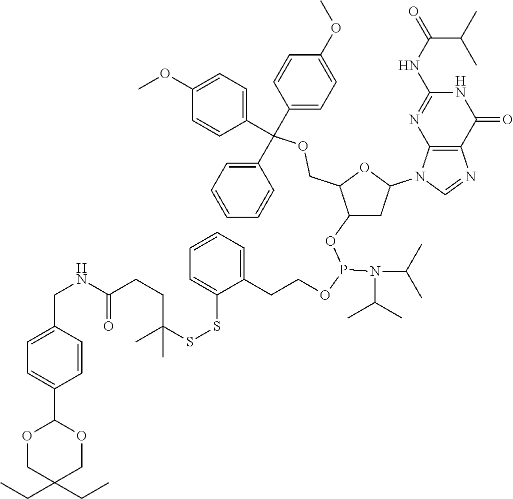 [SMILES: CCC1(CC)COC(c2ccc(CNC(=O)CCC(C)(C)SSc3ccccc3CCOP(OC3CC(n4cnc5c(=O)[nH]c(NC(=O)C(C)C)nc54)OC3COC(c3ccccc3)(c3ccc(OC)cc3)c3ccc(OC)cc3)N(C(C)C)C(C)C)cc2)OC1] | — | |
| dT7 | 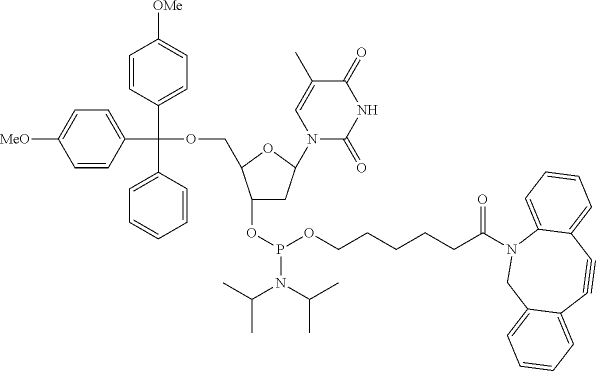 [SMILES: COc1ccc(C(OCC2OC(n3cc(C)c(=O)[nH]c3=O)CC2OP(OCCCCCC(=O)N2Cc3ccccc3C#Cc3ccccc32)N(C(C)C)C(C)C)(c2ccccc2)c2ccc(OC)cc2)cc1] | ³¹P NMR (202 MHz, CDCl₃): δ 147.05 (d, J 8.08 Hz), 146.58 (d, J 8.08 Hz) ESI MS calculated 992.45, observed 994.3 (M + H), 992.0 (M − H) | 42 |
| dT8 | 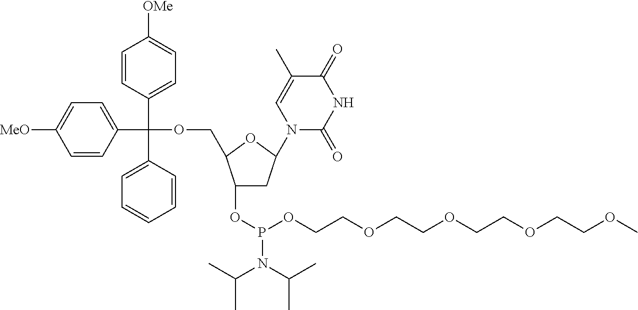 [SMILES: COCCOCCOCCOCCOP(OC1CC(n2cc(C)c(=O)[nH]c2=O)OC1COC(c1ccccc1)(c1ccc(OC)cc1)c1ccc(OC)cc1)N(C(C)C)C(C)C] | ³¹P NMR (202 MHz, CDCl₃): δ 147.80 ESI MS calculated 881.99, observed 880.9 (M − H), 904.9 (M + Na) | 14 |
| dU7 (Rp) | 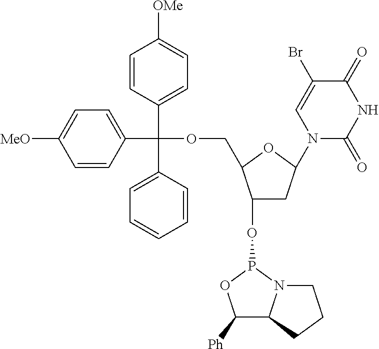 | ³¹P NMR (202 MHz, CDCl₃): δ156.19 (s) | 31 |
| dU8 (Sp) | 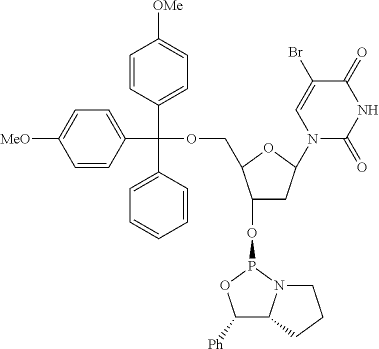 | ³¹P NMR (202 MHz, CDCl₃): δ155.78 (s) | 27 |
| dC7 (Rp) | 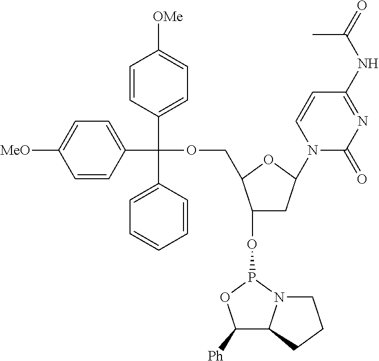 | ³¹P NMR (202 MHz, CDCl₃): δ156.75 (s) | 21 |
| dC8 (Sp) | 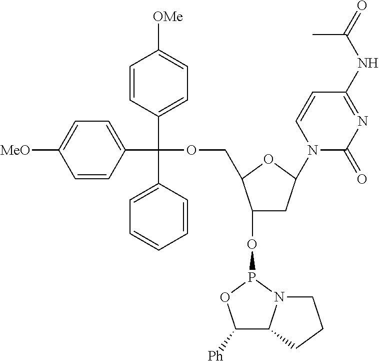 | ³¹P NMR (202 MHz, CDCl₃): δ156.08 (s) | 25 |

Chiral Abasic Spacers-Compounds X7, X8, X9 and X10:

| | |
|---|---|
| 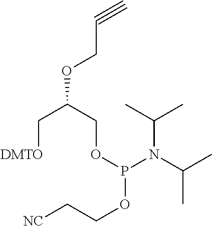 | X7 |
| 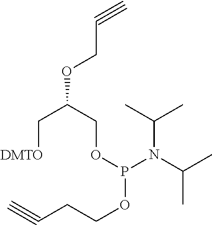 | X8 |
| 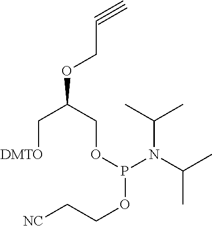 | X9 |
| 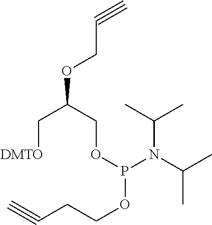 | X10 |

X7 and X8 Synthesis:

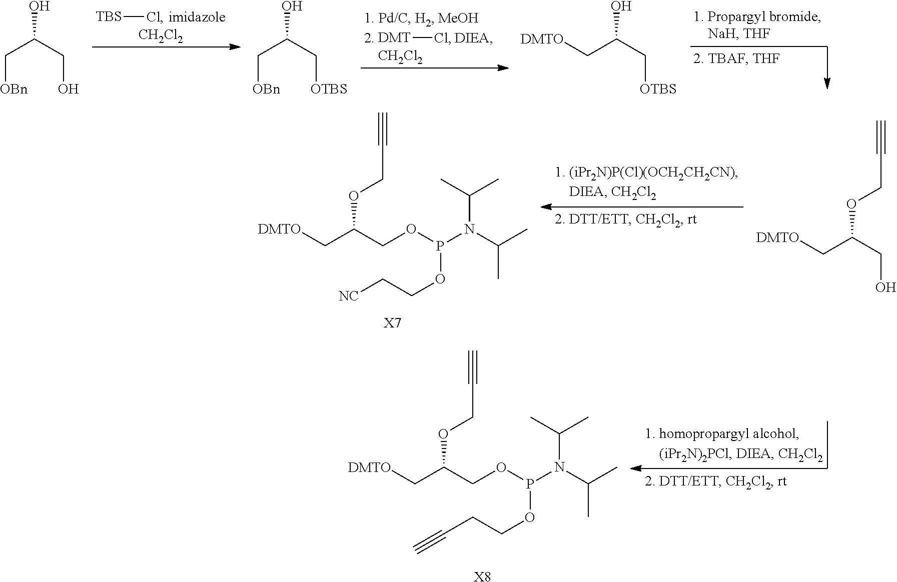

X9 and X10 Synthesis:

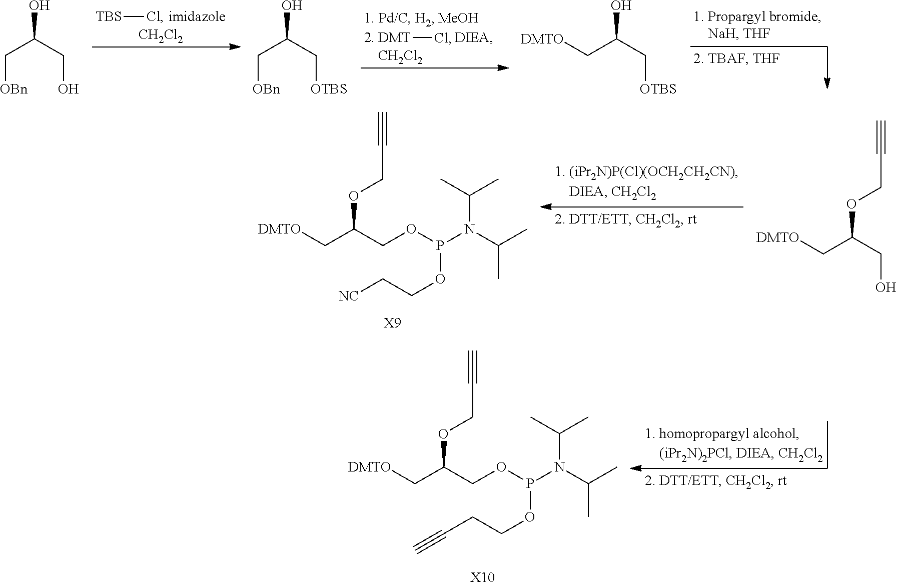

The following are further hydrophilic nucleoside phosphoramidites that can be prepared using methods known in the art and methods described herein:

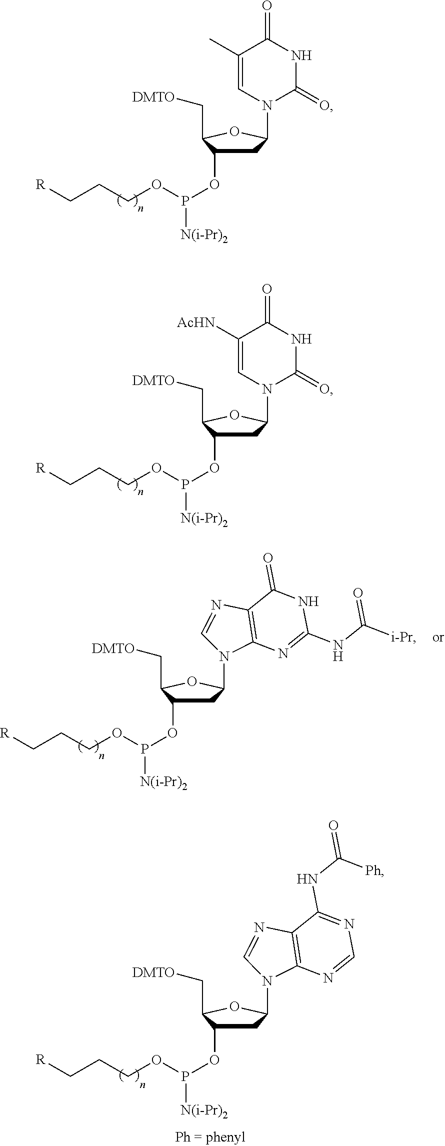

where R is OH, optionally substituted amino, or —CO₂R¹ (R¹ is H or a counterion), and n is an integer from 1 to 4;

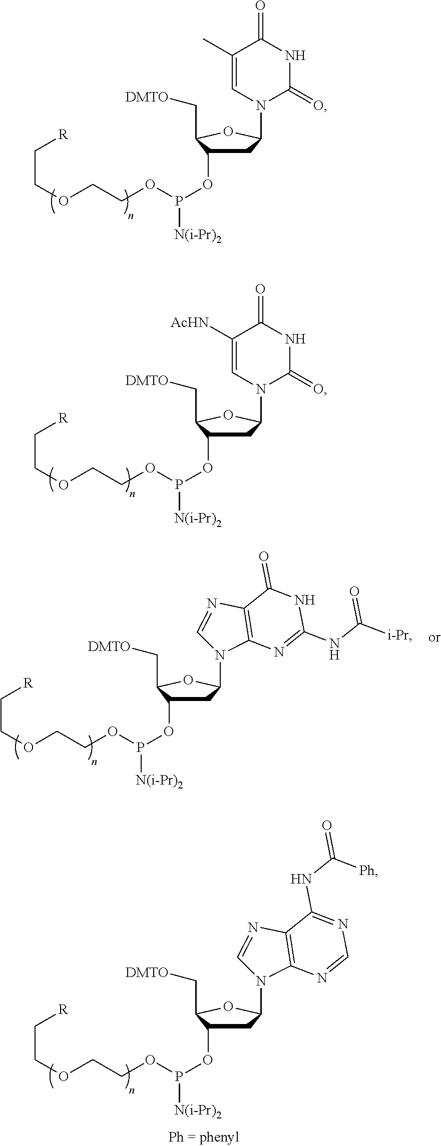

where R is OH, OAc, OMe, optionally substituted amino, or CO₂R¹ (R¹ is H or a counterion), and n is an integer from 1 to 51.

The following are further substituted nucleoside phosphoramidites that can be prepared using methods known in the art and methods described herein:

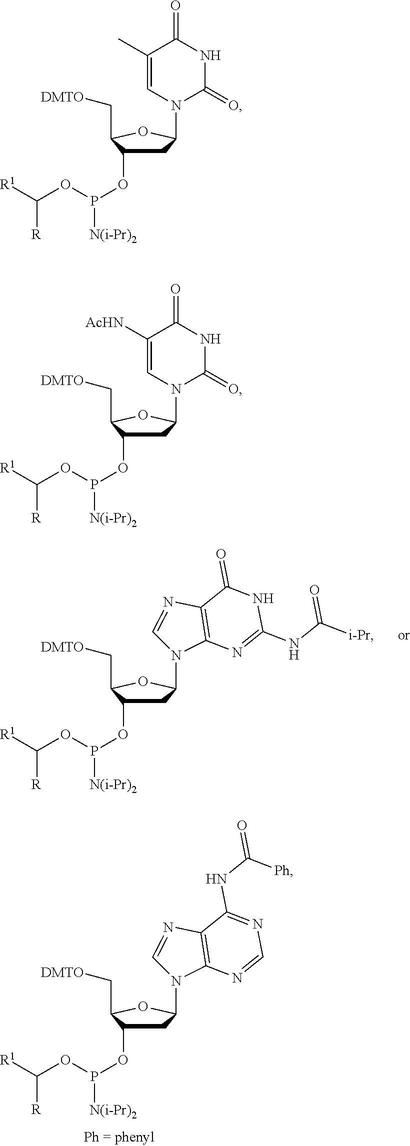

where each of R and R¹ is independently H or optionally substituted C₁₋₆ alkyl (e.g., Me, Et, i-Pr, or n-Bu).

The following phosphoramidites are purchased from Glen Research (Sterling, Va.) or ChemGenes (Wilmington, Mass.) or prepared using standard protocols described herein:

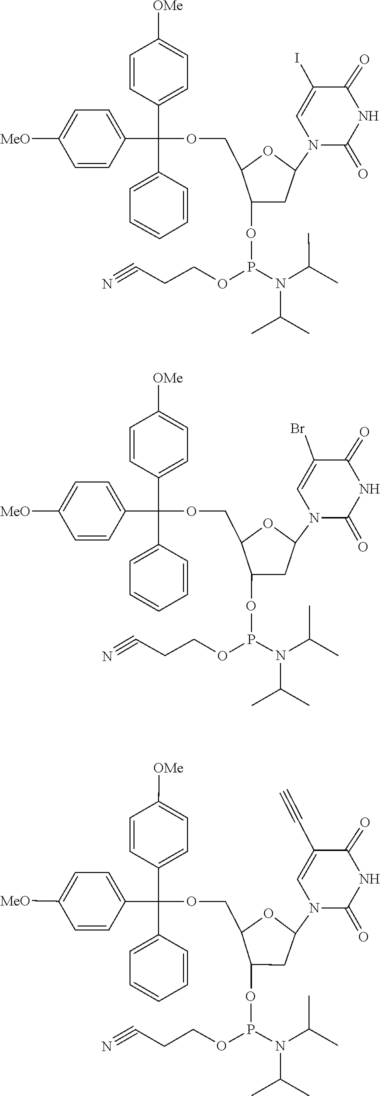 [SMILES: COc1ccc(C(OCC2OC(n3cc(I)c(=O)[nH]c3=O)CC2OP(OCCC#N)N(C(C)C)C(C)C)(c2ccccc2)c2ccc(OC)cc2)cc1 ; COc1ccc(C(OCC2OC(n3cc(Br)c(=O)[nH]c3=O)CC2OP(OCCC#N)N(C(C)C)C(C)C)(c2ccccc2)c2ccc(OC)cc2)cc1 ; C#Cc1cn(C2CC(OP(OCCC#N)N(C(C)C)C(C)C)C(COC(c3ccccc3)(c3ccc(OC)cc3)c3ccc(OC)cc3)O2)c(=O)[nH]c1=O]

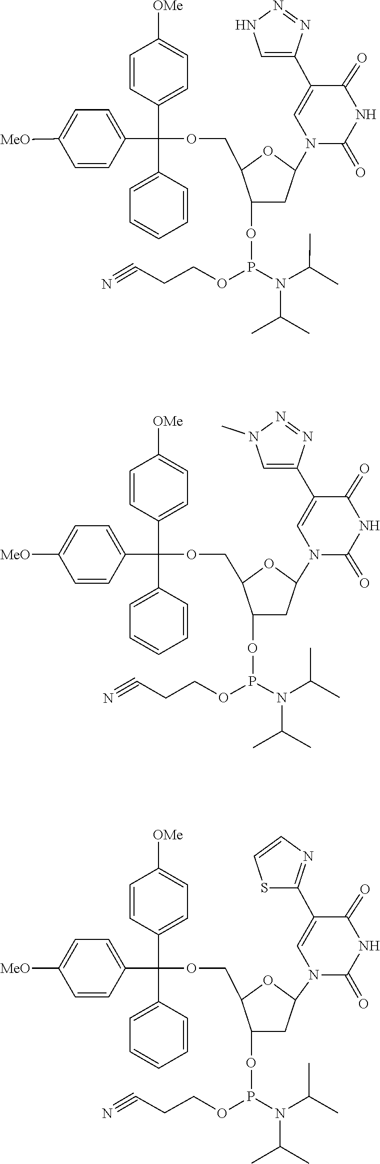 [SMILES: COc1ccc(C(OCC2OC(n3cc(-c4c[nH]nn4)c(=O)[nH]c3=O)CC2OP(OCCC#N)N(C(C)C)C(C)C)(c2ccccc2)c2ccc(OC)cc2)cc1 ; COc1ccc(C(OCC2OC(n3cc(-c4cn(C)nn4)c(=O)[nH]c3=O)CC2OP(OCCC#N)N(C(C)C)C(C)C)(c2ccccc2)c2ccc(OC)cc2)cc1 ; COc1ccc(C(OCC2OC(n3cc(-c4nccs4)c(=O)[nH]c3=O)CC2OP(OCCC#N)N(C(C)C)C(C)C)(c2ccccc2)c2ccc(OC)cc2)cc1]

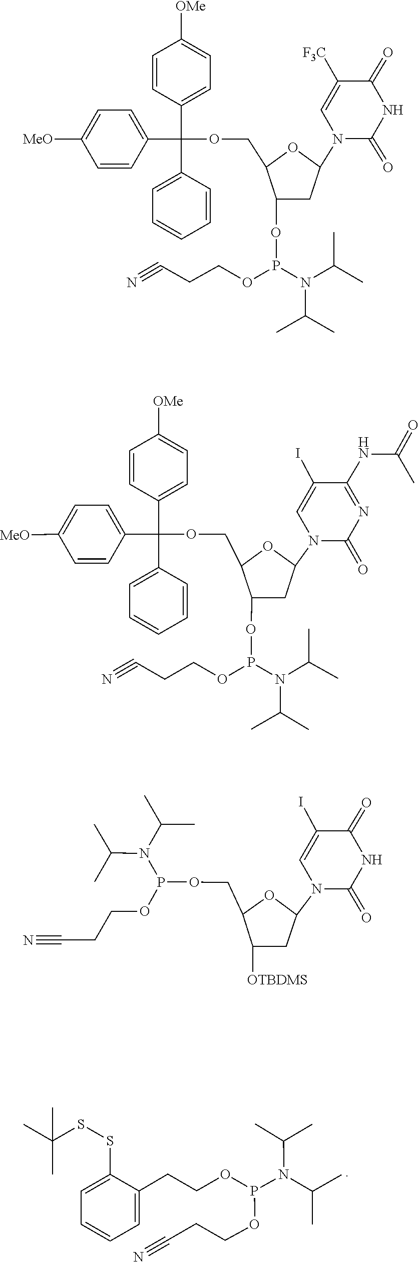

These intermediates may be used in the preparation of polynucleotides of the invention (e.g., polynucleotides containing a 5′-terminal modified nucleoside). Non-limiting examples of 5′-terminal modified nucleosides are 5-halouridine, 5-alkynyluridine, 5-heteroaryluridine, and 5-halocytidine.

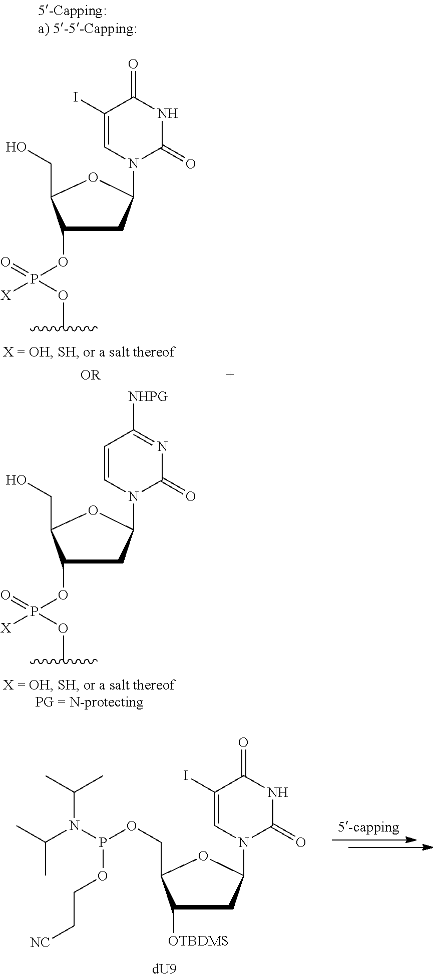

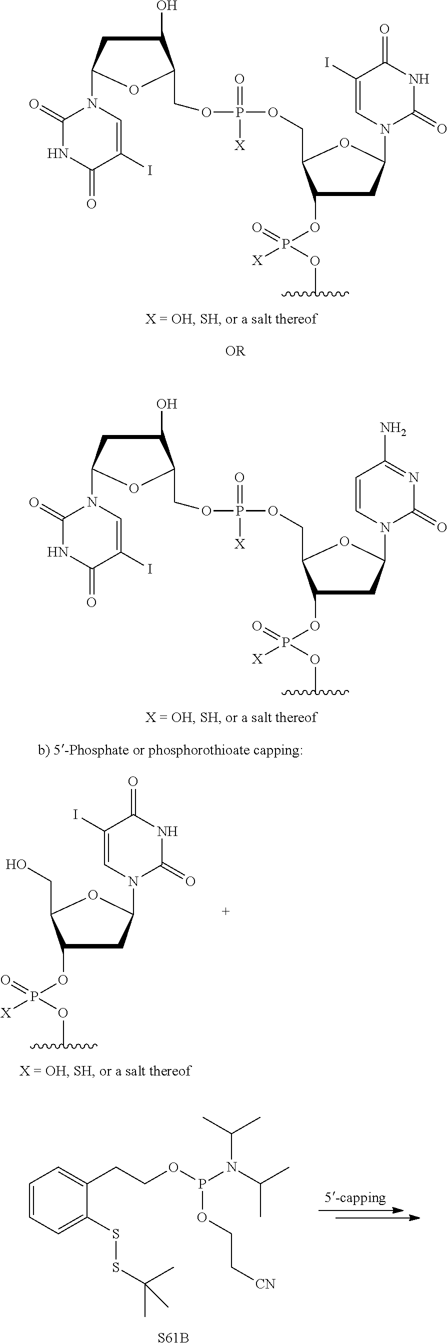

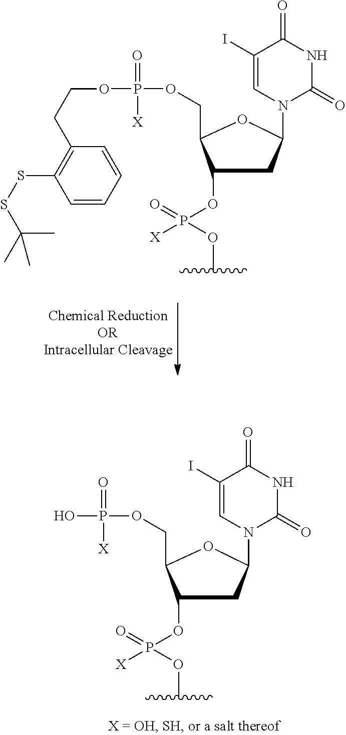

### Synthesis of Small Molecule-based Targeting Moieties

Exemplary compounds useful for the preparation of small molecule-based targeting moieties are described in WO 2015/188197 (e.g., compounds M1-M30 described in WO 2015/188197).

### Synthesis of Glucitol Auxiliary Moieties

Exemplary compounds useful for the preparation of glucitol-based auxiliary moieties are described in WO 2015/188197 (e.g., compounds POH1-POH10 described in WO 2015/188197).

### General Polynucleotide Synthesis

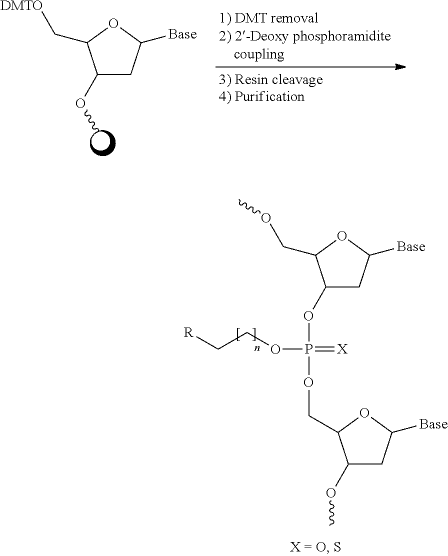

### Experimental Details

Automated polynucleotide synthesis (1 μmol scale) was carried out on MerMade 6 or 12 with the following reagents and solvents:
Oxidizer—0.02M 12 in THF/pyridine/H₂O (60 s oxidation per cycle),
Sulfurizing Reagent II—dithiazole derivative/pyridine/acetonitrile (0.05 M, in 6:4 pyridine:acetonitrile) (60 s per cycle)
Deblock—3% trichloroacetic acid (2×40 s deblocks per cycle),
Cap Mix A—THF/2,6-lutidine/Ac20 (60 s capping per cycle), and
Cap Mix B—16% methyl imidazole in THE (60 s capping per cycle)

Exceptions to standard polynucleotide synthesis conditions were as follows:
CPG supports with a non-nucleosidic linker called Uny-linker was used.
All 2′-deoxyribose-phosphoramidites were resuspended to 100 mM in 100% anhydrous acetonitrile prior to synthesis, except some of the modified 2′-deoxy-phosphoramidites were dissolved to 100 mM in THE/acetonitrile mixture (1:4) depend on the solubility of the starting material.
Phosphoramidite activation was performed with a 2.5-fold molar excess of 5-benzylthio-1H-tetrazole (BTT). Activated 2′-deoxyribose-phosphoramidites were coupled for 2×1 minute coupling per insertion and modified phosphoramidites were coupled for 2×3 minute coupling per insertion.
Sulfurization of the backbone was performed with 0.05M Sulfurizing Reagent II in pyridine/acetonitrile (6:4) for 1 min.

### Polynucleotide Deprotection & Purification Protocol

Following automated polynucleotide synthesis, solid support and base protecting groups (such as A-Bz, C-Ac, G-iBu, etc.) and methyl esters of phosphotriesters were cleaved and deprotected in 1 mL of AMA (1:1 ratio of 36% aq. ammonia and 40% methylamine in methanol) for 2 h or more at room temperature followed by centrifugal evaporation.

Crude polynucleotide pellets were resuspended in 100 μL of 50% acetonitrile, briefly heated to 65° C. and vortexed thoroughly.

For polynucleotide purification, 100 μL crude polynucleotides were injected onto R^{P}-HPLC with the following buffers/gradient:
Buffer A=50 mM TEAA in Water;
Buffer B=90% Acetontrile; and
Flow Rate=1 mL/min;
Gradient:
   0-2 min (100% Buffer A/0% Buffer B),
   2-42 min (0% to 60% Buffer B), and
   42-55 min (60% to 100% Buffer B).

### DBCO Conjugation and Purification Protocol:

DBCO NHS ester was conjugated to the crude 2′-deoxy DMT-polynucleotide as described here. The crude polynucleotide pellet was suspended into 45 μL DMSO, briefly heated to 65° C. and vortexed thoroughly. 5 μL of DIPEA was added followed by DBCO-NHS ester (30 eq), which was pre-dissolved in DMSO (1 M). The reaction was allowed to stand for 10 minutes or until product formation was confirmed by MALDI. Total 80 μL of crude polynucleotide samples were injected onto R^{P}-HPLC with the following buffers/gradient:
Buffer A=50 mM TEAA in Water
Buffer B=90% Acetonitrile
Flow Rate=1 mL/min
Gradient:
   0-2 min (90% Buffer A/10% Buffer B)
   2-42 min (0% to 60% Buffer B)
   42-55 min (60% to 100% Buffer B).

Across the dominant R^{P}-HPLC peaks, 0.5 mL fractions were collected and analyzed by MALDI-TOF mass spectrometry to confirm presence of desired mass. Mass-selected, purified fractions were frozen and lyophilized. Once dry, fractions were re-suspended, combined with corresponding fractions, frozen and lyophilized.

DMT Cleavage: lyophilized pellets were suspended in 20 μL of 50% acetonitrile and added 80 μL of acetic acid, samples were kept standing at room temperature for 1 h, frozen and lyophilized. The dried samples were re-dissolved in 20% acetonitrile and desalted through NAP 10 (Sephadex™-G25 DNA Grade) columns. Collected, pure fractions were frozen and lyophilized for final product.

### General Conjugation Schemes Using Abasic Spacers:

Click Reaction—General Scheme:

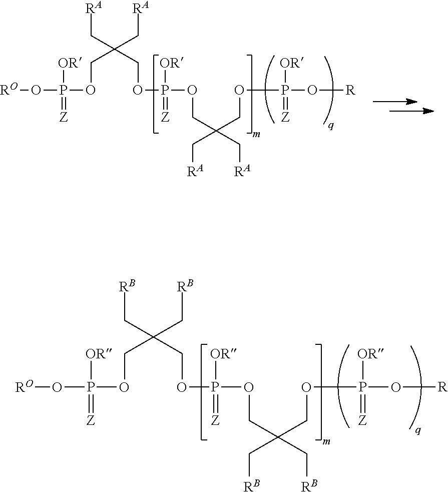

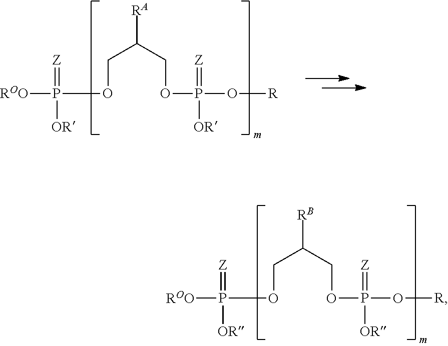

where:

each q is 0 or 1;

each m is an integer from 0 to 5;

Z isO or S;

R^{O} is a bond to a nucleoside in a polynucleotide;

R is a bond to H, a nucleoside in a polynucleotide, to solid support, or to a capping group (e.g., —(CH₂)₃—OH);

each R′ is independently H, -Q¹-Q^{A1}, a bioreversible group, or a non-bioreversible group;

each R″ is independently H, -Q¹-Q^{A}-Q²-T, a bioreversible group, or a non-bioreversible group;

each R^{A} is independently H or —OR^{C}, where R^{C} is -Q¹-Q^{A1}, a bioreversible group, a non-bioreversible group, or a bond to solid support;

each R^{B} is independently H or —OR^{D}, where R^{D} is -Q¹-Q^{A}-Q²-T, a bioreversible group, or a non-bioreversible group;

where:
each Q¹ is independently a divalent, trivalent, tetravalent, or pentavalent group, in which one valency is bonded to Q^{A} or Q^{A1}; a second valency is open, and each of the remaining valencies, when present, is independently bonded to an auxiliary moiety;
each Q² is independently a divalent, trivalent, tetravalent, or pentavalent group, in which one valency is bonded to Q^{A}; a second valency is bonded to T, and each of the remaining valencies, when present, is independently bonded to an auxiliary moiety;
Q^{A} is 1,2,3-triazole-1,4-diyl, optionally substituted C₆₋₁₆ triazoloheterocyclylene (e.g.,

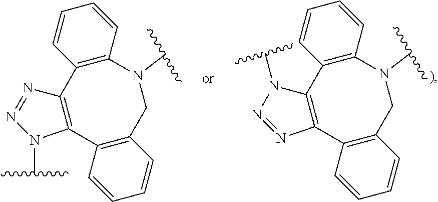 [SMILES: *N1Cc2ccccc2-c2c(nnn2*)-c2ccccc21 ; *N1Cc2ccccc2-c2nnn(*)c2-c2ccccc21]

optionally substituted C₈₋₁₆ triazolocycloalkenylene (e.g.,

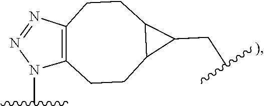 [SMILES: *CC1C2CCc3nnn(*)c3CCC12]

or a dihydropyridazine group (e.g.,

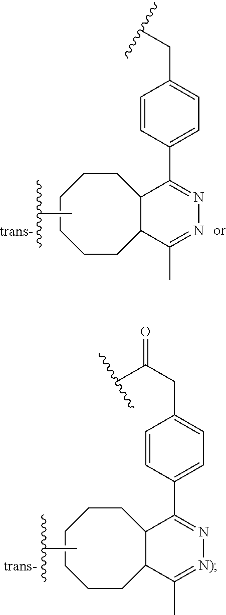

Q^{A1} is optionally substituted C₂₋₁₂ alkynyl, optionally substituted C₆₋₁₆ heterocyclyl containing an endocyclic carbon-carbon triple bond (e.g.,

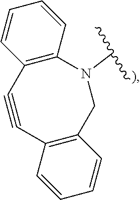 [SMILES: *N1Cc2ccccc2C#Cc2ccccc21]

optionally substituted C₈₋₁₆ cycloalkynyl (e.g.,

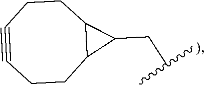 [SMILES: *CC1C2CCC#CCCC12]

or optionally substituted C₄₋₈ strained cycloalkenyl (e.g., trans-cyclooctenyl); and
T is a targeting moiety,
provided that the starting materials contain at least one -Q¹-Q^{A1}, and products contain -Q¹-Q^{A}-Q²-T; and
provided that the starting materials and products contain 0 or 1 bonds to a solid support.

### Conjugation Methods

### Cu-Catalyzed Click Reaction

Copper-THPTA Complex Preparation

A 5 mM aqueous solution of copper sulfate pentahydrate (CuSO₄-5H₂O) and a 10 mM aqueous solution of tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition, e.g., as shown in the general conjugation schemes below.

General Procedure (100 nM Scale):

To a solution of 710 μL of water and 100 μL tert-butanol (10% of final volume) in a 1.7 mL Eppendorf tube was added 60 μL of the copper-THPTA complex followed by 50 μL of a 2 mM solution of the oligo, 60 μL of a 20 mM aqueous sodium ascorbate solution and 20 μL of a 10 mM solution of targeting moiety-azide. After thorough mixing the solution was allowed to stand at room temperature for 1 hour. Completion of the reaction was confirmed by gel analysis. The reaction mixture is added to a screw cap vial containing 5-10 fold molar excess of SiliaMetS® TAAcONa (resin bound EDTA sodium salt). The mixture is stirred for 1 hour. This mixture is then eluted through an Illustra™Nap™-10 column Sephadex™. The resulting solution is then frozen and lyophilized overnight.

### Conjugation Through Amide Linkage:

Conjugation through amidation may be performed under the amidation reaction conditions known in the art. See, e.g., Aaronson et al., Bioconjugate Chem. 22:1723-1728, 2011.

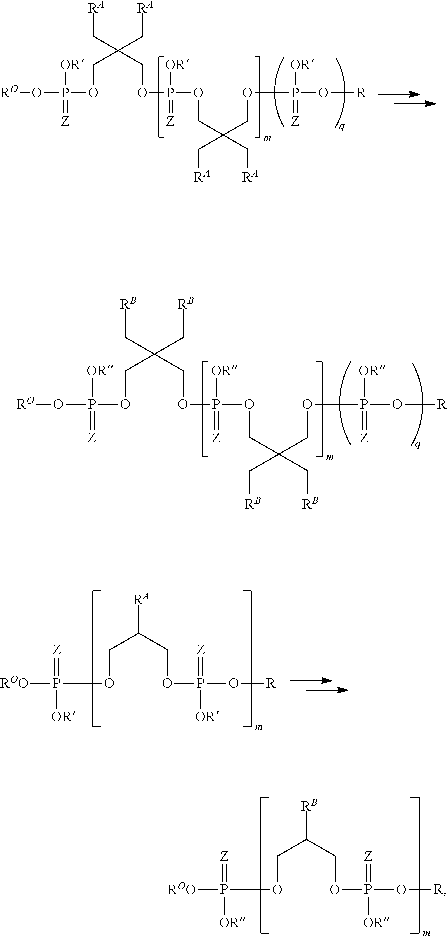

where:

each q is 0 or 1;

each m is an integer from 0 to 5;

Z is O or S;

R^{O} is a bond to a nucleoside in a polynucleotide;

R is a bond to H, a nucleoside in a polynucleotide, to solid support, or to a capping group (e.g., —(CH₂)₃—OH);

each R′ is independently H, -Q¹-Q^{A1}, a bioreversible group, or a non-bioreversible group;

each R″ is independently H, -Q¹-Q^{A}-Q¹-T, a bioreversible group, or a non-bioreversible group;

each R^{A} is independently H or —OR^{c}, where R^{c} is -Q¹-Q^{A1}, a bioreversible group, or a non-bioreversible group;

each R^{B} is independently H or —OR^{D}, where R^{D} is -Q¹-Q^{A}-Q²-T, a bioreversible group, or a non-bioreversible group;

where:
each Q¹ is independently a divalent, trivalent, tetravalent, or pentavalent group, in which one valency is bonded to Q^{A} or Q^{A1}, the second valency is open, and each of the remaining valencies, when present, is independently bonded to an auxiliary moiety;
each Q² is independently a divalent, trivalent, tetravalent, or pentavalent group, in which one valency is bonded to Q^{A}, the second valency is bonded to T, and each of the remaining valencies, when present, is independently bonded to an auxiliary moiety;
Q^{A} is optionally substituted C₂₋₁₂ heteroalkylene containing —C(O)—N(H)— or —N(H)—C(O)—;
Q^{A1} is —NHR^{N1} or —COOR¹², where R^{N1} is H, N-protecting group, or optionally substituted C₁₋₆ alkyl, and R¹² is H, optionally substituted C₁₋₆ alkyl, or O-protecting group; and
T is a targeting moiety,
provided that the starting materials contain at least one -Q¹-Q^{A1}, and products contain -Q¹-Q^{A}-Q²-T.

Solution Phase Conjugation:

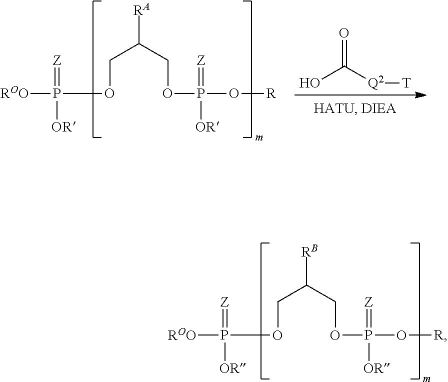

where:

m is an integer from 0 to 5;

Z is O or S;

R^{O} is a bond to a nucleoside in a polynucleotide;

R is a bond to H, a nucleoside in a polynucleotide, or to a capping group;

each R′ is independently H, -Q¹-NH₂, a bioreversible group, or a non-bioreversible group;

each R″ is independently H, -Q¹-NH—CO-Q²-T, a bioreversible group, or a non-bioreversible group;

each R^{A} is independently H or —OR^{C}, where R^{C} is -Q¹-NH₂, a bioreversible group, or a non-bioreversible group;

each R^{B} is independently H or —OR^{D}, where R^{D} is -Q¹-NH—CO-Q²-T, a bioreversible group, or a non-bioreversible group;

where:
each Q¹ is independently a divalent, trivalent, tetravalent, or pentavalent group, in which one valency is bonded to —NH—CO— or —NH₂, the second valency is open, and each of the remaining valencies, when present, is independently bonded to an auxiliary moiety;
each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
T is a targeting moiety,
provided that the starting material contains -Q¹-NH₂, and the product contains -Q¹-NH—CO-Q²-T.

On-support Conjugation:

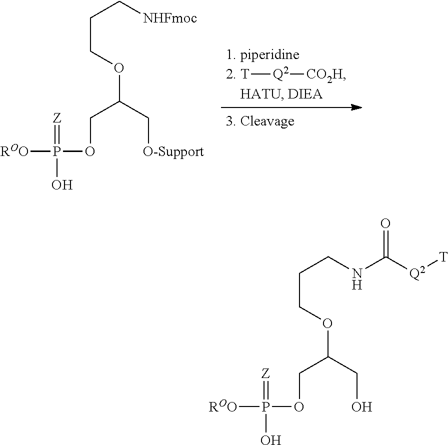

where:

Z is O or S;

R^{O} is a bond to a nucleoside in a polynucleotide;

each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and

T is a targeting moiety.

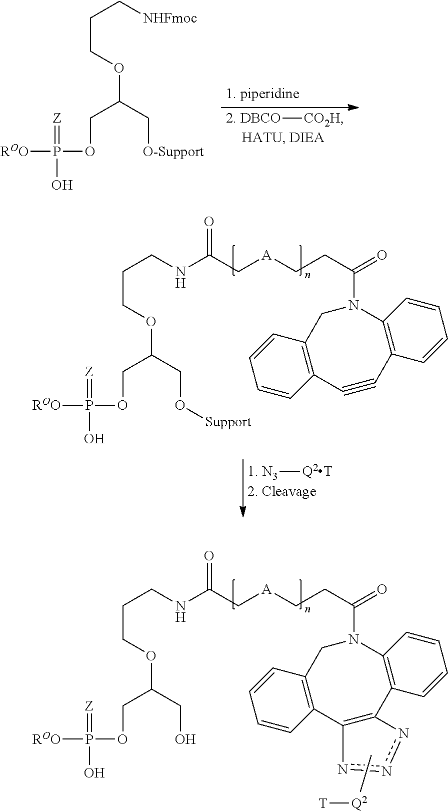

where:

n is an integer from 1 to 8;

A is O or —CH₂—;

Z is O or S;

R^{O} is a bond to a nucleoside in a polynucleotide;

each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and

T is a targeting moiety.

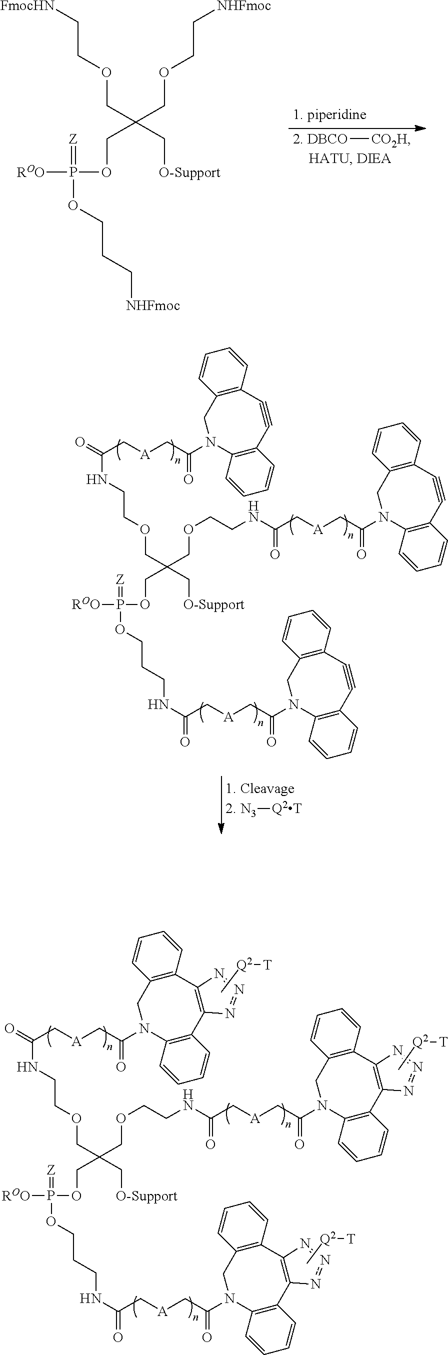

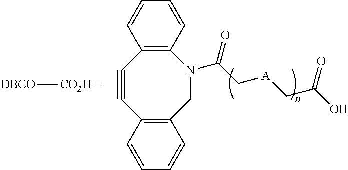

where:

n is an integer from 1 to 8;

A is O or —CH₂—;

Z is O or S;

R^{O} is a bond to a nucleoside in a polynucleotide;

each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and

T is a targeting moiety.

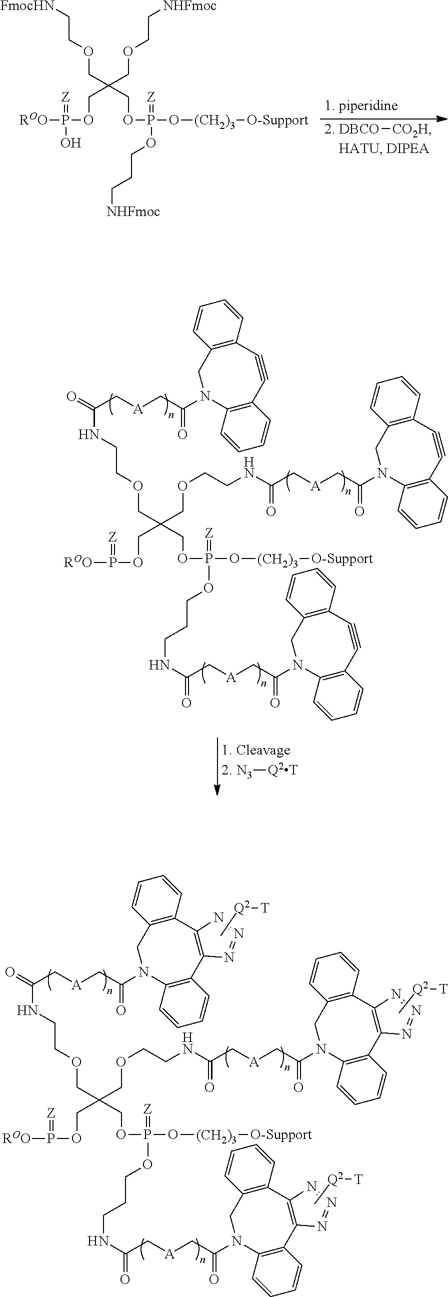

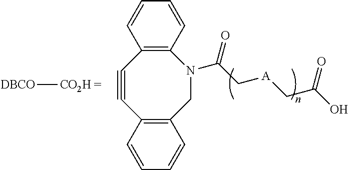

where:

n is an integer from 1 to 8;

A is O or —CH₂—;

Z is O or S;

R^{O} is a bond to a nucleoside in a polynucleotide;

each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and

each T is independently a targeting moiety.

### Representative Example of Fmoc Deprotection of a Phosphotriester:

A polynucleotide including a phosphotriester with Fmoc-protected amine was subjected to deprotection conditions resulting in Fmoc deprotection without observable conversion of the phosphotriester into a phosphodiester.

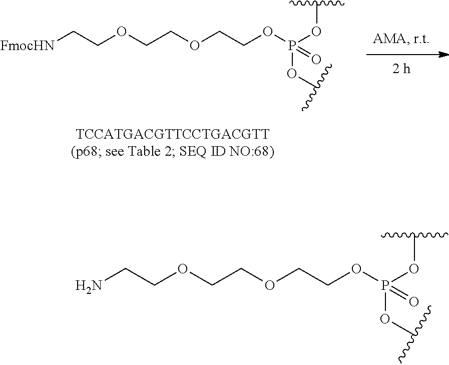

DBCO-NHS Conjugation to p68—Representative Example:

DBCO-NHS conjugation to the amino group in the phosphotriester was complete in 10 min at room temperature, as evidenced by mass spectrometric analysis.

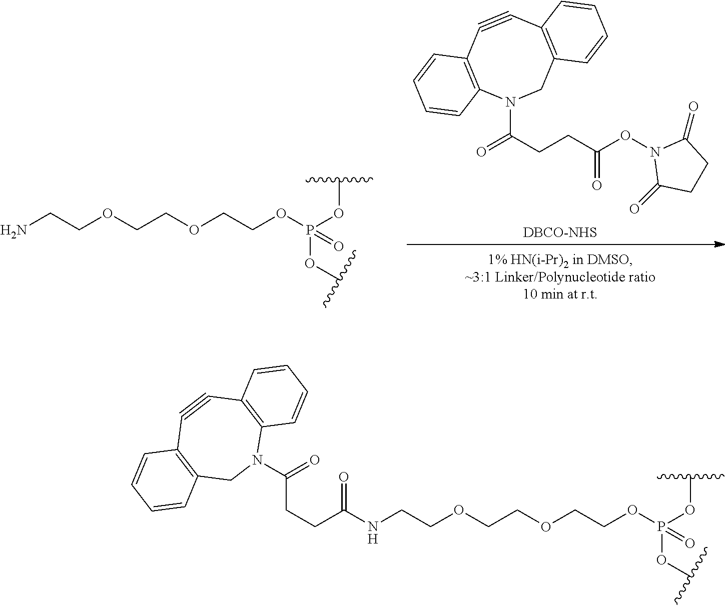

R^{P}-HPLC purification of p68 (see Table 2) containing a DBCO conjugating group was performed using the following conditions:
Buffer A=50 mM TEAA in Water;
Buffer B=90% Acetontrile; and
Flow Rate=1 mL/min;
Gradient:
   0-2 min (100% Buffer A/0% Buffer B),
   2-22 min (0% to 100% Buffer B), and
   22-25 min (100% Buffer B).

A similar procedure may be used to prepare a polynucleotide using, e.g., 2′-modified nucleoside phosphoramidites, such as those described herein. Such a procedure is provided in International Patent application PCT/US2015/034749; the disclosure of the disulfide phosphotriester oligonucleotide synthesis in PCT/US2015/034749 is hereby incorporated by reference.

The general procedure described herein was followed to prepare immunomodulating polynucleotides listed in Table 2.

TABLE 2

| Compound # | Sequence (5′ to 3′) | SEQ ID NO: | A | B | C | D | E | F | G | H | I | J |
|---|---|---|---|---|---|---|---|---|---|---|---|---|
| p1 | tcgtcgttttgtcgttttgtcgtt | 1 | 120 | 110 | 175 | 50 | >1000 | | | | | |
| p2 | NH2-C6- | 2 | | | | | | | | | | |
| | tcgtcgttttgtcgttttgtcgtt | | | | | | | | | | | |
| P3 | NH2-C6-S-S-C6- | 3 | | | | | | | | | | |
| | tcgtcgttttgtcgttttgtcgtt | | | | | | | | | | | |
| p4 | DBCO- | 4 | >1000 | | >1000 | | | | | | | |
| | tcgtcgttttgtcgttttgtcgtt | | | | | | | | | | | |
| P5 | DBCO-C6-S-S-C6- | 5 | | | | | | | | | | |
| | tcgtcgttttgtcgttttgtcgtt | | | | | | | | | | | |
| p6 | TCGTCGTTTTGTCGTTT | 6 | | | >1000 | >1000 | >1000 | | | | | |
| | TGTCGTT | | | | | | | | | | | |
| P7 | DBCO-C6-S-S-C6- | 7 | | | | | | | | | | |
| | TCGTCGTTTTGTCGTTT | | | | | | | | | | | |
| | TGTCGTT | | | | | | | | | | | |
| p8 | tgctgcttttgtgcttttgtgctt | 8 | | | >1000 | >1000 | >1000 | | | | | |
| P9 | DBCO-C6-S-S-C6- | 9 | | | | | | | | | | |
| | tgctgcttttgtgcttttgtgctt | | | | | | | | | | | |
| p10 | tcgtcgttttgtcgttttgtcgtt | 10 | | | | | | | | | | |
| p11 | TCGTCGTTTTGTCGTTT | 11 | | | | | | | | | | |
| | TGTCGTT | | | | | | | | | | | |
| p12 | DBCO-C6-S-S-C6- | 12 | | | | | | | | | | |
| | tcgtcgttttgtcgttttgtcgtt | | | | | | | | | | | |
| p13 | DBCO-C6-S-S-C6- | 13 | | | | | | | | | | |
| | TCGTCGTTTTGTCGTTT | | | | | | | | | | | |
| | TGTCGTT | | | | | | | | | | | |
| p14 | DBCO- | 14 | | | | | | | | | | |
| | tcgtcgttttgtcgttttgtcgtt | | | | | | | | | | | |
| p15 | DBCO- | 15 | | | | | | | | | | |
| | TCGTCGTTTTGTCGTTT | | | | | | | | | | | |
| | TGTCGTT | | | | | | | | | | | |
| p16 | DBCO- | 16 | | | | | | | | | | |
| | tccatgacgttcctgacgtt | | | | | | | | | | | |
| p17 | DBCO- | 17 | | | | | | | | | | |
| | TCCATGACGTTCCTGA | | | | | | | | | | | |
| | CGTT | | | | | | | | | | | |
| p18 | tccatgacgttcctgacgtt | 18 | | | 1000 | >1000 | 40 | 22.6 | 24.4 | 26 | | |
| p19 | DBCO- | 19 | | | | | | >1000 | | >1000 | | |
| | tccatgacgttcctgacgtt | | | | | | | | | | | |
| p20 | TCCATGACGTTCCTGA | 20 | | | | >1000 | >1000 | >1000 | | | | |
| | CGTT | | | | | | | | | | | |
| p21 | DBCO- | 21 | | | | | | | | | | |
| | TCCATGACGTTCCTGA | | | | | | | | | | | |
| | CGTT | | | | | | | | | | | |
| p22 | tccatgagcttcctgagctt | 22 | | | | | | >1000 | >1000 | | | |
| p23 | DBCO- | 23 | | | | | | | | | | |
| | tccatgagcttcctgagctt | | | | | | | | | | | |
| p24 | dtcgtcgttttgtcgttttgtcgtt | 24 | 500 | | >1000 | | | | | | | |
| p25 | tcgtcgttdttgtcgttttgtcgtt | 25 | >1000 | | 800 | | | | | | | |
| p26 | tcgtcgttttgdtcgttttgtcgtt | 26 | >1000 | | >1000 | | | | | | | |
| p27 | tcgtcgttttgtcgttdttgtcgtt | 27 | 500 | | 800 | | | | | | | |
| p28 | tcgtcgttttgtcgttttgtcgtdt | 28 | 300 | | 800 | | | | | | | |
| p29 | tcgcgacgttcgcccgacgttcg | 29 | | | 300 | >1000 | >1000 | 92.8 | | 58 | | |
| | gta | | | | | | | | | | | |
| p30 | DBCO- | 30 | | | | | | 183.7 | | 28 | | |
| | tcgcgacgttcgcccgacgttcg | | | | | | | | | | | |
| | gta | | | | | | | | | | | |
| p31 | tccatgacgttcctgatgct | 31 | | | 1000 | >1000 | 40 | 29.6 | | 27 | | |
| p32 | DBCO- | 32 | | | | >1000 | >1000 | | | | | |
| | tccatgacgttcctgatgct | | | | | | | | | | | |
| p33 | tcgacgttcgtcgttcgtcgttc | 33 | | | 450 | | | 103.2 | | 275 | | |
| p34 | DBCO- | 34 | | | | | | | | | | |
| | tcgacgttcgtcgttcgtcgttc | | | | | | | | | | | |
| p35 | tcgtcgttttgtcgttttgtcgtt | 35 | | | | | | | | | | |
| p36 | DBCO- | 36 | | | | | | | | | | |
| | tcgtcgttttgtcgttttgtcgtt | | | | | | | | | | | |
| p37 | tccatgacgttcctgacgtt | 37 | | | | | | 164.3 | 180 | 28.3 | | |
| p38 | DBCO- | 38 | | | | | | | | | | |
| | tccatgacgttcctgacgtt | | | | | | | | | | | |
| p39 | tccatgacgttcctgacgtt-C3 | 39 | | | | | | 122.2 | 130.8 | | | |
| p40 | TCCATGACGTTCCTGA | 40 | | | | | | >1000 | | | | |
| | CGTT | | | | | | | | | | | |
| p41 | TCCATGACGTTCCTGA | 41 | | | | | | >1000 | | | | |
| | CGTT-C3 | | | | | | | | | | | |
| p42 | tccatgacgttcctgacgtt | 42 | | | | | | 22.6 | 25.6 | | | |
| p43 | tccatgacgttcctgacgtt-C3 | 43 | | | | | | 19.2 | | | | |
| p44 | TCCATGACGTTCCTGA | 44 | | | | | | >1000 | | | | |
| | CGTT | | | | | | | | | | | |
| p45 | TCCATGACGTTCCTGA | 45 | | | | | | | | | | |
| | CGTT-C3 | | | | | | | | | | | |
| p46 | tccatgacgttcctgacgtt | 46 | | | | | | 876 | | | | |
| p47 | tccatgacgttcctgacgtt | 47 | | | | | | 615 | | | | |
| p48 | tccatgacgttcctgacgtt | 48 | | | | | | 197.2 | | | | |
| p49 | tccatgacgttcctgacgtt | 49 | | | | | | 75.2 | | | | |
| p50 | tccatgacgttcctgacgtt | 50 | | | | | | 71.3 | | | | |
| p51 | tccatgacgttcctgacgtt | 51 | | | | | | 9.3 | | | | |
| p52 | tccatgacgttcctgacgtt | 52 | | | | | | 29.1 | | | | |
| p53 | GGgggacgatcgtcGGGGG | 53 | | | | | | | | | | |
| | G | | | | | | | | | | | |
| p54 | tcgtcgtcgttcgaacgacgttgat | 54 | | | | | | | | 816 | | |
| p55 | tcgtcgttttcggcgcgcgccg | 55 | | | | | | | | 31.4 | | |
| p56 | tcgcgaacgttcgccgcgttcga | 56 | | | | | | | | | | |
| | acgcgg | | | | | | | | | 45.5 | | |
| p57 | tcgtcgacgatcggcgcgcgcc | 57 | | | | | | | | | | |
| | g | | | | | | | | | | | |
| p58 | tccatgacgttcctgacgtt | 58 | | | | | | | | | | |
| p59 | tccatgacgttcctgacgtt | 59 | | | | | | | | | | |
| p60 | tccatgacgttcctgacgtt | 60 | | | | | | | | | | |
| p61 | tccatgacgttcctgacgtt | 61 | | | | | | | | | | |
| p62 | tccatgacgttcctgacgtt | 62 | | | | | | | | | | |
| p63 | tccatgacgttcctgacgtt | 63 | | | | | | | | | | |
| p64 | tccatgacgttcctgacgtt | 64 | | | | | | | | | | |
| p65 | TCCATGACGTTCCTGA | 65 | | | | | | | | | | |
| | CGTT | | | | | | | | | | | |
| | TCCATGACGTTCCTGA | 66 | | | | | | | | | | |
| p66 | CGTT | | | | | | | | | | | |
| | TCCATGACGTTCCTGA | 67 | | | | | | | | | | |
| p67 | CGTT | | | | | | | | | | | |
| | TCCATGACGTTCCTGA | 68 | | | | | | | | | | |
| p68 | CGTT | | | | | | | | | | | |
| | TCCATGACGTTCCTGA | 69 | | | | | | | | | | |
| p69 | CGTT | | | | | | | | | | | |
| | TCCATGACGTTCCTGA | 70 | | | | | | | | | | |
| p70 | CGTT | | | | | | | | | | | |
| | TCCATGACGTTCCTGA | 71 | | | | | | | | | | |
| p71 | CGTT | | | | | | | | | | | |
| p72 | tcgtcgttttgtcgttttgtcgtt | 72 | | | | | | | | | | |
| | TCGTCGTTTTGTCGTTT | 73 | | | | | | | | | | |
| p73 | TGTCGTT | | | | | | | | | | | |
| p74 | tccatgacgttcctgatgct | 74 | | | | | | | | | | |
| | TCCATGACGTTCCTGA | 75 | | | | | | | | | | |
| p75 | TGCT | | | | | | | | | | | |
| | TCGTCGTTTTGTCGTTT | 76 | | | | | | | | | | |
| p76 | TGTCGTT | | | | | | | | | | | |
| | TCGTCGTTTTGTCGTTT | 77 | | | | | | | | | | |
| p77 | TGTCGTT | | | | | | | | | | | |
| | TCGTCGTTTTGTCGTTT | 78 | | | | | | | | | | |
| p78 | TGTCGTT | | | | | | | | | | | |
| | TCGTCGTTTTGTCGTTT | 79 | | | | | | | | | | |
| p79 | TGTCGTT | | | | | | | | | | | |
| | TCGTCGTTTTGTCGTTT | 80 | | | | | | | | | | |
| p80 | TGTCGTT | | | | | | | | | | | |
| | TCGTCGTTTTGTCGTTT | 81 | | | | | | | | | | |
| p81 | TGTCGTT | | | | | | | | | | | |
| | TCGTCGTTTTGTCGTTT | 82 | | | | | | | | | | |
| p82 | TGTCGTT | | | | | | | | | | | |
| | TCGTCGTTTTGTCGTTT | 83 | | | | | | | | | | |
| p83 | TGTCGTT | | | | | | | | | | | |
| | TCGTCGTTTTGTCGTTT | 84 | | | | | | | | | | |
| p84 | TGTCGTT | | | | | | | | | | | |
| | TCGTCGTTTTGTCGTTT | 85 | | | | | | | | | | |
| p85 | TGTCGTT | | | | | | | | | | | |
| | TCGTCGTTTTGTCGTTT | 86 | | | | | | | | | | |
| p86 | TGTCGTT | | | | | | | | | | | |
| | TCGTCGTTTTGTCGTTT | 87 | | | | | | | | | | |
| p87 | TGTCGTT | | | | | | | | | | | |
| | TCGTCGTTTTGTCGTTT | 88 | | | | | | | | | | |
| p88 | TGTCGTT | | | | | | | | | | | |
| | tccatGACGTTCCTGACG | 89 | | | | | | | | | | |
| p89 | TT | | | | | | >1000 | | | | | |
| p90 | tccatgacgtTCCTGACGTT | 90 | | | | | | 1000 | | | | |
| p91 | tccatgacgttcctgACGTT | 91 | | | | | | 49 | | | | |
| | tccatGACGTTCCTGACG | 92 | | | | | | | | | | |
| p92 | TT | | | | | | | >1000 | | | | |
| p93 | tccatgacgtTCCTGACGTT | 93 | | | | | | >1000 | | | | |
| p94 | tccatgacgttcctgACGTT | 94 | | | | | | 145 | | | | |
| | TCGTCGTTTTGTCGTTT | 95 | | | | | | | | | | |
| p95 | TGTCGTT | | | | | | | | | | | |
| | TCGTCGTTTTGTCGTTT | 96 | | | | | | | | | | |
| p96 | TGTCGTT | | | | | | | | | | | |
| | TCGTCGTTTTGTCGTTT | 97 | | | | | | | | | | |
| p97 | TGTCGTT | | | | | | | | | | | |
| | TCGTCGTTTTGTCGTTT | 98 | | | | | | | | | | |
| p98 | TGTCGTT | | | | | | | | | | | |
| | TCGTCGTTTTGTCGTTT | 99 | | | | | | | | | | |
| p99 | TGTCGTT | | | | | | | | | | | |
| | TCGTCGTTTTGTCGTTT | 100 | | | | | | | | | | |
| p100 | TGTCGTT | | | | | | | | | | | |
| | TCGTCGTTTTGTCGTTT | 101 | | | | | | | | | | |
| p101 | TGTCGTT | | | | | | | | | | | |
| | TCGTCGTTTTGTCGTTT | 102 | | | | | | | | | | |
| p102 | TGTCGTT | | | | | | | | | | | |
| | TCGTCGTTTTGTCGTTT | 103 | | | | | | | | | | |
| p103 | TGTCGTT | | | | | | | | | | | |
| | TCGTCGTTTTGTCGTTT | 104 | | | | | | | | | | |
| p104 | TGTCGTT | | | | | | | | | | | |
| | TCCATGACGTTCCTGA | 105 | | | | | | | | | | |
| p105 | CGTT | | | | | | | | | | | |
| | TCCATGACGTTCCTGA | 106 | | | | | | | | | | |
| p106 | CGTT | | | | | | | | | | | |
| | TCCATGACGTTCCTGA | 107 | | | | | | | | | | |
| p107 | CGTT | | | | | | | | | | | |
| | TCCATGACGTTCCTGA | 108 | | | | | | | | | | |
| p108 | CGTT | | | | | | | | | | | |
| | TCCATGACGTTCCTGA | 109 | | | | | | | | | | |
| p109 | CGTT | | | | | | | | | | | |
| | TCCATGACGTTCCTGA | 110 | | | | | | | | | | |
| p110 | CGTT | | | | | | | | | | | |
| | TCCATGACGTTCCTGA | 111 | | | | | | | | | | |
| p111 | CGTT | | | | | | | | | | | |
| | TCCATGACGTTCCTGA | 112 | | | | | | | | | | |
| p112 | CGTT | | | | | | | | | | | |
| | TCCATGACGTTCCTGA | 113 | | | | | | | | | | |
| p113 | CGTT | | | | | | | | | | | |
| | TCCATGACGTTCCTGA | 114 | | | | | | | | | | |
| p114 | CGTT | | | | | | | | | | | |
| | TCCATGACGTTCCTGA | 115 | | | | | | | | | | |
| p115 | CGTT | | | | | | | | | | | |
| | TCCATGACGTTCCTGA | 116 | | | | | | | | | | |
| p116 | CGTT | | | | | | | | | | | |
| | TCCATGACGTTCCTGA | 117 | | | | | | | | | | |
| p117 | CGTT | | | | | | | | | | | |
| | TCCATGACGTTCCTGA | 118 | | | | | | | | | | |
| p118 | CGTT | | | | | | | | | | | |
| | TCCATGACGTTCCTGA | 119 | | | | | | | | | | |
| p119 | CGTT | | | | | | | | | | | |
| | TCCATGACGTTCCTGA | 120 | | | | | | | | | | |
| p120 | CGTT | | | | | | | | | | | |
| | TCCATGACGTTCCTGA | 121 | | | | | | | | | | |
| p121 | CGTT | | | | | | | | | | | |
| | TCCATGACGTTCCTGA | 122 | | | | | | | | | | |
| p122 | CGTT | | | | | | | | | | | |
| | IR700- | 123 | | | | | | | | | | |
| p123 | tccatgacgttcctgacgft | | | | | | | | | | | |
| | IR700- | 124 | | | | | | | | | | |
| p124 | TCCATGACGTTCCTGA | | | | | | | | | | | |
| | CGTT | | | | | | | | | | | |
| p125 | tcgtcgtttcgtcgttttgtcgtt | 125 | | | | | | | | | | |
| | DBCO- | 126 | | | | | | | | | | |
| p126 | TCGTCGTTTTGTCGTTT | | | | | | | | | | | |
| | TGTCGTT | | | | | | | | | | | |
| | DBCO- | 127 | | | | | | | | | | |
| p127 | TCGTCGTTTTGTCGTTT | | | | | | | | | | | |
| | TGTCGTT | | | | | | | | | | | |
| | TGCTGCTTTTGTGCTTT | 128 | | | | | | | | | | |
| p128 | TGTGCTT | | | | | | | | | | | |
| | tcattgGAAAACGTTCTTC | 129 | | | | | | | | | | |
| p129 | GGGGCGTTctt | | | | | | | | | | | |
| | tcattgGAAAAGCTTCTTG | 130 | | | | | | | | | | |
| p130 | CGGGGCTTctt | | | | | | | | | | | |
| | TCATTGGAAAACGTTC | 131 | | | | | | | | | | |
| p131 | TTCGGGGCGTTCTT | | | | | | | | | | | |
| | AAGAACGCCCCGAAGA | 132 | | | | | | | | | | |
| p132 | ACGTTTTCCAATGA | | | | | | | | | | | |
| | TCATTGGAAAACGTTC | 133 | | | | | | | | | | |
| p133 | TTCGGGGCGTTCTT | | | | | | | | | | | |
| | AAGAACGCCCCGAAGA | 134 | | | | | | | | | | |
| p134 | ACGTTTTCCAATGA | | | | | | | | | | | |
| | TCATTGGAAAACGTTC | 135 | | | | | | | | | | |
| p135 | TTCGGGGCGTTCTT | | | | | | | | | | | |
| | AAGAACGCCCCGAAG | 136 | | | | | | | | | | |
| p136 | AACGTTTTCCAATGA | | | | | | | | | | | |
| | TCATTGGAAAACGTTC | 137 | | | | | | | | | | |
| p137 | TTCGGGGCGTTCTT | | | | | | | | | | | |
| | AAGAACGCCCCGAAG | 138 | | | | | | | | | | |
| p138 | AACGTTTTCCAATGA | | | | | | | | | | | |
| p139 | tccatGACGTTCCTGAcgtt | 139 | | | | | | | | | | |
| | TCCATGACGTTCCTGA | 140 | | | | | | | | | | |
| p140 | cgtt | | | | | | | | | | | |
| | tccatGACGTTCCTGACG | 141 | | | | | | | | | | |
| p141 | tt | | | | | | | | | | | |
| | tccatGACGTTCCTGACG | 142 | | | | | | | | | | |
| p142 | TT | | | | | | | | | | | |
| | tccatGACGTTCCTGACG | 143 | | | | | | | | | | |
| p143 | TT | | | | | | | | | | | |
| | AACGACAAAACGACAA | 144 | | | | | | | | | | |
| p144 | AACGACGA | | | | | | | | | | | |
| | AACGACAAAACGACAA | 145 | | | | | | | | | | |
| p145 | AACGACGA | | | | | | | | | | | |
| | TCGTCGTTTTGTCGTTT | 146 | | | | | | | | | | |
| p146 | TGTCGtT | | | | | | | | | | | |
| | TCGTCGTTTTGTCGTTT | 147 | | | | | | | | | | |
| p147 | TgtcgtT | | | | | | | | | | | |
| | TCGTCGTTTTGTCGTTT | 148 | | | | | | | | | | |
| p148 | TGTCGtT | | | | | | | | | | | |
| | TCGTCGTTTTGTCGTTT | 149 | | | | | | | | | | |
| p149 | TgtcgtT | | | | | | | | | | | |
| | tcgtcGTTTTGTCGTTTT | 150 | | | | | | | | | | |
| p150 | GTCGTT | | | | | | | | | | | |
| p151 | tcgtcgttttgtcgttttgtcgif | 151 | | | | | | | | | | |
| | tcgtcGTTTTGTCGTTTT | 152 | | | | | | | | | | |
| p152 | GTCGtT | | | | | | | | | | | |
| | tcgtcGTTTTGTCGTTTT | 153 | | | | | | | | | | |
| p153 | GTCGtt | | | | | | | | | | | |
| | tcgtcGTTTTGTCGTTTT | 154 | | | | | | | | | | |
| p154 | GTCGTt | | | | | | | | | | | |
| | TTCGTCGTTTTGTCGTT | 155 | | | | | | | | | | |
| p155 | TTGTCGTT | | | | | | | | | | | |
| | TTTCGTCGTTTTGTCGT | 156 | | | | | | | | | | |
| p156 | TTTGTCGTT | | | | | | | | | | | |
| | GTTTCGTCGTTTTGTC | 157 | | | | | | | | | | |
| p157 | GTTTTGTCGTT | | | | | | | | | | | |
| | GTTTCGTCGTTTTGTC | 158 | | | | | | | | | | |
| p158 | GTTTTGTCGTT | | | | | | | | | | | |
| | GTTTCGTCGTTTTGTC | 159 | | | | | | | | | | |
| p159 | GTTTTGTCGTT | | | | | | | | | | | |
| | GTTTCGTCGTTTTGTC | 160 | | | | | | | | | | |
| p160 | GTTTTGTCGTT | | | | | | | | | | | |
| | TCGTCGTTTTGTCGTTT | 161 | | | | | | | | | | |
| p161 | TGTCGTT-C3 | | | | | | | | | | | |
| | TCGTCGTTTTGTCGTTT | 162 | | | | | | | | | | |
| p162 | T | | | | | | | | | | | |
| | UCGTCGTTTTGTCGTT | 163 | | | | | | | | | | |
| p163 | TTGTCGtt-C3 | | | | | | | | | | | |
| | 03- | 164 | | | | | | | | | | |
| p164 | UCGTCGTTTTGTCGTT | | | | | | | | | | | |
| | TTGTCGTT-C3 | | | | | | | | | | | |
| | TCGUCGTTTTGTCGTT | 165 | | | | | | | | | | |
| p165 | TTGTCGTT-C3 | | | | | | | | | | | |
| | 03- | 166 | | | | | | | | | | |
| p166 | TCGUCGTTTTGTCGTT | | | | | | | | | | | |
| | TTGTCGTT-C3 | | | | | | | | | | | |
| | UCGUCGTTTTGTCGTT | 167 | | | | | | | | | | |
| p167 | TTGTCGTT-C3 | | | | | | | | | | | |
| | 03- | 168 | | | | | | | | | | |
| p168 | UCGUCGTTTTGTCGTT | | | | | | | | | | | |
| | TTGTCGTT-C3 | | | | | | | | | | | |
| | UCGTCGTTTTGTCGTT | 169 | | | | | | | | | | |
| p169 | TTGTCGTT-C3 | | | | | | | | | | | |
| | 03- | 170 | | | | | | | | | | |
| p170 | UCGTCGTTTTGTCGTT | | | | | | | | | | | |
| | TTGTCGTT-C3 | | | | | | | | | | | |
| | TCGTCGTTTTGTCGTTT | 171 | | | | | | | | | | |
| p171 | T | | | | | | | | | | | |
| p172 | TCGTCGTTTTGTCGTT | 172 | | | | | | | | | | |
| p173 | TCGTCGTTTTGTCG | 173 | | | | | | | | | | |
| p174 | TCGTCGTTTTGT | 174 | | | | | | | | | | |
| | UCGTCGTTTTGTCGTT | 175 | | | | | | | | | | |
| p175 | TT | | | | | | | | | | | |
| | UTCGTCGTTTTGTCGT | 176 | | | | | | | | | | |
| p176 | T | | | | | | | | | | | |
| p177 | UCGTCGTTTTGTCG | 177 | | | | | | | | | | |
| p178 | UCGTCGTTTTGT | 178 | | | | | | | | | | |
| | UCGUCGTTTTGTCGTT | 179 | | | | | | | | | | |
| p179 | TTGTCGTT-C3 | | | | | | | | | | | |
| | UCGTCGTTTTGTCGTT | 180 | | | | | | | | | | |
| p180 | TTGTCGTT-C3 | | | | | | | | | | | |
| | UCGTCGTTTTGTCGTT | 181 | | | | | | | | | | |
| p181 | TTGTCGTT-C3 | | | | | | | | | | | |
| | UCGTCGTTTTGTCGTT | 182 | | | | | | | | | | |
| p182 | TTGTCGTT-C3 | | | | | | | | | | | |
| | UCGTCGTTTTGTCGTT | 183 | | | | | | | | | | |
| p183 | TTGTCGTT-C3 | | | | | | | | | | | |
| | TCCATGACGTTCCTGA | 184 | | | | | | | | | | |
| p184 | TGCT-C3 | | | | | | | | | | | |
| p185 | tccatgacgttcctgatgct-C3 | 185 | | | | | | | | | | |
| p186 | tccatgacgttcctgatgct-C3 | 186 | | | | | | | | | | |
| | UCGTCGTTTGTCGTT- | 187 | | | | | | | | | | |
| p187 | C3 | | | | | | | | | | | |
| p188 | UCGTCGTTGTCGTT-C3 | 188 | | | | | | | | | | |
| p189 | UCGTCGTGTCGTT-C3 | 189 | | | | | | | | | | |
| p190 | UCGTCGTTCGTT-C3 | 190 | | | | | | | | | | |
| p191 | UCGTCGTCGTT-C3 | 191 | | | | | | | | | | |
| | UGCTGCTTTTGTGCTT | 192 | | | | | | | | | | |
| p192 | TTGTGCTT | | | | | | | | | | | |
| | TCCATGACGTTCCTGA | 193 | | | | | | | | | | |
| p193 | CGTT-C3 | | | | | | | | | | | |
| p194 | tccatgacgttcctgacgq-C3 | 194 | | | | | | | | | | |
| | TCCATGACGTTCCTGA | 195 | | | | | | | | | | |
| p195 | CGTT-C3 | | | | | | | | | | | |
| p196 | tccatgacgttcctgacgtt-C3 | 196 | | | | | | | | | | |
| | TAACGACAAAACGAC | 197 | | | | | | | | | | |
| p197 | AAAACGACGA | | | | | | | | | | | |
| | AACGACAAAACGACA | 198 | | | | | | | | | | |
| p198 | AAACGACGAT-C3 | | | | | | | | | | | |
| p199 | UCGTCGttttgtCGTT-C3 | 199 | | | | | | | | | | |
| p200 | UCGTCGttttgtCGTT-C3 | 200 | | | | | | | | | | |
| | UCGTCGttTTGTCGTT- | 201 | | | | | | | | | | |
| p201 | C3 | | | | | | | | | | | |
| | UCGTCGTTttGTCGTT- | 202 | | | | | | | | | | |
| p202 | C3 | | | | | | | | | | | |
| | UCGTCGTTTTgtCGTT- | 203 | | | | | | | | | | |
| p203 | C3 | | | | | | | | | | | |
| | UCGTCGTTTTGTCGTT- | 204 | | | | | | | | | | |
| p204 | C3 | | | | | | | | | | | |
| | UCGTCGTTTTGTCGTT- | 205 | | | | | | | | | | |
| p205 | C3 | | | | | | | | | | | |
| | UCGTCGTTTTGTCGTT- | 206 | | | | | | | | | | |
| p206 | C3 | | | | | | | | | | | |
| | UCGTCGTTTTGTCGTT- | 207 | | | | | | | | | | |
| p207 | C3 | | | | | | | | | | | |
| | UCGTCGTTTTGTCGTT- | 208 | | | | | | | | | | |
| p208 | C3 | | | | | | | | | | | |
| p209 | UCGTCGTT-C3 | 209 | | | | | | | | | | |
| p210 | UCGTCGTT-C3 | 210 | | | | | | | | | | |
| p211 | UCGTTT-C3 | 211 | | | | | | | | | | |
| p212 | UCGTTT-C3 | 212 | | | | | | | | | | |
| p213 | UCGTCGTGTCGTT-C3 | 213 | | | | | | | | | | |
| p214 | UCGTCGTGTTTTT-C3 | 214 | | | | | | | | | | |
| p215 | UCGTTTTGTCGTT-C3 | 215 | | | | | | | | | | |
| p216 | UCGTTTGTCGTT-C3 | 216 | | | | | | | | | | |
| p217 | UCGTTGTCGTT-C3 | 217 | | | | | | | | | | |
| p218 | UCGTGTCGTT-C3 | 218 | | | | | | | | | | |
| | UGCTGCTTTTGTGCTT- | 219 | | | | | | | | | | |
| p219 | C3 | | | | | | | | | | | |
| | UCGTCGTTTTGTCGTT- | 220 | | | | | | | | | | |
| p220 | C3 | | | | | | | | | | | |
| | UCGTCGTTTTGTCGTT- | 221 | | | | | | | | | | |
| p221 | C3 | | | | | | | | | | | |
| p222 | GGGACGATCGTCT | 222 | | | | | | | | | | |
| p223 | ggGACGATCGTCTgg | 223 | | | | | | | | | | |
| p224 | ggGACGATCGTCTgg | 224 | | | | | | | | | | |
| p225 | UCGTCGTGTCGTT-C3 | 225 | | | | | | | | | | |
| p226 | UCGTCGTGTCGTT-C3 | 226 | | | | | | | | | | |
| p227 | UCGTCGTGTCGTT-C3 | 227 | | | | | | | | | | |
| p228 | UCGTCGTGTCGTT-C3 | 228 | | | | | | | | | | |
| p229 | UCGTCGTGTCGTT-C3 | 229 | | | | | | | | | | |
| p230 | UCGTCGTGTCGTT-C3 | 230 | | | | | | | | | | |
| p231 | UCGTCgtgtCGTT-C3 | 231 | | | | | | | | | | |
| p232 | tcgtcgttttgtcgttttgtcgtT-C3 | 232 | | | | | | | | | | |
| | ucgtcgttttgtcgttttgtcgtT- | 233 | | | | | | | | | | |
| p233 | C3 | | | | | | | | | | | |
| p235 | tcgtcgttttgtcgtT-C3 | 235 | | | | | | | | | | |
| p236 | ucgtcgttttgtcgtT-C3 | 236 | | | | | | | | | | |
| p237 | tcgtcgtgtcgtT-C3 | 237 | | | | | | | | | | |
| p238 | ucgtcgtgtcgtT-C3 | 238 | | | | | | | | | | |
| p239 | UCgtCgtgtCgTT-C3 | 239 | | | | | | | | | | |
| p240 | UCgtCgtgtCgtt-C3 | 240 | | | | | | | | | | |
| p241 | UCgtcgtgtcgtt-C3 | 241 | | | | | | | | | | |
| p242 | Ucgtcgtgtcgtt-C3 | 242 | | | | | | | | | | |
| p243 | ucgtcgtgtcgtt-C3 | 243 | | | | | | | | | | |
| p244 | UCgtcgtgtcgtt-C3 | 244 | | | | | | | | | | |
| p245 | Ucgtcgtgtcgtt-C3 | 245 | | | | | | | | | | |
| p246 | ucgtcgtgtcgtt-C3 | 246 | | | | | | | | | | |
| | UCgtcgttttgtcgttttgtcgtt- | 247 | | | | | | | | | | |
| p247 | C3 | | | | | | | | | | | |
| p248 | Ucgtcgttttgtcgttttgtcgtt-C3 | 248 | | | | | | | | | | |
| p249 | ucgtcgttttgtcgttttgtcgtt-C3 | 249 | | | | | | | | | | |
| p250 | UCGTCgtgtCGTT-C3 | 250 | | | | | | | | | | |
| p251 | UCGTCgtgtCgtt-C3 | 251 | | | | | | | | | | |
| p252 | UCgTCgtgtCgtt-C3 | 252 | | | | | | | | | | |
| p253 | UCG′TCgtgtCGTT-C3 | 253 | | | | | | | | | | |
| p254 | UCGTCgtgtCG′TT-C3 | 254 | | | | | | | | | | |
| p255 | UCGTCgtgtCGT′T-C3 | 255 | | | | | | | | | | |
| p256 | UCGTCgtgtCGTT′-C3 | 256 | | | | | | | | | | |
| p257 | UCGTCgtgtCGT′T′-C3 | 257 | | | | | | | | | | |
| p258 | UCGTCgtgtCG′T′T′-C3 | 258 | | | | | | | | | | |
| p259 | UCGT′CgtgtCGTT-C3 | 259 | | | | | | | | | | |
| p260 | UCGTCgtgtCGTT′-C3 | 260 | | | | | | | | | | |
| p261 | UCGT′CgtgtCGTT′-C3 | 261 | | | | | | | | | | |
| p262 | Ucgucgtgtcgtt-C3 | 262 | | | | | | | | | | |
| p263 | Ucgtcgtgucgtt-C3 | 263 | | | | | | | | | | |
| p264 | TAACGACACGACGA | 264 | | | | | | | | | | |
| p265 | AACGACACGACGAT | 265 | | | | | | | | | | |
| p266 | ucgtcgtgucgtt-C3 | 266 | | | | | | | | | | |
| p267 | cgtcgtgtcgtt-C3 | 267 | | | | | | | | | | |
| p268 | cgtcgtgucgtt-C3 | 268 | | | | | | | | | | |
| p269 | Tcgtcgtgtcgtt-C3 | 269 | | | | | | | | | | |
| p270 | tcgtcgtgtcgtt-C3 | 270 | | | | | | | | | | |
| p271 | Ucgtcgtgtcgtt-C3 | 271 | | | | | | | | | | |
| p272 | ucgtcgtgtcgtt-C3 | 272 | | | | | | | | | | |
| p273 | ugctgctgtgctt-C3 | 273 | | | | | | | | | | |
| p274 | ucgagctgtcgtt-C3 | 274 | | | | | | | | | | |
| p275 | ucgtcgtgacgtt-C3 | 275 | | | | | | | | | | |
| p276 | ucgacgtgacgtt-C3 | 276 | | | | | | | | | | |
| p277 | acgacgtgacgtt-C3 | 277 | | | | | | | | | | |
| p278 | acgacgtgacgtt-C3 | 278 | | | | | | | | | | |
| p279 | ucngtcgtgtcgtt-C3 | 279 | | | | | | | | | | |
| p280 | ucgtcngtgtcgtt-C3 | 280 | | | | | | | | | | |
| p281 | ucgtcgtgtcngtt-C3 | 281 | | | | | | | | | | |
| p282 | ucngtcngtgtcngtt-C3 | 282 | | | | | | | | | | |
| p283 | acngacngtgacngtt-C3 | 283 | | | | | | | | | | |
| p284 | acngacngtgacngtt-C3 | 284 | | | | | | | | | | |
| p285 | ucgtcgtgtcgtT-OH | 285 | | | | | | | | | | |
| p286 | ucgtcgtgtcgtt-C3 | 286 | | | | | | | | | | |
| p287 | ucgtcgtgtcgtT | 287 | | | | | | | | | | |
| p288 | ucgtcgtgtcgtt-C3 | 288 | | | | | | | | | | |
| p289 | ucgtcgtgtcgtT | 289 | | | | | | | | | | |
| p290 | tcgtcgtgtcgtt-C3 | 290 | | | | | | | | | | |
| p291 | tcgtcgtgtcgtT | 291 | | | | | | | | | | |
| p292 | ucgtcgtgacgtt-C3 | 292 | | | | | | | | | | |
| p293 | ucgacgtgacgtt-C3 | 293 | | | | | | | | | | |
| p294 | tccatgucgttccttgatt-C3 | 294 | | | | | | | | | | |
| p295 | tccatgucgttccttt-C3 | 295 | | | | | | | | | | |
| p296 | tccatgucgttctt-C3 | 296 | | | | | | | | | | |
| p297 | tccatgucgtt-C3 | 297 | | | | | | | | | | |
| p298 | tucgtcgtgtcgtt-C3 | 298 | | | | | | | | | | |
| p299 | uucgtcgtgtcgtt-C3 | 299 | | | | | | | | | | |
| p300 | uucgtcgtgtcgtt-C3 | 300 | | | | | | | | | | |
| p301 | tcgucgtgtcgtt-C3 | 301 | | | | | | | | | | |
| p302 | tcgUcgtgtcgtt-C3 | 302 | | | | | | | | | | |
| p303 | tcgUcgtgtcgtt-C3 | 303 | | | | | | | | | | |
| p304 | ucgtcgtgacgtt-C3 | 304 | | | | | | | | | | |
| p305 | ucgacgtgacgtt-C3 | 305 | | | | | | | | | | |
| p306 | C3-PO-ucgtcgtgtcgtt-C3 | 306 | | | | | | | | | | |
| p307 | fucgtcgtgtcgtt-C3 | 307 | | | | | | | | | | |
| p308 | bucgtcgtgtcgtt-C3 | 308 | | | | | | | | | | |
| p309 | C3-PS-ucgtcgtgtcgtt-C3 | 309 | | | | | | | | | | |
| p310 | ucgtcgtgtcgtt-C3 | 310 | | | | | | | | | | |
| p311 | ucgtcgtgtcgtt-C3 | 311 | | | | | | | | | | |
| p312 | tcgucgtgtcgtt-C3 | 312 | | | | | | | | | | |
| p313 | tucgtcgtgacgtt-C3 | 313 | | | | | | | | | | |
| p314 | uucgtcgtgacgtt-C3 | 314 | | | | | | | | | | |
| p315 | NH2C6-ucgtcgtgacgtt-C3 | 315 | | | | | | | | | | |
| p316 | C3-uucgtcgtgacgtt-C3 | 316 | | | | | | | | | | |
| p317 | tcgacgtgucgtt-C3 | 317 | | | | | | | | | | |
| p318 | tcgacgtgacgtt-C3 | 318 | | | | | | | | | | |
| p319 | ucgacgtgucgtt-C3 | 319 | | | | | | | | | | |
| p320 | ucgtccatgacgtt-C3 | 320 | | | | | | | | | | |
| p321 | ucgtccatgucgtt-C3 | 321 | | | | | | | | | | |
| p322 | tcgtccatgucgtt-C3 | 322 | | | | | | | | | | |
| p323 | bucgtcgtgacgtt-C3 | 323 | | | | | | | | | | |
| p324 | catgucgttccttt-C3 | 324 | | | | | | | | | | |
| p325 | tgucgttccttt-C3 | 325 | | | | | | | | | | |
| p326 | tatgucgttccttt-C3 | 326 | | | | | | | | | | |
| p327 | tccatgacgttccttt-C3 | 327 | | | | | | | | | | |
| p328 | ugctgctgagctt-C3 | 328 | | | | | | | | | | |
| p329 | ugcagctgagctt-C3 | 329 | | | | | | | | | | |
| p330 | fTcgtcgtgtcgtt-C3 | 330 | | | | | | | | | | |
| p331 | ftcgtcgtgtcgtt-C3 | 331 | | | | | | | | | | |
| p332 | ucgtcgtgtcgtt-C3 | 332 | | | | | | | | | | |
| p333 | ucgtcgtgtcgtt-C3 | 333 | | | | | | | | | | |
| p334 | ucgtcgtgtcgtt-C3 | 334 | | | | | | | | | | |
| p335 | ucgtcgtgtcgtt-C3 | 335 | | | | | | | | | | |
| p336 | ucgtcgtgtcgtt-C3 | 336 | | | | | | | | | | |
| p337 | ucgtcgtgtcgtt-C3 | 337 | | | | | | | | | | |
| p338 | tatgugcttccttt-C3 | 338 | | | | | | | | | | |
| p339 | bucgttgggtcggt-C3 | 339 | | | | | | | | | | |
| p340 | bucgtcgtggcgtg-C3 | 340 | | | | | | | | | | |
| p341 | bucgtcgtgtcgtg-C3 | 341 | | | | | | | | | | |
| p342 | bucgtcgtggcgtt-C3 | 342 | | | | | | | | | | |
| p343 | bucgttgggtcgtt-C3 | 343 | | | | | | | | | | |
| p344 | bucgtcgtgtcgptpt-C3 | 344 | | | | | | | | | | |
| p345 | tugctgctgagctt-C3 | 345 | | | | | | | | | | |
| p346 | tugctgctgagctt-C3 | 346 | | | | | | | | | | |
| p347 | tugctgctgagctt-C3 | 347 | | | | | | | | | | |
| p348 | ucgtcgtgtcgtt-C3 | 348 | | | | | | | | | | |
| p349 | ucgtcgtgtcgtt-C3 | 349 | | | | | | | | | | |
| p350 | ucgtcgtgtcgtt-C3 | 350 | | | | | | | | | | |
| p351 | ucgtcgtgtcgtt-C3 | 351 | | | | | | | | | | |
| p352 | ucgtcgtgtcgtt-C3 | 352 | | | | | | | | | | |
| p353 | tucgtcgtgacgtt-C3 | 353 | | | | | | | | | | |
| p354 | tugctgctgagctt-C3 | 354 | | | | | | | | | | |
| p355 | ucgTcgtgtcgTt-C3 | 355 | | | | | | | | | | |
| p356 | ucgTcgtgtcgtt-C3 | 356 | | | | | | | | | | |
| p357 | ucgTcgtgtcgTt-C3 | 357 | | | | | | | | | | |
| p358 | ucgtcgtgtcgtt-C3 | 358 | | | | | | | | | | |
| p359 | ucgTcgtgtcgtt-C3 | 359 | | | | | | | | | | |
| p360 | ucgtcgtgtcgTt-C3 | 360 | | | | | | | | | | |
| p361 | ucgTcgtgtcgtt-C3 | 361 | | | 102 | | | | | | | |
| p362 | ucgTcgtgtcgTet-C3 | 362 | | | 175 | | | | | | | |
| p363 | ucgTcgtgtcGett-C3 | 363 | | | 365 | | | | | | | |
| p364 | ucgTcgtgtCegtt-C3 | 364 | | | 523 | | | | | | | |
| p365 | ucgTcgtgTecgtt-C3 | 365 | | | 260 | | | | | | | |
| p366 | ucgTcgtGetcgtt-C3 | 366 | | | 390 | | | | | | | |
| p367 | ucgTcgTegtcgtt-C3 | 367 | | | 287 | | | | | | | |
| p368 | ucgTcGetgtcgtt-C3 | 368 | | | 223 | | | | | | | |
| p369 | ucgTCegtgtcgtt-C3 | 369 | | | 242 | | | | | | | |
| p370 | ucGeTcgtgtcgtt-C3 | 370 | | | 158 | | | | | | | |
| p371 | uCegTcgtgtcgtt-C3 | 371 | | | 160 | | | | | | | |
| p372 | ucgTecgtgtcgTt-C3 | 372 | | | 194 | | | | | | | |
| p373 | tucgtcgtgacgttX5-C3 | 373 | | | | | | | | | | |
| p374 | tucgtcgtgacgtX5t-C3 | 374 | | | | | | | | | | |
| p375 | tucgtcgtgacgX5tt-C3 | 375 | | | | | | | | | | |
| p376 | tucgtcgtgacX5gtt-C3 | 376 | | | | | | | | | | |
| p377 | tucgtcgtgaX5cgtt-C3 | 377 | | | | | | | | | | |
| p378 | tucgtcgtgX5acgtt-C3 | 378 | | | | | | | | | | |
| p379 | tucgtcgtX5gacgtt-C3 | 379 | | | | | | | | | | |
| p380 | tucgtcgX5tgacgtt-C3 | 380 | | | | | | | | | | |
| p381 | tucgtcX5gtgacgtt-C3 | 381 | | | | | | | | | | |
| p382 | tucgtX5cgtgacgtt-C3 | 382 | | | | | | | | | | |
| p383 | tucgX5tcgtgacgtt-C3 | 383 | | | | | | | | | | |
| p384 | tucX5gtcgtgacgtt-C3 | 384 | | | | | | | | | | |
| p385 | tuX5cgtcgtgacgtt-C3 | 385 | | | | | | | | | | |
| p386 | tX5ucgtcgtgacgtt-C3 | 386 | | | | | | | | | | |
| p387 | X5tucgtcgtgacgtt-C3 | 387 | | | | | | | | | | |
| p388 | tucgx5cgtgacgtt-C3 | 388 | | | | | | | | | | |
| p389 | tucgx5cgtgacgtt-C3 | 389 | | | | | | | | | | |
| p390 | UecgTcgtgtcgtt-C3 | 390 | | | 533 | | | | | | | |
| p391 | UeCegTcgtgtcgtt-C3 | 391 | | | 1080 | | | | | | | |
| p392 | UeCeGeTcgtgtcgtt-C3 | 392 | | | 1691 | | | | | | | |
| p393 | ucgTcgtgtCeGeTeTe-C3 | 393 | | | 2211 | | | | | | | |
| | UeCeGeTcgtgtCeGeTeT | 394 | | | | | | | | | | |
| p394 | e-C3 | | | | inact. | | | | | | | |
| | UeCeGeTCeGeTeGeTe | 395 | | | | | | | | | | |
| p395 | CeGeTeTe-C3 | | | | | | | | | | | |
| p396 | uCegTCegtgtCegtt-C3 | 396 | | | 704 | | | | | | | |
| p397 | ucgTcGetGetcGett-C3 | 397 | | | 3494 | | | | | | | |
| p398 | ucgTcgTegTecgTet-C3 | 398 | | | 2423 | | | | | | | |
| p399 | uCegTcgTegTecgTet-C3 | 399 | | | 4261 | | | | | | | |
| p400 | ucgTecgTegTecgTt-C3 | 400 | | | 1805 | | | | | | | |
| p401 | uCegTecgTegTecgTt-C3 | 401 | | | 2509 | | | | | | | |
| p402 | uCegTicgtgtcGett-C3 | 402 | | | 356 | | | | | | | |
| p403 | uCegTcgtgtCegtt-C3 | 403 | | | 482 | | | | | | | |
| p404 | uCegTcgttgtcgTet-C3 | 404 | | | 203 | | | | | | | |
| p405 | uCegTcgtTegtcgTet-C3 | 405 | | | 809 | | | | | | | |
| p406 | uCegTcgTetgtcgTet-C3 | 406 | | | 510 | | | | | | | |
| p407 | uCegTcgtX3gtcgTet-C3 | 407 | | | 286 | | | | | | | |
| p408 | uCegTcgX3tgtcgTet-C3 | 408 | | | 266 | | | | | | | |
| p409 | uCegTcgtTegtcgTet-C3 | 409 | | | 875 | | | | | | | |
| p410 | uCegTcgtX3gtcgTet-C3 | 410 | | | 193 | | | | | | | |
| p411 | X3ucgTcgtgtcgtt-C3 | 411 | | | 124 | | | | | | | |
| p412 | uX3cgTcgtgtcgtt-C3 | 412 | | | inact. | | | | | | | |
| p413 | ucX3gTcgtgtcgtt-C3 | 413 | | | 225 | | | | | | | |
| p414 | ucgX3Tcgtgtcgtt-C3 | 414 | | | 131 | | | | | | | |
| p415 | ucgTX3cgtgtcgtt-C3 | 415 | | | 124 | | | | | | | |
| p416 | ucgTcX3gtgtcgtt-C3 | 416 | | | 85 | | | | | | | |
| p417 | ucgTcgX3tgtcgtt-C3 | 417 | | | 92 | | | | | | | |
| p418 | ucgTcgtX3gtcgtt-C3 | 418 | | | 93 | | | | | | | |
| p419 | ucgTcgtgX3tcgtt-C3 | 419 | | | 189 | | | | | | | |
| p420 | ucgTcgtgtX3cgtt-C3 | 420 | | | 227 | | | | | | | |
| p421 | ucgTcgtgtcX3gtt-C3 | 421 | | | 95 | | | | | | | |
| p422 | ucgTcgtgtcgX3tt-C3 | 422 | | | 135 | | | | | | | |
| p423 | ucgTcgtgtcgtX3t-C3 | 423 | | | 202 | | | | | | | |
| p424 | ucgTcgtgtcgttX3-C3 | 424 | | | 113 | | | | | | | |
| p425 | bucgTcgtgtcgtt-C3 | 425 | | | | | | | | | | |
| p426 | [symbol] cgTcgtgtcgtt-C3 | 426 | | | | | | | | | | |
| p427 | [symbol] cgTcgtgtcgtt-C3 | 427 | | | | | | | | | | |
| p428 | [symbol] cgTcgtgtcgtt-C3 | 428 | | | | | | | | | | |
| p429 | oducgTcgtgtcgtt-c3 | 429 | | | | | | | | | | |
| p430 | o [symbol] cgTcgtgtcgtt-c3 | 430 | | | | | | | | | | |
| p431 | ods [symbol] cgTcgtgtcgtt-c3 | 431 | | | | | | | | | | |
| p432 | s [symbol] cgTcgtgtcgtt-c3 | 432 | | | | | | | | | | |
| p433 | bu^{r}cgTcgtgtcgtt-C3 | 433 | | | 96 | | | | | | | |
| p434 | bu^{s}cgTcgtgtcgtt-C3 | 434 | | | 125 | | | | | | | |
| p435 | buc^{r}gTcgtgtcgtt-C3 | 435 | | | 148 | | | | | | | |
| p436 | buc^{s}gTcgtgtcgtt-C3 | 436 | | | 112 | | | | | | | |
| p437 | bu^{s}c^{r}gTcgtgtcgtt-C3 | 437 | | | | | | | | | | |
| p438 | bu^{s}c^{s}gTcgtgtcgtt-C3 | 438 | | | | | | | | | | |
| p439 | buCegTcgtgtcgtt-C3 | 439 | | | | | | | | | | |
| p440 | buCegTcgtgtCegtt-C3 | 440 | | | | | | | | | | |
| p441 | buCegTCegtgtCegtt-C3 | 441 | | | | | | | | | | |
| p442 | buCegTcgtgtcgTet-C3 | 442 | | | | | | | | | | |
| p443 | buCegTcgTegtcgTet-C3 | 443 | | | | | | | | | | |
| | Biotin- | | | | | | | | | | | |
| p444 | AfAfCfGfAfCfAfCfGfAfCf | 444 | | | | | | | | | | |
| | GfAf | | | | | | | | | | | |
| p445 | buCsigTcgtgtcgtt-c3 | 445 | | | | | | | | | | |
| p446 | bucgTcgtgtcgTsit-c3 | 446 | | | | | | | | | | |
| p447 | buCsigTCsigtgtCsigtt-c3 | 447 | | | | | | | | | | |
| p448 | buCsigTcgtgtcgTsit-c3 | 448 | | | | | | | | | | |
| p449 | buCsigTcgTsigtcgTsit-c3 | 449 | | | | | | | | | | |
| p450 | t [symbol] cgtcgtgacgtt-c3 | 450 | | | | | | | | | | |
| p451 | t [symbol] cgacgtgacgtt-c3 | 451 | | | | | | 60 | | | 18 | 100 |
| p452 | t [symbol] cgacgtt-c3 | 452 | | | | | | inact. | | | 100 | inact. |
| p453 | t [symbol] acgtt-c3 | 453 | | | | | | inact. | | | inact. | inact. |
| p454 | tacgtt-c3 | 454 | | | | | | inact. | | | inact. | inact. |
| p455 | t [symbol] cgtt-c3 | 455 | | | | | | inact. | | | inact. | inact. |
| p456 | tacgt-c3 | 456 | | | | | | inact. | | | inact. | inact. |
| p457 | t [symbol] cgt-c3 | 457 | | | | | | inact. | | | inact. | inact. |
| p458 | t [symbol] cg [symbol] cgtgacgtt-c3 | 458 | | | | | | | | | 36 | 119 |
| p459 | t [symbol] cg [symbol] cgtl-c3 | 459 | | | | | | | | | 132 | inact. |
| p460 | t [symbol] acg [symbol] t-c3 | 460 | | | | | | | | | inact. | inact. |
| p461 | tacg [symbol] t-c3 | 461 | | | | | | | | | inact. | inact. |
| p462 | t [symbol] cg [symbol] t-c3 | 462 | | | | | | | | | 223 | inact. |
| p463 | g [symbol] cgti-c3 | 463 | | | | | | | | | inact. | inact. |
| p464 | gacggtt-c3 | 464 | | | | | | | | | inact. | inact. |
| p465 | g [symbol] cg [symbol] t-c3 | 465 | | | | | | | | | inact. | inact. |
| p466 | gacg [symbol] t-c3 | 466 | | | | | | | | | inact. | inact. |
| p469 | tbucgtcgtgacgtt-c3 | 469 | | | | | | | | | | |
| p470 | bucgTcgtgtcg-c3 | 470 | | | | | | | | | | |
| p471 | bucgTcgtgt-c3 | 471 | | | | | | | | | | |
| p472 | bucgtcgtgT-c3 | 472 | | | | | | | | | | |
| p473 | bucgTcgt-c3 | 473 | | | | | | | | | | |
| p474 | bucgtcgT-c3 | 474 | | | | | | | | | | |
| p475 | bucgTt-c3 | 475 | | | | | | | | | | |
| p476 | bucgtT-c3 | 476 | | | | | | | | | | |
| p477 | t [symbol] cgtcgtgacgtmtm-c3 | 477 | | | | | | | | | | |
| p478 | tmtm [symbol] cgtcgtgacgtt-c3 | 478 | | | | | | | | | | |
| p479 | tmtm [symbol] cgtcgtgacgtmtm-c3 | 479 | | | | | | | | | | |
| p480 | t [symbol] cgtcgtgacgt(m)t(m)-c3 | 480 | | | | | | | | | | |
| p481 | t(m)t(m) [symbol] cgtcgtgacgtt-c3 | 481 | | | | | | | | | | |
| p482 | t [symbol] c^{r}gtcgtgacgtt-c3 | 482 | | | | | | 399 | | | | |
| p483 | t [symbol] c^{s}gtcgtgacgtt-c3 | 483 | | | | | | 577 | | | | |
| p484 | t [symbol] ^{r}cgtcgtgacgtt-c3 | 484 | | | | | | 410 | | | | |
| p485 | t [symbol] ^{s}cgtcgtgacgtt-c3 | 485 | | | | | | 245 | | | | |
| | t(m)t(m)ucgtcgtgacgt(m)t | | | | | | | | | | | |
| p486 | (m)-c3 | 486 | | | | | | | | | | |
| p487 | bucgtcgtgtcgtt(m)-c3 | 487 | | | | | | | | | | |
| p488 | bucgtcgtgtcgt(m)t(m)-c3 | 488 | | | | | | | | | | |
| p489 | bucgtcgtgtcgt(m)T-c3 | 489 | | | | | | | | | | |

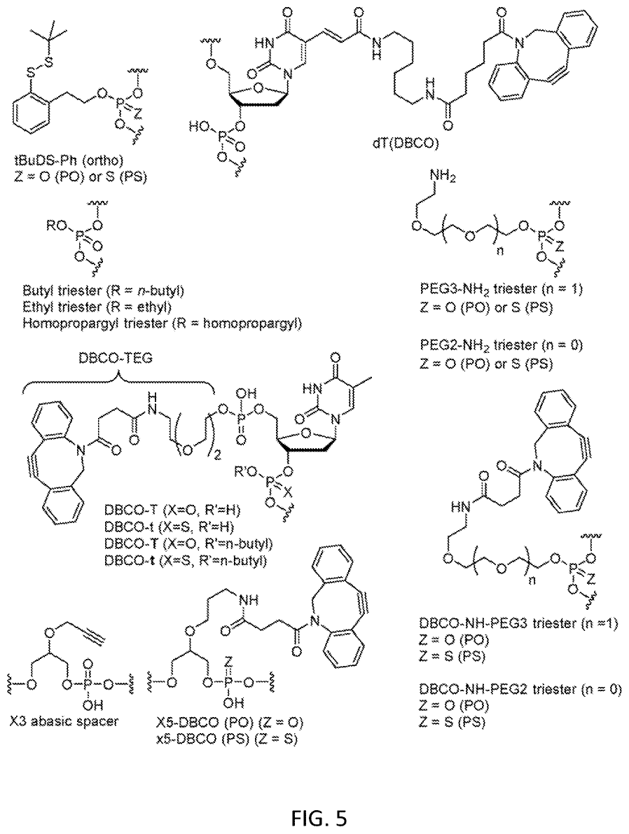

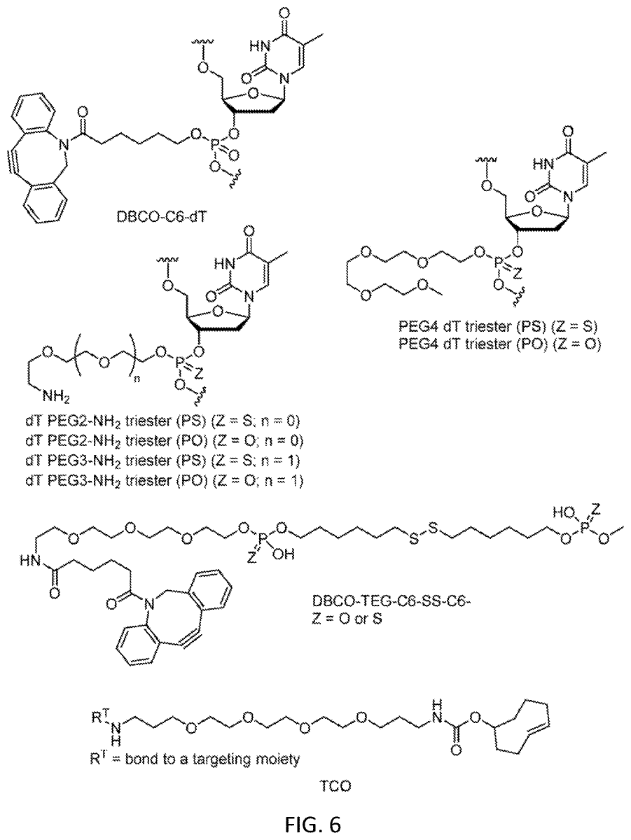

In table 2, column A provides IL-6 expression in DB cells (EC50, nM); column B provides IL-10 expression in DB cells (EC50, nM); column C
provides NFκB activation in Ramos blue cells (EC50, nM); column D provides NFκB activation Hela-hTLR9-NFκB-luc cells (EC50, nM); column E
provides NFκB activation Hela-mTLR9-NFκB-luc cells (EC50, nM); column F provides IL-6 secretion in mouse splenocytes (EC50, nM); column G
provides IL-6 secretion in mouse splenocytes after 24 h preincubation in 95% mouse plasma (EC50, nM); column H provides IL-6 secretion in
mouse bone marrow differentiated DC (EC50, nM); Column I provides NFκB activation in mouse HEK-Blue cells after 2h transfection with RNAiMax
(EC50, nM); and Column J provides NFκB activation in human HEK-Blue cells after 2h transfection with RNAiMax (EC50, nM).
The key descriptors for the sequences provided throughout the Tables included herein are as follows: lower case = nucleoside-3′-
phosphorothioate; UPPER CASE = nucleoside-3′-phosphate; italics lower case = nucleoside having a 3′ tBuDS-Ph (ortho) triester (PS); ITALICS
UPPER CASE = nucleoside having a 3′ tBuDS-Ph (ortho) triester (PO); dt = dT(DBCo); bold double underlined t = DBCO-C6-dT; bold lower
case = nucleoside having a 3′ n-butyl triester (PS); BOLD UPPER CASE = nucleoside having a 3′ n-butyl triester (PO); italic bold lower case =
nucleoside having a 3′ homopropargyl triester (hPro)(PS); italic underlined lower case = nucleoside having a 3′ DBCO-NH-PEG2 triester (N1)
(PS); ITALIC UNDERLINED UPPER CASE = nucleoside having a 3′ DBCO-NH-PEG2 triester (N1)(PO); double underlined t = dT PEG2-NH2
triester (PS); double underlined T = dT PEG2-NH2 triester (PO); italic double underlined lower case = nucleoside having a 3′ PEG2-NH2 triester
(N1)(PS); ITALIC DOUBLE UNDERLINED UPPER CASE = nucleoside having a 3′ PEG2-NH2 triester (N1)(PO); BOLD ITALIC UNDERLINED
UPPER CASE U = 5-iodo-2′-deoxyuridine (PO); bold italic underlined lower case u = 5-iodo-2′-deoxyuridine (PS); BOLD UNDERLINED = 2′-
fluoronucleotide (PO); an apostrophe indicates that the nucleotide identified by a letter to the left of the apostrophe contains a 2′-OMe-modified
ribose; underlined ng = 7-deaza-2′-deoxyguanosine (PS); underlined pT = PEG4 dT triester (PO); underlined pt = PEG4 dT triester (PS); fT = 5-
trifluoromethyl-thymidine (PO); fU = 5-fluoro-2′-deoxyuridine (PO); bU = 5-bromo-2′-deoxyuridine (PO); ft = 5-trifluoromethyl-thymidine (PS); fu =
5-fluoro-2′-deoxyuridine (PS); bu = 5-bromo-2′-deoxyuridine (PS); C3 = C3 spacer (-(CH2)3-OH)(PO); c3 = C3 spacer (-(CH2)3-OH)(PS); C6 =
hexane-1,6-diyl; NH2C6 = 6-aminohex-1-yl; Te = thymidine having a 3′ ethyl triester (PO); Ge = guanosine having a 3′ ethyl triester (PO); Ce =
cytidine having a 3′ ethyl triester (PO); Ue = 5-iodouridine having a 3′ ethyl triester (PO); ue = 5-iodouridine having a 3′ ethyl triester (PS); iu = 5′-
5′ cap based on 5-iodo-2′-deoxyuridine (PS); iU= 5′-5′ cap based on 5-iodo-2′-deoxyuridine (PO); X5 = X5-DBCO (PO); x5 = x5-DBCO (PS); X3 =
X3 abasic spacer (PO); and IR700 is a dye. Here, the descriptor (PO) stands for 3′-phosphate; and (PS) stands for 3′-phosphorothioate; od = 5′-
orthodisulfide phosphodiester; o = 5′-phosphate (PO); ods = 5′-orthodisulfide phosphorothioate; s = 5′-phosphorothioate (PS); superscript “r” = Rp
PS; superscript “s” = Sp PS; Af = 2′-fluoro-adenosine (PO); Csi = dC O-silyltriester (PO); Tsi = dT O-silyltriester (PO); tm = 2′-OMe thymidine
(PS); t(m) = 2′-OMOE thymidine (PS). Structures are shown in FIGS. 5 and 6.

### Double-Stranded CpGs:

### Annealing and Gel Analysis:

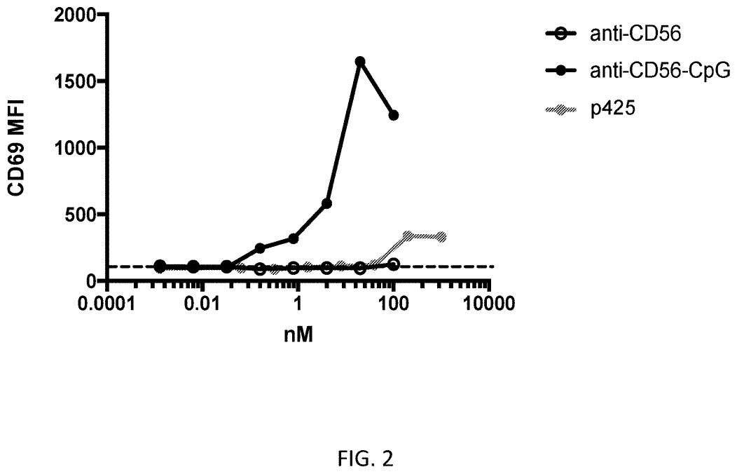

Polynucleotide p88 (1 mL, 5 mM stock) was added to p144 (3.3 mL, 2 mM stock) with DPBS (24.7 mL). Polynucleotide p88 was treated with p145 in a similar manner. The mixtures were heated to 65° C. for 10 min. Analysis by TBE urea gel showed complete annealing of the p88 (see FIG. 2). 1 μL of each sample was removed, added to 5 μL of formamide loading buffer, and loaded per well onto a 15% TBE-urea gel, 200 volts for 40 min followed by ethidium bromide (EtBr) staining. See Table 2 for structures of p88, p144, and p145.

Double stranded-CpG using p88/p144—Representative example (1):

(SEQ ID NO: 234)
TCGTCGTTTTGTCGTTTTGTCGTT
(SEQ ID NO: 468)
AGCAGCAAAACAGCAAAACAGCAA

Double stranded-CpG using p88/p145—Representative example (2):

(SEQ ID NO: 467)
TCGTCGTTTTGTCGTTTTGTCGTT
(SEQ ID NO: 468)
AGCAGCAAAACAGCAAAACAGCAA

### Example 2: Preparation of Antibody-CpG Conjugates

### A. Preparation of Anti-SIRPα Antibody-CpG Nucleotide Conjugates

Two anti-SIRPα antibodies were selected. One of the anti-SIRPα antibodies blocks the binding of CD47 and its epitope overlaps with the binding site of CD47 (blocking). The other anti-SIRPα antibody binds to an epitope distinct from the binding site of CD47 (non-blocking). See WO 2018/057669, the disclosure of which is incorporated herein by reference in its entirety. The anti-SIRPα antibodies were conjugated via a transglutaminase (“mTGase”) reaction.

The VH and VL of the blocking antibody, anti-SIRPα 1, selected for conjugation is

(SEQ ID NO: 521)
EVQLVESGGGVVQPGGSLRLSCAASGFTFSSNAMSWVRQAPGKGLEWVAG
ISAGGSDTYYPASVKGRFTISRDNSKNTLYLQMNSLRAEDTAVYYCARET
WNHLFDYWGQGTLVTVSS
and
(SEQ ID NO: 525)
SYELTQPPSVSVSPGQTARITCSGGSYSSYYYAWYQQKPGQAPVTLIYSD
DKRPSNIPERFSGSSSGTTVTLTISGVQAEDEADYYCGGYDQSSYTNPFG
GGTKLTVL,

respectively.

The VH and VL of the non-blocking antibody, anti-SIRPα 2, selected for conjugation is

(SEQ ID NO: 548)
EVQLVESGGGVVQPGRSLRLSCAASGFTFSSYDVNWVRQAPGKGLEWVS
LISGSGEIIYYADSVKGRFTISRDNSKNTLYLQMNSLRAEDTAVYYCAK
ENNRYRFFDDWGQGTLVTVSS
and
(SEQ ID NO: 553)
ETVLTQSPGTLSLSPGERATLSCRASQSVYTYLAWYQQKPGQAPRLLIY
GASSRATGIPDRFSGSGSGTEFTLTISSLQSEDFAVYYCQQYYDRPPLT
FGGGTKVEIK,

respective.

An anti-SIRPα antibody carrying an N297 A mutation in human IgG1 was buffer exchanged to 25 mM Tris, 150 mM NaCl, pH 8 using a desalting column. To the SIRPα antibody solution was added mTGase and N₃—PEG23-NH₂ linker having the structure of N₃—CH₂CH₂(OCH₂CH₂)₂₃—NH₂. Given the N297 A mutation, conjugation can occur via the side chain of glutamine 295 (EU numbering). The resulting mixture was left at room temperature overnight. In the mTGase reaction mixture, the final antibody concentration was 50 μM; the ratio of the antibody to mTGase was about 10, and the ratio of the linker to the antibody was about 5. The mTGase and free PEG linker were removed by Protein A purification. The modified antibody was buffer exchanged into 1×PBS using a desalting column. Subsequent Huisgen cycloaddition of an alkyne in a CpG nucleotide of SEQ ID NO: 425 with an azido group in the modified antibody furnished an anti-SIRPα-CpG nucleotide conjugate having the structure of Formula (D) or (E):

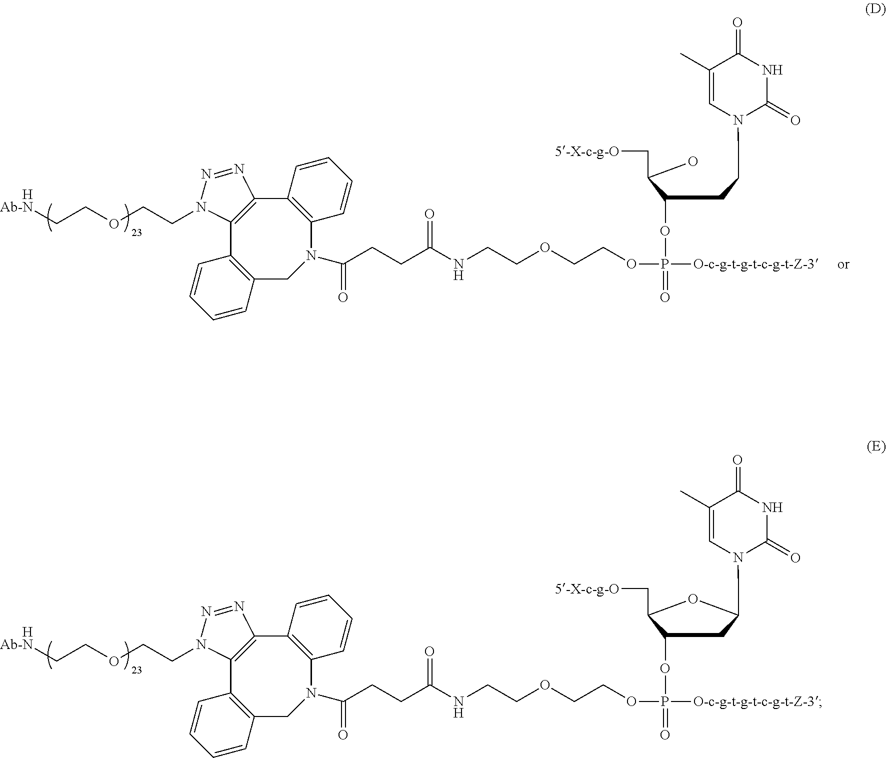

wherein Ab is a blocking or a non-blocking anti-SIRPα antibody; c is 2′-deoxycytidine; g is 2′-deoxyguanosine; t is thymidine; X is 5-bromo-2′-deoxyuridine; and Z is

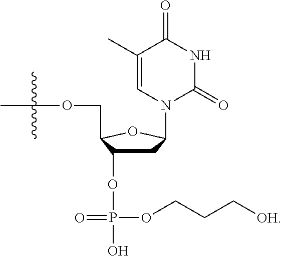

### B. Preparation of an Anti-CD56 Antibody-CpG Nucleotide Conjugate

The murine monoclonal anti-CD56 antibody (clone 5.1 H11) was obtained commercially. The anti-CD56 antibody was conjugated through an activated pentafluorophenyl (PFP) ester. To a solution of the anti-CD56 antibody in Dulbecco's phosphate-buffered saline (DPBS) buffer (˜2.5 μg/μL) was added an azido-PEG8-PFP ester having the structure of N₃—CH₂CH₂(OCH₂CH₂)₈—CH₂CO—PFP with a ratio of the linker to the antibody of 20. The resulting mixture was left overnight at room temperature to form an azido-containing antibody. The excess azido-PEG8-PFP was then removed by buffer exchanging through an Amicon 30kD spin concentrator using DPBS as an eluent. Subsequent Huisgen cycloaddition of an alkyne in a CpG nucleotide of SEQ ID NO: 425 with an azido group in the modified antibody furnished an anti-CD56-CpG nucleotide conjugate having the structure of Formula (F) or (G):

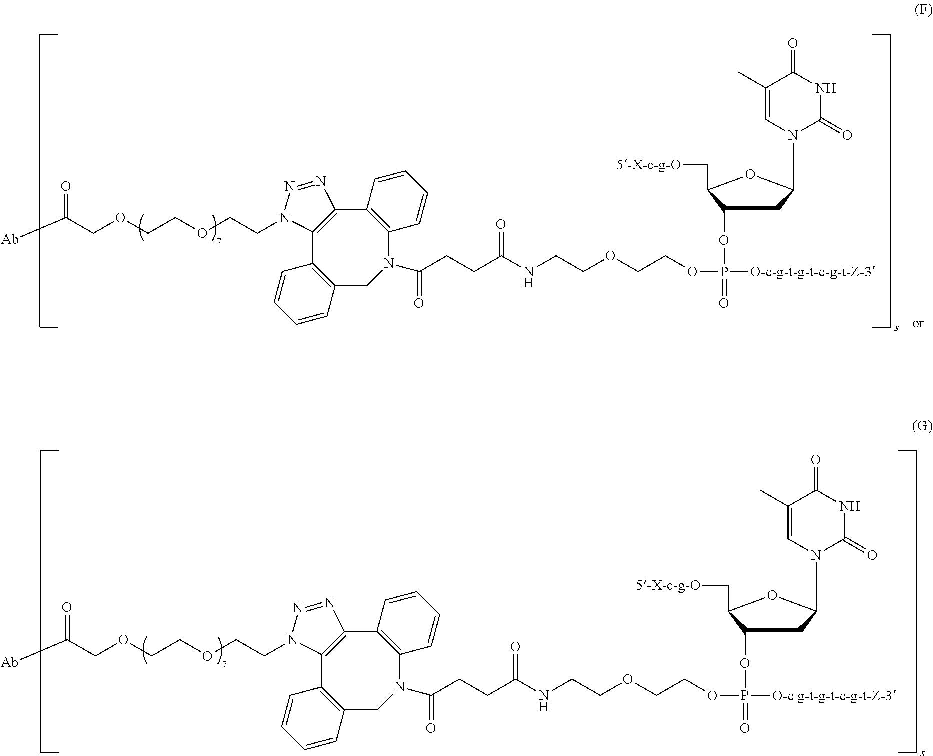

wherein Ab is an anti-CD56 antibody; s is an integer of about 3 or about 4; c is 2′-deoxycytidine; g is 2′-deoxyguanosine; t is thymidine; X is 5-bromo-2′-deoxyuridine; and Z is

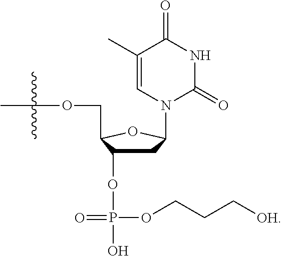

### Example 3. Biological Evaluation of Antibody-CpG Nucleotide Conjugates

Trima residuals were received from Blood Centers of the Pacific and diluted 1:4 with Phosphate Buffered Saline (PBS). The diluted blood was split into four tubes and underplayed with 15 mL Ficoll® Paque density gradient media (GE Healthcare Life Sciences). The tubes were centrifuged for 30 minutes at 400×g. PBMC₅ were collected from the interface and resuspended in a FACS buffer (PBS with 0.5% Bovine Serum Albumin). CD14⁺ monocytes were purified by negative selection using the Monocyte Isolation Kit II (Miltenyi Biotec) and LS columns (Miltenyi Biotec) according to manufacturer's protocol.

PBMCs or CD14⁺ cells were immediately plated onto a 96-well format (500K/well) in complete Roswell Park Memorial Institute medium (RPMI). Five-fold serial dilutions were added to the cells from 100 nM to 6.4 pM of an antibody and an antibody-CpG nucleotide conjugate and 1 μM to 64 pM of CpG polynucleotide of SEQ ID NO: 425 at 37° C. under 5% CO₂ for 24 or 48 hours. Cells were pelleted by centrifugation for five minutes at 400×g and stained at 4° C. in 100 μL Fixable Viability Dye eFluor 780 (Thermo Fisher) diluted 1:2000 in PBS. Cells were centrifuged and stained at 4° C. for 30 minutes in 100 μL FACS buffer for 30 minutes containing 2 μL FcγR Blocking Reagent, 1.25 μL anti-CD14, anti-CD3, anti-CD19 for anti-SIRPα assays and anti-CD56, anti-CD16, anti-CD69, anti-CD14, anti-CD3 and anti-CD19 for anti-CD56 assays. Cells were centrifuged and washed twice in 200 μL FACS buffer and fixed in 100 μL 0.5% paraformaldehyde. CountBright Absolute Counting Beads were added to each well to count the number of cells. Cells were analyzed on Attune NxT Flow Cytometer with subsequent data analysis by Flowjo 10.7. Dead cells were excluded by gating on the eFluor 780-negative population. Lineage specific cells were first excluded (CD19, CD3) prior to gating CD14⁺ cells and (CD19, CD3, CD14) prior to gating CD56⁺CD16⁺ cells.

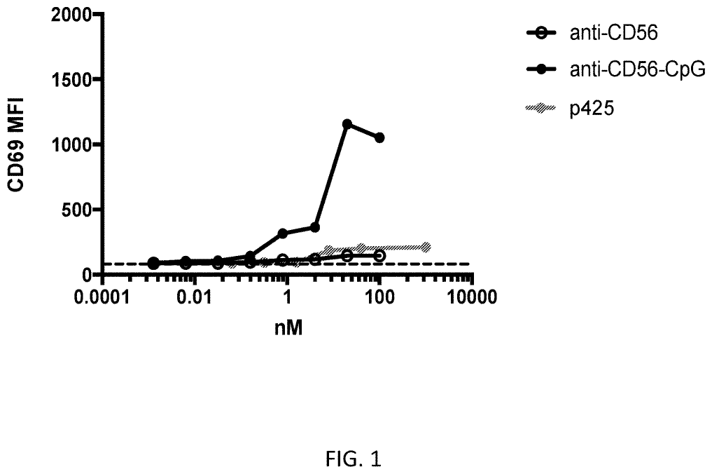

As shown in FIGS. 1 and 2, the anti-CD56-CpG nucleotide conjugate enhanced the activation of NK cells as compared to the CpG nucleotide and anti-CD56 alone at 24 and 48 hours, respectively. As determined by CD69 expression, the anti-CD56-CpG nucleotide conjugate was able to induce NK cell activation.

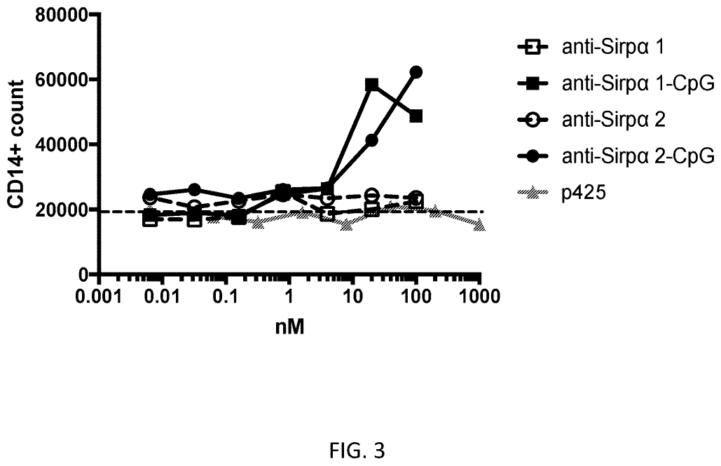

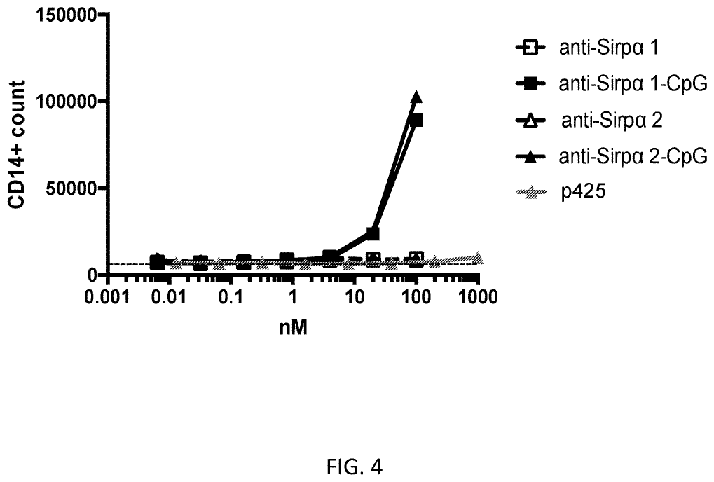

As shown in FIGS. 3 and 4, the anti-SIRPα-CpG nucleotide conjugates with either a blocking antibody (anti-SIRPα 1) or non-blocking antibody (anti-SIRPα 2) induced the proliferation of CD14⁺ monocytes within whole PBMC and purified CD14⁺ population. Using counting beads to determine the absolute number of CD14⁺ monocytes, the anti-SIRPα-CpG nucleotide conjugates with either a blocking antibody (anti-SIRPα 1) or non-blocking antibody (anti-SIRPα 2) induced proliferation as compared to CpG nucleotide and anti-SIRPα antibodies alone. The experiments using purified CD14⁺ monocytes show that the increase in cell numbers is the result of delivering the CpG nucleotide into the cells by the anti-SIRP antibody. These data collectively indicate that anti-SIRPα antibodies binding to different epitopes, blocking and non-blocking, are shown to deliver CpG nucleotide into CD14⁺ monocytes and result in its expansion in cell numbers.

### Example 4: Determination of K_{D}

The interactions of anti-SIRPα antibodies with SIPRα from various species (human v1, human v2, cynomolgus, mouse 129, BL6, BALBc, NOD), SIRPβ, and SIRPywere analyzed using two methods, direct immobilization of the antibodies (via a GLC chip) according to the following protocols. All experiments were performed at 25° C. using a SPR-based ProteOn XPR36 biosensor (BioRad, Inc., Hercules, Calif.) equipped with GLC or NLC sensor chips. Antibodies were expressed using FREESTYLE™ 293-FS cells (Thermo Fisher). Purification was carried out by standard protein A affinity column chromatography and eluted antibodies were stored in a PBS buffer.

The running buffer was PBS pH 7.4 with 0.01% TWEEN-20 (PBST+). All analytes were used at their nominal concentrations as determined by A280 absorbance and using their molar calculated extinction coefficient. Analytes were injected in a “one-shot” kinetic mode as described (see, e.g., Bravman et al., Anal. Biochem. 2006, 358, 281-288).

For the method using a GLC chip, the analytes were injected and flowed over anti-SIRPα antibodies immobilized (˜1000 RUs) on GLC chips using Proteon Amine Coupling Kit. For the immobilization step, the GLC chip was activated with EDAC/Sulpho-NHS 1:1 (Biorad) diluted 1/100 for 300 s at 25 μL/min. Anti-SIRPα antibodies were diluted to 80 nM concentration in 10 mM sodium acetate buffer pH 4.5 and immobilized to the chip at 30 μL/min for 50 s. Chip was inactivated with ethanolamine for 300 s at 25 μL/min. The analytes (e.g., SIRP-α from different species, SIRP-β, SIRP-γ) were injected in a “one-shot” kinetic mode at nominal concentrations of 100, 33, 11, 3.7, 1.2, and 0 nM. Association times were monitored for 90 s at 100 uL/min, and dissociation times were monitored for 1200 s. The surfaces were regenerated with a 2:1 v/v blend of Pierce IgG elution buffer/4M NaCl.

Biosensor data were double-referenced by subtracting the interspot data (containing no immobilized protein) from the reaction spot data (immobilized protein) and then subtracting the response of a buffer “blank” analyte injection from that of an analyte injection. Double-referenced data were fit globally to a simple Langmuir model and the K_{D} value was calculated from the ratio of the apparent kinetic rate constants (K_{D}=k_{d}/kₐ).

Binding kinetics of blockers 119, 135 and non-blocker 136 human antibodies to various SIRP-α from different species, SIRP-β, SIRP-γ) were determined. These antibodies bind with high affinities to SIRP-α from human v1, human v2, and cynomologous monkey. They do not bind to various mouse SIRP-α. However, they exhibited high affinity binding to human SIRP-β and human SIRP-γ. Therefore, these antibodies will be useful pan anti-SIRPs for conjugation and delivering of CpG immunomodulating polynucleotide to modulate the activities of various myeloid cell populations. Results are summarized in Table 3.

Binding kinectics of humanized AB21 blocking antibodies to various SIRP-α from different species, SIRP-β, SIRP-7) were determined. AB21 antibodies bind with high affinities to SIRP-α from human v1, human v2, cynomologous monkey, various mouse SIRP-α (NOD, BL6, and BALBc), human SIRP-β and human SIRP-γ. Therefore, the AB21 blocking antibodies will be useful pan anti-SIRPs for conjugation and delivering of CpG immunomodulating polynucleotide to modulate the activities of various myeloid cell populations. Results are summarized in Table 4.

TABLE 3
Affinities of Anti-SIRP-α Antibody Germline/Liability Mutation

| | Human | Human | | K_{D} (nM) | | | | | | | |
|---|---|---|---|---|---|---|---|---|---|---|---|
| | Light | Heavy | Type of | Human | Human | | Mouse | Mouse | Mouse | Human | Human |
| Antibody | Chain | Chain | Binding | v1 | v2 | Cyno | NOD | BL6 | BALBc | SIRPβ | SIRPγ |
| 119 | wt | wt | B | 0.18 | 0.068 | 0.11 | NLB | NLB | NLB | 0.34 | 0.27 |
| 119 | Mut | wt | B | 0.21 | 0.086 | 0.14 | NLB | NLB | NLB | NT | 0.23 |
| 119 | wt | Mut | B | 0.25 | 0.069 | 0.16 | NLB | NLB | NLB | NT | NT |
| 119 | Mut | Mut | B | 0.32 | 0.088 | 0.20 | NLB | NLB | NLB | 0.46 | 0.34 |
| 119 | Mut | Mut_V34M | B | 0.22 | 0.069 | 0.12 | NLB | NLB | NLB | 0.34 | 0.26 |
| 135 | wt | wt | B | 0.15 | 0.029 | 0.097 | NLB | NLB | NLB | | 0.54 |
| 135 | Mut | wt | B | 0.15 | 0.027 | 0.10 | NLB | NLB | NLB | | 0.52 |
| 135 | wt | Mut | B | 0.18 | 0.02 | 0.13 | NLB | NLB | NLB | | 0.78 |
| 135 | Mut | Mut | B | 0.19 | 0.018 | 0.13 | NLB | NLB | NLB | 0.19 | 0.73 |
| 135 | wt | Mut_V34M | B | 0.15 | 0.016 | 0.080 | NLB | NLB | NLB | 0.16 | 0.53 |
| 136 | wt | wt | NB | 0.46 | 1.6 | 2.2 | 0.55 | 13 | 0.35 | 4.4 | 24 |
| 136 | Mut | wt | NB | 7.3 | 17 | 11 | 4.1 | 33 | 2.8 | | 20 |
| 136 | wt | Mut | NB | 0.56 | 1.7 | 2.3 | 0.68 | 23 | 0.42 | 3.5 | 17 |
| 136 | Mut | Mut | NB | 7.3 | 20 | 13 | 5.2 | 32 | 3.1 | | 1.7 |
| 136 | Mut_I2T | Mut | NB | 0.72 | 1.8 | 2.3 | 0.68 | 15 | 0.42 | 3.2 | 40 |
| 136 | Mut_S12T | Mut | NB | 4.9 | 11 | 8.0 | 2.9 | 16 | 2.1 | | 67 |
| 136 | Mut_S22T | Mut | NB | 5.0 | 8.3 | 6.8 | 2.4 | 13 | 1.6 | | 62 |
| 136 | Mut_Q38E | Mut | NB | 6.2 | 13 | 10 | 6.0 | 33 | 3.0 | | 19 |
| 136 | Mut_I2T | Mut_V34M | NB | 0.55 | 17 | 2.1 | 0.60 | 14 | 0.36 | 2.2 | 34 |

B = blocker; NB = non-blocker.
NT or blank = not tested;
NA = not applicable (antibodies do not cross-react);
NLB = no binding
119 heavy chain mut = D1E, E43K, L112Q, M34V
119 light chain mut = F21L, R39K, E60A, T76S
135 heavy chain mut = D1E, R13Q, E16G, M34V, E43K, L112Q
135 light chain mut = F21L, D60A
136 heavy chain mut = D1E, R13Q, E16R, M34V, E43K, L111Q
136 light chain mut = T2I, T12S, T22S, E38Q

TABLE 4
Affinities of Anti-SIRP-α Antibody

| | | | K_{D} | | | | | | | | |
|---|---|---|---|---|---|---|---|---|---|---|---|
| Antibody | | | Human | Human | | Mouse | Mouse | Mouse | | Human | Human |
| Designation | VL | VH | V1 | V2 | Cyno | NOD | BL6 | BALBc | | SIRPβ | SIRPγ |
| Hum1/ | Hum1 | Human | 5.3 pM | 4.6 pM | 29 pM | 3.7 nM | 9.5 nM | 7.9 | nM | 6.7 pM | 1.0 pM |
| AB21_HC_Mutall | Humanized | (AB21_HC_Mutall) | | | | | | | | | |
| Hum8/ | Hum8 | Human | 20 pM | NT | NT | 28 nM | 0.42 mM | 71 | nM | NT | NT |
| AB21_HC_Mutall | Humanized | (AB21_HC_Mutall) | | | | | | | | | |
| Hum9/ | Hum9 | Human | 12 pM | 0.12 nM | 0.22 nM | 24 nM | 0.53 mM | 0.14 | μM | 57 pM | 35 pM |
| AB21_HC_Mutall | Humanized | (AB21_HC_Mutall) | | | | | | | | | |

NT = not tested.

### Example 5: In Vivo Evaluation of Antibody-CpG Nucleotide Conjugates

CT26 and MC38 cells were injected into the right flank of BALB/c and C57BL/6 female mice, respectively, at a concentration of 2×10⁶ cells per mouse in RPMI 1640 (for CT26) or DMEM (for MC38). Tumors were monitored until the average size of tumors reached between 75-300 mm³ depending on the study. Mice were randomized into PBS control, anti-SIRPα-CpG nucleotide conjugate with blocking antibody (anti-SIRPα 1), and anti-SIRPα-CpG nucleotide conjugate with non-blocking antibody (anti-SIRPα 2) groups with 5-7 mice per cohort depending on the study. Sequences of anti-SIRPα antibodies are described in Example 2; CpG corresponded to p313. Anti-SIRPα-CpG nucleotide conjugate-treated mice were dosed with 0.1-10 mg/kg two times in total, three days apart. Both drugs were administered intraperitoneally. Tumors were measured in two dimensions with calipers, and tumor volume was calculated as: length×width×width×0.5, where length was the larger of the two measurements.

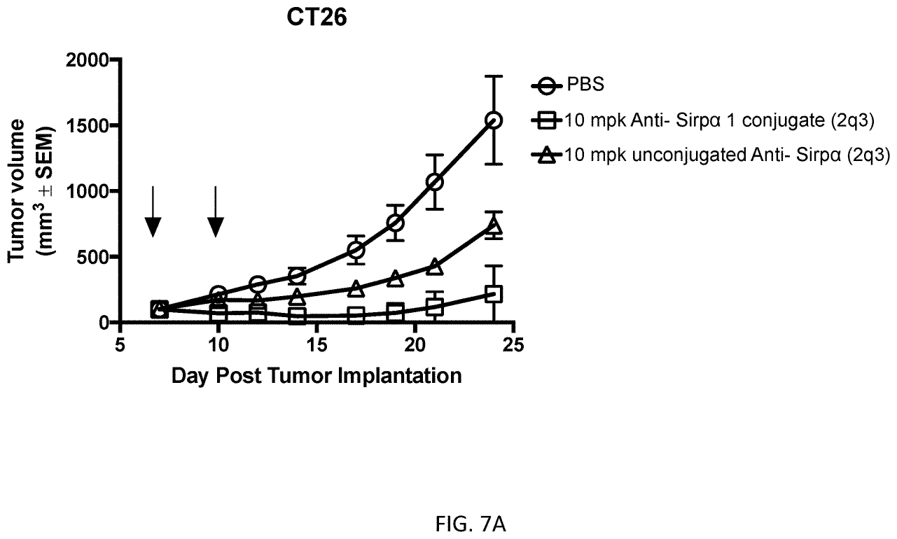

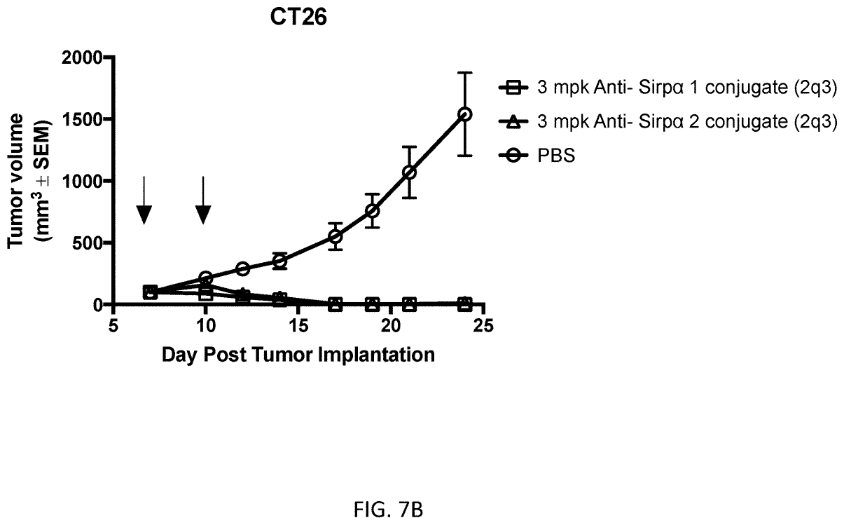

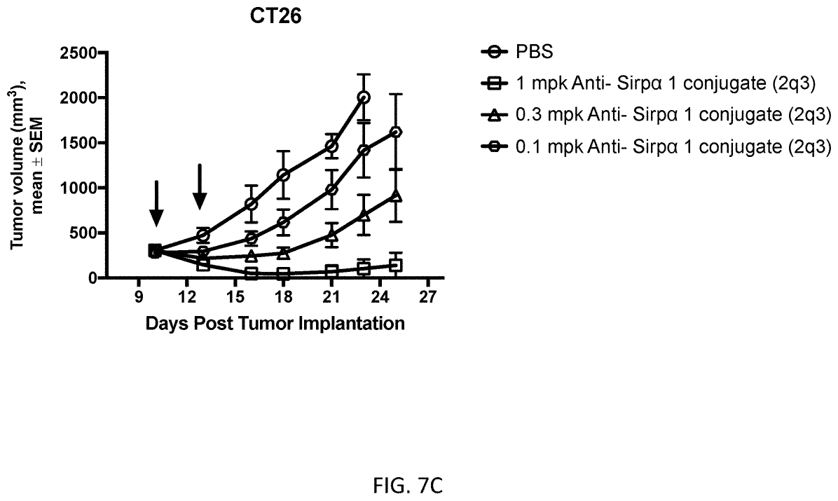

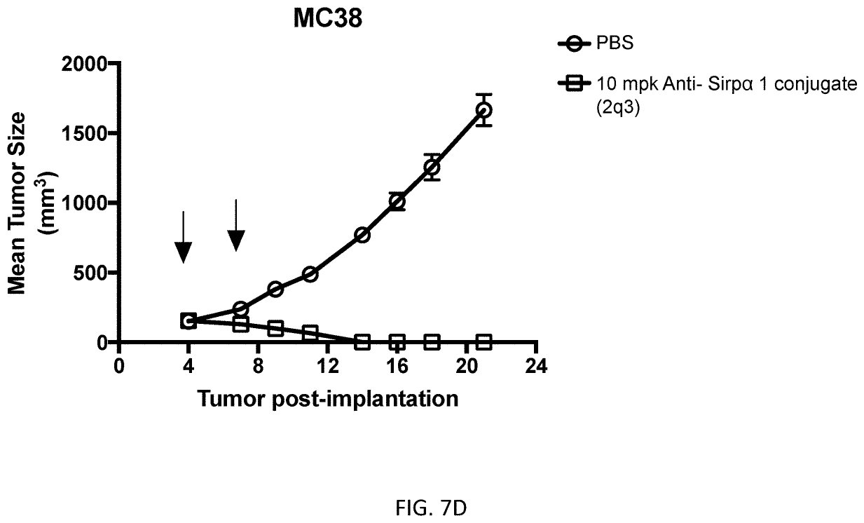

CT26-tumor bearing mice were measured and randomized by tumor volume. On day 4, each cohort of 5 mice had an average tumor size of 75 mm³. 10 mg/kg anti-SIRPα 1 conjugate-treated mice dosed twice, three days apart showed tumor eradication (4/5 mice) while mice treated with 10 mg/kg unconjugated control anti-SIRPα antibody dose twice, three days apart showed sub-optimal tumor inhibition as compared to PBS (FIG. 7A). CT26 tumor-bearing mice were measured and randomized by tumor volume. On day 8, tumors had an average tumor size of 100 mm³ with 7 mice per group, and two treatments with 3 mg/kg anti-SIRPα 1 conjugate and anti-SIRPα 2 conjugate, three days apart, showed complete tumor eradication (FIG. 7B). On day 24, 7 out of 7 mice treated with anti-SIRPα 1 conjugate (anti-SIRPα blocking antibody conjugate) and 6 out of 7 mice treated with anti-SIRPα 2 conjugate (anti-SIRPα non-blocking antibody conjugate) had complete tumor eradication. As shown in FIG. 7C, CT26 tumor-bearing mice with an average tumor size of 300 mm³ and cohort of 5 mice were treated with 0.1, 0.3 and 1 mg/kg anti-SIRPα 1 conjugate twice, three days apart. A dose response in tumor inhibition was observed with 1 mg/kg being the most potent. 4 out of 5 mice showed tumor eradication on day 21 for the group treated with 1 mg/kg anti-SIRPα 1 conjugate. As shown in FIG. 7D, MC38 tumor-bearing mice with an average of 155 mm³ tumor volume treated with two doses of 10 mg/kg anti-SIRPα 1 conjugate three days apart showed complete eradication of tumor on day 21. Collectively, these data show eradication of tumors in multiple tumor models, specific activity of SIRPα-CpG as compared to unconjugated SIRPα antibody, tumor eradication with both SIRPα blocking and non-blocking antibody CpG conjugates, and eradication of tumors when mice are treated with anti-SIRPα 1 conjugate as low as 1 mg/kg.

Tumors were monitored until the average size of tumors reached 300 mm³. Mice were randomized into PBS control and anti-SIRPα-CpG nucleotide conjugate with blocking antibody (anti-SIRPα 1) with 5 mice per cohort. Sequences of anti-SIRPα antibodies are described in Example 2; CpG corresponded to p313. Anti-SIRPα-CpG nucleotide conjugate-treated mice were dosed with 1 mg/kg for CT26 model two times in total, three or 7 days apart. Anti-SIRPα-CpG nucleotide conjugate was administered intraperitoneally. Tumors were measured in two dimensions with calipers, and tumor volume was calculated as: length×width×width×0.5, where length was the larger of the two measurements.

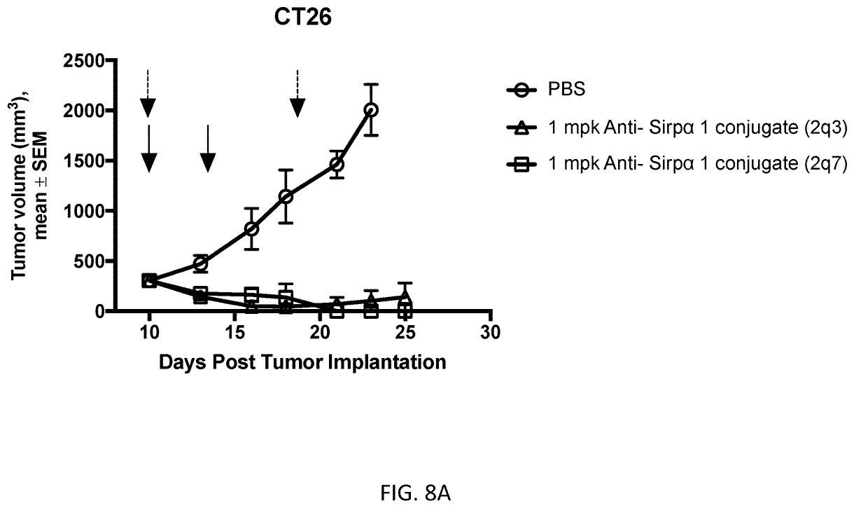

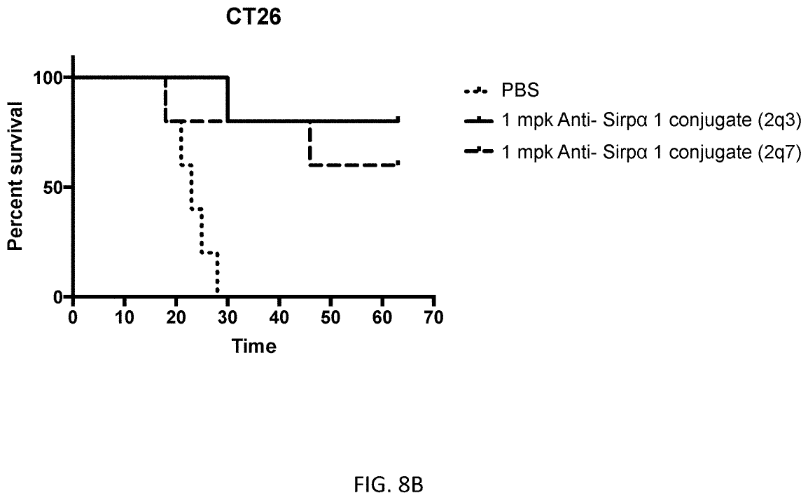

CT26 tumor-bearing mice were measured and randomized by tumor volume. On day 10, tumors had an average tumor size of 300 mm³ and treatment with 1 mg/kg anti-SIRPα 1 conjugate, two doses, three or seven days apart showed tumor eradication (FIG. 8A). On day 25, four out of five mice were tumor free in both groups. On day 63, of the four surviving mice treated three days apart, all were tumor free, while mice treated seven days apart also had four surviving mice but only two were still tumor free (FIG. 8B).

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference..

What is claimed is:

1. A conjugate comprising a targeting moiety, an immunomodulating polynucleotide, and a linker; wherein the targeting moiety binds to an antigen expressed by a NK cell or a myeloid cell; and the linker connects the targeting moiety covalently to the immunomodulating polynucleotide.

2. The conjugate of claim 1, wherein the immunomodulating polynucleotide comprises an internucleoside phosphotriester.

3. The conjugate of claim 1 or 2, wherein the immunomodulating polynucleotide comprises a nucleotide with a modified nucleobase.

4. The conjugate of any one of claims 1 to 3, wherein the conjugate has the structure of Formula (C): 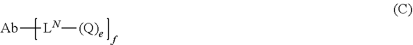
or a stereoisomer, a mixture of two or more diastereomers, a tautomer, or a mixture of two or more tautomers thereof; or a pharmaceutically acceptable salt, solvate, or hydrate thereof;
wherein:
Ab is a targeting moiety;
each L^{N} is independently a linker;
each Q is independently an immunomodulating polynucleotide;
each e is independently an integer of about 1, about 2, about 3, or about 4; and
f is an integer of about 1, about 2, about 3, or about 4.

5. The conjugate of claim 4, wherein f is an integer of about 1.

6. The conjugate of claim 4 or 5, wherein L^{N} is a linker comprising a polyethylene glycol.

7. The conjugate of claim 6, wherein L^{N} is 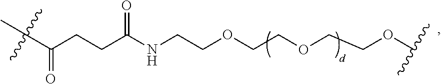
wherein d is an integer ranging from about 0 to about 50.

8. The conjugate of claim 6, wherein L^{N} is 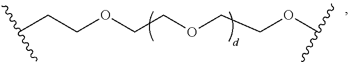
wherein d is an integer ranging from about 0 to about 50.

9. The conjugate of claim 7 or 8, wherein d is an integer ranging from about 0 to about 10.

10. The conjugate of claim 7 or 8, wherein d is an integer ranging from about 0 to about 5.

11. The conjugate of claim 7 or 8, wherein d is an integer of about 0, about 1, or about 3.

12. The conjugate of any one of claims 4 to 11, wherein e is an integer of about 1.

13. The conjugate of any one of claims 4 to 12, wherein each Q independently has the structure of Formula (D): 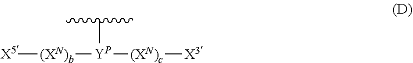
wherein:
each X^{N} is independently a nucleotide;
X^{3′} is a 3′ terminal nucleotide;
X^{5′} is a 5′ terminal nucleotide;
Y^{P} is a residue of an internucleoside phosphotriester; and
b and c are each an integer ranging from about 0 to about 25; with the proviso that their sum is no less than 5.

14. The conjugate of claim 13, wherein b is an integer ranging from about 1 to about 15.

15. The conjugate of claim 13, wherein b is an integer of about 3, about 4, about 11, or about 14.

16. The conjugate of claim 13, wherein b is an integer of about 3.

17. The conjugate of claim 13, wherein b is an integer of about 4.

18. The conjugate of claim 13, wherein b is an integer of about 11.

19. The conjugate of claim 13, wherein b is an integer of about 14.

20. The conjugate of any one of claims 13 to 19, wherein c is an integer ranging from about 0 to about 10.

21. The conjugate of claim 20, wherein c is an integer of about 0 or about 8.

22. The conjugate of claim 20, wherein c is an integer of about 0.

23. The conjugate of claim 20, wherein c is an integer of about 8.

24. The conjugate of any one of claims 13 to 23, wherein the sum of b and c is ranging from about 5 to about 20.

25. The conjugate of claim 24, wherein the sum of b and c is ranging from about 5 to about 15.

26. The conjugate of claim 24, wherein the sum of b and c is about 8, about 9, about 10, about 11, about 12, about 13, or about 14.

27. The conjugate of any one of claims 13 to 26, wherein each X^{N} is independently a 2′-deoxyribonucleotide.

28. The conjugate of claim 27, wherein each X^{N} is independently 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine.

29. The conjugate of claim 27, wherein each X^{N} is independently 2′-deoxyadenosine, 2′-deoxyguanosine, 2′-deoxycytidine, 2′-deoxythymidine, 5-bromo-2′-deoxyuridine, or 5-iodo-2′-deoxyuridine.

30. The conjugate of any one of claims 13 to 29, wherein X^{3′} is a 2′-deoxyribonucleotide.

31. The conjugate of claim 30, wherein X^{3′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine.

32. The conjugate of claim 30, wherein X^{3′} is 2′-deoxythymidine.

33. The conjugate of claim 30, wherein X^{3′} is a 2′-modified ribonucleotide.

34. The conjugate of claim 30, wherein X³, is a 2′-methoxy ribonucleotide or 2′-ethoxymethoxy ribonucleotide.

35. The conjugate of any one of claims 13 to 34, wherein X^{5′} is a 2′-deoxyribonucleotide.

36. The conjugate of claim 35, wherein X^{5′} is 2′-deoxyadenosine, 2′-deoxyguanosine, 2′-deoxycytidine, 5-halo-2′-deoxycytidine, 2′-deoxythymidine, 2′-deoxyuridine, or 5-halo-2′-deoxyuridine.

37. The conjugate of claim 35, wherein X^{5′} is a 2′-deoxyribonucleotide with a substituted pyrimidine base.

38. The conjugate of claim 35, wherein X^{5′} is a 2′-deoxyribonucleotide with a 5-substituted pyrimidine base.

39. The conjugate of claim 35, wherein X^{5′} is 2′-deoxythymidine, a 5-halo-2′-deoxycytidine, or a 5-halo-2′-deoxyuridine.

40. The conjugate of claim 35, wherein X^{5′} is 2′-deoxythymidine, 5-bromo-2′-deoxycytidine, 5-iodo-2′-deoxycytidine, 5-bromo-2′-deoxyuridine, or 5-iodo-2′-deoxyuridine.

41. The conjugate of claim 35, wherein X^{5′} is 2′-deoxythymidine, 5-bromo-2′-deoxyuridine, or 5-iodo-2′-deoxyuridine.

42. The conjugate of any one of claims 13 to 41, wherein X^{5′} has a 3′-phosphorothioate group.

43. The conjugate of claim 42, wherein the 3′-phosphorothioate is chiral.

44. The conjugate of claim 43, wherein the 3′-phosphorothioate has a chirality of Rp.

45. The conjugate of claim 43, wherein the 3′-phosphorothioate has a chirality of Sp.

46. The conjugate of any one of claims 13 to 43, wherein X^{5′} has a 3′-phosphorothioate group having a chirality of Rp and X^{3′} is a 2′-methoxy ribonucleotide or 2′-ethoxymethoxy ribonucleotide.

47. The conjugate of any one of claims 13 to 43, wherein X^{5′} has a 3′-phosphorothioate group having a chirality of Sp and X^{3′} is a 2′-methoxy ribonucleotide or 2′-ethoxymethoxy ribonucleotide.

48. The conjugate of any one of claims 13 to 47, wherein Y^{P} is: 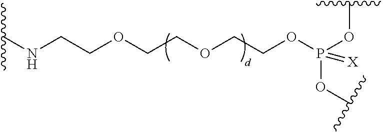
wherein Z is O or S; and d is an integer ranging from about 0 to about 50.

49. The conjugate of claim 48, wherein Y^{P} is: 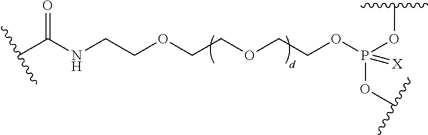
wherein Z is O or S; and d is an integer ranging from about 0 to about 50.

50. The conjugate of claim 48 or claim 49, wherein Z is O.

51. The conjugate of claim 48 or claim 49, wherein Z is S.

52. The conjugate of any one of claims 48-51, wherein wherein d is an integer ranging from about 0 to about 10.

53. The conjugate of claim 52, wherein d is an integer ranging from about 0 to about 5.

54. The conjugate of claim 52, wherein d is an integer of about 0, about 1, or about 3.

55. The conjugate of any one of claims 1 to 54, wherein the immunomodulating polynucleotide comprises an additional internucleoside phosphotriester.

56. The conjugate of claim 55, wherein the additional internucleoside phosphotriester is an alkylphosphotriester.

57. The conjugate of claim 55, wherein the additional internucleoside phosphotriester is ethylphosphotriester.

58. The conjugate of any one of claims 1 to 57, wherein the immunomodulating polynucleotide comprises one 5-halo-2′-deoxyuridine.

59. The conjugate of claim 58, wherein the 5-halo-2′-deoxyuridine is 5-bromo-2′-deoxyuridine or 5-iodo-2′-deoxyuridine.

60. The conjugate of any one of claims 1 to 59, wherein the immunomodulating polynucleotide comprises three or more 2′-deoxycytidines.

61. The conjugate of claim 60, wherein the immunomodulating polynucleotide comprises three 2′-deoxycytidines.

62. The conjugate of any one of claims 1 to 61, wherein the immunomodulating polynucleotide comprises four or more 2′-deoxyguanosines.

63. The conjugate of claim 62, wherein the immunomodulating polynucleotide comprises four 2′-deoxyguanosines.

64. The conjugate of any one of claims 1 to 63, wherein the immunomodulating polynucleotide comprises three 2′-deoxycytidines and four 2′-deoxycytidines.

65. The conjugate of any one of claims 1 to 63, wherein the immunomodulating polynucleotide comprises three or more 2′-deoxythymidines.

66. The conjugate of claim 65, wherein the immunomodulating polynucleotide comprises three, four, five, six, seven, or eight 2′-deoxythymidines.

67. The conjugate of claim 65, wherein the immunomodulating polynucleotide comprises three, four, five, or eight 2′-deoxythymidines.

68. The conjugate of any one of claims 1 to 67, wherein the immunomodulating polynucleotide comprises zero, one, or two 2′-deoxyadenosines.

69. The conjugate of any one of claims 1 to 68, wherein the immunomodulating polynucleotide comprises one or more internucleoside phosphorothioates.

70. The conjugate of claim 69, wherein the immunomodulating polynucleotide comprises about 12 internucleoside phosphorothioates.

71. The conjugate of any one of claims 1 to 70, wherein the targeting moiety is an antibody to an antigen expressed on the NK cell.

72. The conjugate of any one of claims 1 to 70, wherein the targeting moiety is an antibody to an antigen expressed on the myeloid cell.

73. The conjugate of any one of claims 1 to 72, wherein the targeting moiety is a human antibody.

74. The conjugate of any one of claims 1 to 73, wherein the targeting moiety is a human anti-CD56 antibody.

75. The conjugate of claim 74, wherein the antibody is a human anti-CD56 antibody derived from clone 5.1 H11.

76. The conjugate of any one of claims 1 to 73, wherein the antibody is an anti-SIRPα antibody.

77. The conjugate of claim 76, wherein the targeting moiety is a blocking anti-SIRPα antibody.

78. The conjugate of claim 77, wherein the blocking anti-SIRPα antibody comprises a heavy chain variable (VH) domain comprising an HVR-H1 comprising a sequence selected from the group consisting of SEQ ID NOs:498-500, an HVR-H2 comprising the sequence of SEQ ID NO:501, and an HVR-H3 comprising the sequence of SEQ ID NO:502; and a light chain variable (VL) domain comprising an HVR-L1 comprising the sequence of SEQ ID NO:503, an HVR-L2 comprising the sequence of SEQ ID NO:504, and an HVR-L3 comprising the sequence of SEQ ID NO:505.

79. The conjugate of claim 78, wherein the blocking anti-SIRPα antibody comprises a VH domain comprising a sequence selected from the group consisting of SEQ ID NOs:490-495 and a VL domain comprising the sequence of SEQ ID NO:496 or 497.

80. The conjugate of claim 77, wherein the blocking anti-SIRPα antibody comprises a heavy chain variable (VH) domain comprising an HVR-H1 comprising a sequence selected from the group consisting of SEQ ID NOs:512-514, an HVR-H2 comprising the sequence of SEQ ID NO:515, and an HVR-H3 comprising the sequence of SEQ ID NO:516; and a light chain variable (VL) domain comprising an HVR-L1 comprising the sequence of SEQ ID NO:517, an HVR-L2 comprising the sequence of SEQ ID NO:518, and an HVR-L3 comprising the sequence of SEQ ID NO:519.

81. The conjugate of claim 80, wherein the blocking anti-SIRPα antibody comprises a VH domain comprising a sequence selected from the group consisting of SEQ ID NOs:506-509 and a VL domain comprising the sequence of SEQ ID NO:510 or 511.

82. The conjugate of claim 77, wherein the blocking anti-SIRPα antibody comprises a heavy chain variable (VH) domain comprising an HVR-H1 comprising a sequence selected from the group consisting of SEQ ID NOs:533-535, an HVR-H2 comprising the sequence of SEQ ID NO:536, and an HVR-H3 comprising the sequence of SEQ ID NO:537; and a light chain variable (VL) domain comprising an HVR-L1 comprising a sequence selected from the group consisting of SEQ ID NOs:538-542, an HVR-L2 comprising the sequence of SEQ ID NO:543, and an HVR-L3 comprising a sequence selected from the group consisting of SEQ ID NOs:544-546.

83. The conjugate of claim 82, wherein the blocking anti-SIRPα antibody comprises a VH domain comprising a sequence selected from the group consisting of SEQ ID NOs:520-523 and a VL domain comprising a sequence selected from the group consisting of SEQ ID NOs:525-532.

84. The conjugate of claim 76, wherein the targeting moiety is a non-blocking anti-SIRPα antibody.

85. The conjugate of claim 84, wherein the non-blocking anti-SIRPα antibody comprises a heavy chain variable (VH) domain comprising an HVR-H1 comprising a sequence selected from the group consisting of SEQ ID NOs:554-556, an HVR-H2 comprising the sequence of SEQ ID NO:557, and an HVR-H3 comprising the sequence of SEQ ID NO:558; and a light chain variable (VL) domain comprising an HVR-L1 comprising the sequence of SEQ ID NO:559, an HVR-L2 comprising the sequence of SEQ ID NO:560, and an HVR-L3 comprising the sequence of SEQ ID NO:561.

86. The conjugate of claim 85, wherein the non-blocking anti-SIRPα antibody comprises a VH domain comprising a sequence selected from the group consisting of SEQ ID NOs:547-550 and a VL domain comprising a sequence selected from the group consisting of SEQ ID NOs:551-553.

87. The conjugate of claim 84, wherein the non-blocking anti-SIRPα antibody comprises a VH domain comprising the sequence of SEQ ID NO:584 and a VL domain comprising a sequence selected from the group consisting of SEQ ID NOs:585, 562, and 563.

88. The conjugate of any one of claims 1 to 73, wherein the antibody is an anti-SIRPβ antibody.

89. The conjugate of claim 88, wherein the targeting moiety is a blocking anti-SIRPβ antibody.

90. The conjugate of claim 88, wherein the targeting moiety is a non-blocking anti-SIRPβ antibody.

91. The conjugate of any one of claims 1 to 73, wherein the antibody is an anti-SIRPγ antibody.

92. The conjugate of claim 91, wherein the targeting moiety is a blocking anti-SIRPγ antibody.

93. The conjugate of claim 91, wherein the targeting moiety is a non-blocking anti-SIRPγ antibody.

94. The conjugate of any one of claims 71-93, wherein the antibody comprises a human Fc region.

95. The conjugate of claim 94, wherein the Fc region is a human IgG1, IgG2, or IgG4 Fc region.

96. The conjugate of claim 94, wherein the Fc region is:
(i) a human IgG1 Fc region comprising L234 A, L235 A, and G237 A mutations, according to EU numbering;
(ii) a human IgG1 Fc region comprising L234 A, L235 A, G237 A, and N297 A mutations, according to EU numbering;
(iii) a human IgG1 Fc region comprising an N297 A mutation, according to EU numbering;
(iv) a human IgG1 Fc region comprising a D265 A mutation, according to EU numbering;
(v) a human IgG1 Fc region comprising D265 A and N297 A mutations, according to EU numbering;
(vi) a human IgG2 Fc region comprising A330 S and P331 S mutations, according to EU numbering;
(vii) a human IgG2 Fc region comprising A330 S, P331 S, and N297 A mutations, according to EU numbering;
(viii) a human IgG2 Fc region comprising an N297 A mutation, according to EU numbering;
(ix) a human IgG4 Fc region comprising an S228P mutation, according to EU numbering;
(x) a human IgG4 Fc region comprising S228P and D265 A mutations, according to EU numbering;
(xi) a human IgG4 Fc region comprising S228P and L235E mutations, according to EU numbering;
(xii) a human IgG4 Fc region comprising S228P and N297 A mutations, according to EU numbering; or
(xiii) a human IgG4 Fc region comprising S228P, E233P, F234V, L235 A, delG236, and N297 A mutations, according to EU numbering.

97. The conjugate of claim 94, wherein the Fc region comprises a sequence selected from the group consisting of SEQ ID NOs:564-578.

98. The conjugate of any one of claims 71-97, wherein the antibody comprises a human kappa light chain constant domain.

99. The conjugate of claim 98, wherein the antibody comprises a light chain constant domain comprising the sequence of SEQ ID NO:579.

100. The conjugate of any one of claims 71-97, wherein the antibody comprises a human lambda light chain constant domain.

101. The conjugate of claim 100, wherein the antibody comprises a light chain constant domain comprising the sequence of SEQ ID NO:580 or 581.

102. The conjugate of any one of claims 1 to 101, wherein the linker has the structure of: 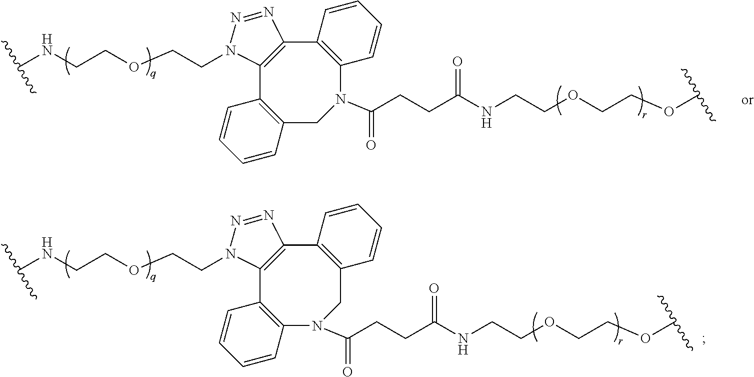
wherein each q is independently an integer ranging from about 0 to about 50; each r is independently an integer ranging from about 0 to about 50; the amino group is connected to an amino acid residue on the targeting moiety; and the hydroxyl group is connected to an internucleoside phosphorothioate of the immunomodulating polynucleotide.

103. The conjugate of claim 102, wherein each q is independently an integer ranging from 0 to about 25.

104. The conjugate of claim 102, wherein each q is independently an integer ranging from about 20 to about 25.

105. The conjugate of any one of claims 102 to 104, wherein each r is independently an integer ranging from 0 to about 5.

106. The conjugate of any one of claims 1 to 101, wherein the linker has the structure of: 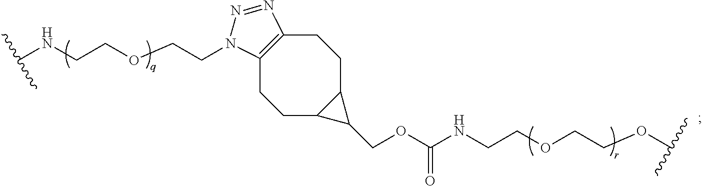
wherein each q is independently an integer ranging from about 0 to about 50; each r is independently an integer ranging from about 0 to about 50; the carbonyl group is connected to an amino acid residue on the targeting moiety; and the hydroxyl group is connected to an internucleoside phosphorothioate of the immunomodulating polynucleotide.

107. The conjugate of claim 106, wherein each q is independently an integer ranging from 0 to about 25.

108. The conjugate of claim 106, wherein each q is independently an integer ranging from about 5 to about 10.

109. The conjugate of any one of claims 106 to 108, wherein each r is independently an integer ranging from 0 to about 5.

110. The conjugate of any one of claims 1 to 101, wherein the linker has the structure of: 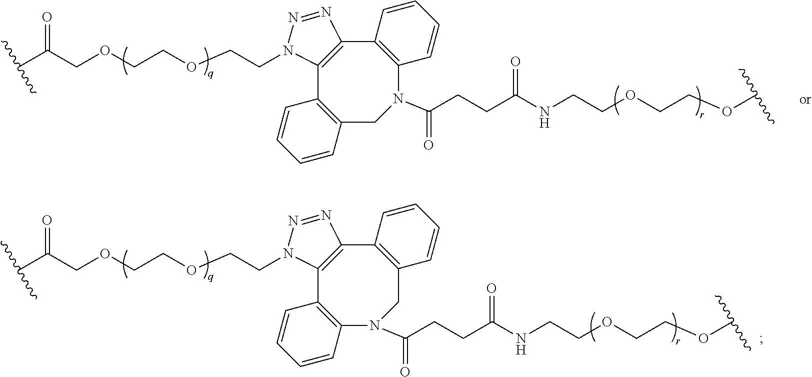
wherein q is an integer ranging from about 0 to about 50; r is an integer ranging from about 0 to about 50; the amino group is connected to an amino acid residue on the targeting moiety; and the hydroxyl group is connected to an internucleoside phosphorothioate of the immunomodulating polynucleotide.

111. The conjugate of any one of claims 1 to 101, wherein the linker has the structure of: 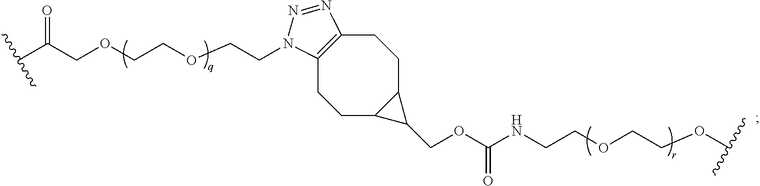
wherein q is an integer ranging from about 0 to about 50; r is an integer ranging from about 0 to about 50; the carbonyl group is connected to an amino acid residue on the targeting moiety; and the hydroxyl group is connected to an internucleoside phosphorothioate of the immunomodulating polynucleotide.

112. The conjugate of any one of claims 1 to 111, wherein the antibody-nucleotide conjugate has a DAR ranging from about 1 to about 8.

113. The conjugate of claim 112, wherein the antibody-nucleotide conjugate has a DAR of about 1.

114. The conjugate of claim 112, wherein the antibody-nucleotide conjugate has a DAR ranging from about 3 to about 4.

115. The conjugate of any one of claims 1 to 114, having the structure of: 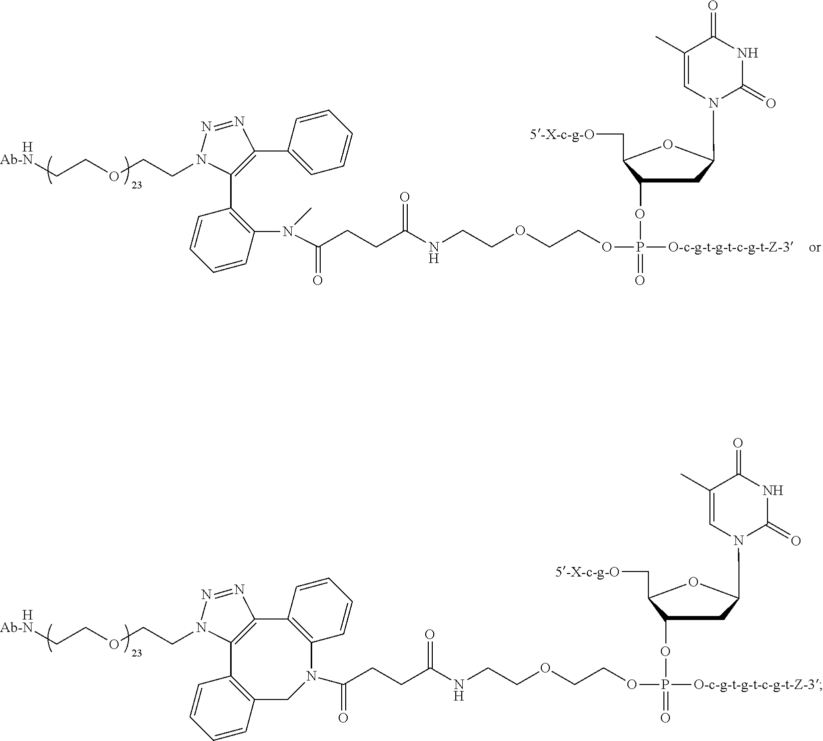
wherein c is 2′-deoxycytidine; g is 2′-deoxyguanosine; t is thymidine; X is 5-bromo-2′-deoxyuridine; and Z is

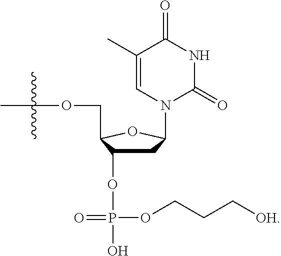

116. The conjugate of any one of claims 1 to 114, having the structure of: 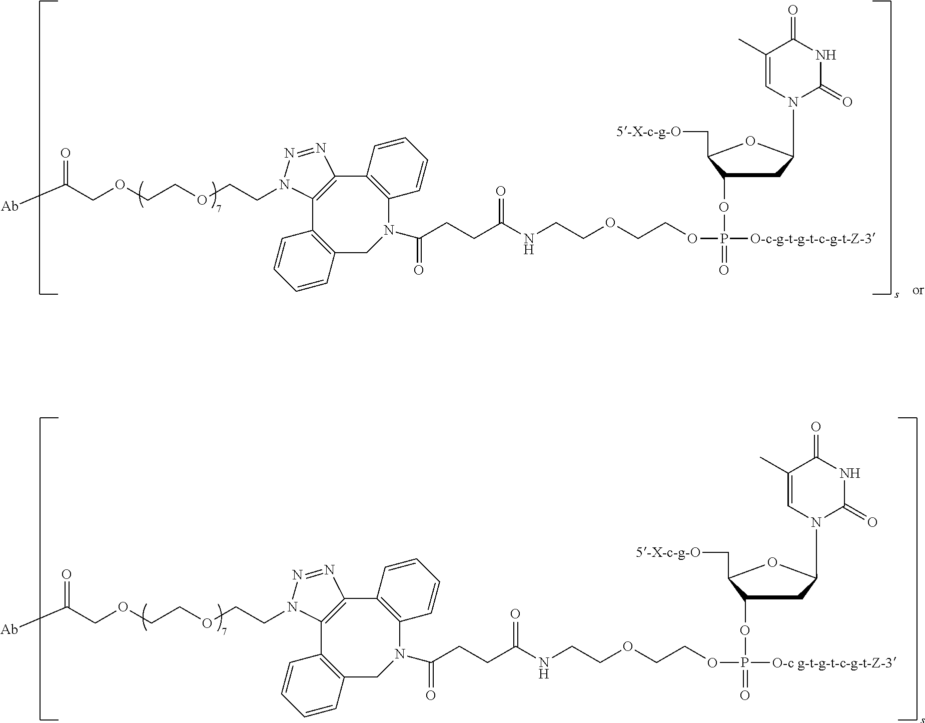
wherein s is an integer of about 3 or about 4; c is 2′-deoxycytidine; g is 2′-deoxyguanosine; t is thymidine; X is 5-bromo-2′-deoxyuridine; and Z is

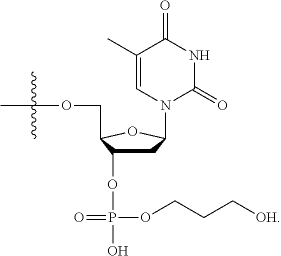

117. A pharmaceutical composition comprising the conjugate of any one of claims 1 to 116, and a pharmaceutically acceptable excipient.

118. The pharmaceutical composition of claim 117, wherein the composition is formulated for parenteral administration.

119. The pharmaceutical composition of claim 117 or 118, wherein the composition is formulated as a single dosage form.

120. The pharmaceutical composition of any one of claims 117 to 119, wherein the composition is formulated as an intravenous dosage form.

121. The pharmaceutical composition of any one of claims 117 to 120, further comprising a second therapeutic agent.

122. A method for treating, preventing, or ameliorating one or more symptoms of a proliferative disease in a subject, comprising administering to the subject the conjugate of any one of claims 1 to 116.

123. The method of claim 122, wherein the proliferative disease is cancer.

124. A method of modulating a natural killer cell in a subject, comprising administering to the subject the conjugate of any one of claims 1 to 116.

125. A method of modulating a myeloid cell in a subject, comprising administering to the subject the conjugate of any one of claims 1 to 116.

126. The method of claim 125, wherein the myeloid cell is a monocyte.


SEQUENCE LISTING (586 sequences)

SEQ ID NO: 1 - DNA, 24 bp, Artificial Sequence
  Synthetic Construct
  misc_feature (1)..(24): nucleoside-3′-phosphorothioate
tcgtcgttttgtcgttttgtcgtt

SEQ ID NO: 2 - DNA, 24 bp, Artificial Sequence
  Synthetic Construct
  misc_feature (1)..(24): nucleoside-3′-phosphorothioate
  misc_feature 1: modified by NH2-C6 as described in Table 2 of the specification
tcgtcgttttgtcgttttgtcgtt

SEQ ID NO: 3 - DNA, 24 bp, Artificial Sequence
  Synthetic Construct
  misc_feature (1)..(24): nucleoside-3′-phosphorothioate
  misc_feature 1: modified by NH2-C6-S-S-C6 as described in Table 2 of the specification
tcgtcgttttgtcgttttgtcgtt

SEQ ID NO: 4 - DNA, 24 bp, Artificial Sequence
  Synthetic Construct
  misc_feature (1)..(24): nucleoside-3′-phosphorothioate
  misc_feature 1: modified by DBCO
tcgtcgttttgtcgttttgtcgtt

SEQ ID NO: 5 - DNA, 24 bp, Artificial Sequence
  Synthetic Construct
  misc_feature (1)..(24): nucleoside-3′-phosphorothioate
  misc_feature 1: modified by DBCO-C6-S-S-C6 as described in Table 2 of the specification
tcgtcgttttgtcgttttgtcgtt

SEQ ID NO: 6 - DNA, 24 bp, Artificial Sequence
  Synthetic Construct
  misc_feature (1)..(24): nucleoside-3′-phosphate
tcgtcgttttgtcgttttgtcgtt

SEQ ID NO: 7 - DNA, 24 bp, Artificial Sequence
  Synthetic Construct
  misc_feature (1)..(24): nucleoside-3′-phosphate
  misc_feature 1: modified by DBCO-C6-S-S-C6 as described in Table 2 of the specification
tcgtcgttttgtcgttttgtcgtt

SEQ ID NO: 8 - DNA, 24 bp, Artificial Sequence
  Synthetic Construct
  misc_feature (1)..(24): nucleoside-3′-phosphorothioate
tgctgcttttgtgcttttgtgctt

SEQ ID NO: 9 - DNA, 24 bp, Artificial Sequence
  Synthetic Construct
  misc_feature (1)..(24): nucleoside-3′-phosphorothioate
  misc_feature 1: modified by DBCO-C6-S-S-C6 as described in Table 2 of the specification
tgctgcttttgtgcttttgtgctt

SEQ ID NO: 10 - DNA, 24 bp, Artificial Sequence
  Synthetic Construct
  misc_feature (1)..(24): nucleoside-3′-phosphorothioate
  misc_feature 1, 23: nucleoside having a 3′ tBuDS-Ph (ortho) triester
tcgtcgttttgtcgttttgtcgtt

SEQ ID NO: 11 - DNA, 24 bp, Artificial Sequence
  Synthetic Construct
  misc_feature (1)..(24): nucleoside-3′-phosphate
  misc_feature 1, 23: nucleoside having a 3′ tBuDS-Ph (ortho) triester
tcgtcgttttgtcgttttgtcgtt

SEQ ID NO: 12 - DNA, 24 bp, Artificial Sequence
  Synthetic Construct
  misc_feature (1)..(24): nucleoside-3′-phosphorothioate
  misc_feature 1: modified by DBCO-C6-S-S-C6 as described in Table 2 of the specification
  misc_feature 1, 23: nucleoside having a 3′ tBuDS-Ph (ortho) triester
tcgtcgttttgtcgttttgtcgtt

SEQ ID NO: 13 - DNA, 24 bp, Artificial Sequence
  Synthetic Construct
  misc_feature (1)..(24): nucleoside-3′-phosphate
  misc_feature 1: modified by DBCO-C6-S-S-C6 as described in Table 2 of the specification
  misc_feature 1, 23: nucleoside having a 3′ tBuDS-Ph (ortho) triester
tcgtcgttttgtcgttttgtcgtt

SEQ ID NO: 14 - DNA, 24 bp, Artificial Sequence
  Synthetic Construct
  misc_feature (1)..(24): nucleoside-3′-phosphorothioate
  misc_feature 1: modified by DBCO
  misc_feature 1, 23: nucleoside having a 3′ tBuDS-Ph (ortho) triester
tcgtcgttttgtcgttttgtcgtt

SEQ ID NO: 15 - DNA, 24 bp, Artificial Sequence
  Synthetic Construct
  misc_feature (1)..(24): nucleoside-3′-phosphate
  misc_feature 1: modified by DBCO
  misc_feature 1, 23: nucleoside having a 3′ tBuDS-Ph (ortho) triester
tcgtcgttttgtcgttttgtcgtt

SEQ ID NO: 16 - DNA, 20 bp, Artificial Sequence
  Synthetic Construct
  misc_feature (1)..(20): nucleoside-3′-phosphorothioate
  misc_feature 1: modified by DBCO
  misc_feature 1, 19: nucleoside having a 3′ tBuDS-Ph (ortho) triester
tccatgacgttcctgacgtt

SEQ ID NO: 17 - DNA, 20 bp, Artificial Sequence
  Synthetic Construct
  misc_feature (1)..(20): nucleoside-3′-phosphate
  misc_feature 1: modified by DBCO
  misc_feature 1, 19: nucleoside having a 3′ tBuDS-Ph (ortho) triester
tccatgacgttcctgacgtt

SEQ ID NO: 18 - DNA, 20 bp, Artificial Sequence
  Synthetic Construct
  misc_feature (1)..(20): nucleoside-3′-phosphorothioate
tccatgacgttcctgacgtt

SEQ ID NO: 19 - DNA, 20 bp, Artificial Sequence
  Synthetic Construct
  misc_feature (1)..(20): nucleoside-3′-phosphorothioate
  misc_feature 1: modified by DBCO
tccatgacgttcctgacgtt

SEQ ID NO: 20 - DNA, 20 bp, Artificial Sequence
  Synthetic Construct
  misc_feature (1)..(20): nucleoside-3′-phosphate
tccatgacgttcctgacgtt

SEQ ID NO: 21 - DNA, 20 bp, Artificial Sequence
  Synthetic Construct
  misc_feature (1)..(20): nucleoside-3′-phosphate
  misc_feature 1: modified by DBCO
tccatgacgttcctgacgtt

SEQ ID NO: 22 - DNA, 20 bp, Artificial Sequence
  Synthetic Construct
  misc_feature (1)..(20): nucleoside-3′-phosphorothioate
tccatgagcttcctgagctt

SEQ ID NO: 23 - DNA, 20 bp, Artificial Sequence
  Synthetic Construct
  misc_feature (1)..(20): nucleoside-3′-phosphorothioate
  misc_feature 1: modified by DBCO
tccatgagcttcctgagctt

SEQ ID NO: 24 - DNA, 24 bp, Artificial Sequence
  Synthetic Construct
  misc_feature (1)..(24): nucleoside-3′-phosphorothioate
  misc_feature 1: dT(DBCO)
tcgtcgttttgtcgttttgtcgtt

SEQ ID NO: 25 - DNA, 24 bp, Artificial Sequence
  Synthetic Construct
  misc_feature (1)..(24): nucleoside-3′-phosphorothioate
  misc_feature 9: dT(DBCO)
tcgtcgttttgtcgttttgtcgtt

SEQ ID NO: 26 - DNA, 24 bp, Artificial Sequence
  Synthetic Construct
  misc_feature (1)..(24): nucleoside-3′-phosphorothioate
  misc_feature 12: dT(DBCO)
tcgtcgttttgtcgttttgtcgtt

SEQ ID NO: 27 - DNA, 24 bp, Artificial Sequence
  Synthetic Construct
  misc_feature (1)..(24): nucleoside-3′-phosphorothioate
  misc_feature 17: dT(DBCO)
tcgtcgttttgtcgttttgtcgtt

SEQ ID NO: 28 - DNA, 24 bp, Artificial Sequence
  Synthetic Construct
  misc_feature (1)..(24): nucleoside-3′-phosphorothioate
  misc_feature 24: dT(DBCO)
tcgtcgttttgtcgttttgtcgtt

SEQ ID NO: 29 - DNA, 26 bp, Artificial Sequence
  Synthetic Construct
  misc_feature (1)..(26): nucleoside-3′-phosphorothioate
tcgcgacgttcgcccgacgttcggta

SEQ ID NO: 30 - DNA, 26 bp, Artificial Sequence
  Synthetic Construct
  misc_feature (1)..(26): nucleoside-3′-phosphorothioate
  misc_feature 1: modified by DBCO
tcgcgacgttcgcccgacgttcggta

SEQ ID NO: 31 - DNA, 20 bp, Artificial Sequence
  Synthetic Construct
  misc_feature (1)..(20): nucleoside-3′-phosphorothioate
tccatgacgttcctgatgct

SEQ ID NO: 32 - DNA, 20 bp, Artificial Sequence
  Synthetic Construct
  misc_feature (1)..(20): nucleoside-3′-phosphorothioate
  misc_feature 1: modified by DBCO
tccatgacgttcctgatgct

SEQ ID NO: 33 - DNA, 23 bp, Artificial Sequence
  Synthetic Construct
  misc_feature (1)..(23): nucleoside-3′-phosphorothioate
tcgacgttcgtcgttcgtcgttc

SEQ ID NO: 34 - DNA, 23 bp, Artificial Sequence
  Synthetic Construct
  misc_feature (1)..(23): nucleoside-3′-phosphorothioate
  misc_feature 1: modified by DBCO
tcgacgttcgtcgttcgtcgttc

SEQ ID NO: 35 - DNA, 24 bp, Artificial Sequence
  Synthetic Construct
  misc_feature (1)..(24): nucleoside-3′-phosphorothioate
  misc_feature 1, 23: nucleoside having a 3′ n-butyl triester
tcgtcgttttgtcgttttgtcgtt

SEQ ID NO: 36 - DNA, 24 bp, Artificial Sequence
  Synthetic Construct
  misc_feature (1)..(24): nucleoside-3′-phosphorothioate
  misc_feature 1: modified by DBCO
  misc_feature 1, 23: nucleoside having a 3′ n-butyl triester
tcgtcgttttgtcgttttgtcgtt

SEQ ID NO: 37 - DNA, 20 bp, Artificial Sequence
  Synthetic Construct
  misc_feature (1)..(20): nucleoside-3′-phosphorothioate
  misc_feature 1, 19: nucleoside having a 3′ n-butyl triester
tccatgacgttcctgacgtt

SEQ ID NO: 38 - DNA, 20 bp, Artificial Sequence
  Synthetic Construct
  misc_feature (1)..(20): nucleoside-3′-phosphorothioate
  misc_feature 1: modified by DBCO
  misc_feature 1, 19: nucleoside having a 3′ n-butyl triester
tccatgacgttcctgacgtt

SEQ ID NO: 39 - DNA, 20 bp, Artificial Sequence
  Synthetic Construct
  misc_feature (1)..(20): nucleoside-3′-phosphorothioate
  misc_feature 1, 20: nucleoside having a 3′ n-butyl triester
  misc_feature 20: modified by C3 spacer (-(CH2)3-OH) (3′ phosphate)
tccatgacgttcctgacgtt

SEQ ID NO: 40 - DNA, 20 bp, Artificial Sequence
  Synthetic Construct
  misc_feature (1)..(20): nucleoside-3′-phosphate
  misc_feature 1, 19: nucleoside having a 3′ n-butyl triester
tccatgacgttcctgacgtt

SEQ ID NO: 41 - DNA, 20 bp, Artificial Sequence
  Synthetic Construct
  misc_feature (1)..(20): nucleoside-3′-phosphate
  misc_feature 1, 20: nucleoside having a 3′ n-butyl triester
  misc_feature 20: modified by C3 spacer (-(CH2)3-OH) (3′ phosphate)
tccatgacgttcctgacgtt

SEQ ID NO: 42 - DNA, 20 bp, Artificial Sequence
  Synthetic Construct
  misc_feature (1)..(20): nucleoside-3′-phosphorothioate
  misc_feature 19: nucleoside having a 3′ n-butyl triester
tccatgacgttcctgacgtt

SEQ ID NO: 43 - DNA, 20 bp, Artificial Sequence
  Synthetic Construct
  misc_feature (1)..(20): nucleoside-3′-phosphorothioate
  misc_feature 20: modified by C3 spacer (-(CH2)3-OH) (3′ phosphate)
tccatgacgttcctgacgtt

SEQ ID NO: 44 - DNA, 20 bp, Artificial Sequence
  Synthetic Construct
  misc_feature (1)..(20): nucleoside-3′-phosphate
  misc_feature 19: nucleoside having a 3′ n-butyl triester
tccatgacgttcctgacgtt

SEQ ID NO: 45 - DNA, 20 bp, Artificial Sequence
  Synthetic Construct
  misc_feature (1)..(20): nucleoside-3′-phosphate
  misc_feature 20: nucleoside having a 3′ n-butyl triester
  misc_feature 20: modified by C3 spacer (-(CH2)3-OH) (3′ phosphate)
tccatgacgttcctgacgtt

SEQ ID NO: 46 - DNA, 20 bp, Artificial Sequence
  Synthetic Construct
  misc_feature (1)..(20): nucleoside-3′-phosphorothioate
  misc_feature 1: nucleoside having a 3′ homopropargyl triester (hPro)
tccatgacgttcctgacgtt

SEQ ID NO: 47 - DNA, 20 bp, Artificial Sequence
  Synthetic Construct
  misc_feature (1)..(20): nucleoside-3′-phosphorothioate
  misc_feature 5: nucleoside having a 3′ homopropargyl triester (hPro)
tccatgacgttcctgacgtt

SEQ ID NO: 48 - DNA, 20 bp, Artificial Sequence
  Synthetic Construct
  misc_feature (1)..(20): nucleoside-3′-phosphorothioate
  misc_feature 10: nucleoside having a 3′ homopropargyl triester (hPro)
tccatgacgttcctgacgtt

SEQ ID NO: 49 - DNA, 20 bp, Artificial Sequence
  Synthetic Construct
  misc_feature (1)..(20): nucleoside-3′-phosphorothioate
  misc_feature 11: nucleoside having a 3′ homopropargyl triester (hPro)
tccatgacgttcctgacgtt

SEQ ID NO: 50 - DNA, 20 bp, Artificial Sequence
  Synthetic Construct
  misc_feature (1)..(20): nucleoside-3′-phosphorothioate
  misc_feature 14: nucleoside having a 3′ homopropargyl triester (hPro)
tccatgacgttcctgacgtt

SEQ ID NO: 51 - DNA, 20 bp, Artificial Sequence
  Synthetic Construct
  misc_feature (1)..(20): nucleoside-3′-phosphorothioate
  misc_feature 19: nucleoside having a 3′ homopropargyl triester (hPro)
tccatgacgttcctgacgtt

SEQ ID NO: 52 - DNA, 20 bp, Artificial Sequence
  Synthetic Construct
  misc_feature (1)..(20): nucleoside-3′-phosphorothioate
  misc_feature 20: nucleoside having a 3′ homopropargyl triester (hPro)
tccatgacgttcctgacgtt

SEQ ID NO: 53 - DNA, 20 bp, Artificial Sequence
  Synthetic Construct
  misc_feature (1)..(2): nucleoside-3′-phosphate
  misc_feature (3)..(14): nucleoside-3′-phosphorothioate
  misc_feature (15)..(20): nucleoside-3′-phosphate
gggggacgatcgtcgggggg

SEQ ID NO: 54 - DNA, 25 bp, Artificial Sequence
  Synthetic Construct
  misc_feature (1)..(25): nucleoside-3′-phosphorothioate
tcgtcgtcgttcgaacgacgttgat

SEQ ID NO: 55 - DNA, 22 bp, Artificial Sequence
  Synthetic Construct
  misc_feature (1)..(22): nucleoside-3′-phosphorothioate
tcgtcgttttcggcgcgcgccg

SEQ ID NO: 56 - DNA, 29 bp, Artificial Sequence
  Synthetic Construct
  misc_feature (1)..(29): nucleoside-3′-phosphorothioate
tcgcgaacgttcgccgcgttcgaacgcgg

SEQ ID NO: 57 - DNA, 23 bp, Artificial Sequence
  Synthetic Construct
  misc_feature (1)..(23): nucleoside-3′-phosphorothioate
tcgtcgacgatcggcgcgcgccg

SEQ ID NO: 58 - DNA, 20 bp, Artificial Sequence
  Synthetic Construct
  misc_feature (1)..(20): nucleoside-3′-phosphorothioate
  misc_feature 1: nucleoside having a 3′ DBCO-NH-PEG2 triester (N1)
tccatgacgttcctgacgtt

SEQ ID NO: 59 - DNA, 20 bp, Artificial Sequence
  Synthetic Construct
  misc_feature (1)..(20): nucleoside-3′-phosphorothioate
  misc_feature 5: nucleoside having a 3′ DBCO-NH-PEG2 triester (N1)
tccatgacgttcctgacgtt

SEQ ID NO: 60 - DNA, 20 bp, Artificial Sequence
  Synthetic Construct
  misc_feature (1)..(20): nucleoside-3′-phosphorothioate
  misc_feature 10: nucleoside having a 3′ DBCO-NH-PEG2 triester (N1)
tccatgacgttcctgacgtt

SEQ ID NO: 61 - DNA, 20 bp, Artificial Sequence
  Synthetic Construct
  misc_feature (1)..(20): nucleoside-3′-phosphorothioate
  misc_feature 11: nucleoside having a 3′ DBCO-NH-PEG2 triester (N1)
tccatgacgttcctgacgtt

SEQ ID NO: 62 - DNA, 20 bp, Artificial Sequence
  Synthetic Construct
  misc_feature (1)..(20): nucleoside-3′-phosphorothioate
  misc_feature 14: nucleoside having a 3′ DBCO-NH-PEG2 triester (N1)
tccatgacgttcctgacgtt

SEQ ID NO: 63 - DNA, 20 bp, Artificial Sequence
  Synthetic Construct
  misc_feature (1)..(20): nucleoside-3′-phosphorothioate
  misc_feature 19: nucleoside having a 3′ DBCO-NH-PEG2 triester (N1)
tccatgacgttcctgacgtt

SEQ ID NO: 64 - DNA, 20 bp, Artificial Sequence
  Synthetic Construct
  misc_feature (1)..(20): nucleoside-3′-phosphorothioate
  misc_feature 20: nucleoside having a 3′ DBCO-NH-PEG2 triester (N1)
tccatgacgttcctgacgtt

SEQ ID NO: 65 - DNA, 20 bp, Artificial Sequence
  Synthetic Construct
  misc_feature (1)..(20): nucleoside-3′-phosphate
  misc_feature 1: nucleoside having a 3′ DBCO-NH-PEG2 triester (N1)
tccatgacgttcctgacgtt

SEQ ID NO: 66 - DNA, 20 bp, Artificial Sequence
  Synthetic Construct
  misc_feature (1)..(20): nucleoside-3′-phosphate
  misc_feature 5: nucleoside having a 3′ DBCO-NH-PEG2 triester (N1)
tccatgacgttcctgacgtt

SEQ ID NO: 67 - DNA, 20 bp, Artificial Sequence
  Synthetic Construct
  misc_feature (1)..(20): nucleoside-3′-phosphate
  misc_feature 10: nucleoside having a 3′ DBCO-NH-PEG2 triester (N1)
tccatgacgttcctgacgtt

SEQ ID NO: 68 - DNA, 20 bp, Artificial Sequence
  Synthetic Construct
  misc_feature (1)..(20): nucleoside-3′-phosphate
  misc_feature 11: nucleoside having a 3′ DBCO-NH-PEG2 triester (N1)
tccatgacgttcctgacgtt

SEQ ID NO: 69 - DNA, 20 bp, Artificial Sequence
  Synthetic Construct
  misc_feature (1)..(20): nucleoside-3′-phosphate
  misc_feature 14: nucleoside having a 3′ DBCO-NH-PEG2 triester (N1)
tccatgacgttcctgacgtt

SEQ ID NO: 70 - DNA, 20 bp, Artificial Sequence
  Synthetic Construct
  misc_feature (1)..(20): nucleoside-3′-phosphate
  misc_feature 19: nucleoside having a 3′ DBCO-NH-PEG2 triester (N1)
tccatgacgttcctgacgtt

SEQ ID NO: 71 - DNA, 20 bp, Artificial Sequence
  Synthetic Construct
  misc_feature (1)..(20): nucleoside-3′-phosphate
  misc_feature 20: nucleoside having a 3′ DBCO-NH-PEG2 triester (N1)
tccatgacgttcctgacgtt

SEQ ID NO: 72 - DNA, 24 bp, Artificial Sequence
  Synthetic Construct
  misc_feature (1)..(24): nucleoside-3′-phosphorothioate
  misc_feature 24: nucleoside having a 3′ DBCO-NH-PEG2 triester (N1)
tcgtcgttttgtcgttttgtcgtt

SEQ ID NO: 73 - DNA, 24 bp, Artificial Sequence
  Synthetic Construct
  misc_feature (1)..(24): nucleoside-3′-phosphate
  misc_feature 24: nucleoside having a 3′ DBCO-NH-PEG2 triester (N1)
tcgtcgttttgtcgttttgtcgtt

SEQ ID NO: 74 - DNA, 20 bp, Artificial Sequence
  Synthetic Construct
  misc_feature (1)..(20): nucleoside-3′-phosphorothioate
  misc_feature 20: nucleoside having a 3′ DBCO-NH-PEG2 triester (N1)
tccatgacgttcctgatgct

SEQ ID NO: 75 - DNA, 20 bp, Artificial Sequence
  Synthetic Construct
  misc_feature (1)..(20): nucleoside-3′-phosphate
  misc_feature 20: nucleoside having a 3′ DBCO-NH-PEG2 triester (N1)
tccatgacgttcctgatgct

SEQ ID NO: 76 - DNA, 24 bp, Artificial Sequence
  Synthetic Construct
  misc_feature (1)..(24): nucleoside-3′-phosphate
  misc_feature 1: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 77 - DNA, 24 bp, Artificial Sequence
  Synthetic Construct
  misc_feature (1)..(24): nucleoside-3′-phosphate
  misc_feature 4: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 78 - DNA, 24 bp, Artificial Sequence
  Synthetic Construct
  misc_feature (1)..(24): nucleoside-3′-phosphate
  misc_feature 7: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 79 - DNA, 24 bp, Artificial Sequence
  Synthetic Construct
  misc_feature (1)..(24): nucleoside-3′-phosphate
  misc_feature 8: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 80 - DNA, 24 bp, Artificial Sequence
  Synthetic Construct
  misc_feature (1)..(24): nucleoside-3′-phosphate
  misc_feature 9: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 81 - DNA, 24 bp, Artificial Sequence
  Synthetic Construct
  misc_feature (1)..(24): nucleoside-3′-phosphate
  misc_feature 10: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 82 - DNA, 24 bp, Artificial Sequence
  Synthetic Construct
  misc_feature (1)..(24): nucleoside-3′-phosphate
  misc_feature 12: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 83 - DNA, 24 bp, Artificial Sequence
  Synthetic Construct
  misc_feature (1)..(24): nucleoside-3′-phosphate
  misc_feature 15: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 84 - DNA, 24 bp, Artificial Sequence
  Synthetic Construct
  misc_feature (1)..(24): nucleoside-3′-phosphate
  misc_feature 16: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 85 - DNA, 24 bp, Artificial Sequence
  Synthetic Construct
  misc_feature (1)..(24): nucleoside-3′-phosphate
  misc_feature 17: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 86 - DNA, 24 bp, Artificial Sequence
  Synthetic Construct
  misc_feature (1)..(24): nucleoside-3′-phosphate
  misc_feature 18: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 87 - DNA, 24 bp, Artificial Sequence
  Synthetic Construct
  misc_feature (1)..(24): nucleoside-3′-phosphate
  misc_feature 20: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 88 - DNA, 24 bp, Artificial Sequence
  Synthetic Construct
  misc_feature (1)..(24): nucleoside-3′-phosphate
  misc_feature 23: nucleoside having a 3′ DBCO-NH-PEG2 triester (N1)
tcgtcgttttgtcgttttgtcgtt

SEQ ID NO: 89 - DNA, 20 bp, Artificial Sequence
  Synthetic Construct
  misc_feature (1)..(5): nucleoside-3′-phosphorothioate
  misc_feature (6)..(20): nucleoside-3′-phosphate
  misc_feature 19: nucleoside having a 3′ n-butyl triester
tccatgacgttcctgacgtt

SEQ ID NO: 90 - DNA, 20 bp, Artificial Sequence
  Synthetic Construct
  misc_feature (1)..(10): nucleoside-3′-phosphorothioate
  misc_feature (11)..(20): nucleoside-3′-phosphate
  misc_feature 19: nucleoside having a 3′ n-butyl triester
tccatgacgttcctgacgtt

SEQ ID NO: 91 - DNA, 20 bp, Artificial Sequence
  Synthetic Construct
  misc_feature (1)..(15): nucleoside-3′-phosphorothioate
  misc_feature (16)..(20): nucleoside-3′-phosphate
  misc_feature 19: nucleoside having a 3′ n-butyl triester
tccatgacgttcctgacgtt

SEQ ID NO: 92 - DNA, 20 bp, Artificial Sequence
  Synthetic Construct
  misc_feature (1)..(5): nucleoside-3′-phosphorothioate
  misc_feature (6)..(20): nucleoside-3′-phosphate
tccatgacgttcctgacgtt

SEQ ID NO: 93 - DNA, 20 bp, Artificial Sequence
  Synthetic Construct
  misc_feature (1)..(10): nucleoside-3′-phosphorothioate
  misc_feature (11)..(20): nucleoside-3′-phosphate
tccatgacgttcctgacgtt

SEQ ID NO: 94 - DNA, 20 bp, Artificial Sequence
  Synthetic Construct
  misc_feature (1)..(15): nucleoside-3′-phosphorothioate
  misc_feature (16)..(20): nucleoside-3′-phosphate
tccatgacgttcctgacgtt

SEQ ID NO: 95 - DNA, 24 bp, Artificial Sequence
  Synthetic Construct
  misc_feature (1)..(24): nucleoside-3′-phosphate
  misc_feature 2: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 96 - DNA, 24 bp, Artificial Sequence
  Synthetic Construct
  misc_feature (1)..(24): nucleoside-3′-phosphate
  misc_feature 3: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 97 - DNA, 24 bp, Artificial Sequence
  Synthetic Construct
  misc_feature (1)..(24): nucleoside-3′-phosphate
  misc_feature 5: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 98 - DNA, 24 bp, Artificial Sequence
  Synthetic Construct
  misc_feature (1)..(24): nucleoside-3′-phosphate
  misc_feature 6: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 99 - DNA, 24 bp, Artificial Sequence
  Synthetic Construct
  misc_feature (1)..(24): nucleoside-3′-phosphate
  misc_feature 11: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 100 - DNA, 24 bp, Artificial Sequence
  Synthetic Construct
  misc_feature (1)..(24): nucleoside-3′-phosphate
  misc_feature 13: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 101 - DNA, 24 bp, Artificial Sequence
  Synthetic Construct
  misc_feature (1)..(24): nucleoside-3′-phosphate
  misc_feature 14: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 102 - DNA, 24 bp, Artificial Sequence
  Synthetic Construct
  misc_feature (1)..(24): nucleoside-3′-phosphate
  misc_feature 19: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 103 - DNA, 24 bp, Artificial Sequence
  Synthetic Construct
  misc_feature (1)..(24): nucleoside-3′-phosphate
  misc_feature 21: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 104 - DNA, 24 bp, Artificial Sequence
  Synthetic Construct
  misc_feature (1)..(24): nucleoside-3′-phosphate
  misc_feature 22: nucleoside having a 3′ DBCO-NH-PEG2 triester (N1)
  misc_feature 23: nucleoside having a 3′ n-butyl triester
tcgtcgttttgtcgttttgtcgtt

SEQ ID NO: 105 - DNA, 20 bp, Artificial Sequence
  Synthetic Construct
  misc_feature (1)..(20): nucleoside-3′-phosphate
  misc_feature 1: nucleoside having a 3′ DBCO-NH-PEG2 triester (N1)
  misc_feature 19: nucleoside having a 3′ n-butyl triester
tccatgacgttcctgacgtt

SEQ ID NO: 106 - DNA, 20 bp, Artificial Sequence
  Synthetic Construct
  misc_feature (1)..(20): nucleoside-3′-phosphate
  misc_feature 2: nucleoside having a 3′ DBCO-NH-PEG2 triester (N1)
  misc_feature 19: nucleoside having a 3′ n-butyl triester
tccatgacgttcctgacgtt

SEQ ID NO: 107 - DNA, 20 bp, Artificial Sequence
  Synthetic Construct
  misc_feature (1)..(20): nucleoside-3′-phosphate
  misc_feature 3: nucleoside having a 3′ DBCO-NH-PEG2 triester (N1)
  misc_feature 19: nucleoside having a 3′ n-butyl triester
tccatgacgttcctgacgtt

SEQ ID NO: 108 - DNA, 20 bp, Artificial Sequence
  Synthetic Construct
  misc_feature (1)..(20): nucleoside-3′-phosphate
  misc_feature 4: nucleoside having a 3′ DBCO-NH-PEG2 triester (N1)
  misc_feature 19: nucleoside having a 3′ n-butyl triester
tccatgacgttcctgacgtt

SEQ ID NO: 109 - DNA, 20 bp, Artificial Sequence
  Synthetic Construct
  misc_feature (1)..(20): nucleoside-3′-phosphate
  misc_feature 5: nucleoside having a 3′ DBCO-NH-PEG2 triester (N1)
  misc_feature 19: nucleoside having a 3′ n-butyl triester
tccatgacgttcctgacgtt

SEQ ID NO: 110 - DNA, 20 bp, Artificial Sequence
  Synthetic Construct
  misc_feature (1)..(20): nucleoside-3′-phosphate
  misc_feature 6: nucleoside having a 3′ DBCO-NH-PEG2 triester (N1)
  misc_feature 19: nucleoside having a 3′ n-butyl triester
tccatgacgttcctgacgtt

SEQ ID NO: 111 - DNA, 20 bp, Artificial Sequence
  Synthetic Construct
  misc_feature (1)..(20): nucleoside-3′-phosphate
  misc_feature 7: nucleoside having a 3′ DBCO-NH-PEG2 triester (N1)
  misc_feature 19: nucleoside having a 3′ n-butyl triester
tccatgacgttcctgacgtt

SEQ ID NO: 112 - DNA, 20 bp, Artificial Sequence
  Synthetic Construct
  misc_feature (1)..(20): nucleoside-3′-phosphate
  misc_feature 8: nucleoside having a 3′ DBCO-NH-PEG2 triester (N1)
  misc_feature 19: nucleoside having a 3′ n-butyl triester
tccatgacgttcctgacgtt

SEQ ID NO: 113 - DNA, 20 bp, Artificial Sequence
  Synthetic Construct
  misc_feature (1)..(20): nucleoside-3′-phosphate
  misc_feature 9: nucleoside having a 3′ DBCO-NH-PEG2 triester (N1)
  misc_feature 19: nucleoside having a 3′ n-butyl triester
tccatgacgttcctgacgtt

SEQ ID NO: 114 - DNA, 20 bp, Artificial Sequence
  Synthetic Construct
  misc_feature (1)..(20): nucleoside-3′-phosphate
  misc_feature 10: nucleoside having a 3′ DBCO-NH-PEG2 triester (N1)
  misc_feature 19: nucleoside having a 3′ n-butyl triester
tccatgacgttcctgacgtt

SEQ ID NO: 115 - DNA, 20 bp, Artificial Sequence
  Synthetic Construct
  misc_feature (1)..(20): nucleoside-3′-phosphate
  misc_feature 11: nucleoside having a 3′ DBCO-NH-PEG2 triester (N1)
  misc_feature 19: nucleoside having a 3′ n-butyl triester
tccatgacgttcctgacgtt

SEQ ID NO: 116 - DNA, 20 bp, Artificial Sequence
  Synthetic Construct
  misc_feature (1)..(20): nucleoside-3′-phosphate
  misc_feature 12: nucleoside having a 3′ DBCO-NH-PEG2 triester (N1)
  misc_feature 19: nucleoside having a 3′ n-butyl triester
tccatgacgttcctgacgtt

SEQ ID NO: 117 - DNA, 20 bp, Artificial Sequence
  Synthetic Construct
  misc_feature (1)..(20): nucleoside-3′-phosphate
  misc_feature 13: nucleoside having a 3′ DBCO-NH-PEG2 triester (N1)
  misc_feature 19: nucleoside having a 3′ n-butyl triester
tccatgacgttcctgacgtt

SEQ ID NO: 118 - DNA, 20 bp, Artificial Sequence
  Synthetic Construct
  misc_feature (1)..(20): nucleoside-3′-phosphate
  misc_feature 14: nucleoside having a 3′ DBCO-NH-PEG2 triester (N1)
  misc_feature 19: nucleoside having a 3′ n-butyl triester
tccatgacgttcctgacgtt

SEQ ID NO: 119 - DNA, 20 bp, Artificial Sequence
  Synthetic Construct
  misc_feature (1)..(20): nucleoside-3′-phosphate
  misc_feature 15: nucleoside having a 3′ DBCO-NH-PEG2 triester (N1)
  misc_feature 19: nucleoside having a 3′ n-butyl triester
tccatgacgttcctgacgtt

SEQ ID NO: 120 - DNA, 20 bp, Artificial Sequence
  Synthetic Construct
  misc_feature (1)..(20): nucleoside-3′-phosphate
  misc_feature 16: nucleoside having a 3′ DBCO-NH-PEG2 triester (N1)
  misc_feature 19: nucleoside having a 3′ n-butyl triester
tccatgacgttcctgacgtt

SEQ ID NO: 121 - DNA, 20 bp, Artificial Sequence
  Synthetic Construct
  misc_feature (1)..(20): nucleoside-3′-phosphate
  misc_feature 17: nucleoside having a 3′ DBCO-NH-PEG2 triester (N1)
  misc_feature 19: nucleoside having a 3′ n-butyl triester
tccatgacgttcctgacgtt

SEQ ID NO: 122 - DNA, 20 bp, Artificial Sequence
  Synthetic Construct
  misc_feature (1)..(20): nucleoside-3′-phosphate
  misc_feature 18: nucleoside having a 3′ DBCO-NH-PEG2 triester (N1)
  misc_feature 19: nucleoside having a 3′ n-butyl triester
tccatgacgttcctgacgtt

SEQ ID NO: 123 - DNA, 20 bp, Artificial Sequence
  Synthetic Construct
  misc_feature (1)..(20): nucleoside-3′-phosphorothioate
  misc_feature 1: modified by IR700 dye
  misc_feature 19: nucleoside having a 3′ DBCO-NH-PEG2 triester (N1)
tccatgacgttcctgacgtt

SEQ ID NO: 124 - DNA, 20 bp, Artificial Sequence
  Synthetic Construct
  misc_feature (1)..(20): nucleoside-3′-phosphate
  misc_feature 1: modified by IR700 dye
  misc_feature 19: nucleoside having a 3′ DBCO-NH-PEG2 triester (N1)
tccatgacgttcctgacgtt

SEQ ID NO: 125 - DNA, 24 bp, Artificial Sequence
  Synthetic Construct
  misc_feature (1)..(24): nucleoside-3′-phosphorothioate
tcgtcgtttcgtcgttttgtcgtt

SEQ ID NO: 126 - DNA, 24 bp, Artificial Sequence
  Synthetic Construct
  misc_feature (1)..(24): nucleoside-3′-phosphate
  misc_feature 1: modified by DBCO
tcgtcgttttgtcgttttgtcgtt

SEQ ID NO: 127 - DNA, 24 bp, Artificial Sequence
  Synthetic Construct
  misc_feature (1)..(24): nucleoside-3′-phosphate
  misc_feature 1: modified by DBCO
  misc_feature 23: nucleoside having a 3′ n-butyl
tcgtcgttttgtcgttttgtcgtt

SEQ ID NO: 128 - DNA, 24 bp, Artificial Sequence
  Synthetic Construct
  misc_feature (1)..(24): nucleoside-3′-phosphate
  misc_feature 23: nucleoside having a 3′ DBCO-NH-PEG2 triester (N1)
tgctgcttttgtgcttttgtgctt

SEQ ID NO: 129 - DNA, 30 bp, Artificial Sequence
  Synthetic Construct
  misc_feature (1)..(6): nucleoside-3′-phosphorothioate
  misc_feature (7)..(27): nucleoside-3′-phosphate
  misc_feature (28)..(30): nucleoside-3′-phosphorothioate
tcattggaaaacgttcttcggggcgttctt

SEQ ID NO: 130 - DNA, 30 bp, Artificial Sequence
  Synthetic Construct
  misc_feature (1)..(6): nucleoside-3′-phosphorothioate
  misc_feature (7)..(27): nucleoside-3′-phosphate
  misc_feature (28)..(30): nucleoside-3′-phosphorothioate
tcattggaaaagcttcttgcggggcttctt

SEQ ID NO: 131 - DNA, 30 bp, Artificial Sequence; identical to SEQ ID NO: 129
  Synthetic Construct
  misc_feature (1)..(30): nucleoside-3′-phosphate

SEQ ID NO: 132 - DNA, 30 bp, Artificial Sequence
  Synthetic Construct
  misc_feature (1)..(30): nucleoside-3′-phosphate
aagaacgccccgaagaacgttttccaatga

SEQ ID NO: 133 - DNA, 30 bp, Artificial Sequence; identical to SEQ ID NO: 129
  Synthetic Construct
  misc_feature (1)..(30): nucleoside-3′-phosphate
  misc_feature 29: nucleoside having a 3′ n-butyl triester

SEQ ID NO: 134 - DNA, 30 bp, Artificial Sequence; identical to SEQ ID NO: 132
  Synthetic Construct
  misc_feature (1)..(30): nucleoside-3′-phosphate
  misc_feature 29: nucleoside having a 3′ n-butyl triester

SEQ ID NO: 135 - DNA, 30 bp, Artificial Sequence; identical to SEQ ID NO: 129
  Synthetic Construct
  misc_feature (1)..(30): nucleoside-3′-phosphate
  misc_feature 1, 29: nucleoside having a 3′ n-butyl triester

SEQ ID NO: 136 - DNA, 30 bp, Artificial Sequence; identical to SEQ ID NO: 132
  Synthetic Construct
  misc_feature (1)..(30): nucleoside-3′-phosphate
  misc_feature 1, 29: nucleoside having a 3′ n-butyl triester

SEQ ID NO: 137 - DNA, 30 bp, Artificial Sequence; identical to SEQ ID NO: 129
  Synthetic Construct
  misc_feature (1)..(30): nucleoside-3′-phosphate
  misc_feature 1: nucleoside having a 3′ n-butyl triester

SEQ ID NO: 138 - DNA, 30 bp, Artificial Sequence; identical to SEQ ID NO: 132
  Synthetic Construct
  misc_feature (1)..(30): nucleoside-3′-phosphate
  misc_feature 1: nucleoside having a 3′ n-butyl triester

SEQ ID NO: 139 - DNA, 20 bp, Artificial Sequence
  Synthetic Construct
  misc_feature (1)..(5): nucleoside-3′-phosphorothioate
  misc_feature (6)..(16): nucleoside-3′-phosphate
  misc_feature (17)..(20): nucleoside-3′-phosphorothioate
tccatgacgttcctgacgtt

SEQ ID NO: 140 - DNA, 20 bp, Artificial Sequence
  Synthetic Construct
  misc_feature (1)..(16): nucleoside-3′-phosphate
  misc_feature (17)..(20): nucleoside-3′-phosphorothioate
tccatgacgttcctgacgtt

SEQ ID NO: 141 - DNA, 20 bp, Artificial Sequence
  Synthetic Construct
  misc_feature (1)..(5): nucleoside-3′-phosphorothioate
  misc_feature (6)..(18): nucleoside-3′-phosphate
  misc_feature (19)..(20): nucleoside-3′-phosphorothioate
tccatgacgttcctgacgtt

SEQ ID NO: 142 - DNA, 20 bp, Artificial Sequence
  Synthetic Construct
  misc_feature (1)..(5): nucleoside-3′-phosphorothioate
  misc_feature (6)..(20): nucleoside-3′-phosphate
  misc_feature 19: nucleoside having a 3′ tBuDS-Ph (ortho) triester
tccatgacgttcctgacgtt

SEQ ID NO: 143 - DNA, 20 bp, Artificial Sequence
  Synthetic Construct
  misc_feature (1)..(5): nucleoside-3′-phosphorothioate
  misc_feature (6)..(20): nucleoside-3′-phosphate
  misc_feature 19: nucleoside having a 3′ tBuDS-Ph (ortho) triester
tccatgacgttcctgacgtt

SEQ ID NO: 144 - DNA, 24 bp, Artificial Sequence
  Synthetic Construct
  misc_feature (1)..(24): nucleoside-3′-phosphate
aacgacaaaacgacaaaacgacga

SEQ ID NO: 145 - DNA, 24 bp, Artificial Sequence
  Synthetic Construct
  misc_feature (1)..(24): nucleoside-3′-phosphate
  misc_feature 23: nucleoside having a 3′ n-butyl triester
aacgacaaaacgacaaaacgacga

SEQ ID NO: 146 - DNA, 24 bp, Artificial Sequence
  Synthetic Construct
  misc_feature (1)..(22): nucleoside-3′-phosphate
  misc_feature 23: nucleoside-3′-phosphorothioate
  misc_feature 24: nucleoside-3′-phosphate
  misc_feature 7: nucleoside having a 3′ DBCO-NH-PEG2 triester (N1)
tcgtcgttttgtcgttttgtcgtt

SEQ ID NO: 147 - DNA, 24 bp, Artificial Sequence
  Synthetic Construct
  misc_feature (1)..(18): nucleoside-3′-phosphate
  misc_feature (19)..(23): nucleoside-3′-phosphorothioate
  misc_feature 24: nucleoside-3′-phosphate
  misc_feature 7: nucleoside having a 3′ DBCO-NH-PEG2 triester (N1)
tcgtcgttttgtcgttttgtcgtt

SEQ ID NO: 148 - DNA, 24 bp, Artificial Sequence
  Synthetic Construct
  misc_feature (1)..(22): nucleoside-3′-phosphate
  misc_feature 23: nucleoside-3′-phosphorothioate
  misc_feature 24: nucleoside-3′-phosphate
  misc_feature 17: nucleoside having a 3′ DBCO-NH-PEG2 triester (N1)
tcgtcgttttgtcgttttgtcgtt

SEQ ID NO: 149 - DNA, 24 bp, Artificial Sequence
  Synthetic Construct
  misc_feature (1)..(18): nucleoside-3′-phosphate
  misc_feature (19)..(23): nucleoside-3′-phosphorothioate
  misc_feature 24: nucleoside-3′-phosphate
  misc_feature 17: nucleoside having a 3′ DBCO-NH-PEG2 triester (N1)
tcgtcgttttgtcgttttgtcgtt

SEQ ID NO: 150 - DNA, 24 bp, Artificial Sequence
  Synthetic Construct
  misc_feature (1)..(5): nucleoside-3′-phosphorothioate
  misc_feature (6)..(24): nucleoside-3′-phosphate
  misc_feature 23: nucleoside having a 3′ DBCO-NH-PEG2 triester (N1)
tcgtcgttttgtcgttttgtcgtt

SEQ ID NO: 151 - DNA, 24 bp, Artificial Sequence
  Synthetic Construct
  misc_feature (1)..(23): nucleoside-3′-phosphorothioate
  misc_feature 23: nucleoside having a 3′ DBCO-NH-PEG2 triester (N1)
  misc_feature 24: nucleoside-3′-phosphate
tcgtcgttttgtcgttttgtcgtt

SEQ ID NO: 152 - DNA, 24 bp, Artificial Sequence
  Synthetic Construct
  misc_feature (1)..(5): nucleoside-3′-phosphorothioate
  misc_feature (6)..(22): nucleoside-3′-phosphate
  misc_feature 23: nucleoside-3′-phosphorothioate
  misc_feature 23: nucleoside having a 3′ DBCO-NH-PEG2 triester (N1)
  misc_feature 24: nucleoside-3′-phosphate
tcgtcgttttgtcgttttgtcgtt

SEQ ID NO: 153 - DNA, 24 bp, Artificial Sequence
  Synthetic Construct
  misc_feature (1)..(5): nucleoside-3′-phosphorothioate
  misc_feature (6)..(22): nucleoside-3′-phosphate
  misc_feature 23: nucleoside having a 3′ DBCO-NH-PEG2 triester (N1)
  misc_feature (23)..(24): nucleoside-3′-phosphorothioate
tcgtcgttttgtcgttttgtcgtt

SEQ ID NO: 154 - DNA, 24 bp, Artificial Sequence
  Synthetic Construct
  misc_feature (1)..(5): nucleoside-3′-phosphorothioate
  misc_feature (6)..(23): nucleoside-3′-phosphate
  misc_feature 23: nucleoside having a 3′ DBCO-NH-PEG2 triester (N1)
  misc_feature 24: nucleoside-3′-phosphorothioate
tcgtcgttttgtcgttttgtcgtt

SEQ ID NO: 155 - DNA, 25 bp, Artificial Sequence
  Synthetic Construct
  misc_feature (1)..(25): nucleoside-3′-phosphate
  misc_feature 5: nucleoside having a 3′ DBCO-NH-PEG2 triester (N1)
  misc_feature 24: nucleoside having a 3′ n-butyl triester
ttcgtcgttttgtcgttttgtcgtt

SEQ ID NO: 156 - DNA, 26 bp, Artificial Sequence
  Synthetic Construct
  misc_feature (1)..(26): nucleoside-3′-phosphate
  misc_feature 6: nucleoside having a 3′ DBCO-NH-PEG2 triester (N1)
  misc_feature 25: nucleoside having a 3′ n-butyl triester
tttcgtcgttttgtcgttttgtcgtt

SEQ ID NO: 157 - DNA, 27 bp, Artificial Sequence
  Synthetic Construct
  misc_feature (1)..(27): nucleoside-3′-phosphate
  misc_feature 7: nucleoside having a 3′ DBCO-NH-PEG2 triester (N1)
  misc_feature 26: nucleoside having a 3′ n-butyl triester
gtttcgtcgttttgtcgttttgtcgtt

SEQ ID NO: 158 - DNA, 27 bp, Artificial Sequence
  Synthetic Construct
  misc_feature (1)..(27): nucleoside-3′-phosphate
  misc_feature 3: nucleoside having a 3′ n-butyl triester
  misc_feature 7: nucleoside having a 3′ DBCO-NH-PEG2 triester (N1)
  misc_feature 26: nucleoside having a 3′ n-butyl triester
gtttcgtcgttttgtcgttttgtcgtt

SEQ ID NO: 159 - DNA, 27 bp, Artificial Sequence
  Synthetic Construct
  misc_feature (1)..(27): nucleoside-3′-phosphate
  misc_feature 2: nucleoside having a 3′ n-butyl triester
  misc_feature 7: nucleoside having a 3′ DBCO-NH-PEG2 triester (N1)
  misc_feature 26: nucleoside having a 3′ n-butyl triester
gtttcgtcgttttgtcgttttgtcgtt

SEQ ID NO: 160 - DNA, 27 bp, Artificial Sequence
  Synthetic Construct
  misc_feature (1)..(27): nucleoside-3′-phosphate
  misc_feature 2: nucleoside having a 3′ n-butyl triester
  misc_feature 3: nucleoside having a 3′ n-butyl triester
  misc_feature 7: nucleoside having a 3′ DBCO-NH-PEG2 triester (N1)
  misc_feature 26: nucleoside having a 3′ n-butyl triester
gtttcgtcgttttgtcgttttgtcgtt

SEQ ID NO: 161 - DNA, 24 bp, Artificial Sequence
  Synthetic Construct
  misc_feature (1)..(24): nucleoside-3′-phosphate
  misc_feature 4: nucleoside having a 3′ DBCO-NH-PEG2 triester (N1)
  misc_feature 23: nucleoside having a 3′ n-butyl triester
  misc_feature 24: modified by C3 spacer (-(CH2)3-OH) (3′ phosphate)
tcgtcgttttgtcgttttgtcgtt

SEQ ID NO: 162 - DNA, 18 bp, Artificial Sequence
  Synthetic Construct
  misc_feature (1)..(18): nucleoside-3′-phosphate
  misc_feature 17: nucleoside having a 3′ DBCO-NH-PEG2 triester (N1)
tcgtcgttttgtcgtttt

SEQ ID NO: 163 - DNA, 24 bp, Artificial Sequence
  Synthetic Construct
  misc_feature (1)..(22): nucleoside-3′-phosphate
  misc_feature 1: 5-iodo-2′-deoxyuridine
  misc_feature (23)..(24): nucleoside-3′-phosphorothioate
  misc_feature 23: nucleoside having a 3′ DBCO-NH-PEG2 triester (N1)
  misc_feature 24: modified by C3 spacer (-(CH2)3-OH) (3′ phosphate)
ucgtcgttttgtcgttttgtcgtt

SEQ ID NO: 164 - DNA, 24 bp, Artificial Sequence
  Synthetic Construct
  misc_feature (1)..(24): nucleoside-3′-phosphate
  misc_feature 1: modified by C3 spacer (-(CH2)3-OH) (3′ phosphate)
  misc_feature 1: 5-iodo-2′-deoxyuridine
  misc_feature 23: nucleoside having a 3′ DBCO-NH-PEG2 triester (N1)
  misc_feature 24: modified by C3 spacer (-(CH2)3-OH) (3′ phosphate)
ucgtcgttttgtcgttttgtcgtt

SEQ ID NO: 165 - DNA, 24 bp, Artificial Sequence
  Synthetic Construct
  misc_feature (1)..(24): nucleoside-3′-phosphate
  misc_feature 4: 5-iodo-2′-deoxyuridine
  misc_feature 23: nucleoside having a 3′ DBCO-NH-PEG2 triester (N1)
  misc_feature 24: modified by C3 spacer (-(CH2)3-OH) (3′ phosphate)
tcgucgttttgtcgttttgtcgtt

SEQ ID NO: 166 - DNA, 24 bp, Artificial Sequence
  Synthetic Construct
  misc_feature (1)..(24): nucleoside-3′-phosphate
  misc_feature 1: modified by C3 spacer (-(CH2)3-OH) (3′ phosphate)
  misc_feature 4: 5-iodo-2′-deoxyuridine
  misc_feature 23: nucleoside having a 3′ DBCO-NH-PEG2 triester (N1)
  misc_feature 24: modified by C3 spacer (-(CH2)3-OH) (3′ phosphate)
tcgucgttttgtcgttttgtcgtt

SEQ ID NO: 167 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: 5-iodo-2′-deoxyuridine
  misc_feature 23: nucleoside having a 3′ DBCO-NH-PEG2 triester (N1)
  misc_feature 24: modified by C3 spacer (-(CH2)3-OH) (3′ phosphate)
ucgucgttttgtcgttttgtcgtt

SEQ ID NO: 168 - DNA, 24 bp, Artificial Sequence
  Synthetic Construct
  misc_feature (1)..(24): nucleoside-3′-phosphate
  misc_feature 1: modified by C3 spacer (-(CH2)3-OH) (5′ phosphate)
  misc_feature 1: 5-iodo-2′-deoxyuridine
  misc_feature 4: 5-iodo-2′-deoxyuridine
  misc_feature 23: nucleoside having a 3′ DBCO-NH-PEG2 triester (N1)
  misc_feature 24: modified by C3 spacer (-(CH2)3-OH) (3′ phosphate)
ucgucgttttgtcgttttgtcgtt

SEQ ID NO: 169 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: nucleoside having a 3′ DBCO-NH-PEG2 triester (N1)
  misc_feature 24: modified by C3 spacer (-(CH2)3-OH) (3′ phosphate)
ucgtcgttttgtcgttttgtcgtt

SEQ ID NO: 170 - DNA, 24 bp, Artificial Sequence
  Synthetic Construct
  misc_feature (1)..(24): nucleoside-3′-phosphate
  misc_feature 1: modified by C3 spacer (-(CH2)3-OH) (5′ phosphate)
  misc_feature 1: 5-iodo-2′-deoxyuridine
  misc_feature 4: nucleoside having a 3′ DBCO-NH-PEG2 triester (N1)
  misc_feature 24: modified by C3 spacer (-(CH2)3-OH) (3′ phosphate)
ucgtcgttttgtcgttttgtcgtt

SEQ ID NO: 171 - DNA, 18 bp, Artificial Sequence
  Synthetic Construct
  misc_feature (1)..(18): nucleoside-3′-phosphate
  misc_feature 4: nucleoside having a 3′ DBCO-NH-PEG2 triester (N1)
tcgtcgttttgtcgtttt

SEQ ID NO: 172 - DNA, 16 bp, Artificial Sequence
  Synthetic Construct
  misc_feature (1)..(16): nucleoside-3′-phosphate
  misc_feature 4: nucleoside having a 3′ DBCO-NH-PEG2 triester (N1)
tcgtcgttttgtcgtt

SEQ ID NO: 173 - DNA, 14 bp, Artificial Sequence
  Synthetic Construct
  misc_feature (1)..(14): nucleoside-3′-phosphate
  misc_feature 4: nucleoside having a 3′ DBCO-NH-PEG2 triester (N1)
tcgtcgttttgtcg

SEQ ID NO: 174 - DNA, 12 bp, Artificial Sequence
  Synthetic Construct
  misc_feature (1)..(12): nucleoside-3′-phosphate
  misc_feature 4: nucleoside having a 3′ DBCO-NH-PEG2 triester (N1)
tcgtcgttttgt

SEQ ID NO: 175 - DNA, 18 bp, Artificial Sequence
  Synthetic Construct
  misc_feature (1)..(18): nucleoside-3′-phosphate
  misc_feature 1: 5-iodo-2′-deoxyuridine
  misc_feature 4: nucleoside having a 3′ DBCO-NH-PEG2 triester (N1)
ucgtcgttttgtcgtttt

SEQ ID NO: 176 - DNA, 17 bp, Artificial Sequence
  Synthetic Construct
  misc_feature (1)..(17): nucleoside-3′-phosphate
  misc_feature 1: 5-iodo-2′-deoxyuridine
  misc_feature 5: nucleoside having a 3′ DBCO-NH-PEG2 triester (N1)
utcgtcgttttgtcgtt

SEQ ID NO: 177 - DNA, 14 bp, Artificial Sequence
  Synthetic Construct
  misc_feature (1)..(14): nucleoside-3′-phosphate
  misc_feature 1: 5-iodo-2′-deoxyuridine
  misc_feature 4: nucleoside having a 3′ DBCO-NH-PEG2 triester (N1)
ucgtcgttttgtcg

SEQ ID NO: 178 - DNA, 12 bp, Artificial Sequence
  Synthetic Construct
  misc_feature (1)..(12): nucleoside-3′-phosphate
  misc_feature 1: 5-iodo-2′-deoxyuridine
  misc_feature 4: nucleoside having a 3′ DBCO-NH-PEG2 triester (N1)
ucgtcgttttgt

SEQ ID NO: 179 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: 5-iodo-2′-deoxyuridine
  misc_feature 7: nucleoside having a 3′ n-butyl triester
  misc_feature 23: nucleoside having a 3′ DBCO-NH-PEG2 triester (N1)
  misc_feature 24: modified by C3 spacer (-(CH2)3-OH) (3′ phosphate)
ucgucgttttgtcgttttgtcgtt

SEQ ID NO: 180 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: nucleoside having a 3′ n-butyl triester
  misc_feature 23: nucleoside having a 3′ DBCO-NH-PEG2 triester (N1)
  misc_feature 24: modified by C3 spacer (-(CH2)3-OH) (3′ phosphate)
ucgtcgttttgtcgttttgtcgtt

SEQ ID NO: 181 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 7: nucleoside having a 3′ DBCO-NH-PEG2 triester (N1)
  misc_feature 23: nucleoside having a 3′ n-butyl triester
  misc_feature 24: modified by C3 spacer (-(CH2)3-OH) (3′ phosphate)
ucgtcgttttgtcgttttgtcgtt

SEQ ID NO: 182 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: nucleoside having a 3′ n-butyl triester
  misc_feature 7: nucleoside having a 3′ DBCO-NH-PEG2 triester (N1)
  misc_feature 23: nucleoside having a 3′ n-butyl triester
  misc_feature 24: modified by C3 spacer (-(CH2)3-OH) (3′ phosphate)
ucgtcgttttgtcgttttgtcgtt

SEQ ID NO: 183 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: nucleoside having a 3′ DBCO-NH-PEG2 triester (N1)
  misc_feature 23: nucleoside having a 3′ n-butyl triester
  misc_feature 24: modified by C3 spacer (-(CH2)3-OH) (3′ phosphate)
ucgtcgttttgtcgttttgtcgtt

SEQ ID NO: 184 - DNA, 20 bp, Artificial Sequence
  Synthetic Construct
  misc_feature (1)..(20): nucleoside-3′-phosphate
  misc_feature 20: nucleoside having a 3′ DBCO-NH-PEG2 triester (N1)
  misc_feature 20: modified by C3 spacer (-(CH2)3-OH) (3′ phosphate)
tccatgacgttcctgatgct

SEQ ID NO: 185 - DNA, 20 bp, Artificial Sequence
  Synthetic Construct
  misc_feature (1)..(20): nucleoside-3′-phosphorothioate
  misc_feature 20: nucleoside having a 3′ DBCO-NH-PEG2 triester (N1)
  misc_feature 20: modified by C3 spacer (-(CH2)3-OH) (3′ phosphate)
tccatgacgttcctgatgct

SEQ ID NO: 186 - DNA, 20 bp, Artificial Sequence
  Synthetic Construct
  misc_feature (1)..(20): nucleoside-3′-phosphorothioate
  misc_feature 1: nucleoside having a 3′ DBCO-NH-PEG2 triester (N1)
  misc_feature 20: modified by C3 spacer (-(CH2)3-OH) (3′ phosphate)
tccatgacgttcctgatgct

SEQ ID NO: 187 - DNA, 15 bp, Artificial Sequence
  Synthetic Construct
  misc_feature (1)..(15): nucleoside-3′-phosphate
  misc_feature 1: 5-iodo-2′-deoxyuridine
  misc_feature 4: nucleoside having a 3′ DBCO-NH-PEG2 triester (N1)
  misc_feature 15: modified by C3 spacer (-(CH2)3-OH) (3′ phosphate)
ucgtcgtttgtcgtt

SEQ ID NO: 188 - DNA, 14 bp, Artificial Sequence
  Synthetic Construct
  misc_feature (1)..(14): nucleoside-3′-phosphate
  misc_feature 1: 5-iodo-2′-deoxyuridine
  misc_feature 4: nucleoside having a 3′ DBCO-NH-PEG2 triester (N1)
  misc_feature 14: modified by C3 spacer (-(CH2)3-OH) (3′ phosphate)
ucgtcgttgtcgtt

SEQ ID NO: 189 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′ phosphate)
ucgtcgtgtcgtt

SEQ ID NO: 190 - DNA, 12 bp, Artificial Sequence
  Synthetic Construct
  misc_feature (1)..(12): nucleoside-3′-phosphate
  misc_feature 1: 5-iodo-2′-deoxyuridine
  misc_feature 4: nucleoside having a 3′ DBCO-NH-PEG2 triester (N1)
  misc_feature 12: modified by C3 spacer (-(CH2)3-OH) (3′ phosphate)
ucgtcgttcgtt

SEQ ID NO: 191 - DNA, 11 bp, Artificial Sequence
  Synthetic Construct
  misc_feature (1)..(11): nucleoside-3′-phosphate
  misc_feature 1: 5-iodo-2′-deoxyuridine
  misc_feature 4: nucleoside having a 3′ DBCO-NH-PEG2 triester (N1)
  misc_feature 11: modified by C3 spacer (-(CH2)3-OH) (3′ phosphate)
ucgtcgtcgtt

SEQ ID NO: 192 - DNA, 24 bp, Artificial Sequence
  Synthetic Construct
  misc_feature (1)..(24): nucleoside-3′-phosphate
  misc_feature 1: 5-iodo-2′-deoxyuridine
  misc_feature 4: nucleoside having a 3′ DBCO-NH-PEG2 triester (N1)
  misc_feature 23: nucleoside having a 3′ n-butyl triester
ugctgcttttgtgcttttgtgctt

SEQ ID NO: 193 - DNA, 20 bp, Artificial Sequence
  Synthetic Construct
  misc_feature (1)..(20): nucleoside-3′-phosphate
  misc_feature 20: nucleoside having a 3′ DBCO-NH-PEG2 triester (N1)
  misc_feature 20: modified by C3 spacer (-(CH2)3-OH) (3′ phosphate)
tccatgacgttcctgacgtt

SEQ ID NO: 194 - DNA, 20 bp, Artificial Sequence
  Synthetic Construct
  misc_feature (1)..(20): nucleoside-3′-phosphorothioate
  misc_feature 20: nucleoside having a 3′ DBCO-NH-PEG2 triester (N1)
  misc_feature 20: modified by C3 spacer (-(CH2)3-OH) (3′ phosphate)
tccatgacgttcctgacgtt

SEQ ID NO: 195 - DNA, 20 bp, Artificial Sequence
  Synthetic Construct
  misc_feature (1)..(20): nucleoside-3′-phosphate
  misc_feature 1: nucleoside having a 3′ DBCO-NH-PEG2 triester (N1)
  misc_feature 20: modified by C3 spacer (-(CH2)3-OH) (3′ phosphate)
tccatgacgttcctgacgtt

SEQ ID NO: 196 - DNA, 20 bp, Artificial Sequence
  Synthetic Construct
  misc_feature (1)..(20): nucleoside-3′-phosphorothioate
  misc_feature 1: nucleoside having a 3′ DBCO-NH-PEG2 triester (N1)
  misc_feature 20: modified by C3 spacer (-(CH2)3-OH) (3′ phosphate)
tccatgacgttcctgacgtt

SEQ ID NO: 197 - DNA, 25 bp, Artificial Sequence
  Synthetic Construct
  misc_feature (1)..(25): nucleoside-3′-phosphate
  misc_feature 1: nucleoside having a 3′ DBCO-NH-PEG2 triester (N1)
  misc_feature (2)..(25): 2′-fluoronucleotide
taacgacaaaacgacaaaacgacga

SEQ ID NO: 198 - DNA, 25 bp, Artificial Sequence
  Synthetic Construct
  misc_feature (1)..(25): nucleoside-3′-phosphate
  misc_feature (1)..(24): 2′-fluoronucleotide
  misc_feature 25: nucleoside having a 3′ DBCO-NH-PEG2 triester (N1)
  misc_feature 25: modified by C3 spacer (-(CH2)3-OH) (3′ phosphate)
aacgacaaaacgacaaaacgacgat

SEQ ID NO: 199 - DNA, 16 bp, Artificial Sequence
  Synthetic Construct
  misc_feature (1)..(6): nucleoside-3′-phosphate
  misc_feature 1: 5-iodo-2′-deoxyuridine
  misc_feature 4: nucleoside having a 3′ DBCO-NH-PEG2 triester (N1)
  misc_feature (7)..(12): nucleoside-3′-phosphorothioate
  misc_feature (13)..(16): nucleoside-3′-phosphate
  misc_feature 16: modified by C3 spacer (-(CH2)3-OH) (3′ phosphate)
ucgtcgttttgtcgtt

SEQ ID NO: 200 - DNA, 16 bp, Artificial Sequence
  Synthetic Construct
  misc_feature (1)..(6): nucleoside-3′-phosphate
  misc_feature 1: 5-iodo-2′-deoxyuridine
  misc_feature 4: nucleoside having a 3′ DBCO-NH-PEG2 triester (N1)
  misc_feature (7)..(12): nucleoside-3′-phosphorothioate
  misc_feature (13)..(16): nucleoside-3′-phosphate
  misc_feature 16: modified by C3 spacer (-(CH2)3-OH) (3′ phosphate)
ucgtcgttttgtcgtt

SEQ ID NO: 201 - DNA, 16 bp, Artificial Sequence
  Synthetic Construct
  misc_feature (1)..(6): nucleoside-3′-phosphate
  misc_feature 1: 5-iodo-2′-deoxyuridine
  misc_feature 4: nucleoside having a 3′ DBCO-NH-PEG2 triester (N1)
  misc_feature (7)..(8): nucleoside-3′-phosphorothioate
  misc_feature (9)..(16): nucleoside-3′-phosphate
  misc_feature 16: modified by C3 spacer (-(CH2)3-OH) (3′ phosphate)
ucgtcgttttgtcgtt

SEQ ID NO: 202 - DNA, 16 bp, Artificial Sequence
  Synthetic Construct
  misc_feature (1)..(8): nucleoside-3′-phosphate
  misc_feature 1: 5-iodo-2′-deoxyuridine
  misc_feature 4: nucleoside having a 3′ DBCO-NH-PEG2 triester (N1)
  misc_feature (9)..(10): nucleoside-3′-phosphorothioate
  misc_feature (11)..(16): nucleoside-3′-phosphate
  misc_feature 16: modified by C3 spacer (-(CH2)3-OH) (3′ phosphate)
ucgtcgttttgtcgtt

SEQ ID NO: 203 - DNA, 16 bp, Artificial Sequence
  Synthetic Construct
  misc_feature (1)..(10): nucleoside-3′-phosphate
  misc_feature 1: 5-iodo-2′-deoxyuridine
  misc_feature 4: nucleoside having a 3′ DBCO-NH-PEG2 triester (N1)
  misc_feature (11)..(12): nucleoside-3′-phosphorothioate
  misc_feature (13)..(16): nucleoside-3′-phosphate
  misc_feature 16: modified by C3 spacer (-(CH2)3-OH) (3′ phosphate)
ucgtcgttttgtcgtt

SEQ ID NO: 204 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 6: nucleoside having a 3′ n-butyl triester
  misc_feature 16: modified by C3 spacer (-(CH2)3-OH) (3′ phosphate)
ucgtcgttttgtcgtt

SEQ ID NO: 205 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 7: nucleoside having a 3′ n-butyl triester
  misc_feature 16: modified by C3 spacer (-(CH2)3-OH) (3′ phosphate)
ucgtcgttttgtcgtt

SEQ ID NO: 206 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 9: nucleoside having a 3′ n-butyl triester
  misc_feature 16: modified by C3 spacer (-(CH2)3-OH) (3′ phosphate)
ucgtcgttttgtcgtt

SEQ ID NO: 207 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 11: nucleoside having a 3′ n-butyl triester
  misc_feature 16: modified by C3 spacer (-(CH2)3-OH) (3′ phosphate)
ucgtcgttttgtcgtt

SEQ ID NO: 208 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 14: nucleoside having a 3′ n-butyl triester
  misc_feature 16: modified by C3 spacer (-(CH2)3-OH) (3′ phosphate)
ucgtcgttttgtcgtt

SEQ ID NO: 209 - DNA, 8 bp, Artificial Sequence
  Synthetic Construct
  misc_feature (1)..(8): nucleoside-3′-phosphate
  misc_feature 1: 5-iodo-2′-deoxyuridine
  misc_feature 4: nucleoside having a 3′ DBCO-NH-PEG2 triester (N1)
  misc_feature 8: modified by C3 spacer (-(CH2)3-OH) (3′ phosphate)
ucgtcgtt

SEQ ID NO: 210 - DNA, 8 bp, Artificial Sequence
  Synthetic Construct
  misc_feature (1)..(8): nucleoside-3′-phosphate
  misc_feature 1: 5-iodo-2′-deoxyuridine
  misc_feature 8: nucleoside having a 3′ DBCO-NH-PEG2 triester (N1)
  misc_feature 8: modified by C3 spacer (-(CH2)3-OH) (3′ phosphate)
ucgtcgtt

SEQ ID NO: 211 - DNA, 6 bp, Artificial Sequence
  Synthetic Construct
  misc_feature (1)..(6): nucleoside-3′-phosphate
  misc_feature 1: 5-iodo-2′-deoxyuridine
  misc_feature 4: nucleoside having a 3′ DBCO-NH-PEG2 triester (N1)
  misc_feature 6: modified by C3 spacer (-(CH2)3-OH) (3′ phosphate)
ucgttt

SEQ ID NO: 212 - DNA, 6 bp, Artificial Sequence
  Synthetic Construct
  misc_feature (1)..(6): nucleoside-3′-phosphate
  misc_feature 1: 5-iodo-2′-deoxyuridine
  misc_feature 6: nucleoside having a 3′ DBCO-NH-PEG2 triester (N1)
  misc_feature 6: modified by C3 spacer (-(CH2)3-OH) (3′ phosphate)
ucgttt

SEQ ID NO: 213 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′ phosphate)
ucgtcgtgtcgtt

SEQ ID NO: 214 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 13: nucleoside having a 3′ DBCO-NH-PEG2 triester (N1)
  misc_feature 13: modified by C3 spacer (-(CH2)3-OH) (3′ phosphate)
ucgtcgtgttttt

SEQ ID NO: 215 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 13: nucleoside having a 3′ DBCO-NH-PEG2 triester (N1)
  misc_feature 13: modified by C3 spacer (-(CH2)3-OH) (3′ phosphate)
ucgttttgtcgtt

SEQ ID NO: 216 - DNA, 12 bp, Artificial Sequence
  Synthetic Construct
  misc_feature (1)..(12): nucleoside-3′-phosphate
  misc_feature 1: 5-iodo-2′-deoxyuridine
  misc_feature 12: nucleoside having a 3′ DBCO-NH-PEG2 triester (N1)
  misc_feature 12: modified by C3 spacer (-(CH2)3-OH) (3′ phosphate)
ucgtttgtcgtt

SEQ ID NO: 217 - DNA, 11 bp, Artificial Sequence
  Synthetic Construct
  misc_feature (1)..(11): nucleoside-3′-phosphate
  misc_feature 1: 5-iodo-2′-deoxyuridine
  misc_feature 11: nucleoside having a 3′ DBCO-NH-PEG2 triester (N1)
  misc_feature 11: modified by C3 spacer (-(CH2)3-OH) (3′ phosphate)
ucgttgtcgtt

SEQ ID NO: 218 - DNA, 10 bp, Artificial Sequence
  Synthetic Construct
  misc_feature (1)..(10): nucleoside-3′-phosphate
  misc_feature 1: 5-iodo-2′-deoxyuridine
  misc_feature 10: nucleoside having a 3′ DBCO-NH-PEG2 triester (N1)
  misc_feature 10: modified by C3 spacer (-(CH2)3-OH) (3′ phosphate)
ucgtgtcgtt

SEQ ID NO: 219 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ DBCO-NH-PEG2 triester (N1)
  misc_feature 16: modified by C3 spacer (-(CH2)3-OH) (3′ phosphate)
ugctgcttttgtgctt

SEQ ID NO: 220 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 15: nucleoside having a 3′ DBCO-NH-PEG2 triester (N1)
  misc_feature 16: modified by C3 spacer (-(CH2)3-OH) (3′ phosphate)
ucgtcgttttgtcgtt

SEQ ID NO: 221 - DNA, 16 bp, Artificial Sequence
  Synthetic Construct
  misc_feature (1)..(16): nucleoside-3′-phosphate
  misc_feature 1: 5-iodo-2′-deoxyuridine
  misc_feature 4: nucleoside having a 3′ n-butyl triester
  misc_feature 15: nucleoside having a 3′ DBCO-NH-PEG2 triester (N1)
  misc_feature 16: modified by C3 spacer (-(CH2)3-OH) (3′ phosphate)
ucgtcgttttgtcgtt

SEQ ID NO: 222 - DNA, 13 bp, Artificial Sequence
  Synthetic Construct
  misc_feature (1)..(13): nucleoside-3′-phosphate
  misc_feature 13: nucleoside having a 3′ DBCO-NH-PEG2 triester (N1)
gggacgatcgtct

SEQ ID NO: 223 - DNA, 15 bp, Artificial Sequence
  Synthetic Construct
  misc_feature 1, 2, 14, 15: nucleoside-3′-phosphorothioate
  misc_feature (3)..(13): nucleoside-3′-phosphate
  misc_feature 13: nucleoside having a 3′ DBCO-NH-PEG2 triester (N1)
gggacgatcgtctgg

SEQ ID NO: 224 - DNA, 15 bp, Artificial Sequence
  Synthetic Construct
  misc_feature 1, 2, 14, 15: nucleoside-3′-phosphorothioate
  misc_feature (3)..(13): nucleoside-3′-phosphate
  misc_feature 8: nucleoside having a 3′ DBCO-NH-PEG2 triester (N1)
gggacgatcgtctgg

SEQ ID NO: 225 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 4: nucleoside having a 3′ DBCO-NH-PEG2 triester (N1)
  misc_feature 6: nucleoside having a 3′ n-butyl triester
  misc_feature 13: modified by C3 spacer (-(CH2)3-OH) (3′ phosphate)
ucgtcgtgtcgtt

SEQ ID NO: 226 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 4: nucleoside having a 3′ DBCO-NH-PEG2 triester (N1)
  misc_feature 7: nucleoside having a 3′ n-butyl triester
  misc_feature 13: modified by C3 spacer (-(CH2)3-OH) (3′ phosphate)
ucgtcgtgtcgtt

SEQ ID NO: 227 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 4: nucleoside having a 3′ DBCO-NH-PEG2 triester (N1)
  misc_feature 9: nucleoside having a 3′ n-butyl triester
  misc_feature 13: modified by C3 spacer (-(CH2)3-OH) (3′ phosphate)
ucgtcgtgtcgtt

SEQ ID NO: 228 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 4: nucleoside having a 3′ DBCO-NH-PEG2 triester (N1)
  misc_feature 12: nucleoside having a 3′ n-butyl triester
  misc_feature 13: modified by C3 spacer (-(CH2)3-OH) (3′ phosphate)
ucgtcgtgtcgtt

SEQ ID NO: 229 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 3: nucleoside having a 3′ n-butyl triester
  misc_feature 12: nucleoside having a 3′ DBCO-NH-PEG2 triester (N1)
  misc_feature 13: modified by C3 spacer (-(CH2)3-OH) (3′ phosphate)
ucgtcgtgtcgtt

SEQ ID NO: 230 - DNA, 13 bp, Artificial Sequence
  Synthetic Construct
  misc_feature (1)..(13): nucleoside-3′-phosphate
  misc_feature 1: 5-iodo-2′-deoxyuridine
  misc_feature 11: nucleoside having a 3′ n-butyl triester
  misc_feature 12: nucleoside having a 3′ DBCO-NH-PEG2 triester (N1)
  misc_feature 13: modified by C3 spacer (-(CH2)3-OH) (3′ phosphate)
ucgtcgtgtcgtt

SEQ ID NO: 231 - DNA, 13 bp, Artificial Sequence
  Synthetic Construct
  misc_feature (1)..(5): nucleoside-3′-phosphate
  misc_feature (6)..(9): nucleoside-3′-phosphorothioate
  misc_feature (10)..(13): nucleoside-3′-phosphate
  misc_feature 1: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′ phosphate)
ucgtcgtgtcgtt

SEQ ID NO: 232 - DNA, 24 bp, Artificial Sequence
  Synthetic Construct
  misc_feature (1)..(23): nucleoside-3′-phosphorothioate
  misc_feature 23: nucleoside having a 3′ DBCO-NH-PEG2 triester (N1)
  misc_feature 24: nucleoside-3′-phosphate
  misc_feature 24: modified by C3 spacer (-(CH2)3-OH) (3′ phosphate)
tcgtcgttttgtcgttttgtcgtt

SEQ ID NO: 233 - DNA, 24 bp, Artificial Sequence
  Synthetic Construct
  misc_feature (1)..(23): nucleoside-3′-phosphorothioate
  misc_feature 1: 5-iodo-2′-deoxyuridine
  misc_feature 23: nucleoside having a 3′ DBCO-NH-PEG2 triester (N1)
  misc_feature 24: nucleoside-3′-phosphate
  misc_feature 24: modified by C3 spacer (-(CH2)3-OH) (3′ phosphate)
ucgtcgttttgtcgttttgtcgtt

SEQ ID NO: 234 - DNA, 24 bp, Artificial Sequence
  Synthetic Construct
tcgtcgttttgtcgttttgtcgtt

SEQ ID NO: 235 - DNA, 16 bp, Artificial Sequence
  Synthetic Construct
  misc_feature (1)..(15): nucleoside-3′-phosphorothioate
  misc_feature 15: nucleoside having a 3′ DBCO-NH-PEG2 triester (N1)
  misc_feature 16: nucleoside-3′-phosphate
  misc_feature 16: modified by C3 spacer (-(CH2)3-OH) (3′ phosphate)
tcgtcgttttgtcgtt

SEQ ID NO: 236 - DNA, 16 bp, Artificial Sequence
  Synthetic Construct
  misc_feature (1)..(15): nucleoside-3′-phosphorothioate
  misc_feature 1: 5-iodo-2′-deoxyuridine
  misc_feature 15: nucleoside having a 3′ DBCO-NH-PEG2 triester (N1)
  misc_feature 16: nucleoside-3′-phosphate
  misc_feature 16: modified by C3 spacer (-(CH2)3-OH) (3′ phosphate)
ucgtcgttttgtcgtt

SEQ ID NO: 237 - DNA, 13 bp, Artificial Sequence
  Synthetic Construct
  misc_feature (1)..(12): nucleoside-3′-phosphorothioate
  misc_feature 12: nucleoside having a 3′ DBCO-NH-PEG2 triester (N1)
  misc_feature 13: nucleoside-3′-phosphate
  misc_feature 13: modified by C3 spacer (-(CH2)3-OH) (3′ phosphate)
tcgtcgtgtcgtt

SEQ ID NO: 238 - DNA, 13 bp, Artificial Sequence
  Synthetic Construct
  misc_feature (1)..(12): nucleoside-3′-phosphorothioate
  misc_feature 1: 5-iodo-2′-deoxyuridine
  misc_feature 12: nucleoside having a 3′ DBCO-NH-PEG2 triester (N1)
  misc_feature 13: nucleoside-3′-phosphate
  misc_feature 13: modified by C3 spacer (-(CH2)3-OH) (3′- phosphate)
ucgtcgtgtcgtt

SEQ ID NO: 239 - DNA, 13 bp, Artificial Sequence
  Synthetic Construct
  misc_feature 1, 2, 5, 10, 12, 13: nucleoside-3′-phosphate
  misc_feature 1: 5-iodo-2′-deoxyuridine
  misc_feature 3, 4, 6, 7, 8, 9, 11: nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 240 - DNA, 13 bp, Artificial Sequence
  Synthetic Construct
  misc_feature 1, 2, 5, 10: nucleoside-3′-phosphate
  misc_feature 1: 5-iodo-2′-deoxyuridine
  misc_feature 3, 4, 6, 7, 8, 9, 11, 12, 13: nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 241 - DNA, 13 bp, Artificial Sequence
  Synthetic Construct
  misc_feature (1)..(2): nucleoside-3′-phosphate
  misc_feature 1: 5-iodo-2′-deoxyuridine
  misc_feature (3)..(13): nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 242 - DNA, 13 bp, Artificial Sequence
  Synthetic Construct
  misc_feature 1: nucleoside-3′-phosphate
  misc_feature 1: 5-iodo-2′-deoxyuridine
  misc_feature (2)..(13): nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 243 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 244 - DNA, 13 bp, Artificial Sequence
  Synthetic Construct
  misc_feature (1)..(2): nucleoside-3′-phosphate
  misc_feature 1: 5-iodo-2′-deoxyuridine
  misc_feature (3)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 245 - DNA, 13 bp, Artificial Sequence
  Synthetic Construct
  misc_feature 1: nucleoside-3′-phosphate
  misc_feature 1: 5-iodo-2′-deoxyuridine
  misc_feature (2)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 246 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 247 - DNA, 24 bp, Artificial Sequence
  Synthetic Construct
  misc_feature (1)..(2): nucleoside-3′-phosphate
  misc_feature 1: 5-iodo-2′-deoxyuridine
  misc_feature (3)..(24): nucleoside-3′-phosphorothioate
  misc_feature 4: nucleoside having a 3′ DBCO-NH-PEG2 triester (N1)
  misc_feature 24: modified by C3 spacer (-(CH2)3-OH) (3′- phosphate)
ucgtcgttttgtcgttttgtcgtt

SEQ ID NO: 248 - DNA, 24 bp, Artificial Sequence
  Synthetic Construct
  misc_feature 1: nucleoside-3′-phosphate
  misc_feature 1: 5-iodo-2′-deoxyuridine
  misc_feature (2)..(24): nucleoside-3′-phosphorothioate
  misc_feature 4: nucleoside having a 3′ DBCO-NH-PEG2 triester (N1)
  misc_feature 24: modified by C3 spacer (-(CH2)3-OH) (3′- phosphate)
ucgtcgttttgtcgttttgtcgtt

SEQ ID NO: 249 - DNA, 24 bp, Artificial Sequence
  Synthetic Construct
  misc_feature (1)..(24): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 24: modified by C3 spacer (-(CH2)3-OH) (3′- phosphate)
ucgtcgttttgtcgttttgtcgtt

SEQ ID NO: 250 - DNA, 13 bp, Artificial Sequence
  Synthetic Construct
  misc_feature 1, 2, 3, 4, 5, 10, 11, 12, 13: nucleoside-3′-phosphate
  misc_feature 1: 5-iodo-2′-deoxyuridine
  misc_feature 6, 7, 8, 9: nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 251 - DNA, 13 bp, Artificial Sequence
  Synthetic Construct
  misc_feature 1, 2, 3, 4, 5, 10: nucleoside-3′-phosphate
  misc_feature 1: 5-iodo-2′-deoxyuridine
  misc_feature 6, 7, 8, 9, 11, 12, 13: nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 252 - DNA, 13 bp, Artificial Sequence
  Synthetic Construct
  misc_feature 1, 2, 4, 5, 10: nucleoside-3′-phosphate
  misc_feature 1: 5-iodo-2′-deoxyuridine
  misc_feature 3, 6, 7, 8, 9, 11, 12, 13: nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 253 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 3: contains a 2′-OMe-modified ribose
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature 13: modified by C3 spacer (-(CH2)3-OH) (3′- phosphate)
ucgtcgtgtcgtt

SEQ ID NO: 254 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature 11: contains a 2′-OMe-modified ribose
  misc_feature 13: modified by C3 spacer (-(CH2)3-OH) (3′- phosphate)
ucgtcgtgtcgtt

SEQ ID NO: 255 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature 12: contains a 2′-OMe-modified ribose
  misc_feature 13: modified by C3 spacer (-(CH2)3-OH) (3′- phosphate)
ucgtcgtgtcgtt

SEQ ID NO: 256 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature 13: contains a 2′-OMe-modified ribose
  misc_feature 13: modified by C3 spacer (-(CH2)3-OH) (3′- phosphate)
ucgtcgtgtcgtt

SEQ ID NO: 257 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature 12, 13: contains a 2′-OMe-modified ribose
  misc_feature 13: modified by C3 spacer (-(CH2)3-OH) (3′- phosphate)
ucgtcgtgtcgtt

SEQ ID NO: 258 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: nucleoside having a 3′ DBCO-NH-PEG2 triester (N1)
  misc_feature (6)..(9): nucleoside-3′-phosphorothioate
  misc_feature (10)..(13): nucleoside-3′-phosphate
  misc_feature (11)..(13): each contains a 2′-OMe-modified ribose
  misc_feature 13: modified by C3 spacer (-(CH2)3-OH) (3′- phosphate)
ucgtcgtgtcgtt

SEQ ID NO: 259 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature 4: contains a 2′-OMe-modified ribose
  misc_feature (6)..(9): nucleoside-3′-phosphorothioate
  misc_feature (10)..(13): nucleoside-3′-phosphate
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 260 - DNA, 13 bp, Artificial Sequence
  Synthetic Construct
  misc_feature (1)..(5): nucleoside-3′-phosphate
  misc_feature 1: 5-iodo-2′-deoxyuridine
  misc_feature (6)..(9): nucleoside-3′-phosphorothioate
  misc_feature (10)..(13): nucleoside-3′-phosphate
  misc_feature 12: nucleoside having a 3′ DBCO-NH-PEG2 triester (N1)
  misc_feature 13: contains a 2′-OMe-modified ribose
  misc_feature 13: modified by C3 spacer (-(CH2)3-OH) (3′- phosphate)
ucgtcgtgtcgtt

SEQ ID NO: 261 - DNA, 13 bp, Artificial Sequence
  Synthetic Construct
  misc_feature (1)..(5): nucleoside-3′-phosphate
  misc_feature 1: nucleoside having a 3′ n-butyl triester
  misc_feature (6)..(9): nucleoside-3′-phosphorothioate
  misc_feature (10)..(13): nucleoside-3′-phosphate
  misc_feature 12: nucleoside having a 3′ DBCO-NH-PEG2 triester (N1)
  misc_feature 13: contains a 2′-OMe-modified ribose
  misc_feature 13: modified by C3 spacer (-(CH2)3-OH) (3′- phosphate)
ucgtcgtgtcgtt

SEQ ID NO: 262 - DNA, 13 bp, Artificial Sequence
  Synthetic Construct
  misc_feature 1: nucleoside-3′-phosphate
  misc_feature 1, 4: 5-iodo-2′-deoxyuridine
  misc_feature (2)..(13): nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
ucgucgtgtcgtt

SEQ ID NO: 263 - DNA, 13 bp, Artificial Sequence
  Synthetic Construct
  misc_feature 1: nucleoside-3′-phosphate
  misc_feature 1, 9: 5-iodo-2′-deoxyuridine
  misc_feature 4: nucleoside having a 3′ DBCO-NH-PEG2 triester (N1)
  misc_feature (2)..(13): nucleoside-3′-phosphorothioate
  misc_feature 13: modified by C3 spacer (-(CH2)3-OH) (3′- phosphate)
ucgtcgtgucgtt

SEQ ID NO: 264 - DNA, 14 bp, Artificial Sequence
  Synthetic Construct
  misc_feature (1)..(14): nucleoside-3′-phosphate
  misc_feature 1: nucleoside having a 3′ DBCO-NH-PEG2 triester (N1)
  misc_feature (2)..(14): 2′-fluoronucleotide
taacgacacgacga

SEQ ID NO: 265 - DNA, 14 bp, Artificial Sequence
  Synthetic Construct
  misc_feature (1)..(14): nucleoside-3′-phosphate
  misc_feature (1)..(13): 2′-fluoronucleotide
  misc_feature 14: nucleoside having a 3′ DBCO-NH-PEG2 triester (N1)
aacgacacgacgat

SEQ ID NO: 266 - DNA, 13 bp, Artificial Sequence
  Synthetic Construct
  misc_feature (1)..(13): nucleoside-3′-phosphorothioate
  misc_feature 1, 9: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgucgtt

SEQ ID NO: 267 - DNA, 12 bp, Artificial Sequence
  Synthetic Construct
  misc_feature (1)..(12): nucleoside-3′-phosphorothioate
  misc_feature 3: nucleoside having a 3′ DBCO-NH-PEG2 triester (N1)
  misc_feature 12: modified by C3 spacer (-(CH2)3-OH) (3′- phosphate)
cgtcgtgtcgtt

SEQ ID NO: 268 - DNA, 12 bp, Artificial Sequence
  Synthetic Construct
  misc_feature (1)..(12): nucleoside-3′-phosphorothioate
  misc_feature 3: nucleoside having a 3′ DBCO-NH-PEG2 triester (N1)
  misc_feature 8: 5-iodo-2′-deoxyuridine
  misc_feature 12: modified by C3 spacer (-(CH2)3-OH) (3′- phosphate)
cgtcgtgucgtt

SEQ ID NO: 269 - DNA, 13 bp, Artificial Sequence
  Synthetic Construct
  misc_feature 1: nucleoside-3′-phosphate
  misc_feature (2)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
tcgtcgtgtcgtt

SEQ ID NO: 270 - DNA, 13 bp, Artificial Sequence
  Synthetic Construct
  misc_feature (1)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
tcgtcgtgtcgtt

SEQ ID NO: 271 - DNA, 13 bp, Artificial Sequence
  Synthetic Construct
  misc_feature 1: nucleoside-3′-phosphate
  misc_feature 1: 5-iodo-2′-deoxyuridine
  misc_feature (2)..(13): nucleoside-3′-phosphorothioate
  misc_feature 4: nucleoside having a 3′ n-butyl triester
  misc_feature 13: modified by C3 spacer (-(CH2)3-OH) (3′- phosphate)
ucgtcgtgtcgtt

SEQ ID NO: 272 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ n-butyl triester
  misc_feature 13: modified by C3 spacer (-(CH2)3-OH) (3′- phosphate)
ucgtcgtgtcgtt

SEQ ID NO: 273 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ugctgctgtgctt

SEQ ID NO: 274 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgagctgtcgtt

SEQ ID NO: 275 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgacgtt

SEQ ID NO: 276 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgacgtgacgtt

SEQ ID NO: 277 - DNA, 13 bp, Artificial Sequence
  Synthetic Construct
  misc_feature (1)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
acgacgtgacgtt

SEQ ID NO: 278 - DNA, 13 bp, Artificial Sequence
  Synthetic Construct
  misc_feature (1)..(13): nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
acgacgtgacgtt

SEQ ID NO: 279 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 3: 7-deaza-2′-deoxyguanosine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 280 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 6: 7-deaza-2′-deoxyguanosine
  misc_feature 13: modified by C3 spacer (-(CH2)3-OH) (3′- phosphate)
ucgtcgtgtcgtt

SEQ ID NO: 281 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 11: 7-deaza-2′-deoxyguanosine
  misc_feature 13: modified by C3 spacer (-(CH2)3-OH) (3′- phosphate)
ucgtcgtgtcgtt

SEQ ID NO: 282 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 3, 6, 11: 7-deaza-2′-deoxyguanosine
  misc_feature 4: nucleoside having a 3′ tBuDS-Ph (ortho) triester (PS)
  misc_feature 13: modified by C3 spacer (-(CH2)3-OH) (3′- phosphate)
ucgtcgtgtcgtt

SEQ ID NO: 283 - DNA, 13 bp, Artificial Sequence
  Synthetic Construct
  misc_feature (1)..(13): nucleoside-3′-phosphorothioate
  misc_feature 3, 6, 11: 7-deaza-2′-deoxyguanosine
  misc_feature 4: nucleoside having a 3′ DBCO-NH-PEG2 triester (N1)
  misc_feature 13: modified by C3 spacer (-(CH2)3-OH) (3′- phosphate)
acgacgtgacgtt

SEQ ID NO: 284 - DNA, 13 bp, Artificial Sequence
  Synthetic Construct
  misc_feature (1)..(13): nucleoside-3′-phosphorothioate
  misc_feature 3, 6, 11: 7-deaza-2′-deoxyguanosine
  misc_feature 12: nucleoside having a 3′ DBCO-NH-PEG2 triester (N1)
  misc_feature 13: modified by C3 spacer (-(CH2)3-OH) (3′- phosphate)
acgacgtgacgtt

SEQ ID NO: 285 - DNA, 13 bp, Artificial Sequence
  Synthetic Construct
  misc_feature (1)..(12): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: nucleoside-3′-phosphate
  misc_feature 13: modified by OH
ucgtcgtgtcgtt

SEQ ID NO: 286 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 13: modified by C3 spacer (-(CH2)3-OH) (3′- phosphate)
ucgtcgtgtcgtt

SEQ ID NO: 287 - DNA, 13 bp, Artificial Sequence
  Synthetic Construct
  misc_feature (1)..(12): nucleoside-3′-phosphorothioate
  misc_feature 1: 5-iodo-2′-deoxyuridine
  misc_feature 13: nucleoside-3′-phosphate
ucgtcgtgtcgtt

SEQ ID NO: 288 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 289 - DNA, 13 bp, Artificial Sequence
  Synthetic Construct
  misc_feature (1)..(12): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: nucleoside-3′-phosphate
ucgtcgtgtcgtt

SEQ ID NO: 290 - DNA, 13 bp, Artificial Sequence
  Synthetic Construct
  misc_feature (1)..(13): nucleoside-3′-phosphorothioate
  misc_feature 13: modified by C3 spacer (-(CH2)3-OH) (3′- phosphate)
tcgtcgtgtcgtt

SEQ ID NO: 291 - DNA, 13 bp, Artificial Sequence
  Synthetic Construct
  misc_feature (1)..(12): nucleoside-3′-phosphorothioate
  misc_feature 13: nucleoside-3′-phosphate
tcgtcgtgtcgtt

SEQ ID NO: 292 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 13: nucleoside having a 3′ DBCO-NH-PEG2 triester (N1)
  misc_feature 13: modified by C3 spacer (-(CH2)3-OH) (3′- phosphate)
ucgtcgtgacgtt

SEQ ID NO: 293 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 13: nucleoside having a 3′ DBCO-NH-PEG2 triester (N1)
  misc_feature 13: modified by C3 spacer (-(CH2)3-OH) (3′- phosphate)
ucgacgtgacgtt

SEQ ID NO: 294 - DNA, 19 bp, Artificial Sequence
  Synthetic Construct
  misc_feature (1)..(19): nucleoside-3′-phosphorothioate
  misc_feature 7: 5-iodo-2′-deoxyuridine
  misc_feature 19: nucleoside having a 3′ DBCO-NH-PEG2 triester (N1)
  misc_feature 19: modified by C3 spacer (-(CH2)3-OH) (3′- phosphate)
tccatgucgttccttgatt

SEQ ID NO: 295 - DNA, 16 bp, Artificial Sequence
  Synthetic Construct
  misc_feature (1)..(16): nucleoside-3′-phosphorothioate
  misc_feature 7: 5-iodo-2′-deoxyuridine
  misc_feature 16: nucleoside having a 3′ DBCO-NH-PEG2 triester (N1)
  misc_feature 16: modified by C3 spacer (-(CH2)3-OH) (3′- phosphate)
tccatgucgttccttt

SEQ ID NO: 296 - DNA, 14 bp, Artificial Sequence
  Synthetic Construct
  misc_feature (1)..(14): nucleoside-3′-phosphorothioate
  misc_feature 7: 5-iodo-2′-deoxyuridine
  misc_feature 14: nucleoside having a 3′ DBCO-NH-PEG2 triester (N1)
  misc_feature 14: modified by C3 spacer (-(CH2)3-OH) (3′- phosphate)
tccatgucgttctt

SEQ ID NO: 297 - DNA, 11 bp, Artificial Sequence
  Synthetic Construct
  misc_feature (1)..(11): nucleoside-3′-phosphorothioate
  misc_feature 7: 5-iodo-2′-deoxyuridine
  misc_feature 11: nucleoside having a 3′ DBCO-NH-PEG2 triester (N1)
  misc_feature 11: modified by C3 spacer (-(CH2)3-OH) (3′- phosphate)
tccatgucgtt

SEQ ID NO: 298 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
tucgtcgtgtcgtt

SEQ ID NO: 299 - DNA, 14 bp, Artificial Sequence
  Synthetic Construct
  misc_feature (1)..(14): nucleoside-3′-phosphorothioate
  misc_feature 1, 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
uucgtcgtgtcgtt

SEQ ID NO: 300 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
uucgtcgtgtcgtt

SEQ ID NO: 301 - DNA, 13 bp, Artificial Sequence
  Synthetic Construct
  misc_feature (1)..(13): nucleoside-3′-phosphorothioate
  misc_feature 4: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′- phosphate)
tcgucgtgtcgtt

SEQ ID NO: 302 - DNA, 13 bp, Artificial Sequence
  Synthetic Construct
  misc_feature (1)..(3): nucleoside-3′-phosphorothioate
  misc_feature 4: nucleoside-3′-phosphate
  misc_feature (5)..(13): nucleoside-3′-phosphorothioate
  misc_feature 4: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′- phosphate)
tcgucgtgtcgtt

SEQ ID NO: 303 - DNA, 13 bp, Artificial Sequence
  Synthetic Construct
  misc_feature (1)..(3): nucleoside-3′-phosphorothioate
  misc_feature 4: nucleoside-3′-phosphate
  misc_feature (5)..(13): nucleoside-3′-phosphorothioate
  misc_feature 4: 5-iodo-2′-deoxyuridine
  misc_feature 7: nucleoside having a 3′ DBCO-NH-PEG2 triester (N1)
  misc_feature 13: modified by C3 spacer (-(CH2)3-OH) (3′- phosphate)
tcgucgtgtcgtt

SEQ ID NO: 304 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′- phosphate)
ucgtcgtgacgtt

SEQ ID NO: 305 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 12: nucleoside having a 3′ DBCO-NH-PEG2 triester (N1)
  misc_feature 13: modified by C3 spacer (-(CH2)3-OH) (3′- phosphate)
ucgacgtgacgtt

SEQ ID NO: 306 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 1: modified by C3 spacer (-(CH2)3-OH) ( 5′- phosphate)
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 307 - DNA, 13 bp, Artificial Sequence
  Synthetic Construct
  misc_feature (1)..(13): nucleoside-3′-phosphorothioate
  misc_feature 1: 5-fluoro-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 308 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 309 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 1: modified by C3 spacer (-(CH2)3-OH) (5′- phosphorothioate)
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 310 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: dT PEG2-NH2 triester
  misc_feature 13: modified by C3 spacer (-(CH2)3-OH) (3′- phosphate)
ucgtcgtgtcgtt

SEQ ID NO: 311 - DNA, 13 bp, Artificial Sequence
  Synthetic Construct
  misc_feature (1)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 312 - DNA, 13 bp, Artificial Sequence
  Synthetic Construct
  misc_feature (1)..(13): nucleoside-3′-phosphorothioate
  misc_feature 12: nucleoside having a 3′ DBCO-NH-PEG2 triester (N1)
  misc_feature 13: modified by C3 spacer (-(CH2)3-OH) (3′- phosphate)
tcgucgtgtcgtt

SEQ ID NO: 313 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
tucgtcgtgacgtt

SEQ ID NO: 314 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
uucgtcgtgacgtt

SEQ ID NO: 315 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 1: modified by 6-aminohex-1-yl
  misc_feature 4: nucleoside having a 3′ DBCO-NH-PEG2 triester (N1)
  misc_feature 13: modified by C3 spacer (-(CH2)3-OH) (3′- phosphate)
ucgtcgtgacgtt

SEQ ID NO: 316 - DNA, 14 bp, Artificial Sequence
  Synthetic Construct
  misc_feature (1)..(14): nucleoside-3′-phosphorothioate
  misc_feature 1: modified by C3 spacer (-(CH2)3-OH) (5′- phosphate)
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
uucgtcgtgacgtt

SEQ ID NO: 317 - DNA, 13 bp, Artificial Sequence
  Synthetic Construct
  misc_feature (1)..(13): nucleoside-3′-phosphorothioate
  misc_feature 4: nucleoside having a 3′ DBCO-NH-PEG2 triester (N1)
  misc_feature 9: 5-iodo-2′-deoxyuridine
  misc_feature 13: modified by C3 spacer (-(CH2)3-OH) (3′- phosphate)
tcgacgtgucgtt

SEQ ID NO: 318 - DNA, 13 bp, Artificial Sequence
  Synthetic Construct
  misc_feature (1)..(13): nucleoside-3′-phosphorothioate
  misc_feature 4: nucleoside having a 3′ DBCO-NH-PEG2 triester (N1)
  misc_feature 13: modified by C3 spacer (-(CH2)3-OH) (3′- phosphate)
tcgacgtgacgtt

SEQ ID NO: 319 - DNA, 13 bp, Artificial Sequence
  Synthetic Construct
  misc_feature (1)..(13): nucleoside-3′-phosphorothioate
  misc_feature 1, 9: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgacgtgucgtt

SEQ ID NO: 320 - DNA, 14 bp, Artificial Sequence
  Synthetic Construct
  misc_feature (1)..(14): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 14: modified by C3 spacer (-(CH2)3-OH) (3′- phosphate)
ucgtccatgacgtt

SEQ ID NO: 321 - DNA, 14 bp, Artificial Sequence
  Synthetic Construct
  misc_feature (1)..(14): nucleoside-3′-phosphorothioate
  misc_feature 1, 10: 5-iodo-2′-deoxyuridine
  misc_feature 4: nucleoside having a 3′ DBCO-NH-PEG2 triester (N1)
  misc_feature 14: modified by C3 spacer (-(CH2)3-OH) (3′- phosphate)
ucgtccatgucgtt

SEQ ID NO: 322 - DNA, 14 bp, Artificial Sequence
  Synthetic Construct
  misc_feature (1)..(14): nucleoside-3′-phosphorothioate
  misc_feature 4: nucleoside having a 3′ DBCO-NH-PEG2 triester (N1)
  misc_feature 10: 5-iodo-2′-deoxyuridine
  misc_feature 14: modified by C3 spacer (-(CH2)3-OH) (3′- phosphate)
tcgtccatgucgtt

SEQ ID NO: 323 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ucgtcgtgacgtt

SEQ ID NO: 324 - DNA, 14 bp, Artificial Sequence
  Synthetic Construct
  misc_feature (1)..(14): nucleoside-3′-phosphorothioate
  misc_feature 5: 5-iodo-2′-deoxyuridine
  misc_feature 14: nucleoside having a 3′ DBCO-NH-PEG2 triester (N1)
  misc_feature 14: modified by C3 spacer (-(CH2)3-OH) (3′- phosphate)
catgucgttccttt

SEQ ID NO: 325 - DNA, 12 bp, Artificial Sequence
  Synthetic Construct
  misc_feature (1)..(12): nucleoside-3′-phosphorothioate
  misc_feature 3: 5-iodo-2′-deoxyuridine
  misc_feature 12: nucleoside having a 3′ DBCO-NH-PEG2 triester (N1)
  misc_feature 12: modified by C3 spacer (-(CH2)3-OH) (3′- phosphate)
tgucgttccttt

SEQ ID NO: 326 - DNA, 14 bp, Artificial Sequence
  Synthetic Construct
  misc_feature (1)..(14): nucleoside-3′-phosphorothioate
  misc_feature 5: 5-iodo-2′-deoxyuridine
  misc_feature 14: nucleoside having a 3′ DBCO-NH-PEG2 triester (N1)
  misc_feature 14: modified by C3 spacer (-(CH2)3-OH) (3′- phosphate)
tatgucgttccttt

SEQ ID NO: 327 - DNA, 16 bp, Artificial Sequence
  Synthetic Construct
  misc_feature (1)..(16): nucleoside-3′-phosphorothioate
  misc_feature 16: nucleoside having a 3′ DBCO-NH-PEG2 triester (N1)
  misc_feature 16: modified by C3 spacer (-(CH2)3-OH) (3′- phosphate)
tccatgacgttccttt

SEQ ID NO: 328 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ugctgctgagctt

SEQ ID NO: 329 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 13: modified by C3 spacer (-(CH2)3-OH) (3′- phosphate)
ugcagctgagctt

SEQ ID NO: 330 - DNA, 13 bp, Artificial Sequence
  Synthetic Construct
  misc_feature 1: nucleoside-3′-phosphate
  misc_feature (2)..(13): nucleoside-3′-phosphorothioate
  misc_feature 1: 5-trifluoromethyl-thymidine
  misc_feature 4: nucleoside having a 3′ DBCO-NH-PEG2 triester (N1)
  misc_feature 13: modified by C3 spacer (-(CH2)3-OH) (3′- phosphate)
tcgtcgtgtcgtt

SEQ ID NO: 331 - DNA, 13 bp, Artificial Sequence
  Synthetic Construct
  misc_feature (1)..(13): nucleoside-3′-phosphorothioate
  misc_feature 1: 5-trifluoromethyl-thymidine
  misc_feature 4: nucleoside having a 3′ DBCO-NH-PEG2 triester (N1)
  misc_feature 13: modified by C3 spacer (-(CH2)3-OH) (3′- phosphate)
tcgtcgtgtcgtt

SEQ ID NO: 332 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 7, 13: dT PEG2-NH2 triester
  misc_feature 13: modified by C3 spacer (-(CH2)3-OH) (3′- phosphate)
ucgtcgtgtcgtt

SEQ ID NO: 333 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 9, 13: dT PEG2-NH2 triester
  misc_feature 13: modified by C3 spacer (-(CH2)3-OH) (3′- phosphate)
ucgtcgtgtcgtt

SEQ ID NO: 334 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 13: dT PEG2-NH2 triester
  misc_feature 13: modified by C3 spacer (-(CH2)3-OH) (3′- phosphate)
ucgtcgtgtcgtt

SEQ ID NO: 335 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 9: dT PEG2-NH2 triester
  misc_feature 13: modified by C3 spacer (-(CH2)3-OH) (3′- phosphate)
ucgtcgtgtcgtt

SEQ ID NO: 336 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 7: dT PEG2-NH2 triester
  misc_feature 13: modified by C3 spacer (-(CH2)3-OH) (3′- phosphate)
ucgtcgtgtcgtt

SEQ ID NO: 337 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: DBCO-C6-dT, where C6 = hexane-1,6-diyl
  misc_feature 12, 13: dT PEG2-NH2 triester
  misc_feature 13: modified by C3 spacer (-(CH2)3-OH) (3′- phosphate)
ucgtcgtgtcgtt

SEQ ID NO: 338 - DNA, 14 bp, Artificial Sequence
  Synthetic Construct
  misc_feature (1)..(14): nucleoside-3′-phosphorothioate
  misc_feature 5: 5-iodo-2′-deoxyuridine
  misc_feature 14: nucleoside having a 3′ DBCO-NH-PEG2 triester (N1)
  misc_feature 14: modified by C3 spacer (-(CH2)3-OH) (3′- phosphate)
tatgugcttccttt

SEQ ID NO: 339 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 7, 12: PEG4 dT triester
  misc_feature 13: modified by C3 spacer (-(CH2)3-OH) (3′- phosphate)
ucgtcgtgtcgtt

SEQ ID NO: 340 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 9, 13: PEG4 dT triester
  misc_feature 13: modified by C3 spacer (-(CH2)3-OH) (3′- phosphate)
ucgtcgtgtcgtt

SEQ ID NO: 341 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 13: PEG4 dT triester
  misc_feature 13: modified by C3 spacer (-(CH2)3-OH) (3′- phosphate)
ucgtcgtgtcgtt

SEQ ID NO: 342 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 9: PEG4 dT triester
  misc_feature 13: modified by C3 spacer (-(CH2)3-OH) (3′- phosphate)
ucgtcgtgtcgtt

SEQ ID NO: 343 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 7: PEG4 dT triester
  misc_feature 13: modified by C3 spacer (-(CH2)3-OH) (3′- phosphate)
ucgtcgtgtcgtt

SEQ ID NO: 344 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ DBCO-NH-PEG2 triester (N1)
  misc_feature 12, 13: PEG4 dT triester
  misc_feature 13: modified by C3 spacer (-(CH2)3-OH) (3′- phosphate)
ucgtcgtgtcgtt

SEQ ID NO: 345 - DNA, 14 bp, Artificial Sequence
  Synthetic Construct
  misc_feature (1)..(14): nucleoside-3′-phosphorothioate
  misc_feature 1, 5: nucleoside having a 3′ DBCO-NH-PEG2 triester (N1)
  misc_feature 2: 5-iodo-2′-deoxyuridine
  misc_feature 14: modified by C3 spacer (-(CH2)3-OH) (3′- phosphate)
tugctgctgagctt

SEQ ID NO: 346 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: nucleoside having a 3′ DBCO-NH-PEG2 triester (N1)
  misc_feature 14: modified by C3 spacer (-(CH2)3-OH) (3′- phosphate)
tugctgctgagctt

SEQ ID NO: 347 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: modified by C3 spacer (-(CH2)3-OH) (3′- phosphate)
tugctgctgagctt

SEQ ID NO: 348 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 7, 12: nucleoside having a 3′ n-butyl triester
  misc_feature 13: modified by C3 spacer (-(CH2)3-OH) (3′- phosphate)
ucgtcgtgtcgtt

SEQ ID NO: 349 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 7, 9: nucleoside having a 3′ n-butyl triester
  misc_feature 13: modified by C3 spacer (-(CH2)3-OH) (3′- phosphate)
ucgtcgtgtcgtt

SEQ ID NO: 350 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 9, 12: nucleoside having a 3′ n-butyl triester
  misc_feature 13: modified by C3 spacer (-(CH2)3-OH) (3′- phosphate)
ucgtcgtgtcgtt

SEQ ID NO: 351 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 7: nucleoside having a 3′ n-butyl triester
  misc_feature 13: modified by C3 spacer (-(CH2)3-OH) (3′- phosphate)
ucgtcgtgtcgtt

SEQ ID NO: 352 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ DBCO-NH-PEG2 triester (N1)
  misc_feature 12: nucleoside having a 3′ n-butyl triester
  misc_feature 13: modified by C3 spacer (-(CH2)3-OH) (3′- phosphate)
ucgtcgtgtcgtt

SEQ ID NO: 353 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: modified by C3 spacer (-(CH2)3-OH) (3′- phosphate)
tucgtcgtgacgtt

SEQ ID NO: 354 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: modified by C3 spacer (-(CH2)3-OH) (3′- phosphate)
tugctgctgagctt

SEQ ID NO: 355 - DNA, 13 bp, Artificial Sequence
  Synthetic Construct
  misc_feature 1,2,3,5,6,7,8,9,10,11,13: nucleoside-3′-phosphorothioate
  misc_feature 4, 12: nucleoside-3′-phosphate
  misc_feature 1: 5-iodo-2′-deoxyuridine
  misc_feature 4: nucleoside having a 3′ DBCO-NH-PEG2 triester (N1)
  misc_feature 12: nucleoside having a 3′ n-butyl triester
  misc_feature 13: modified by C3 spacer (-(CH2)3-OH) (3′- phosphate)
ucgtcgtgtcgtt

SEQ ID NO: 356 - DNA, 13 bp, Artificial Sequence
  Synthetic Construct
  misc_feature 1,2,3,5,6,7,8,9,10,11,12,13: nucleoside-3′-phosphorothioate
  misc_feature 4: nucleoside-3′-phosphate
  misc_feature 1: 5-iodo-2′-deoxyuridine
  misc_feature 4: nucleoside having a 3′ DBCO-NH-PEG2 triester (N1)
  misc_feature 12: nucleoside having a 3′ n-butyl triester
  misc_feature 13: modified by C3 spacer (-(CH2)3-OH) (3′- phosphate)
ucgtcgtgtcgtt

SEQ ID NO: 357 - DNA, 13 bp, Artificial Sequence
  Synthetic Construct
  misc_feature 1,2,3,5,6,7,8,9,10,11,13: nucleoside-3′-phosphorothioate
  misc_feature 4, 12: nucleoside-3′-phosphate
  misc_feature 1: 5-iodo-2′-deoxyuridine
  misc_feature 4: nucleoside having a 3′ DBCO-NH-PEG2 triester (N1)
  misc_feature 12: nucleoside having a 3′ n-butyl triester
  misc_feature 13: modified by C3 spacer (-(CH2)3-OH) (3′- phosphate)
ucgtcgtgtcgtt

SEQ ID NO: 358 - DNA, 13 bp, Artificial Sequence
  Synthetic Construct
  misc_feature (1)..(13): nucleoside-3′-phosphorothioate
  misc_feature 1: 5-iodo-2′-deoxyuridine
  misc_feature 4: nucleoside having a 3′ n-butyl triester
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 359 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ n-butyl triester
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 360 - DNA, 13 bp, Artificial Sequence
  Synthetic Construct
  misc_feature 1,2,3,4,5,6,7,8,9,10,11,13: nucleoside-3′-phosphorothioate
  misc_feature 1: 5-iodo-2′-deoxyuridine
  misc_feature 12: nucleoside-3′-phosphate
  misc_feature 4: nucleoside having a 3′ n-butyl triester
  misc_feature 12: nucleoside having a 3′ DBCO-NH-PEG2 triester (N1)
  misc_feature 13: modified by C3 spacer (-(CH2)3-OH) (3′- phosphate)
ucgtcgtgtcgtt

SEQ ID NO: 361 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 362 - DNA, 13 bp, Artificial Sequence
  Synthetic Construct
  misc_feature 1,2,3,5,6,7,8,9,10,11,13: nucleoside-3′-phosphorothioate
  misc_feature 1: 5-iodo-2′-deoxyuridine
  misc_feature 4, 12: nucleoside-3′-phosphate
  misc_feature 4: nucleoside having a 3′ PEG2-NH2 triester (N1)
  misc_feature 12: thymidine having a 3′ ethyl triester
  misc_feature 13: modified by C3 spacer (-(CH2)3-OH) (3′- phosphate)
ucgtcgtgtcgtt

SEQ ID NO: 363 - DNA, 13 bp, Artificial Sequence
  Synthetic Construct
  misc_feature 1,2,3,5,6,7,8,9,10,12,13: nucleoside-3′-phosphorothioate
  misc_feature 1: 5-iodo-2′-deoxyuridine
  misc_feature 4: nucleoside having a 3′ PEG2-NH2 triester (N1)
  misc_feature 4, 11: nucleoside-3′-phosphate
  misc_feature 11: guanosine having a 3′ ethyl triester
  misc_feature 13: modified by C3 spacer (-(CH2)3-OH) (3′- phosphate)
ucgtcgtgtcgtt

SEQ ID NO: 364 - DNA, 13 bp, Artificial Sequence
  Synthetic Construct
  misc_feature 1,2,3,5,6,7,8,9,11,12,13: nucleoside-3′-phosphorothioate
  misc_feature 1: 5-iodo-2′-deoxyuridine
  misc_feature 4, 10: nucleoside-3′-phosphate
  misc_feature 4: nucleoside having a 3′ PEG2-NH2 triester (N1)
  misc_feature 10: cytidine having a 3′ ethyl triester
  misc_feature 13: modified by C3 spacer (-(CH2)3-OH) (3′- phosphate)
ucgtcgtgtcgtt

SEQ ID NO: 365 - DNA, 13 bp, Artificial Sequence
  Synthetic Construct
  misc_feature 1,2,3,5,6,7,8,10,11,12,13: nucleoside-3′-phosphorothioate
  misc_feature 1: 5-iodo-2′-deoxyuridine
  misc_feature 4, 9: nucleoside-3′-phosphate
  misc_feature 4: nucleoside having a 3′ PEG2-NH2 triester (N1)
  misc_feature 9: thymidine having a 3′ ethyl triester
  misc_feature 13: modified by C3 spacer (-(CH2)3-OH) (3′- phosphate)
ucgtcgtgtcgtt

SEQ ID NO: 366 - DNA, 13 bp, Artificial Sequence
  Synthetic Construct
  misc_feature 1,2,3,5,6,7,9,10,11,12,13: nucleoside-3′-phosphorothioate
  misc_feature 1: 5-iodo-2′-deoxyuridine
  misc_feature 4, 8: nucleoside-3′-phosphate
  misc_feature 4: nucleoside having a 3′ PEG2-NH2 triester (N1)
  misc_feature 8: guanosine having a 3′ ethyl triester
  misc_feature 13: modified by C3 spacer (-(CH2)3-OH) (3′- phosphate)
ucgtcgtgtcgtt

SEQ ID NO: 367 - DNA, 13 bp, Artificial Sequence
  Synthetic Construct
  misc_feature 1,2,3,5,6,8,9,10,11,12,13: nucleoside-3′-phosphorothioate
  misc_feature 1: 5-iodo-2′-deoxyuridine
  misc_feature 4, 7: nucleoside-3′-phosphate
  misc_feature 4: nucleoside having a 3′ PEG2-NH2 triester (N1)
  misc_feature 7: thymidine having a 3′ ethyl triester
  misc_feature 13: modified by C3 spacer (-(CH2)3-OH) (3′- phosphate)
ucgtcgtgtcgtt

SEQ ID NO: 368 - DNA, 13 bp, Artificial Sequence
  Synthetic Construct
  misc_feature 1,2,3,5,7,8,9,10,11,12,13: nucleoside-3′-phosphorothioate
  misc_feature 1: 5-iodo-2′-deoxyuridine
  misc_feature 4, 6: nucleoside-3′-phosphate
  misc_feature 4: nucleoside having a 3′ PEG2-NH2 triester (N1)
  misc_feature 6: guanosine having a 3′ ethyl triester
  misc_feature 13: modified by C3 spacer (-(CH2)3-OH) (3′- phosphate)
ucgtcgtgtcgtt

SEQ ID NO: 369 - DNA, 13 bp, Artificial Sequence
  Synthetic Construct
  misc_feature 1,2,3,6,7,8,9,10,11,12,13: nucleoside-3′-phosphorothioate
  misc_feature 1: 5-iodo-2′-deoxyuridine
  misc_feature 4, 5: nucleoside-3′-phosphate
  misc_feature 4: nucleoside having a 3′ PEG2-NH2 triester (N1)
  misc_feature 5: cytidine having a 3′ ethyl triester
  misc_feature 13: modified by C3 spacer (-(CH2)3-OH) (3′- phosphate)
ucgtcgtgtcgtt

SEQ ID NO: 370 - DNA, 13 bp, Artificial Sequence
  Synthetic Construct
  misc_feature 1,2,5,6,7,8,9,10,11,12,13: nucleoside-3′-phosphorothioate
  misc_feature 1: 5-iodo-2′-deoxyuridine
  misc_feature 3, 4: nucleoside-3′-phosphate
  misc_feature 3: guanosine having a 3′ ethyl triester
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 371 - DNA, 13 bp, Artificial Sequence
  Synthetic Construct
  misc_feature 1,3,5,6,7,8,9,10,11,12,13: nucleoside-3′-phosphorothioate
  misc_feature 1: 5-iodo-2′-deoxyuridine
  misc_feature 2, 4: nucleoside-3′-phosphate
  misc_feature 2: cytidine having a 3′ ethyl triester
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 372 - DNA, 13 bp, Artificial Sequence
  Synthetic Construct
  misc_feature 1,2,3,5,6,7,8,9,10,11,13: nucleoside-3′-phosphorothioate
  misc_feature 1: 5-iodo-2′-deoxyuridine
  misc_feature 4, 12: nucleoside-3′-phosphate
  misc_feature 4: thymidine having a 3′ ethyl triester
  misc_feature 12: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 373 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: modified by X5-C3 as described in Table 2 of the specification
tucgtcgtgacgtt

SEQ ID NO: 374 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 13,14: linked by X5-DBCO as described in Table 2 of the specification
  misc_feature 14: modified by C3 spacer (-(CH2)3-OH) (3′- phosphate)
tucgtcgtgacgtt

SEQ ID NO: 375 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 12, 13: linked by X5-DBCO as described in Table 2 of the specification
  misc_feature 14: modified by C3 spacer (-(CH2)3-OH) (3′- phosphate)
tucgtcgtgacgtt

SEQ ID NO: 376 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 11, 12: linked by X5-DBCO as described in Table 2 of the specification
  misc_feature 14: modified by C3 spacer (-(CH2)3-OH) (3′- phosphate)
tucgtcgtgacgtt

SEQ ID NO: 377 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 10, 11: linked by X5-DBCO as described in Table 2 of the specification
  misc_feature 14: modified by C3 spacer (-(CH2)3-OH) (3′- phosphate)
tucgtcgtgacgtt

SEQ ID NO: 378 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 9, 10: linked by X5-DBCO as described in Table 2 of the specification
  misc_feature 14: modified by C3 spacer (-(CH2)3-OH) (3′- phosphate)
tucgtcgtgacgtt

SEQ ID NO: 379 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 8, 9: linked by X5-DBCO as described in Table 2 of the specification
  misc_feature 14: modified by C3 spacer (-(CH2)3-OH) (3′- phosphate)
tucgtcgtgacgtt

SEQ ID NO: 380 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 7, 8: linked by X5-DBCO as described in Table 2 of the specification
  misc_feature 14: modified by C3 spacer (-(CH2)3-OH) (3′- phosphate)
tucgtcgtgacgtt

SEQ ID NO: 381 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 6, 7: linked by X5-DBCO as described in Table 2 of the specification
  misc_feature 14: modified by C3 spacer (-(CH2)3-OH) (3′- phosphate)
tucgtcgtgacgtt

SEQ ID NO: 382 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5, 6: linked by X5-DBCO as described in Table 2 of the specification
  misc_feature 14: modified by C3 spacer (-(CH2)3-OH) (3′- phosphate)
tucgtcgtgacgtt

SEQ ID NO: 383 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 4, 5: linked by X5-DBCO as described in Table 2 of the specification
  misc_feature 14: modified by C3 spacer (-(CH2)3-OH) (3′- phosphate)
tucgtcgtgacgtt

SEQ ID NO: 384 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 3, 4: linked by X5-DBCO as described in Table 2 of the specification
  misc_feature 14: modified by C3 spacer (-(CH2)3-OH) (3′- phosphate)
tucgtcgtgacgtt

SEQ ID NO: 385 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 2, 3: linked by X5-DBCO as described in Table 2 of the specification
  misc_feature 14: modified by C3 spacer (-(CH2)3-OH) (3′- phosphate)
tucgtcgtgacgtt

SEQ ID NO: 386 - DNA, 14 bp, Artificial Sequence
  Synthetic Construct
  misc_feature (1)..(14): nucleoside-3′-phosphorothioate
  misc_feature 1, 2: linked by X5-DBCO as described in Table 2 of the specification
  misc_feature 2: 5-iodo-2′-deoxyuridine
  misc_feature 14: modified by C3 spacer (-(CH2)3-OH) (3′- phosphate)
tucgtcgtgacgtt

SEQ ID NO: 387 - DNA, 14 bp, Artificial Sequence
  Synthetic Construct
  misc_feature (1)..(14): nucleoside-3′-phosphorothioate
  misc_feature 1: modified by X5-DBCO as described in Table 2 of the specification
  misc_feature 2: 5-iodo-2′-deoxyuridine
  misc_feature 14: modified by C3 spacer (-(CH2)3-OH) (3′- phosphate)
tucgtcgtgacgtt

SEQ ID NO: 388 - DNA, 13 bp, Artificial Sequence
  Synthetic Construct
  misc_feature (1)..(13): nucleoside-3′-phosphorothioate
  misc_feature 4,5: linked by X5-DBCO as described in Table 2 of the specification
  misc_feature 13: modified by C3 spacer (-(CH2)3-OH) (3′- phosphate)
tucgcgtgacgtt

SEQ ID NO: 389 - DNA, 13 bp, Artificial Sequence
  Synthetic Construct
  misc_feature (1)..(13): nucleoside-3′-phosphorothioate
  misc_feature 4,5: linked by X5-DBCO as described in Table 2 of the specification
  misc_feature 13: modified by C3 spacer (-(CH2)3-OH) (3′- phosphate)
tucgcgtgacgtt

SEQ ID NO: 390 - DNA, 13 bp, Artificial Sequence
  Synthetic Construct
  misc_feature 1,4: nucleoside-3′-phosphate
  misc_feature 1: 5-iodouridine having a 3′ ethyl triester
  misc_feature 2,3: nucleoside-3′-phosphorothioate
  misc_feature 4: nucleoside having a 3′ PEG2-NH2 triester (N1)
  misc_feature (5)..(13): nucleoside-3′-phosphorothioate
  misc_feature 13: modified by C3 spacer (-(CH2)3-OH) (3′- phosphate)
ucgtcgtgtcgtt

SEQ ID NO: 391 - DNA, 13 bp, Artificial Sequence
  Synthetic Construct
  misc_feature 1,2,4: nucleoside-3′-phosphate
  misc_feature 1: 5-iodouridine having a 3′ ethyl triester
  misc_feature 2: cytidine having a 3′ ethyl triester
  misc_feature 3,5,6,7,8,9,10,11,12,13: nucleoside-3′-phosphorothioate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 392 - DNA, 13 bp, Artificial Sequence
  Synthetic Construct
  misc_feature (1)..(4): nucleoside-3′-phosphate
  misc_feature 1: 5-iodouridine having a 3′ ethyl triester
  misc_feature 2: cytidine having a 3′ ethyl triester
  misc_feature 3: guanosine having a 3′ ethyl triester
  misc_feature 4: nucleoside having a 3′ PEG2-NH2 triester (N1)
  misc_feature (5)..(13): nucleoside-3′-phosphorothioate
  misc_feature 13: modified by C3 spacer (-(CH2)3-OH) (3′- phosphate)
ucgtcgtgtcgtt

SEQ ID NO: 393 - DNA, 13 bp, Artificial Sequence
  Synthetic Construct
  misc_feature 1,2,3,5,6,7,8,9: nucleoside-3′-phosphorothioate
  misc_feature 1: 5-iodo-2′-deoxyuridine
  misc_feature 4,10,11,12,13: nucleoside-3′-phosphate
  misc_feature 4: nucleoside having a 3′ PEG2-NH2 triester (N1)
  misc_feature 10: cytidine having a 3′ ethyl triester
  misc_feature 11: guanosine having a 3′ ethyl triester
  misc_feature 12,13: thymidine having a 3′ ethyl triester
  misc_feature 13: modified by C3 spacer (-(CH2)3-OH) (3′- phosphate)
ucgtcgtgtcgtt

SEQ ID NO: 394 - DNA, 13 bp, Artificial Sequence
  Synthetic Construct
  misc_feature 1,2,3,4,10,11,12,13: nucleoside-3′-phosphate
  misc_feature (5)..(9): nucleoside-3′-phosphorothioate
  misc_feature 1: 5-iodouridine having a 3′ ethyl triester
  misc_feature 4: nucleoside having a 3′ PEG2-NH2 triester (N1)
  misc_feature 2,10: cytidine having a 3′ ethyl triester
  misc_feature 3,11: guanosine having a 3′ ethyl triester
  misc_feature 12,13: thymidine having a 3′ ethyl triester
  misc_feature 13: modified by C3 spacer (-(CH2)3-OH) (3′- phosphate)
ucgtcgtgtcgtt

SEQ ID NO: 395 - DNA, 13 bp, Artificial Sequence
  Synthetic Construct
  misc_feature (1)..(13): nucleoside-3′-phosphate
  misc_feature 1: 5-iodouridine having a 3′ ethyl triester
  misc_feature 4: nucleoside having a 3′ PEG2-NH2 triester (N1)
  misc_feature 2,5,10: cytidine having a 3′ ethyl triester
  misc_feature 3,6,8,11: guanosine having a 3′ ethyl triester
  misc_feature 7,9,12,13: thymidine having a 3′ ethyl triester
  misc_feature 13: modified by C3 spacer (-(CH2)3-OH) (3′- phosphate)
ucgtcgtgtcgtt

SEQ ID NO: 396 - DNA, 13 bp, Artificial Sequence
  Synthetic Construct
  misc_feature 1,3,6,7,8,9,11,12,13: nucleoside-3′-phosphorothioate
  misc_feature 1: 5-iodo-2′-deoxyuridine
  misc_feature 2,4,5,10: nucleoside-3′-phosphate
  misc_feature 2,5,10: cytidine having a 3′ ethyl triester
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 397 - DNA, 13 bp, Artificial Sequence
  Synthetic Construct
  misc_feature 1,2,3,5,7,9,10,12,13: nucleoside-3′-phosphorothioate
  misc_feature 1: 5-iodo-2′-deoxyuridine
  misc_feature 4,6,8,11: nucleoside-3′-phosphate
  misc_feature 4: nucleoside having a 3′ PEG2-NH2 triester (N1)
  misc_feature 6,8,11: guanosine having a 3′ ethyl triester
  misc_feature 13: modified by C3 spacer (-(CH2)3-OH) (3′- phosphate)
ucgtcgtgtcgtt

SEQ ID NO: 398 - DNA, 13 bp, Artificial Sequence
  Synthetic Construct
  misc_feature 1,2,3,5,6,8,10,11,13: nucleoside-3′-phosphorothioate
  misc_feature 1: 5-iodo-2′-deoxyuridine
  misc_feature 4,7,9,12: nucleoside-3′-phosphate
  misc_feature 4: nucleoside having a 3′ PEG2-NH2 triester (N1)
  misc_feature 7,9,12: thymidine having a 3′ ethyl triester
  misc_feature 13: modified by C3 spacer (-(CH2)3-OH) (3′- phosphate)
ucgtcgtgtcgtt

SEQ ID NO: 399 - DNA, 13 bp, Artificial Sequence
  Synthetic Construct
  misc_feature 1,3,5,6,8,10,11,13: nucleoside-3′-phosphorothioate
  misc_feature 1: 5-iodo-2′-deoxyuridine
  misc_feature 2,4,7,9,12: nucleoside-3′-phosphate
  misc_feature 2: cytidine having a 3′ ethyl triester
  misc_feature 4: nucleoside having a 3′ PEG2-NH2 triester (N1)
  misc_feature 7,9,12: thymidine having a 3′ ethyl triester
  misc_feature 13: modified by C3 spacer (-(CH2)3-OH) (3′- phosphate)
ucgtcgtgtcgtt

SEQ ID NO: 400 - DNA, 13 bp, Artificial Sequence
  Synthetic Construct
  misc_feature 1,2,3,5,6,8,10,11,13: nucleoside-3′-phosphorothioate
  misc_feature 4, 7, 9, 12: nucleoside-3′-phosphate
  misc_feature 1: 5-iodo-2′-deoxyuridine
  misc_feature 4, 7, 9: thymidine having a 3′ ethyl triester
  misc_feature 12: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 401 - DNA, 13 bp, Artificial Sequence
  Synthetic Construct
  misc_feature 1, 3, 5, 6, 8, 10, 11, 13: nucleoside-3′-phosphorothioate
  misc_feature 2, 4, 7, 9, 12: nucleoside-3′-phosphate
  misc_feature 1: 5-iodo-2′-deoxyuridine
  misc_feature 2: cytidine having a 3′ ethyl triester
  misc_feature 4, 7, 9: thymidine having a 3′ ethyl triester
  misc_feature 12: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 402 - DNA, 13 bp, Artificial Sequence
  Synthetic Construct
  misc_feature 1,3,5,6,7,8,9,10,12,13: nucleoside-3′-phosphorothioate
  misc_feature 1: 5-iodo-2′-deoxyuridine
  misc_feature 2, 4, 11: nucleoside-3′-phosphate
  misc_feature 2: cytidine having a 3′ ethyl triester
  misc_feature 4: nucleoside having a 3′ PEG2-NH2 triester (N1)
  misc_feature 11: guanosine having a 3′ ethyl triester
  misc_feature 13: modified by C3 spacer (-(CH2)3-OH) (3′- phosphate)
ucgtcgtgtcgtt

SEQ ID NO: 403 - DNA, 13 bp, Artificial Sequence
  Synthetic Construct
  misc_feature 1,3,5,6,7,8,9,11,12,13: nucleoside-3′-phosphorothioate
  misc_feature 1: 5-iodo-2′-deoxyuridine
  misc_feature 2, 4, 10: nucleoside-3′-phosphate
  misc_feature 2, 10: cytidine having a 3′ ethyl triester
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 404 - DNA, 14 bp, Artificial Sequence
  Synthetic Construct
  misc_feature 1,3,5,6,7,8,9,10,11,12,14: nucleoside-3′-phosphorothioate
  misc_feature 1: 5-iodo-2′-deoxyuridine
  misc_feature 2, 4, 13: nucleoside-3′-phosphate
  misc_feature 2: cytidine having a 3′ ethyl triester
  misc_feature 4: nucleoside having a 3′ PEG2-NH2 triester (N1)
  misc_feature 13: thymidine having a 3′ ethyl triester
  misc_feature 14: modified by C3 spacer (-(CH2)3-OH) (3′- phosphate)
ucgtcgttgtcgtt

SEQ ID NO: 405 - DNA, 14 bp, Artificial Sequence
  Synthetic Construct
  misc_feature 1,3,5,6,7,9,10,11,12,14: nucleoside-3′-phosphorothioate
  misc_feature 1: 5-iodo-2′-deoxyuridine
  misc_feature 2, 4, 8, 13: nucleoside-3′-phosphate
  misc_feature 2: cytidine having a 3′ ethyl triester
  misc_feature 4: nucleoside having a 3′ PEG2-NH2 triester (N1)
  misc_feature 8, 13: thymidine having a 3′ ethyl triester
  misc_feature 14: modified by C3 spacer (-(CH2)3-OH) (3′- phosphate)
ucgtcgttgtcgtt

SEQ ID NO: 406 - DNA, 14 bp, Artificial Sequence
  Synthetic Construct
  misc_feature 1,3,5,6,8,9,10,11,12,14: nucleoside-3′-phosphorothioate
  misc_feature 1: 5-iodo-2′-deoxyuridine
  misc_feature 2, 4, 7, 13: nucleoside-3′-phosphate
  misc_feature 2: cytidine having a 3′ ethyl triester
  misc_feature 4: nucleoside having a 3′ PEG2-NH2 triester (N1)
  misc_feature 7, 13: thymidine having a 3′ ethyl triester
  misc_feature 14: modified by C3 spacer (-(CH2)3-OH) (3′- phosphate)
ucgtcgttgtcgtt

SEQ ID NO: 407 - DNA, 13 bp, Artificial Sequence
  Synthetic Construct
  misc_feature 1,3,5,6,7,8,9,10,11,13: nucleoside-3′-phosphorothioate
  misc_feature 1: 5-iodo-2′-deoxyuridine
  misc_feature 2, 4, 12: nucleoside-3′-phosphate
  misc_feature 2: cytidine having a 3′ ethyl triester
  misc_feature 4: nucleoside having a 3′ PEG2-NH2 triester (N1)
  misc_feature 7,8: linked by X3 abasic spacer as described in Table 2 of the specification
  misc_feature 12: thymidine having a 3′ ethyl triester
  misc_feature 13: modified by C3 spacer (-(CH2)3-OH) (3′- phosphate)
ucgtcgtgtcgtt

SEQ ID NO: 408 - DNA, 13 bp, Artificial Sequence
  Synthetic Construct
  misc_feature 1,3,5,6,7,8,9,10,11,13: nucleoside-3′-phosphorothioate
  misc_feature 1: 5-iodo-2′-deoxyuridine
  misc_feature 2, 4, 12: nucleoside-3′-phosphate
  misc_feature 2: cytidine having a 3′ ethyl triester
  misc_feature 4: nucleoside having a 3′ PEG2-NH2 triester (N1)
  misc_feature 6,7: linked by X3 abasic spacer as described in Table 2 of the specification
  misc_feature 12: thymidine having a 3′ ethyl triester
  misc_feature 13: modified by C3 spacer (-(CH2)3-OH) (3′- phosphate)
ucgtcgtgtcgtt

SEQ ID NO: 409 - DNA, 14 bp, Artificial Sequence
  Synthetic Construct
  misc_feature 1,3,5,6,7,9,10,11,12,14: nucleoside-3′-phosphorothioate
  misc_feature 1: 5-iodo-2′-deoxyuridine
  misc_feature 2, 4, 8, 13: nucleoside-3′-phosphate
  misc_feature 2: cytidine having a 3′ ethyl triester
  misc_feature 4: nucleoside having a 3′ PEG2-NH2 triester (N1)
  misc_feature 8, 13: thymidine having a 3′ ethyl triester
  misc_feature 14: modified by C3 spacer (-(CH2)3-OH) (3′- phosphate)
ucgtcgttgtcgtt

SEQ ID NO: 410 - DNA, 13 bp, Artificial Sequence
  Synthetic Construct
  misc_feature 1,3,5,6,7,8,9,10,11,13: nucleoside-3′-phosphorothioate
  misc_feature 1: 5-iodo-2′-deoxyuridine
  misc_feature 2, 4, 12: nucleoside-3′-phosphate
  misc_feature 2: cytidine having a 3′ ethyl triester
  misc_feature 4: nucleoside having a 3′ PEG2-NH2 triester (N1)
  misc_feature 7,8: linked by X3 abasic spacer as described in Table 2 of the specification
  misc_feature 12: thymidine having a 3′ ethyl triester
  misc_feature 13: modified by C3 spacer (-(CH2)3-OH) (3′- phosphate)
ucgtcgtgtcgtt

SEQ ID NO: 411 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 1: modified by X3 abasic spacer described in Table 2 of the specification
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 412 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 1,2: linked by X3 abasic spacer described in Table 2 of the specification
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 413 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 2,3: linked by X3 abasic spacer as described in Table 2 of the specification
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 414 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 3,4: linked by X3 abasic spacer as described in Table 2 of the specification
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 415 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4,5: linked by X3 abasic spacer as described in Table 2 of the specification
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 416 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 5,6: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 417 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 6,7: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 418 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 7,8: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 419 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 8,9: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 420 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 9,10: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 421 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 10,11: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 422 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 11,12: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 423 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 12,13: linked by X3 abasic spacer as described in Table 2 of the specification
  misc_feature 13: modified by C3 spacer (-(CH2)3-OH) (3′- phosphate)
ucgtcgtgtcgtt

SEQ ID NO: 424 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 13: modified by X3 abasic spacer described in Table 2 of the specification
  misc_feature 13: modified by C3 spacer (-(CH2)3-OH) (3′- phosphate)
ucgtcgtgtcgtt

SEQ ID NO: 425 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 426 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-iodo-2′-deoxycytidine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ccgtcgtgtcgtt

SEQ ID NO: 427 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5′-5′ cap based on 5-iodo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 428 - DNA, 13 bp, Artificial Sequence
  Synthetic Construct
  misc_feature 1,4: nucleoside-3′-phosphate
  misc_feature 1: 5′-5′ cap based on 5-iodo-2′-deoxyuridine
  misc_feature 2,3,5,6,7,8,9,10,11,12,13: nucleoside-3′-phosphorothioate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 429 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5′-orthodisulfide phosphodiester
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphorothioate)
ucgtcgtgtcgtt

SEQ ID NO: 430 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5′-phosphate
  misc_feature 1: 5-iodo-2′-deoxyuridine (3′-phosphorothioate)
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphorothioate)
ucgtcgtgtcgtt

SEQ ID NO: 431 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5′-orthodisulfide phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphorothioate)
ucgtcgtgtcgtt

SEQ ID NO: 432 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5′-phosphorothioate
  misc_feature 1: 5-iodo-2′-deoxyuridine
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphorothioate)
ucgtcgtgtcgtt

SEQ ID NO: 433 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 1: Rp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 434 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 1: Sp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 435 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 2: Rp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 436 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 2: Sp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 437 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 1: Sp conformation
  misc_feature 2: Rp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 438 - DNA, 13 bp, Artificial Sequence
  Synthetic Construct
  misc_feature 1,2,3,5,6,7,8,9,10,11,12,13: nucleoside-3′-phosphorothioate
  misc_feature 1: 5-bromo-2′-deoxyuridine
  misc_feature 1,2: Sp conformation
  misc_feature 4: nucleoside-3′-phosphate
  misc_feature 4: nucleoside having a 3′ PEG2-NH2 triester (N1)
  misc_feature 13: modified by C3 spacer (-(CH2)3-OH) (3′- phosphate)
ucgtcgtgtcgtt

SEQ ID NO: 439 - DNA, 13 bp, Artificial Sequence
  Synthetic Construct
  misc_feature 1,3,5,6,7,8,9,10,11,12,13: nucleoside-3′-phosphorothioate
  misc_feature 1: 5-bromo-2′-deoxyuridine
  misc_feature 2, 4: nucleoside-3′-phosphate
  misc_feature 2: cytidine having a 3′ ethyl triester
  misc_feature 4: dT PEG2-NH2 triester
  misc_feature 13: modified by C3 spacer (-(CH2)3-OH) (3′- phosphate)
ucgtcgtgtcgtt

SEQ ID NO: 440 - DNA, 13 bp, Artificial Sequence
  Synthetic Construct
  misc_feature 1,3,5,6,7,8,9,11,12,13: nucleoside-3′-phosphorothioate
  misc_feature 1: 5-bromo-2′-deoxyuridine
  misc_feature 2, 4, 10: nucleoside-3′-phosphate
  misc_feature 2, 10: cytidine having a 3′ ethyl triester
  misc_feature 4: dT PEG2-NH2 triester
  misc_feature 13: modified by C3 spacer (-(CH2)3-OH) (3′- phosphate)
ucgtcgtgtcgtt

SEQ ID NO: 441 - DNA, 13 bp, Artificial Sequence
  Synthetic Construct
  misc_feature 1,3,6,7,8,9,11,12,13: nucleoside-3′-phosphorothioate
  misc_feature 1: 5-bromo-2′-deoxyuridine
  misc_feature 2, 4, 5, 10: nucleoside-3′-phosphate
  misc_feature 2, 5, 10: cytidine having a 3′ ethyl triester
  misc_feature 4: dT PEG2-NH2 triester
  misc_feature 13: modified by C3 spacer (-(CH2)3-OH) (3′- phosphate)
ucgtcgtgtcgtt

SEQ ID NO: 442 - DNA, 13 bp, Artificial Sequence
  Synthetic Construct
  misc_feature 1,3,5,6,7,8,9,10,11,13: nucleoside-3′-phosphorothioate
  misc_feature 1: 5-bromo-2′-deoxyuridine
  misc_feature 2, 4, 12: nucleoside-3′-phosphate
  misc_feature 2: cytidine having a 3′ ethyl triester
  misc_feature 4: dT PEG2-NH2 triester
  misc_feature 12: thymidine having a 3′ ethyl triester
  misc_feature 13: modified by C3 spacer (-(CH2)3-OH) (3′- phosphate)
ucgtcgtgtcgtt

SEQ ID NO: 443 - DNA, 13 bp, Artificial Sequence
  Synthetic Construct
  misc_feature 1,3,5,6,8,9,10,11,13: nucleoside-3′-phosphorothioate
  misc_feature 1: 5-bromo-2′-deoxyuridine
  misc_feature 2, 4, 7, 12: nucleoside-3′-phosphate
  misc_feature 2: cytidine having a 3′ ethyl triester
  misc_feature 4: dT PEG2-NH2 triester
  misc_feature 7, 12: thymidine having a 3′ ethyl triester
  misc_feature 13: modified by C3 spacer (-(CH2)3-OH) (3′- phosphate)
ucgtcgtgtcgtt

SEQ ID NO: 444 - DNA, 13 bp, Artificial Sequence
  Synthetic Construct
  misc_feature (1)..(13): nucleoside-3′-phosphate
  misc_feature 1: biotinylated
  misc_feature 1,2,5,7,10,13: 2′-fluoro-adenosine
  misc_feature 3,6,8,11: 2′-fluoro-cytosine
  misc_feature 4,9,12: 2′-fluoro-guanine
aacgacacgacga

SEQ ID NO: 445 - DNA, 13 bp, Artificial Sequence
  Synthetic Construct
  misc_feature 1,3,5,6,7,8,9,10,11,12,13: nucleoside-3′-phosphorothioate
  misc_feature 1: 5-bromo-2′-deoxyuridine
  misc_feature 2, 4: nucleoside-3′-phosphate
  misc_feature 2: dC O-silyltriester
  misc_feature 4: dT PEG2-NH2 triester
  misc_feature 13: modified by C3 spacer (-(CH2)3-OH) (3′- phosphorothioate)
ucgtcgtgtcgtt

SEQ ID NO: 446 - DNA, 13 bp, Artificial Sequence
  Synthetic Construct
  misc_feature 1,2,3,5,6,7,8,9,10,11,13: nucleoside-3′-phosphorothioate
  misc_feature 1: 5-bromo-2′-deoxyuridine
  misc_feature 4, 12: nucleoside-3′-phosphate
  misc_feature 4: dT PEG2-NH2 triester
  misc_feature 12: dT O-silyltriester
  misc_feature 13: modified by C3 spacer (-(CH2)3-OH) (3′- phosphorothioate)
ucgtcgtgtcgtt

SEQ ID NO: 447 - DNA, 13 bp, Artificial Sequence
  Synthetic Construct
  misc_feature 1,3,6,7,8,9,11,12,13: nucleoside-3′-phosphorothioate
  misc_feature 1: 5-bromo-2′-deoxyuridine
  misc_feature 2, 4, 5, 10: nucleoside-3′-phosphate
  misc_feature 2, 5, 10: dC O-silyltriester
  misc_feature 4: dT PEG2-NH2 triester
  misc_feature 13: modified by C3 spacer (-(CH2)3-OH) (3′- phosphorothioate)
ucgtcgtgtcgtt

SEQ ID NO: 448 - DNA, 13 bp, Artificial Sequence
  Synthetic Construct
  misc_feature 1,3,5,6,7,8,9,10,11,13: nucleoside-3′-phosphorothioate
  misc_feature 1: 5-bromo-2′-deoxyuridine
  misc_feature 2, 4, 12: nucleoside-3′-phosphate
  misc_feature 2: dC O-silyltriester
  misc_feature 4: dT PEG2-NH2 triester
  misc_feature 12: dT O-silyltriester
  misc_feature 13: modified by C3 spacer (-(CH2)3-OH) (3′- phosphorothioate)
ucgtcgtgtcgtt

SEQ ID NO: 449 - DNA, 13 bp, Artificial Sequence
  Synthetic Construct
  misc_feature 1,3,5,6,8,9,10,11,13: nucleoside-3′-phosphorothioate
  misc_feature 1: 5-bromo-2′-deoxyuridine
  misc_feature 2, 4, 7, 12: nucleoside-3′-phosphate
  misc_feature 2: dC O-silyltriester
  misc_feature 4: dT PEG2-NH2 triester
  misc_feature 7, 12: dT O-silyltriester
  misc_feature 13: modified by C3 spacer (-(CH2)3-OH) (3′- phosphorothioate)
ucgtcgtgtcgtt

SEQ ID NO: 450 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: dT PEG2-NH2 triester
  misc_feature 14: modified by C3 spacer (-(CH2)3-OH) (3′- phosphorothioate)
tucgtcgtgacgtt

SEQ ID NO: 451 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 14: dT PEG2-NH2 triester
  misc_feature 14: modified by C3 spacer (-(CH2)3-OH) (3′- phosphorothioate)
tucgacgtgacgtt

SEQ ID NO: 452 - DNA, 9 bp, Artificial Sequence
  Synthetic Construct
  misc_feature (1)..(9): nucleoside-3′-phosphorothioate
  misc_feature 2: 5-iodo-2′-deoxyuridine
  misc_feature 9: dT PEG2-NH2 triester
  misc_feature 9: modified by C3 spacer (-(CH2)3-OH) (3′- phosphorothioate)
tucgacgtt

SEQ ID NO: 453 - DNA, 7 bp, Artificial Sequence
  Synthetic Construct
  misc_feature (1)..(7): nucleoside-3′-phosphorothioate
  misc_feature 2: 5-iodo-2′-deoxyuridine
  misc_feature 7: dT PEG2-NH2 triester
  misc_feature 7: modified by C3 spacer (-(CH2)3-OH) (3′- phosphorothioate)
tuacgtt

SEQ ID NO: 454 - DNA, 6 bp, Artificial Sequence
  Synthetic Construct
  misc_feature (1)..(6): nucleoside-3′-phosphorothioate
  misc_feature 6: dT PEG2-NH2 triester
  misc_feature 6: modified by C3 spacer (-(CH2)3-OH) (3′- phosphorothioate)
tacgtt

SEQ ID NO: 455 - DNA, 6 bp, Artificial Sequence
  Synthetic Construct
  misc_feature (1)..(6): nucleoside-3′-phosphorothioate
  misc_feature 2: 5-iodo-2′-deoxyuridine
  misc_feature 6: dT PEG2-NH2 triester
  misc_feature 6: modified by C3 spacer (-(CH2)3-OH) (3′- phosphorothioate)
tucgtt

SEQ ID NO: 456 - DNA, 5 bp, Artificial Sequence
  Synthetic Construct
  misc_feature (1)..(5): nucleoside-3′-phosphorothioate
  misc_feature 5: dT PEG2-NH2 triester
  misc_feature 5: modified by C3 spacer (-(CH2)3-OH) (3′- phosphorothioate)
tacgt

SEQ ID NO: 457 - DNA, 5 bp, Artificial Sequence
  Synthetic Construct
  misc_feature (1)..(5): nucleoside-3′-phosphorothioate
  misc_feature 2: 5-iodo-2′-deoxyuridine
  misc_feature 5: dT PEG2-NH2 triester
  misc_feature 5: modified by C3 spacer (-(CH2)3-OH) (3′- phosphorothioate)
tucgt

SEQ ID NO: 458 - DNA, 14 bp, Artificial Sequence
  Synthetic Construct
  misc_feature (1)..(14): nucleoside-3′-phosphorothioate
  misc_feature 2, 5: 5-iodo-2′-deoxyuridine
  misc_feature 14: dT PEG2-NH2 triester
  misc_feature 14: modified by C3 spacer (-(CH2)3-OH) (3′- phosphorothioate)
tucgucgtgacgtt

SEQ ID NO: 459 - DNA, 9 bp, Artificial Sequence
  Synthetic Construct
  misc_feature (1)..(9): nucleoside-3′-phosphorothioate
  misc_feature 2,5: 5-iodo-2′-deoxyuridine
  misc_feature 9: dT PEG2-NH2 triester
  misc_feature 9: modified by C3 spacer (-(CH2)3-OH) (3′- phosphorothioate)
tucgucgtt

SEQ ID NO: 460 - DNA, 7 bp, Artificial Sequence
  Synthetic Construct
  misc_feature (1)..(7): nucleoside-3′-phosphorothioate
  misc_feature 2, 6: 5-iodo-2′-deoxyuridine
  misc_feature 7: dT PEG2-NH2 triester
  misc_feature 7: modified by C3 spacer (-(CH2)3-OH) (3′- phosphorothioate)
tuacgut

SEQ ID NO: 461 - DNA, 6 bp, Artificial Sequence
  Synthetic Construct
  misc_feature (1)..(6): nucleoside-3′-phosphorothioate
  misc_feature 5: 5-iodo-2′-deoxyuridine
  misc_feature 6: dT PEG2-NH2 triester
  misc_feature 6: modified by C3 spacer (-(CH2)3-OH) (3′- phosphorothioate)
tacgut

SEQ ID NO: 462 - DNA, 6 bp, Artificial Sequence
  Synthetic Construct
  misc_feature (1)..(6): nucleoside-3′-phosphorothioate
  misc_feature 2, 5: 5-iodo-2′-deoxyuridine
  misc_feature 6: dT PEG2-NH2 triester
  misc_feature 6: modified by C3 spacer (-(CH2)3-OH) (3′- phosphorothioate)
tucgut

SEQ ID NO: 463 - DNA, 6 bp, Artificial Sequence
  Synthetic Construct
  misc_feature (1)..(6): nucleoside-3′-phosphorothioate
  misc_feature 2: 5-iodo-2′-deoxyuridine
  misc_feature 6: dT PEG2-NH2 triester
  misc_feature 6: modified by C3 spacer (-(CH2)3-OH) (3′- phosphorothioate)
gucgtt

SEQ ID NO: 464 - DNA, 6 bp, Artificial Sequence
  Synthetic Construct
  misc_feature (1)..(6): nucleoside-3′-phosphorothioate
  misc_feature 6: dT PEG2-NH2 triester
  misc_feature 6: modified by C3 spacer (-(CH2)3-OH) (3′- phosphorothioate)
gacgtt

SEQ ID NO: 465 - DNA, 6 bp, Artificial Sequence
  Synthetic Construct
  misc_feature (1)..(6): nucleoside-3′-phosphorothioate
  misc_feature 2, 5: 5-iodo-2′-deoxyuridine
  misc_feature 6: dT PEG2-NH2 triester
  misc_feature 6: modified by C3 spacer (-(CH2)3-OH) (3′- phosphorothioate)
gucgut

SEQ ID NO: 466 - DNA, 6 bp, Artificial Sequence
  Synthetic Construct
  misc_feature (1)..(6): nucleoside-3′-phosphorothioate
  misc_feature 5: 5-iodo-2′-deoxyuridine
  misc_feature 6: dT PEG2-NH2 triester
  misc_feature 6: modified by C3 spacer (-(CH2)3-OH) (3′- phosphorothioate)
gacgut

SEQ ID NO: 467 - DNA, 24 bp, Artificial Sequence
  Synthetic Construct
tcgtcgttttgtcgttttgtcgtt

SEQ ID NO: 468 - DNA, 24 bp, Artificial Sequence
  Synthetic Construct
agcagcaaaacagcaaaacagcaa

SEQ ID NO: 469 - DNA, 14 bp, Artificial Sequence
  Synthetic Construct
  misc_feature (1)..(14): nucleoside-3′-phosphorothioate
  misc_feature 2: 5-bromo-2′-deoxyuridine
  misc_feature 5: DBCO-C6-dT
  misc_feature 14: modified by C3 spacer (-(CH2)3-OH) (3′- phosphorothioate)
tucgtcgtgacgtt

SEQ ID NO: 470 - DNA, 11 bp, Artificial Sequence
  Synthetic Construct
  misc_feature 1,2,3,5,6,7,8,9,10,11: nucleoside-3′-phosphorothioate
  misc_feature 1: 5-bromo-2′-deoxyuridine
  misc_feature 4: nucleoside-3′-phosphate
  misc_feature 4: DBCO-C6-dT
  misc_feature 11: modified by C3 spacer (-(CH2)3-OH) (3′- phosphorothioate)
ucgtcgtgtcg

SEQ ID NO: 471 - DNA, 9 bp, Artificial Sequence
  Synthetic Construct
  misc_feature 1,2,3,5,6,7,8,9: nucleoside-3′-phosphorothioate
  misc_feature 1: 5-bromo-2′-deoxyuridine
  misc_feature 4: nucleoside-3′-phosphate
  misc_feature 4: DBCO-C6-dT
  misc_feature 9: modified by C3 spacer (-(CH2)3-OH) (3′- phosphorothioate)
ucgtcgtgt

SEQ ID NO: 472 - DNA, 9 bp, Artificial Sequence
  Synthetic Construct
  misc_feature (1)..(8): nucleoside-3′-phosphorothioate
  misc_feature 1: 5-bromo-2′-deoxyuridine
  misc_feature 9: nucleoside-3′-phosphate
  misc_feature 9: DBCO-C6-dT
  misc_feature 9: modified by C3 spacer (-(CH2)3-OH) (3′- phosphorothioate)
ucgtcgtgt

SEQ ID NO: 473 - DNA, 7 bp, Artificial Sequence
  Synthetic Construct
  misc_feature 1,2,3,5,6,7: nucleoside-3′-phosphorothioate
  misc_feature 1: 5-bromo-2′-deoxyuridine
  misc_feature 4: nucleoside-3′-phosphate
  misc_feature 4: DBCO-C6-dT
  misc_feature 7: modified by C3 spacer (-(CH2)3-OH) (3′- phosphorothioate)
ucgtcgt

SEQ ID NO: 474 - DNA, 7 bp, Artificial Sequence
  Synthetic Construct
  misc_feature (1)..(6): nucleoside-3′-phosphorothioate
  misc_feature 1: 5-bromo-2′-deoxyuridine
  misc_feature 7: nucleoside-3′-phosphate
  misc_feature 7: DBCO-C6-dT
  misc_feature 7: modified by C3 spacer (-(CH2)3-OH) (3′- phosphorothioate)
ucgtcgt

SEQ ID NO: 475 - DNA, 5 bp, Artificial Sequence
  Synthetic Construct
  misc_feature 1,2,3,5: nucleoside-3′-phosphorothioate
  misc_feature 1: 5-bromo-2′-deoxyuridine
  misc_feature 4: nucleoside-3′-phosphate
  misc_feature 4: DBCO-C6-dT
  misc_feature 5: modified by C3 spacer (-(CH2)3-OH) (3′- phosphorothioate)
ucgtt

SEQ ID NO: 476 - DNA, 5 bp, Artificial Sequence
  Synthetic Construct
  misc_feature (1)..(4): nucleoside-3′-phosphorothioate
  misc_feature 1: 5-bromo-2′-deoxyuridine
  misc_feature 5: nucleoside-3′-phosphate
  misc_feature 5: DBCO-C6-dT
  misc_feature 5: modified by C3 spacer (-(CH2)3-OH) (3′- phosphorothioate)
ucgtt

SEQ ID NO: 477 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: DBCO-C6-dT
  misc_feature 13, 14: 2′-OMe thymidine
  misc_feature 14: modified by C3 spacer (-(CH2)3-OH) (3′- phosphorothioate)
tucgtcgtgacgtt

SEQ ID NO: 478 - DNA, 15 bp, Artificial Sequence
  Synthetic Construct
  misc_feature (1)..(15): nucleoside-3′-phosphorothioate
  misc_feature 1, 2: 2′-OMe thymidine
  misc_feature 3: 5-iodo-2′-deoxyuridine
  misc_feature 6: DBCO-C6-dT
  misc_feature 15: modified by C3 spacer (-(CH2)3-OH) (3′- phosphorothioate)
ttucgtcgtgacgtt

SEQ ID NO: 479 - DNA, 15 bp, Artificial Sequence
  Synthetic Construct
  misc_feature (1)..(15): nucleoside-3′-phosphorothioate
  misc_feature 1, 2, 14, 15: 2′-OMe thymidine
  misc_feature 3: 5-iodo-2′-deoxyuridine
  misc_feature 6: DBCO-C6-dT
  misc_feature 15: modified by C3 spacer (-(CH2)3-OH) (3′- phosphorothioate)
ttucgtcgtgacgtt

SEQ ID NO: 480 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 5: DBCO-C6-dT
  misc_feature 13, 14: 2′-OMOE thymidine
  misc_feature 14: modified by C3 spacer (-(CH2)3-OH) (3′- phosphorothioate)
tucgtcgtgacgtt

SEQ ID NO: 481 - DNA, 15 bp, Artificial Sequence
  Synthetic Construct
  misc_feature (1)..(15): nucleoside-3′-phosphorothioate
  misc_feature 1, 2: 2′-OMOE thymidine
  misc_feature 3: 5-iodo-2′-deoxyuridine
  misc_feature 6: DBCO-C6-dT
  misc_feature 15: modified by C3 spacer (-(CH2)3-OH) (3′- phosphorothioate)
ttucgtcgtgacgtt

SEQ ID NO: 482 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 3: Rp conformation
  misc_feature 5: DBCO-C6-dT
  misc_feature 14: modified by C3 spacer (-(CH2)3-OH) (3′- phosphorothioate)
tucgtcgtgacgtt

SEQ ID NO: 483 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 3: Sp conformation
  misc_feature 5: DBCO-C6-dT
  misc_feature 14: modified by C3 spacer (-(CH2)3-OH) (3′- phosphorothioate)
tucgtcgtgacgtt

SEQ ID NO: 484 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 2: Rp conformation
  misc_feature 5: DBCO-C6-dT
  misc_feature 14: modified by C3 spacer (-(CH2)3-OH) (3′- phosphorothioate)
tucgtcgtgacgtt

SEQ ID NO: 485 - DNA, 14 bp, Artificial Sequence
  Synthetic Construct
  misc_feature (1)..(14): nucleoside-3′-phosphorothioate
  misc_feature 2: 5-iodo-2′-deoxyuridine
  misc_feature 2: Sp conformation
  misc_feature 5: DBCO-C6-dT
  misc_feature 14: modified by C3 spacer (-(CH2)3-OH) (3′- phosphorothioate)
tucgtcgtgacgtt

SEQ ID NO: 486 - DNA, 15 bp, Artificial Sequence
  Synthetic Construct
  misc_feature (1)..(15): nucleoside-3′-phosphorothioate
  misc_feature 1, 2, 14, 15: 2′-OMOE thymidine
  misc_feature 3: 5-iodo-2′-deoxyuridine
  misc_feature 6: DBCO-C6-dT
  misc_feature 15: modified by C3 spacer (-(CH2)3-OH) (3′- phosphorothioate)
ttucgtcgtgacgtt

SEQ ID NO: 487 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ PEG2-NH2 triester (N1)
  misc_feature 13: 2′-OMOE thymidine
  misc_feature 13: modified by C3 spacer (-(CH2)3-OH) (3′- phosphorothioate)
ucgtcgtgtcgtt

SEQ ID NO: 488 - DNA, 13 bp, Artificial Sequence
  Synthetic Construct
  misc_feature (1)..(13): nucleoside-3′-phosphorothioate
  misc_feature 1: 5-bromo-2′-deoxyuridine
  misc_feature 4: nucleoside having a 3′ PEG2-NH2 triester (N1)
  misc_feature 12, 13: 2′-OMOE thymidine
  misc_feature 13: modified by C3 spacer (-(CH2)3-OH) (3′- phosphorothioate)
ucgtcgtgtcgtt

SEQ ID NO: 489 - DNA, 13 bp, Artificial Sequence
  Synthetic Construct
  misc_feature (1)..(12): nucleoside-3′-phosphorothioate
  misc_feature 1: 5-bromo-2′-deoxyuridine
  misc_feature 4: nucleoside having a 3′ PEG2-NH2 triester (N1)
  misc_feature 12: 2′-OMOE thymidine
  misc_feature 13: nucleoside-3′-phosphate
  misc_feature 13: modified by C3 spacer (-(CH2)3-OH) (3′- phosphorothioate)
ucgtcgtgtcgtt

SEQ ID NO: 490 - protein, 120 aa, Artificial Sequence
  Synthetic Construct
EVQLLESGGGVVQPGGSLRLSCAASGFSFSNFAMTWVRQAPGEGLEWVSTIGSGDTYYADSVKGRFTISRDNSKNTLYLQMNSLRAEDTAVYYCAKDSTVSWSGDFFDYWGLGTLVTVSS

SEQ ID NO: 491 - protein, 120 aa, Artificial Sequence
  Synthetic Construct
EVQLLESGGGVVQPGGSLRLSCAASGFSFSNFAVTWVRQAPGKGLEWVSTIGSGDTYYADSVKGRFTISRDNSKNTLYLQMNSLRAEDTAVYYCAKDSTVSWSGDFFDYWGQGTLVTVSS

SEQ ID NO: 492 - protein, 120 aa, Artificial Sequence
  Synthetic Construct
EVQLLESGGGVVQPGGSLRLSCAASGFSFSNFAMTWVRQAPGKGLEWVSTIGSGDTYYADSVKGRFTISRDNSKNTLYLQMNSLRAEDTAVYYCAKDSTVSWSGDFFDYWGQGTLVTVSS

SEQ ID NO: 493 - protein, 120 aa, Artificial Sequence
  Synthetic Construct
EVQLLESGGGVVQPGGSLRLSCAASGFSFSNFALTWVRQAPGKGLEWVSTIGSGDTYYADSVKGRFTISRDNSKNTLYLQMNSLRAEDTAVYYCAKDSTVSWSGDFFDYWGQGTLVTVSS

SEQ ID NO: 494 - protein, 120 aa, Artificial Sequence
  Synthetic Construct
DVQLLESGGGVVQPGGSLRLSCAASGFSFSNFALTWVRQAPGEGLEWVSTIGSGDTYYADSVKGRFTISRDNSKNTLYLQMNSLRAEDTAVYYCAKDSTVSWSGDFFDYWGLGTLVTVSS

SEQ ID NO: 495 - protein, 120 aa, Artificial Sequence
  Synthetic Construct
DVQLLESGGGVVQPGGSLRLSCAASGFSFSNFAVTWVRQAPGEGLEWVSTIGSGDTYYADSVKGRFTISRDNSKNTLYLQMNSLRAEDTAVYYCAKDSTVSWSGDFFDYWGLGTLVTVSS

SEQ ID NO: 496 - protein, 108 aa, Artificial Sequence
  Synthetic Construct
EIVLTQSPATLSVSPGERATFSCRASQNVKNDLAWYQQRPGQAPRLLIYAARIRETGIPERFSGSGSGTEFTLTITSLQSEDFAVYYCQQYYDWPPFTFGGGTKVEIK

SEQ ID NO: 497 - protein, 108 aa, Artificial Sequence
  Synthetic Construct
EIVLTQSPATLSVSPGERATLSCRASQNVKNDLAWYQQKPGQAPRLLIYAARIRETGIPARFSGSGSGTEFTLTISSLQSEDFAVYYCQQYYDWPPFTFGGGTKVEIK

SEQ ID NO: 498 - protein, 10 aa, Artificial Sequence
  Synthetic Construct
GFSFSNFAMT

SEQ ID NO: 499 - protein, 10 aa, Artificial Sequence
  Synthetic Construct
GFSFSNFAVT

SEQ ID NO: 500 - protein, 10 aa, Artificial Sequence
  Synthetic Construct
GFSFSNFALT

SEQ ID NO: 501 - protein, 15 aa, Artificial Sequence
  Synthetic Construct
TIGSGDTYYADSVKG

SEQ ID NO: 502 - protein, 13 aa, Artificial Sequence
  Synthetic Construct
DSTVSWSGDFFDY

SEQ ID NO: 503 - protein, 11 aa, Artificial Sequence
  Synthetic Construct
RASQNVKNDLA

SEQ ID NO: 504 - protein, 7 aa, Artificial Sequence
  Synthetic Construct
AARIRET

SEQ ID NO: 505 - protein, 10 aa, Artificial Sequence
  Synthetic Construct
QQYYDWPPFT

SEQ ID NO: 506 - protein, 120 aa, Artificial Sequence
  Synthetic Construct
DVQLVESGGGVVRPGESLRLSCAASGFSFSIYAMSWVRQAPGEGLEWVSTIGADDTYYADSVKGRFTISRDNSKNTLYLQMNSLRAEDTAVYYCAKDSTVGWSGDFFDYWGLGTLVTVSS

SEQ ID NO: 507 - protein, 120 aa, Artificial Sequence
  Synthetic Construct
EVQLVESGGGVVQPGGSLRLSCAASGFSFSIYAVSWVRQAPGKGLEWVSTIGADDTYYADSVKGRFTISRDNSKNTLYLQMNSLRAEDTAVYYCAKDSTVGWSGDFFDYWGQGTLVTVSS

SEQ ID NO: 508 - protein, 120 aa, Artificial Sequence
  Synthetic Construct
EVQLVESGGGVVQPGGSLRLSCAASGFSFSIYAMSWVRQAPGKGLEWVSTIGADDTYYADSVKGRFTISRDNSKNTLYLQMNSLRAEDTAVYYCAKDSTVGWSGDFFDYWGQGTLVTVSS

SEQ ID NO: 509 - protein, 120 aa, Artificial Sequence
  Synthetic Construct
EVQLVESGGGVVQPGGSLRLSCAASGFSFSIYALSWVRQAPGKGLEWVSTIGADDTYYADSVKGRFTISRDNSKNTLYLQMNSLRAEDTAVYYCAKDSTVGWSGDFFDYWGQGTLVTVSS

SEQ ID NO: 510 - protein, 108 aa, Artificial Sequence
  Synthetic Construct
EIVLTQSPATLSVSPGERVTFSCRASQNVRSDIAWYQQKPGQAPRLLIYAASSRDTGIPDRFSGSGSGTDFTLTISSLQSEDFGVYYCQQYYDWPPFTFGGGTKVEIK

SEQ ID NO: 511 - protein, 108 aa, Artificial Sequence
  Synthetic Construct
EIVLTQSPATLSVSPGERVTLSCRASQNVRSDIAWYQQKPGQAPRLLIYAASSRDTGIPARFSGSGSGTDFTLTISSLQSEDFGVYYCQQYYDWPPFTFGGGTKVEIK

SEQ ID NO: 512 - protein, 10 aa, Artificial Sequence
  Synthetic Construct
GFSFSIYAMS

SEQ ID NO: 513 - protein, 10 aa, Artificial Sequence
  Synthetic Construct
GFSFSIYAVS

SEQ ID NO: 514 - protein, 10 aa, Artificial Sequence
  Synthetic Construct
GFSFSIYALS

SEQ ID NO: 515 - protein, 15 aa, Artificial Sequence
  Synthetic Construct
TIGADDTYYADSVKG

SEQ ID NO: 516 - protein, 13 aa, Artificial Sequence
  Synthetic Construct
DSTVGWSGDFFDY

SEQ ID NO: 517 - protein, 11 aa, Artificial Sequence
  Synthetic Construct
RASQNVRSDIA

SEQ ID NO: 518 - protein, 7 aa, Artificial Sequence
  Synthetic Construct
AASSRDT

SEQ ID NO: 519 - protein, 10 aa, Artificial Sequence
  Synthetic Construct
QQYYDWPPFT

SEQ ID NO: 520 - protein, 118 aa, Artificial Sequence
  Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSS

SEQ ID NO: 521 - protein, 118 aa, Artificial Sequence
  Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSS

SEQ ID NO: 522 - protein, 118 aa, Artificial Sequence
  Synthetic Construct
EVQLVESGGGVVQPGGSLRLSCAASGFTFSSNAVSWVRQAPGKGLEWVAGISAGGSDTYYPASVKGRFTISRDNSKNTLYLQMNSLRAEDTAVYYCARETWNHLFDYWGQGTLVTVSS

SEQ ID NO: 523 - protein, 118 aa, Artificial Sequence
  Synthetic Construct
EVQLVESGGGVVQPGGSLRLSCAASGFTFSSNALSWVRQAPGKGLEWVAGISAGGSDTYYPASVKGRFTISRDNSKNTLYLQMNSLRAEDTAVYYCARETWNHLFDYWGQGTLVTVSS

SEQ ID NO: 524 - protein, 107 aa, Artificial Sequence
  Synthetic Construct
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVL

SEQ ID NO: 525 - protein, 108 aa, Artificial Sequence
  Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVL

SEQ ID NO: 526 - protein, 108 aa, Artificial Sequence
  Synthetic Construct
QSVLTQPPSVSAAPGQKVTISCSGGSYSSYYYAWYQQLPGTAPKTLIYSDDKRPSNIPDRFSGSKSGTSATLGITGLQTGDEADYYCGGYDQSSYTNPFGTGTKVTVL

SEQ ID NO: 527 - protein, 108 aa, Artificial Sequence
  Synthetic Construct
SYELTQPPSVSVSPGQTARITCSGGDYYSTYYAWYQQKPGQAPVTVIHSDDKRPSDIPERFSGSSSGTTVTLTISGVQAEDEADYYCGGYDGRTYINTFGGGTKLTVL

SEQ ID NO: 528 - protein, 108 aa, Artificial Sequence
  Synthetic Construct
QSVLTQPPSVSAAPGQKVTISCSGGDYYSTYYAWYQQLPGTAPKTVIHSDDKRPSDIPDRFSGSKSGTSATLGITGLQTGDEADYYCGGYDGRTYINTFGTGTKVTVL

SEQ ID NO: 529 - protein, 113 aa, Artificial Sequence
  Synthetic Construct
QSALTQPASVSGSPGQSITISCTGTSSDVGSYSSYYYAWYQQHPGKAPKTLIYSDDKRPSNVSNRFSGSKSGNTASLTISGLQAEDEADYYCGGYDQSSYTNPFGGGTKLTVL

SEQ ID NO: 530 - protein, 108 aa, Artificial Sequence
  Synthetic Construct
QSVLTQPPSVSAAPGQKVTISCSGGDYYSYYYGWYQQLPGTAPKTVIYSDDKRPSDIPDRFSGSKSGTSATLGITGLQTGDEADYYCGGYDYSTYANAFGTGTKVTVL

SEQ ID NO: 531 - protein, 108 aa, Artificial Sequence
  Synthetic Construct
SYELTQPPSVSVSPGQTARITCSGGAYSSYYYAWYQQKPGQAPVLVIYSDSKRPSGIPERFSGSSSGTTVTLTISGVQAEDEADYYCGGYDQSSYTNPFGGGTKLTVL

SEQ ID NO: 532 - protein, 108 aa, Artificial Sequence
  Synthetic Construct
SYELTQPPSVSVSPGQTARITCSGGAYSSYYYAWYQQKPGQAPVLVIYSDDKRPSGIPERFSGSSSGTTVTLTISGVQAEDEADYYCGGYDQSSYTNPFGGGTKLTVL

SEQ ID NO: 533 - protein, 10 aa, Artificial Sequence
  Synthetic Construct
GFTFSSNALS

SEQ ID NO: 534 - protein, 10 aa, Artificial Sequence
  Synthetic Construct
GFTFSSNAMS

SEQ ID NO: 535 - protein, 10 aa, Artificial Sequence
  Synthetic Construct
GFTFSSNAVS

SEQ ID NO: 536 - protein, 8 aa, Artificial Sequence
  Synthetic Construct
ISAGGSDT

SEQ ID NO: 537 - protein, 11 aa, Artificial Sequence
  Synthetic Construct
ARETWNHLFDY

SEQ ID NO: 538 - protein, 11 aa, Artificial Sequence
  Synthetic Construct
SGGDYYSYYYG

SEQ ID NO: 539 - protein, 11 aa, Artificial Sequence
  Synthetic Construct
SGGSYSSYYYA

SEQ ID NO: 540 - protein, 11 aa, Artificial Sequence
  Synthetic Construct
SGGDYYSTYYA

SEQ ID NO: 541 - protein, 9 aa, Artificial Sequence
  Synthetic Construct
GSYSSYYYA

SEQ ID NO: 542 - protein, 11 aa, Artificial Sequence
  Synthetic Construct
SGGAYSSYYYA

SEQ ID NO: 543 - protein, 7 aa, Artificial Sequence
  Synthetic Construct
SDDKRPS

SEQ ID NO: 544 - protein, 11 aa, Artificial Sequence
  Synthetic Construct
GGYDYSTYANA

SEQ ID NO: 545 - protein, 11 aa, Artificial Sequence
  Synthetic Construct
GGYDQSSYTNP

SEQ ID NO: 546 - protein, 11 aa, Artificial Sequence
  Synthetic Construct
GGYDGRTYINT

SEQ ID NO: 547 - protein, 119 aa, Artificial Sequence
  Synthetic Construct
DVQLVESGGGVVRPGESLRLSCAASGFTFSSYDMNWVRQAPGEGLEWVSLISGSGEIIYYADSVKGRFTISRDNSKNTLYLQMNSLRAEDTAVYYCAKENNRYRFFDDWGLGTLVTVSS

SEQ ID NO: 548 - protein, 119 aa, Artificial Sequence
  Synthetic Construct
EVQLVESGGGVVQPGRSLRLSCAASGFTFSSYDVNWVRQAPGKGLEWVSLISGSGEIIYYADSVKGRFTISRDNSKNTLYLQMNSLRAEDTAVYYCAKENNRYRFFDDWGQGTLVTVSS

SEQ ID NO: 549 - protein, 119 aa, Artificial Sequence
  Synthetic Construct
EVQLVESGGGVVQPGRSLRLSCAASGFTFSSYDLNWVRQAPGKGLEWVSLISGSGEIIYYADSVKGRFTISRDNSKNTLYLQMNSLRAEDTAVYYCAKENNRYRFFDDWGQGTLVTVSS

SEQ ID NO: 550 - protein, 119 aa, Artificial Sequence
  Synthetic Construct
EVQLVESGGGVVQPGRSLRLSCAASGFTFSSYDMNWVRQAPGKGLEWVSLISGSGEIIYYADSVKGRFTISRDNSKNTLYLQMNSLRAEDTAVYYCAKENNRYRFFDDWGQGTLVTVSS

SEQ ID NO: 551 - protein, 108 aa, Artificial Sequence
  Synthetic Construct
ETVLTQSPGTLTLSPGERATLTCRASQSVYTYLAWYQEKPGQAPRLLIYGASSRATGIPDRFSGSGSGTEFTLTISSLQSEDFAVYYCQQYYDRPPLTFGGGTKVEIK

SEQ ID NO: 552 - protein, 108 aa, Artificial Sequence
  Synthetic Construct
EIVLTQSPGTLSLSPGERATLSCRASQSVYTYLAWYQQKPGQAPRLLIYGASSRATGIPDRFSGSGSGTEFTLTISSLQSEDFAVYYCQQYYDRPPLTFGGGTKVEIK

SEQ ID NO: 553 - protein, 108 aa, Artificial Sequence
  Synthetic Construct
ETVLTQSPGTLSLSPGERATLSCRASQSVYTYLAWYQQKPGQAPRLLIYGASSRATGIPDRFSGSGSGTEFTLTISSLQSEDFAVYYCQQYYDRPPLTFGGGTKVEIK

SEQ ID NO: 554 - protein, 10 aa, Artificial Sequence
  Synthetic Construct
GFTFSSYDMN

SEQ ID NO: 555 - protein, 10 aa, Artificial Sequence
  Synthetic Construct
GFTFSSYDVN

SEQ ID NO: 556 - protein, 10 aa, Artificial Sequence
  Synthetic Construct
GFTFSSYDLN

SEQ ID NO: 557 - protein, 17 aa, Artificial Sequence
  Synthetic Construct
LISGSGEIIYYADSVKG

SEQ ID NO: 558 - protein, 10 aa, Artificial Sequence
  Synthetic Construct
ENNRYRFFDD

SEQ ID NO: 559 - protein, 11 aa, Artificial Sequence
  Synthetic Construct
RASQSVYTYLA

SEQ ID NO: 560 - protein, 7 aa, Artificial Sequence
  Synthetic Construct
GASSRAT

SEQ ID NO: 561 - protein, 10 aa, Artificial Sequence
  Synthetic Construct
QQYYDRPPLT

SEQ ID NO: 562 - protein, 106 aa, Artificial Sequence
  Synthetic Construct
QSALTQPASVSGSPGQSITISCFGSSGNYGLVSWYQQHPGKAPKLMIYYNNKRPSGVSNRFSGSKSGNTASLTISGLQAEDEADYYCGAWETGSATFGGGTKLTVL

SEQ ID NO: 563 - protein, 103 aa, Artificial Sequence
  Synthetic Construct
SYELTQPPSVSVSPGQTASITCFGSSGNYGWYQQKPGQSPVLVIYYNNKRPSGIPERFSGSNSGNTATLTISGTQAMDEADYYCGAWETGSATFGGGTKLTVL

SEQ ID NO: 564 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 566 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 567 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 568 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 569 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 570 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 571 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 572 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 573 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 574 - protein, 326 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 575 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 576 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 577 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 578 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 579 - protein, 107 aa, Artificial Sequence [sequence omitted]

SEQ ID NO: 580 - protein, 106 aa, Artificial Sequence
  Synthetic Construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 581 - protein, 106 aa, Artificial Sequence
  Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 582 - protein, 5 aa, Artificial Sequence
  Synthetic Construct
LLQGG

SEQ ID NO: 583 - protein, 8 aa, Artificial Sequence
  Synthetic Construct
GGGLLQGG

SEQ ID NO: 584 - protein, 114 aa, Artificial Sequence
  Synthetic Construct
DVQLVESGGGVVRPGESLTLSCTASGFTFTSSTMNWVRQAPGEGLDWVSSISTSGVITYYADSVKGRATISRDNSKNTLYLRLFSLRADDTAIYYCATDTFDHWGPGTLVTVSS

SEQ ID NO: 585 - protein, 102 aa, Gallus gallus domesticus
ALTQPASVSANPGETVKITCFGSSGNYGWFQQKSPGSAPVTVIHYNNKRPSDIPSRFSGSKSGSTGTLTITGVRAEDEAVYFCGAWETGSATFGAGTTLTVL

SEQ ID NO: 586 - DNA, 14 bp, Artificial Sequence
  Synthetic Construct
  misc_feature 1: absent or 2′-deoxythymidine
  misc_feature 2: a 2′-deoxyribonucleotide with a modified nucleobase
  misc_feature 3, 6, 11: 2′-deoxycytidine
  misc_feature 4, 7, 9, 12: 2′-deoxyguanosine
  misc_feature 5: 2′-deoxyadenosine or 2′-deoxythymidine, each optionally comprising a 3′-phosphotriester
  misc_feature 8: Can be in repeats of about 1 to about 4
  misc_feature 10: 2′-deoxyadenosine or 2′-deoxythymidine
  misc_feature 13: 2′-deoxythymidine optionally comprising a 3′- phosphotriester
nncgncgtgncgnt